SEQUENCE LISTING (53 sequences)

SEQ ID NO: 1 - RNA, 3995 nt, synthetic construct
  source 1..3995: mol_type = other RNA
gggaaataagagagaaaagaagagtaagaagaaatataagaccccggcgccgccaccatgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtgaagggcttcaactgctacttccctctgcagagctacggcttccagcccaccaacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaccccggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggc

SEQ ID NO: 2 - RNA, 57 nt, synthetic construct
  source 1..57: mol_type = other RNA
gggaaataagagagaaaagaagagtaagaagaaatataagaccccggcgccgccacc

SEQ ID NO: 3 - RNA, 3819 nt, synthetic construct
  source 1..3819: mol_type = other RNA
atgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtgaagggcttcaactgctacttccctctgcagagctacggcttccagcccaccaacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaccccggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacc

SEQ ID NO: 4 - RNA, 119 nt, synthetic construct
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 5 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - RNA, 3995 nt, synthetic construct
  source 1..3995: mol_type = other RNA
gggaaataagagagaaaagaagagtaagaagaaatataagaccccggcgccgccaccatgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaacatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtgaagggcttcaactgctacttccctctgcagagctacggcttccagcccacctacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaccccggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggc

SEQ ID NO: 7 - RNA, 3819 nt, synthetic construct
  source 1..3819: mol_type = other RNA
atgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaacatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtgaagggcttcaactgctacttccctctgcagagctacggcttccagcccacctacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaccccggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacc

SEQ ID NO: 8 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - RNA, 3986 nt, synthetic construct
  source 1..3986: mol_type = other RNA
gggaaataagagagaaaagaagagtaagaagaaatataagaccccggcgccgccaccatgttcgtgttcctggtgctgctgcccctggtgagcagccagtgcgtgaactttaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgccaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcggggcctgccccagggcttctcagccctggagcccctggtggacctgcccatcggcatcaacatcacccggttccagaccctgcacatcagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaacatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtgaagggcttcaactgctacttccctctgcagagctacggcttccagcccacctacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaccccggagggcaaggagcgtggccagccagagcatcatcgcctacaccatgagcctgggcgtggagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggc

SEQ ID NO: 10 - RNA, 3810 nt, synthetic construct
  source 1..3810: mol_type = other RNA
atgttcgtgttcctggtgctgctgcccctggtgagcagccagtgcgtgaactttaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgccaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcggggcctgccccagggcttctcagccctggagcccctggtggacctgcccatcggcatcaacatcacccggttccagaccctgcacatcagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaacatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtgaagggcttcaactgctacttccctctgcagagctacggcttccagcccacctacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaccccggagggcaaggagcgtggccagccagagcatcatcgcctacaccatgagcctgggcgtggagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacc

SEQ ID NO: 11 - protein, 1270 aa, synthetic construct
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - RNA, 3986 nt, synthetic construct
  source 1..3986: mol_type = other RNA
gggaaataagagagaaaagaagagtaagaagaaatataagaccccggcgccgccaccatgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatcagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtggagggcttcaactgctacttccctctgcagagctacggcttccagcccacctacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgacgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcacaccggagggcaaggagcgtggccagccagagcatcatcgcctacaccatgagcctgggcgccgagaacagcgtggcctacagcaacaacagcatcgccatccccatcaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctggcccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccacccacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggc

SEQ ID NO: 13 - RNA, 3810 nt, synthetic construct
  source 1..3810: mol_type = other RNA
atgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatcagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtggagggcttcaactgctacttccctctgcagagctacggcttccagcccacctacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgacgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcacaccggagggcaaggagcgtggccagccagagcatcatcgcctacaccatgagcctgggcgccgagaacagcgtggcctacagcaacaacagcatcgccatccccatcaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctggcccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccacccacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacc

SEQ ID NO: 14 - protein, 1270 aa, synthetic construct
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - RNA, 3977 nt, synthetic construct
  source 1..3977: mol_type = other RNA
gggaaataagagagaaaagaagagtaagaagaaatataagaccccggcgccgccaccatgttcgtgttcctggtgctgctgcccctggtgagcagccagtgcgtgaactttaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatcagcggcaccaacggcaccaagcggttcgccaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcggggcctgccccagggcttctcagccctggagcccctggtggacctgcccatcggcatcaacatcacccggttccagaccctgcacatcagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaacatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtgaagggcttcaactgctacttccctctgcagagctacggcttccagcccacctacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgacgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcacaccggagggcaaggagcgtggccagccagagcatcatcgcctacaccatgagcctgggcgtggagaacagcgtggcctacagcaacaacagcatcgccatccccatcaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctggcccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccacccacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggc

SEQ ID NO: 16 - RNA, 3801 nt, synthetic construct
  source 1..3801: mol_type = other RNA
atgttcgtgttcctggtgctgctgcccctggtgagcagccagtgcgtgaactttaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatcagcggcaccaacggcaccaagcggttcgccaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcggggcctgccccagggcttctcagccctggagcccctggtggacctgcccatcggcatcaacatcacccggttccagaccctgcacatcagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaacatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtgaagggcttcaactgctacttccctctgcagagctacggcttccagcccacctacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgacgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcacaccggagggcaaggagcgtggccagccagagcatcatcgcctacaccatgagcctgggcgtggagaacagcgtggcctacagcaacaacagcatcgccatccccatcaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctggcccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccacccacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacc

SEQ ID NO: 17 - protein, 1267 aa, synthetic construct
MFVFLVLLPLVSSQCVNFTTRTQLPPAYTNSFTRGVYYPDKVFRSSVLHSTQDLFLPFFSNVTWFHAISGTNGTKRFANPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGVENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 18 - RNA, 3995 nt, synthetic construct
  source 1..3995: mol_type = other RNA
gggaaataagagagaaaagaagagtaagaagaaatataagaccccggcgccgccaccatgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtggagggcttcaactgctacttccctctgcagagctacggcttccagcccaccaacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccaggacgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaccccggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggc

SEQ ID NO: 19 - RNA, 3819 nt, synthetic construct
  source 1..3819: mol_type = other RNA
atgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtggagggcttcaactgctacttccctctgcagagctacggcttccagcccaccaacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccaggacgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaccccggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacc

SEQ ID NO: 20 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 21 - RNA, 3995 nt, synthetic construct
  source 1..3995: mol_type = other RNA
gggaaataagagagaaaagaagagtaagaagaaatataagaccccggcgccgccaccatgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcatcgagaagagcaacatcatccggggctggatcttcggcaccaccctggacagcaagacccagagcctgctgatcgtgaataacgccaccaacgtggtgatcaaggtgtgcgagttccagttctgcaacgaccccttcctggacgtgtactaccacaagaacaacaagagctggatgaagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacagataccggctgttccggaagagcaacctgaagcccttcgagcgggacatcagcaccgagatctaccaagccggctccaccccttgcaacggcgtgcagggcttcaactgctacttccctctgcagagctacggcttccagcccaccaacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaagacggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccacgagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggc

SEQ ID NO: 22 - RNA, 3819 nt, synthetic construct
  source 1..3819: mol_type = other RNA
atgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcatcgagaagagcaacatcatccggggctggatcttcggcaccaccctggacagcaagacccagagcctgctgatcgtgaataacgccaccaacgtggtgatcaaggtgtgcgagttccagttctgcaacgaccccttcctggacgtgtactaccacaagaacaacaagagctggatgaagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacagataccggctgttccggaagagcaacctgaagcccttcgagcgggacatcagcaccgagatctaccaagccggctccaccccttgcaacggcgtgcagggcttcaactgctacttccctctgcagagctacggcttccagcccaccaacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaagacggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccacgagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacc

SEQ ID NO: 23 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAHEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 24 - RNA, 3989 nt, synthetic construct
  source 1..3989: mol_type = other RNA
gggaaataagagagaaaagaagagtaagaagaaatataagaccccggcgccgccaccatgttcgtgttcctggtgctgctgcccctggtgagcagccagtgcgtgaacctgagaacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctggacgtgtactaccacaagaacaacaagagctggatggagagcggcgtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacagataccggctgttccggaagagcaacctgaagcccttcgagcgggacatcagcaccgagatctaccaagccggctccaagccttgcaacggcgtggagggcttcaactgctacttccctctgcagagctacggcttccagcccaccaacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaagacggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcagaacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggc

SEQ ID NO: 25 - RNA, 3813 nt, synthetic construct
  source 1..3813: mol_type = other RNA
atgttcgtgttcctggtgctgctgcccctggtgagcagccagtgcgtgaacctgagaacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctggacgtgtactaccacaagaacaacaagagctggatggagagcggcgtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacagataccggctgttccggaagagcaacctgaagcccttcgagcgggacatcagcaccgagatctaccaagccggctccaagccttgcaacggcgtggagggcttcaactgctacttccctctgcagagctacggcttccagcccaccaacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaagacggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcagaacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacc

SEQ ID NO: 26 - protein, 1271 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 27 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 28 - RNA, 3989 nt, synthetic construct
  source 1..3989: mol_type = other RNA
gggaaataagagagaaaagaagagtaagaagaaatataagaccccggcgccgccaccatgttcgtgttcctggtgctgctgcccctggtgagcagccagtgcgtgaacctgagaacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcatcgagaagagcaacatcatccggggctggatcttcggcaccaccctggacagcaagacccagagcctgctgatcgtgaataacgccaccaacgtggtgatcaaggtgtgcgagttccagttctgcaacgaccccttcctggacgtgtactaccacaagaacaacaagagctggatggagagcggcgtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacagataccggctgttccggaagagcaacctgaagcccttcgagcgggacatcagcaccgagatctaccaagccggctccaagccttgcaacggcgtggagggcttcaactgctacttccctctgcagagctacggcttccagcccaccaacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaagacggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcagaacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggc

SEQ ID NO: 29 - RNA, 3813 nt, synthetic construct
  source 1..3813: mol_type = other RNA
atgttcgtgttcctggtgctgctgcccctggtgagcagccagtgcgtgaacctgagaacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcatcgagaagagcaacatcatccggggctggatcttcggcaccaccctggacagcaagacccagagcctgctgatcgtgaataacgccaccaacgtggtgatcaaggtgtgcgagttccagttctgcaacgaccccttcctggacgtgtactaccacaagaacaacaagagctggatggagagcggcgtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacagataccggctgttccggaagagcaacctgaagcccttcgagcgggacatcagcaccgagatctaccaagccggctccaagccttgcaacggcgtggagggcttcaactgctacttccctctgcagagctacggcttccagcccaccaacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaagacggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcagaacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacc

SEQ ID NO: 30 - protein, 1271 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 31 - RNA, 3986 nt, synthetic construct
  source 1..3986: mol_type = other RNA
gggaaataagagagaaaagaagagtaagaagaaatataagaccccggcgccgccaccatgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgtgatcagcggcaccaacggcaccaagcggttcgacaaccccgtgctgcccttcaacgacggcgtgtacttcgccagcatcgagaagagcaacatcatccggggctggatcttcggcaccaccctggacagcaagacccagagcctgctgatcgtgaataacgccaccaacgtggtgatcaaggtgtgcgagttccagttctgcaacgaccccttcctggaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcatcgtgcgggagcccgaggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcgacgaggtgttcaacgccacccggttcgccagcgtgtacgcctggaaccggaagcggatcagcaactgcgtggccgactacagcgtgctgtacaacctggcccccttcttcaccttcaagtgctacggcgtgagccccaccaagctgaacgacctgtgcttcaccaacgtgtacgccgacagcttcgtgatccgtggcgacgaggtgcggcagatcgcacccggccagacaggcaacatcgccgactacaactacaagctgcccgacgacttcaccggctgcgtgatcgcctggaacagcaacaagctcgacagcaaggtgagcggcaactacaactacctgtaccggctgttccggaagagcaacctgaagcccttcgagcgggacatcagcaccgagatctaccaagccggcaacaagccttgcaacggcgtggccggcttcaactgctacttccctctgcggagctacagcttccggcccacctacggcgtgggccaccagccctaccgggtggtggtgctgagcttcgagctgctgcacgccccagccaccgtgtgtggccccaagaagagcaccaacctggtgaagaacaagtgcgtgaacttcaacttcaacggccttaagggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagtacgtgaacaacagctacgagtgcgacatccccatcggcgccggcatctgtgccagctaccagacccagaccaagtcacaccggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaagcgggccctgaccggcatcgccgtggagcaggacaagaacacccaggaggtgttcgcccaggtgaagcagatctacaagacccctcccatcaagtacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaagggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccacaacgcccaggccctgaacaccctggtgaagcagctgagcagcaagttcggcgccatcagcagcgtgctgaacgacatcttcagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggc

SEQ ID NO: 32 - RNA, 3810 nt, synthetic construct
  source 1..3810: mol_type = other RNA
atgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgtgatcagcggcaccaacggcaccaagcggttcgacaaccccgtgctgcccttcaacgacggcgtgtacttcgccagcatcgagaagagcaacatcatccggggctggatcttcggcaccaccctggacagcaagacccagagcctgctgatcgtgaataacgccaccaacgtggtgatcaaggtgtgcgagttccagttctgcaacgaccccttcctggaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcatcgtgcgggagcccgaggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcgacgaggtgttcaacgccacccggttcgccagcgtgtacgcctggaaccggaagcggatcagcaactgcgtggccgactacagcgtgctgtacaacctggcccccttcttcaccttcaagtgctacggcgtgagccccaccaagctgaacgacctgtgcttcaccaacgtgtacgccgacagcttcgtgatccgtggcgacgaggtgcggcagatcgcacccggccagacaggcaacatcgccgactacaactacaagctgcccgacgacttcaccggctgcgtgatcgcctggaacagcaacaagctcgacagcaaggtgagcggcaactacaactacctgtaccggctgttccggaagagcaacctgaagcccttcgagcgggacatcagcaccgagatctaccaagccggcaacaagccttgcaacggcgtggccggcttcaactgctacttccctctgcggagctacagcttccggcccacctacggcgtgggccaccagccctaccgggtggtggtgctgagcttcgagctgctgcacgccccagccaccgtgtgtggccccaagaagagcaccaacctggtgaagaacaagtgcgtgaacttcaacttcaacggccttaagggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagtacgtgaacaacagctacgagtgcgacatccccatcggcgccggcatctgtgccagctaccagacccagaccaagtcacaccggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaagcgggccctgaccggcatcgccgtggagcaggacaagaacacccaggaggtgttcgcccaggtgaagcagatctacaagacccctcccatcaagtacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaagggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccacaacgcccaggccctgaacaccctggtgaagcagctgagcagcaagttcggcgccatcagcagcgtgctgaacgacatcttcagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacc

SEQ ID NO: 33 - protein, 1270 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 34 - RNA, 3986 nt, synthetic construct
  source 1..3986: mol_type = other RNA
gggaaataagagagaaaagaagagtaagaagaaatataagaccccggcgccgccaccatgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgtgatcagcggcaccaacggcaccaagcggttcgacaaccccgtgctgcccttcaacgacggcgtgtacttcgccagcatcgagaagagcaacatcatccggggctggatcttcggcaccaccctggacagcaagacccagagcctgctgatcgtgaataacgccaccaacgtggtgatcaaggtgtgcgagttccagttctgcaacgaccccttcctggaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcatcgtgcgggagcccgaggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcgacgaggtgttcaacgccacccggttcgccagcgtgtacgcctggaaccggaagcggatcagcaactgcgtggccgactacagcgtgctgtacaacctggcccccttcttcaccttcaagtgctacggcgtgagccccaccaagctgaacgacctgtgcttcaccaacgtgtacgccgacagcttcgtgatccgtggcgacgaggtgcggcagatcgcacccggccagacaggcaacatcgccgactacaactacaagctgcccgacgacttcaccggctgcgtgatcgcctggaacagcaacaagctcgacagcaaggtgagcggcaactacaactacctgtaccggctgttccggaagagcaacctgaagcccttcgagcgggacatcagcaccgagatctaccaagccggcaacaagccttgcaacggcgtggccggcttcaactgctacttccctctgcggagctacagcttccggcccacctacggcgtgggccaccagccctaccgggtggtggtgctgagcttcgagctgctgcacgccccagccaccgtgtgtggccccaagaagagcaccaacctggtgaagaacaagtgcgtgaacttcaacttcaacggccttaagggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagtacgtgaacaacagctacgagtgcgacatccccatcggcgccggcatctgtgccagctaccagacccagaccaattcacccggcggcgcaggcagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaagcgggccctgaccggcatcgccgtggagcaggacaagaacacccaggaggtgttcgcccaggtgaagcagatctacaagacccctcccatcaagtacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaagggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccacaacgcccaggccctgaacaccctggtgaagcagctgagcagcaagttcggcgccatcagcagcgtgctgaacgacatcttcagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggc

SEQ ID NO: 35 - RNA, 3810 nt, synthetic construct
  source 1..3810: mol_type = other RNA
atgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgtgatcagcggcaccaacggcaccaagcggttcgacaaccccgtgctgcccttcaacgacggcgtgtacttcgccagcatcgagaagagcaacatcatccggggctggatcttcggcaccaccctggacagcaagacccagagcctgctgatcgtgaataacgccaccaacgtggtgatcaaggtgtgcgagttccagttctgcaacgaccccttcctggaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcatcgtgcgggagcccgaggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcgacgaggtgttcaacgccacccggttcgccagcgtgtacgcctggaaccggaagcggatcagcaactgcgtggccgactacagcgtgctgtacaacctggcccccttcttcaccttcaagtgctacggcgtgagccccaccaagctgaacgacctgtgcttcaccaacgtgtacgccgacagcttcgtgatccgtggcgacgaggtgcggcagatcgcacccggccagacaggcaacatcgccgactacaactacaagctgcccgacgacttcaccggctgcgtgatcgcctggaacagcaacaagctcgacagcaaggtgagcggcaactacaactacctgtaccggctgttccggaagagcaacctgaagcccttcgagcgggacatcagcaccgagatctaccaagccggcaacaagccttgcaacggcgtggccggcttcaactgctacttccctctgcggagctacagcttccggcccacctacggcgtgggccaccagccctaccgggtggtggtgctgagcttcgagctgctgcacgccccagccaccgtgtgtggccccaagaagagcaccaacctggtgaagaacaagtgcgtgaacttcaacttcaacggccttaagggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagtacgtgaacaacagctacgagtgcgacatccccatcggcgccggcatctgtgccagctaccagacccagaccaattcacccggcggcgcaggcagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaagcgggccctgaccggcatcgccgtggagcaggacaagaacacccaggaggtgttcgcccaggtgaagcagatctacaagacccctcccatcaagtacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaagggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccacaacgcccaggccctgaacaccctggtgaagcagctgagcagcaagttcggcgccatcagcagcgtgctgaacgacatcttcagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacc

SEQ ID NO: 36 - protein, 1270 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPGGAGSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 37 - RNA, 3986 nt, synthetic construct
  source 1..3986: mol_type = other RNA
gggaaataagagagaaaagaagagtaagaagaaatataagaccccggcgccgccaccatgttcgtgttcctggtgctgctgcccctggtgagcagccagtgcgtgaacctgaccacccggacccagctgccaccagcctacaccaacagcttcacccggggcgtgtactaccccgacaaggtgttccggagcagcgtgctgcacagcacccaggacctgttcctgcccttcttcagcaacgtgacctggttccacgtgatcagcggcaccaacggcaccaagcggttcgacaaccccgtgctgcccttcaacgacggcgtgtacttcgccagcatcgagaagagcaacatcatccggggctggatcttcggcaccaccctggacagcaagacccagagcctgctgatcgtgaacaacgccaccaacgtggtgatcaaggtgtgcgagttccagttctgcaacgaccccttcctggaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcatcgtgcgggagcccgaggacctgccccagggcttcagcgccctggagcccctggtggacctgcccatcggcatcaacatcacccggttccagaccctgctggccctgcaccggagctacctgaccccaggcgacagcagcagcggctggaccgccggcgccgccgcctactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcgacgaggtgttcaacgccacccggttcgccagcgtgtacgcctggaaccggaagcggatcagcaactgcgtggccgactacagcgtgctgtacaacctggcccctttcttcaccttcaagtgctacggcgtgagccccaccaagctgaacgacctgtgcttcaccaacgtgtacgccgacagcttcgtgatccggggcgacgaggtgcggcagatcgccccaggccagaccggcaacatcgccgactacaactacaagctgcccgacgacttcaccggctgcgtgatcgcctggaacagcaacaagctggacagcaaggtgagcggcaactacaactacctgtaccggctgttccggaagagcaacctgaagcccttcgagcgggacatcagcaccgagatctaccaggccggcaacaagccctgcaacggcgtggccggcttcaactgctacttccctctgcggagctacagcttccggcccacctacggcgtgggccaccagccctaccgggtggtggtgctgagcttcgagctgctgcacgccccagccaccgtgtgcggccccaagaagagcaccaacctggtgaagaacaagtgcgtgaacttcaacttcaacggcctgaagggcaccggcgtgctgaccgagagcaacaagaagttcctgcccttccagcagttcggccgggacatcgccgacaccaccgacgccgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgaccccaacctggcgggtgtacagcaccggcagcaacgtgttccagacccgggccggctgcctgatcggcgccgagtacgtgaacaacagctacgagtgcgacatccccatcggcgccggcatctgcgccagctaccagacccagaccaacagccccggcggcgccggcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagcgggccctgaccggcatcgccgtggagcaggacaagaacacccaggaggtgttcgcccaggtgaagcagatctacaagacccctcccatcaagtacttcggcggcttcaacttcagccagatcctgcccgaccccagcaagcccagcaagcggagcttcatcgaggacctgctgttcaacaaggtgaccctggccgacgccggcttcatcaagcagtacggcgactgcctgggcgacatcgccgcccgggacctgatctgcgcccagaagttcaagggcctgaccgtgctgcctcctctgctgaccgacgagatgatcgcccagtacaccagcgccctgctggccggcaccatcaccagcggctggaccttcggcgccggcgccgccctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcttcagccggctggaccctcccgaggccgaggtgcagatcgaccggctgatcaccggccggctgcagagcctgcagacctacgtgacccagcagctgatccgggccgccgagatccgggccagcgccaacctggccgccaccaagatgagcgagtgcgtgctgggccagagcaagcgggtggacttctgcggcaagggctaccacctgatgagctttccccagagcgcaccccacggcgtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggc

SEQ ID NO: 38 - RNA, 3810 nt, synthetic construct
  source 1..3810: mol_type = other RNA
atgttcgtgttcctggtgctgctgcccctggtgagcagccagtgcgtgaacctgaccacccggacccagctgccaccagcctacaccaacagcttcacccggggcgtgtactaccccgacaaggtgttccggagcagcgtgctgcacagcacccaggacctgttcctgcccttcttcagcaacgtgacctggttccacgtgatcagcggcaccaacggcaccaagcggttcgacaaccccgtgctgcccttcaacgacggcgtgtacttcgccagcatcgagaagagcaacatcatccggggctggatcttcggcaccaccctggacagcaagacccagagcctgctgatcgtgaacaacgccaccaacgtggtgatcaaggtgtgcgagttccagttctgcaacgaccccttcctggaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcatcgtgcgggagcccgaggacctgccccagggcttcagcgccctggagcccctggtggacctgcccatcggcatcaacatcacccggttccagaccctgctggccctgcaccggagctacctgaccccaggcgacagcagcagcggctggaccgccggcgccgccgcctactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcgacgaggtgttcaacgccacccggttcgccagcgtgtacgcctggaaccggaagcggatcagcaactgcgtggccgactacagcgtgctgtacaacctggcccctttcttcaccttcaagtgctacggcgtgagccccaccaagctgaacgacctgtgcttcaccaacgtgtacgccgacagcttcgtgatccggggcgacgaggtgcggcagatcgccccaggccagaccggcaacatcgccgactacaactacaagctgcccgacgacttcaccggctgcgtgatcgcctggaacagcaacaagctggacagcaaggtgagcggcaactacaactacctgtaccggctgttccggaagagcaacctgaagcccttcgagcgggacatcagcaccgagatctaccaggccggcaacaagccctgcaacggcgtggccggcttcaactgctacttccctctgcggagctacagcttccggcccacctacggcgtgggccaccagccctaccgggtggtggtgctgagcttcgagctgctgcacgccccagccaccgtgtgcggccccaagaagagcaccaacctggtgaagaacaagtgcgtgaacttcaacttcaacggcctgaagggcaccggcgtgctgaccgagagcaacaagaagttcctgcccttccagcagttcggccgggacatcgccgacaccaccgacgccgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgaccccaacctggcgggtgtacagcaccggcagcaacgtgttccagacccgggccggctgcctgatcggcgccgagtacgtgaacaacagctacgagtgcgacatccccatcggcgccggcatctgcgccagctaccagacccagaccaacagccccggcggcgccggcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagcgggccctgaccggcatcgccgtggagcaggacaagaacacccaggaggtgttcgcccaggtgaagcagatctacaagacccctcccatcaagtacttcggcggcttcaacttcagccagatcctgcccgaccccagcaagcccagcaagcggagcttcatcgaggacctgctgttcaacaaggtgaccctggccgacgccggcttcatcaagcagtacggcgactgcctgggcgacatcgccgcccgggacctgatctgcgcccagaagttcaagggcctgaccgtgctgcctcctctgctgaccgacgagatgatcgcccagtacaccagcgccctgctggccggcaccatcaccagcggctggaccttcggcgccggcgccgccctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcttcagccggctggaccctcccgaggccgaggtgcagatcgaccggctgatcaccggccggctgcagagcctgcagacctacgtgacccagcagctgatccgggccgccgagatccgggccagcgccaacctggccgccaccaagatgagcgagtgcgtgctgggccagagcaagcgggtggacttctgcggcaagggctaccacctgatgagctttccccagagcgcaccccacggcgtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacc

SEQ ID NO: 39 - protein, 1270 aa, synthetic construct; identical to SEQ ID NO: 36

SEQ ID NO: 40 - RNA, 3986 nt, synthetic construct
  source 1..3986: mol_type = other RNA
gggaaataagagagaaaagaagagtaagaagaaatataagaccccggcgccgccaccatgttcgtgttcctggtgctgctgcccctggtgagcagccagtgcgtgaacctgaccacccggacccagctgccaccagcctacaccaacagcttcacccggggcgtgtactaccccgacaaggtgttccggagcagcgtgctgcacagcacccaggacctgttcctgcccttcttcagcaacgtgacctggttccacgtgatcagcggcaccaacggcaccaagcggttcgacaaccccgtgctgcccttcaacgacggcgtgtacttcgccagcatcgagaagagcaacatcatccggggctggatcttcggcaccaccctggacagcaagacccagagcctgctgatcgtgaacaacgccaccaacgtggtgatcaaggtgtgcgagttccagttctgcaacgaccccttcctggaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcatcgtgcgggagcccgaggacctgccccagggcttcagcgccctggagcccctggtggacctgcccatcggcatcaacatcacccggttccagaccctgctggccctgcaccggagctacctgaccccaggcgacagcagcagcggctggaccgccggcgccgccgcctactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcgacgaggtgttcaacgccacccggttcgccagcgtgtacgcctggaaccggaagcggatcagcaactgcgtggccgactacagcgtgctgtacaacctggcccctttcttcaccttcaagtgctacggcgtgagccccaccaagctgaacgacctgtgcttcaccaacgtgtacgccgacagcttcgtgatccggggcgacgaggtgcggcagatcgccccaggccagaccggcaacatcgccgactacaactacaagctgcccgacgacttcaccggctgcgtgatcgcctggaacagcaacaagctggacagcaaggtgagcggcaactacaactacctgtaccggctgttccggaagagcaacctgaagcccttcgagcgggacatcagcaccgagatctaccaggccggcaacaagccctgcaacggcgtggccggcttcaactgctacttccctctgcggagctacagcttccggcccacctacggcgtgggccaccagccctaccgggtggtggtgctgagcttcgagctgctgcacgccccagccaccgtgtgcggccccaagaagagcaccaacctggtgaagaacaagtgcgtgaacttcaacttcaacggcctgaagggcaccggcgtgctgaccgagagcaacaagaagttcctgcccttccagcagttcggccgggacatcgccgacaccaccgacgccgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgaccccaacctggcgggtgtacagcaccggcagcaacgtgttccagacccgggccggctgcctgatcggcgccgagtacgtgaacaacagctacgagtgcgacatccccatcggcgccggcatctgcgccagctaccagacccagaccaagagccaccggcgggcccggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagcgggccctgaccggcatcgccgtggagcaggacaagaacacccaggaggtgttcgcccaggtgaagcagatctacaagacccctcccatcaagtacttcggcggcttcaacttcagccagatcctgcccgaccccagcaagcccagcaagcggagcttcatcgaggacctgctgttcaacaaggtgaccctggccgacgccggcttcatcaagcagtacggcgactgcctgggcgacatcgccgcccgggacctgatctgcgcccagaagttcaagggcctgaccgtgctgcctcctctgctgaccgacgagatgatcgcccagtacaccagcgccctgctggccggcaccatcaccagcggctggaccttcggcgccggcgccgccctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcttcagccggctggaccctcccgaggccgaggtgcagatcgaccggctgatcaccggccggctgcagagcctgcagacctacgtgacccagcagctgatccgggccgccgagatccgggccagcgccaacctggccgccaccaagatgagcgagtgcgtgctgggccagagcaagcgggtggacttctgcggcaagggctaccacctgatgagctttccccagagcgcaccccacggcgtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggc

SEQ ID NO: 41 - RNA, 3810 nt, synthetic construct
  source 1..3810: mol_type = other RNA
atgttcgtgttcctggtgctgctgcccctggtgagcagccagtgcgtgaacctgaccacccggacccagctgccaccagcctacaccaacagcttcacccggggcgtgtactaccccgacaaggtgttccggagcagcgtgctgcacagcacccaggacctgttcctgcccttcttcagcaacgtgacctggttccacgtgatcagcggcaccaacggcaccaagcggttcgacaaccccgtgctgcccttcaacgacggcgtgtacttcgccagcatcgagaagagcaacatcatccggggctggatcttcggcaccaccctggacagcaagacccagagcctgctgatcgtgaacaacgccaccaacgtggtgatcaaggtgtgcgagttccagttctgcaacgaccccttcctggaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcatcgtgcgggagcccgaggacctgccccagggcttcagcgccctggagcccctggtggacctgcccatcggcatcaacatcacccggttccagaccctgctggccctgcaccggagctacctgaccccaggcgacagcagcagcggctggaccgccggcgccgccgcctactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcgacgaggtgttcaacgccacccggttcgccagcgtgtacgcctggaaccggaagcggatcagcaactgcgtggccgactacagcgtgctgtacaacctggcccctttcttcaccttcaagtgctacggcgtgagccccaccaagctgaacgacctgtgcttcaccaacgtgtacgccgacagcttcgtgatccggggcgacgaggtgcggcagatcgccccaggccagaccggcaacatcgccgactacaactacaagctgcccgacgacttcaccggctgcgtgatcgcctggaacagcaacaagctggacagcaaggtgagcggcaactacaactacctgtaccggctgttccggaagagcaacctgaagcccttcgagcgggacatcagcaccgagatctaccaggccggcaacaagccctgcaacggcgtggccggcttcaactgctacttccctctgcggagctacagcttccggcccacctacggcgtgggccaccagccctaccgggtggtggtgctgagcttcgagctgctgcacgccccagccaccgtgtgcggccccaagaagagcaccaacctggtgaagaacaagtgcgtgaacttcaacttcaacggcctgaagggcaccggcgtgctgaccgagagcaacaagaagttcctgcccttccagcagttcggccgggacatcgccgacaccaccgacgccgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgaccccaacctggcgggtgtacagcaccggcagcaacgtgttccagacccgggccggctgcctgatcggcgccgagtacgtgaacaacagctacgagtgcgacatccccatcggcgccggcatctgcgccagctaccagacccagaccaagagccaccggcgggcccggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagcgggccctgaccggcatcgccgtggagcaggacaagaacacccaggaggtgttcgcccaggtgaagcagatctacaagacccctcccatcaagtacttcggcggcttcaacttcagccagatcctgcccgaccccagcaagcccagcaagcggagcttcatcgaggacctgctgttcaacaaggtgaccctggccgacgccggcttcatcaagcagtacggcgactgcctgggcgacatcgccgcccgggacctgatctgcgcccagaagttcaagggcctgaccgtgctgcctcctctgctgaccgacgagatgatcgcccagtacaccagcgccctgctggccggcaccatcaccagcggctggaccttcggcgccggcgccgccctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcttcagccggctggaccctcccgaggccgaggtgcagatcgaccggctgatcaccggccggctgcagagcctgcagacctacgtgacccagcagctgatccgggccgccgagatccgggccagcgccaacctggccgccaccaagatgagcgagtgcgtgctgggccagagcaagcgggtggacttctgcggcaagggctaccacctgatgagctttccccagagcgcaccccacggcgtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacc

SEQ ID NO: 42 - protein, 1270 aa, synthetic construct; identical to SEQ ID NO: 33

SEQ ID NO: 43 - RNA, 3989 nt, synthetic construct
  source 1..3989: mol_type = other RNA
gggaaataagagagaaaagaagagtaagaagaaatataagaccccggcgccgccaccatgttcgtgttcctggtgctgctgcccctggtgagcagccagtgcgtgaacctgaccacccggacccagctgccaccagcctacaccaacagcttcacccggggcgtgtactaccccgacaaggtgttccggagcagcgtgctgcacagcacccaggacctgttcctgcccttcttcagcaacgtgacctggttccacgtgatcagcggcaccaacggcaccaagcggttcgacaaccccgtgctgcccttcaacgacggcgtgtacttcgccagcatcgagaagagcaacatcatccggggctggatcttcggcaccaccctggacagcaagacccagagcctgctgatcgtgaacaacgccaccaacgtggtgatcaaggtgtgcgagttccagttctgcaacgaccccttcctggaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcatcgtgcgggagcccgaggacctgccccagggcttcagcgccctggagcccctggtggacctgcccatcggcatcaacatcacccggttccagaccctgctggccctgcaccggagctacctgaccccaggcgacagcagcagcggctggaccgccggcgccgccgcctactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcgacgaggtgttcaacgccacccggttcgccagcgtgtacgcctggaaccggaagcggatcagcaactgcgtggccgactacagcgtgctgtacaacctggcccctttcttcaccttcaagtgctacggcgtgagccccaccaagctgaacgacctgtgcttcaccaacgtgtacgccgacagcttcgtgatccggggcgacgaggtgcggcagatcgccccaggccagaccggcaacatcgccgactacaactacaagctgcccgacgacttcaccggctgcgtgatcgcctggaacagcaacaagctggacagcaaggtgagcggcaactacaactacctgtaccggctgttccggaagagcaacctgaagcccttcgagcgggacatcagcaccgagatctaccaggccggcaacaagccctgcaacggcgtggccggcttcaactgctacttccctctgcggagctacagcttccggcccacctacggcgtgggccaccagccctaccgggtggtggtgctgagcttcgagctgctgcacgccccagccaccgtgtgcggccccaagaagagcaccaacctggtgaagaacaagtgcgtgaacttcaacttcaacggcctgaagggcaccggcgtgctgaccgagagcaacaagaagttcctgcccttccagcagttcggccgggacatcgccgacaccaccgacgccgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgaccccaacctggcgggtgtacagcaccggcagcaacgtgttccagacccgggccggctgcctgatcggcgccgagtacgtgaacaacagctacgagtgcgacatccccatcggcgccggcatctgcgccagctaccagacccagaccaagagccaccggcgggcccggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagcgggccctgaccggcatcgccgtggagcaggacaagaacacccaggaggtgttcgcccaggtgaagcagatctacaagacccctcccatcaagtacttcggcggcttcaacttcagccagatcctgcccgaccccagcaagcccagcaagcggagcttcatcgaggacctgctgttcaacaaggtgaccctggccgacgccggcttcatcaagcagtacggcgactgcctgggcgacatcgccgcccgggacctgatctgcgcccagaagttcaagggcctgaccgtgctgcctcctctgctgaccgacgagatgatcgcccagtacaccagcgccctgctggccggcaccatcaccagcggctggaccttcggcgccggcgccgccctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcttcagccggctggaccctcccgaggccgaggtgcagatcgaccggctgatcaccggccggctgcagagcctgcagacctacgtgacccagcagctgatccgggccgccgagatccgggccagcgccaacctggccgccaccaagatgagcgagtgcgtgctgggccagagcaagcgggtggacttctgcggcaagggctaccacctgatgagctttccccagagcgcaccccacggcgtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcaggatagatagcgaagtggtctttgaataaagtctgagtgggcggc

SEQ ID NO: 44 - RNA, 122 nt, synthetic construct
  source 1..122: mol_type = other RNA
tgataataggctggagcctcggtggcctagcttcttgccccttgggcctccccccagcccctcctccccttcctgcaggatagatagcgaagtggtctttgaataaagtctgagtgggcggc

SEQ ID NO: 45 - RNA, 3989 nt, synthetic construct
  source 1..3989: mol_type = other RNA
gggaaataagagagaaaagaagagtaagaagaaatataagaccccggcgccgccaccatgttcgtgttcctggtgctgctgcccctggtgagcagccagtgcgtgaacctgaccacccggacccagctgccaccagcctacaccaacagcttcacccggggcgtgtactaccccgacaaggtgttccggagcagcgtgctgcacagcacccaggacctgttcctgcccttcttcagcaacgtgacctggttccacgtgatcagcggcaccaacggcaccaagcggttcgacaaccccgtgctgcccttcaacgacggcgtgtacttcgccagcatcgagaagagcaacatcatccggggctggatcttcggcaccaccctggacagcaagacccagagcctgctgatcgtgaacaacgccaccaacgtggtgatcaaggtgtgcgagttccagttctgcaacgaccccttcctggaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcatcgtgcgggagcccgaggacctgccccagggcttcagcgccctggagcccctggtggacctgcccatcggcatcaacatcacccggttccagaccctgctggccctgcaccggagctacctgaccccaggcgacagcagcagcggctggaccgccggcgccgccgcctactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcgacgaggtgttcaacgccacccggttcgccagcgtgtacgcctggaaccggaagcggatcagcaactgcgtggccgactacagcgtgctgtacaacctggcccctttcttcaccttcaagtgctacggcgtgagccccaccaagctgaacgacctgtgcttcaccaacgtgtacgccgacagcttcgtgatccggggcgacgaggtgcggcagatcgccccaggccagaccggcaacatcgccgactacaactacaagctgcccgacgacttcaccggctgcgtgatcgcctggaacagcaacaagctggacagcaaggtgagcggcaactacaactacctgtaccggctgttccggaagagcaacctgaagcccttcgagcgggacatcagcaccgagatctaccaggccggcaacaagccctgcaacggcgtggccggcttcaactgctacttccctctgcggagctacagcttccggcccacctacggcgtgggccaccagccctaccgggtggtggtgctgagcttcgagctgctgcacgccccagccaccgtgtgcggccccaagaagagcaccaacctggtgaagaacaagtgcgtgaacttcaacttcaacggcctgaagggcaccggcgtgctgaccgagagcaacaagaagttcctgcccttccagcagttcggccgggacatcgccgacaccaccgacgccgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgaccccaacctggcgggtgtacagcaccggcagcaacgtgttccagacccgggccggctgcctgatcggcgccgagtacgtgaacaacagctacgagtgcgacatccccatcggcgccggcatctgcgccagctaccagacccagaccaagagccaccggcgggcccggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagcgggccctgaccggcatcgccgtggagcaggacaagaacacccaggaggtgttcgcccaggtgaagcagatctacaagacccctcccatcaagtacttcggcggcttcaacttcagccagatcctgcccgaccccagcaagcccagcaagcggagcttcatcgaggacctgctgttcaacaaggtgaccctggccgacgccggcttcatcaagcagtacggcgactgcctgggcgacatcgccgcccgggacctgatctgcgcccagaagttcaagggcctgaccgtgctgcctcctctgctgaccgacgagatgatcgcccagtacaccagcgccctgctggccggcaccatcaccagcggctggaccttcggcgccggcgccgccctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcttcagccggctggaccctcccgaggccgaggtgcagatcgaccggctgatcaccggccggctgcagagcctgcagacctacgtgacccagcagctgatccgggccgccgagatccgggccagcgccaacctggccgccaccaagatgagcgagtgcgtgctgggccagagcaagcgggtggacttctgcggcaagggctaccacctgatgagctttccccagagcgcaccccacggcgtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcaggccacatagcgaagtggtctttgaataaagtctgagtgggcggc

SEQ ID NO: 46 - RNA, 122 nt, synthetic construct
  source 1..122: mol_type = other RNA
tgataataggctggagcctcggtggcctagcttcttgccccttgggcctccccccagcccctcctccccttcctgcaggccacatagcgaagtggtctttgaataaagtctgagtgggcggc

SEQ ID NO: 47 - protein, 10 aa, synthetic construct
TQTNSPRRAR

SEQ ID NO: 48 - protein, 5 aa, synthetic construct
KPSKR

SEQ ID NO: 49 - protein, 4 aa, synthetic construct
GGGS

SEQ ID NO: 50 - RNA, 47 nt, synthetic construct
  source 1..47: mol_type = other RNA
gggaaataagagagaaaagaagagtaagaagaaatataagagccacc

SEQ ID NO: 51 - RNA, 119 nt, synthetic construct
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc

SEQ ID NO: 52 - skipped

SEQ ID NO: 53 - RNA, 11 nt, synthetic construct
  source 1..11: mol_type = other RNA
gggatcctacc

(19) United States
(12) Patent Application Publication
(10) Pub. No.: US 2025/0367278 A1
(43) Pub. Date: Dec. 4, 2025

(54) VARIANT STRAIN-BASED CORONAVIRUS VACCINES

(71) Applicant: ModernaTX, Inc., Cambridge, MA (US)

(72) Inventors: Andrea Carfi, Cambridge, MA (US); Guillaume Stewart-Jones, Cambridge, MA (US); Hamilton Bennett, Cambridge, MA (US); Kai Wu, Cambridge, MA (US); Darin Edwards, Cambridge, MA (US); Gwo-Yu Chuang, Cambridge, MA (US); David Reid, Cambridge, MA (US)

(21) Appl. No.: 19/294,594

(22) Filed: Aug. 8, 2025

Related U.S. Application Data
(63) Continuation of application No. 18/272,496, filed on Jul. 14, 2023.
(60) Provisional application No. 63/138,228, filed on Jan. 15, 2021.
(60) Provisional application No. 63/140,920, filed on Jan. 24, 2021.
(60) Provisional application No. 63/161,433, filed on Mar. 15, 2021.
(60) Provisional application No. 63/173,979, filed on Apr. 12, 2021.
(60) Provisional application No. 63/193,547, filed on May 26, 2021.
(60) Provisional application No. 63/222,925, filed on Jul. 16, 2021.
(60) Provisional application No. 63/241,963, filed on Sep. 8, 2021.
(60) Provisional application No. 63/283,905, filed on Nov. 29, 2021.
(60) Provisional application No. 63/284,570, filed on Nov. 30, 2021.

(51) Int. Cl. A61K 39/215; A61K 39/00; A61P 31/14; C07K 14/005

(52) U.S. Cl. CPC A61K 39/215; A61P 31/14; C07K 14/005; A61K 2039/53; A61K 2039/545; A61K 2039/70; C12N 2770/20034

(57) ABSTRACT

The disclosure provides coronavirus mRNA vaccines, including vaccines directed against spike proteins of one or more variant strains of SARS-CoV-2, as well as methods of using the vaccines.

### RELATED APPLICATIONS

This application is a continuation of U.S. patent application Ser. No. 18/272,496, filed Jul. 14, 2023, which is a 35 U.S.C. § 371 of International Application No. PCT/US2022/012614, filed Jan. 14, 2022, which claims priority under 35 U.S.C. § 119 (e) to U.S. Provisional Patent Application No. 63/138,228, filed Jan. 15, 2021, U.S. Provisional Patent Application No. 63/140,920, filed Jan. 24, 2021, U.S. Provisional Patent Application No. 63/161,433, filed Mar. 15, 2021, U.S. Provisional Patent Application No. 63/173,979, filed Apr. 12, 2021, U.S. Provisional Patent Application No. 63/193,547, filed May 26, 2021, U.S. Provisional Patent Application No. 63/222,925, filed Jul. 16, 2021, U.S. Provisional Patent Application No. 63/241,963, filed Sep. 8, 2021, U.S. Provisional Patent Application No. 63/283,905, filed Nov. 29, 2021, and U.S. Provisional Patent Application No. 63/284,570, filed Nov. 30, 2021, each of which are hereby incorporated by reference in their entireties.

### SEQUENCE LISTING

This application contains a Sequence Listing that has been submitted electronically as an XML file named 45817-0208002_SL_ST26.xml. The XML file, created on Jul. 31, 2025, is 173,789 bytes in size. The material in the XML file is hereby incorporated by reference in its entirety.

### BACKGROUND

Human coronaviruses are highly contagious enveloped, positive sense single-stranded RNA viruses of the Coronaviridae family. Two sub-families of Coronaviridae are known to cause human disease. The most important being the β-coronaviruses (betacoronaviruses). The β-coronaviruses are common etiological agents of mild to moderate upper respiratory tract infections. Outbreaks of novel coronavirus infections such as the infections caused by a coronavirus initially identified from the Chinese city of Wuhan in December 2019; however, have been associated with a high mortality rate death toll. This recently identified coronavirus, referred to as Severe Acute Respiratory Syndrome Coronavirus 2 (SARS-CoV-2) (formerly referred to as a “2019 novel coronavirus,” or a “2019-nCOV”) has rapidly infected hundreds of thousands of people. The pandemic disease that the SARS-CoV-2 virus causes has been named by World Health Organization (WHO) as COVID-19 (Coronavirus Disease 2019). The first genome sequence of a SARS-CoV-2 isolate (Wuhan-Hu-1; USA-WA1/2020 isolate) was released by investigators from the Chinese CDC in Beijing on Jan. 10, 2020 at Virological, a UK-based discussion forum for analysis and interpretation of virus molecular evolution and epidemiology. The sequence was then deposited in GenBank on Jan. 12, 2020, having Genbank Accession number MN908947.1. Subsequently, a number of SARS-CoV-2 strain variants have been identified, some of which are more infectious than the SARS-CoV-2 isolate.

As of the time of worldwide emergency use authorization of the authorized SARS-CoV-2 nucleic acid-based vaccines, there is not yet a strategy for combatting the recently-discovered and later-emerging SARS-CoV-2 variants of concern (VOC). The continuing health problems and mortality associated with coronavirus infections, particularly the SARS-CoV-2 pandemic, are of tremendous concern internationally. The public health crisis caused by SARS-CoV-2 and its variants reinforces the importance of rapidly developing effective and safe vaccine candidates against these viruses.

The emergence of SARS-CoV-2 variants with substitutions in the receptor binding domain (RBD) and N-terminal domain (NTD) of the viral S protein has raised concerns among scientists and health officials. The entry of coronavirus into host cells is mediated by interaction between the RBD of the viral S protein and host angiotensin-converting enzyme 2 (ACE2). Vaccine development has focused on inducing antibody responses against this region of SARS-CoV-2 S protein. More recently, a neutralization “supersite” has also been identified in the NTD. A significant decrease in vaccine efficacy has been correlated with amino acid substitutions in the RBD (eg, K417N, E484K, and N501Y) and NTD (eg, L18F, D80A, D215G, and A242-244) of the S protein. Some of the most recently circulating isolates containing these substitutions from the United Kingdom (B.1.1.7, Alpha), Republic of South Africa (B.1.351, Beta), Brazil (P.1 lineage, Gamma), New York (B.1.526, Iota), and California (B.1.427/B.1.429 or CAL.20C lineage, Epsilon), have shown a reduction in neutralization from convalescent serum in pseudovirus neutralization (PsVN) assays and resistance to certain monoclonal antibodies. In particular, mutations in the NTD subdomain, and specifically the neutralization supersite, are most extensive in the B.1.351 lineage virus. See McCallum, M. et al. N-terminal domain antigenic mapping reveals a site of vulnerability for SARS-CoV-2. Cell, doi: 10.1016/j.cell.2021.03.028 (2021).

Using 2 orthogonal vesicular stomatitis virus (VSV) and lentivirus PsVN assays expressing S variants of 20E (EU1), 20A.EU2, D614G-N439K, mink cluster 5, B.1.1.7, P.1, B.1.427/B.1.429, B.1.1.7+E484K, and B.1.351, the assessment of the neutralizing capacity of sera from Phase 1 participants and non-human primates (NHPs) that received 2 doses of mRNA-1273 was reported. See Wu, K. et al. Serum Neutralizing Activity Elicited by mRNA-1273 Vaccine. N Engl J Med, doi: 10.1056/NEJMc2102179 (2021). Subsequent studies demonstrated reduced neutralization titers against the full B.1.351 variant following mRNA-1273 vaccination, although levels are still significant and expected to be protective. Despite this prediction of continued efficacy of mRNA-1273 against this key variant of concern, the duration of vaccine mediated protection is still unknown.

There remains a need for development and evaluation of further COVID-19 vaccines against SARS-CoV-2 variants encoding the prefusion stabilized S protein of SARS-CoV-2 that incorporates key mutations present in variants, including L18F, D80A, D215G, L242-244del, R246I, K417N, E484K, N501Y, D614G, A701V, A67V, Δ69-70, T95I, G142D/Δ143-145, Δ211/L212I, ins214EPE, G339D, S371L, S373P, S375F, N440K, G446S, S477N, T478K, E484A, Q493K, G496S, Q498R, Y505H, T547K, H655Y, N679K, P681H, N764K, D796Y, N856K, Q954H, N969K, L981F, and any combination thereof. Additional vaccines are necessary to expand the breadth of coverage to multiple circulating variants as well as the ancestral wild-type virus that is still circulating globally.

### SUMMARY

A SARS-CoV-2 vaccine, mRNA-1273 (developed by Moderna Therapeutics), has been shown to elicit high viral neutralizing titers in Phase 1 trial human participants (Jackson et al, 2020; Anderson et al, 2020) and is highly efficacious in prevention of symptomatic COVID-19 disease and severe disease (Baden et al., 2020). However, the recent emergence of SARS-CoV-2 variants in the United Kingdom (B.1.1.7 lineage; alpha) and in South Africa (B.1.351 lineage; beta) have raised concerns due to their increased rates of transmission as well as their potential to circumvent immunity elicited by natural infection or vaccination (Volz et al., 2021; Tegally et al., 2020; Wibmer et al., 2021; Wang et al., 2021; Collier et al., 2021).

First detected in September 2020 in South England, the SARS-CoV-2 B.1.1.7 variant (alpha variant) has spread at a rapid rate and is associated with increased transmission and higher viral burden (Rambaut et al., 2020). This variant has seventeen mutations in the viral genome. Among them, eight mutations are located in the spike(S) protein, including 69-70 del, Y144 del, N501Y, A570D, P681H, T716I, S982A and D1118H. Two key features of this variant, the 69-70 deletion and the N501Y mutation in S protein, have generated concern among scientists and policy makers in the UK based on increased transmission and potentially increased mortality, resulting in further shutdowns. The 69-70 deletion is associated with reduced sensitivity to neutralization by SARS-CoV-2 human convalescent serum samples (Kemp et al, 2021). N501 is one of the six key amino acids interacting with ACE-2 receptor (Starr et al. 2020), and the tyrosine substitution has been shown to have increased binding affinity to the ACE-2 receptor (Chan et al., 2020).

The B.1.351 variant (beta variant) emerged in South Africa over the past few months, and, similar to the B.1.1.7 variant, increased rates of transmission and higher viral burden after infection have been reported (Tegally et al., 2020). The mutations located in the S protein are more extensive than the B.1.1.7 variant with changes of L18F, D80A, D215G, L242-244del, R246I, K417N, E484K, N501Y, D614G, and A701V, with three of these mutations located in the RBD (K417N, E484K, N501Y). B.1.351 shares key mutations in the RBD with a reported variant in Brazil (Tegally et al., 2020; Naveca et al., 2021). As the RBD is the predominant target for neutralizing antibodies, these mutations could impact the effectiveness of monoclonal antibodies already approved and in advanced development as well as of polyclonal antibody elicited by infection or vaccination in neutralizing the virus (Greaney et al., 2021, Wibmer et al, 2021).

Recent data have suggested that the key mutation present in the B.1.351 variant, E484K, confers resistance to SARS-CoV-2 neutralizing antibodies, potentially limiting the therapeutic effectiveness of monoclonal antibody therapies (Wang et al., 2021; Greaney et al., 2020; Weisblum et al., 2020; Liu et al., 2020; Wibmer et al., 2021). Moreover, the E484K mutation was shown to reduce neutralization against a panel of convalescent sera (Weisblum et al., 2020; Liu et al., 2020; Wibmer et al., 2021). In terms of vaccination, it is clear that mRNA-1273 induces significantly higher neutralizing titers than convalescent sera against the USA-WA1/2020 isolate (Jackson et al, 2020). A recent study using a recombinant VSV PsVN assay showed that sera of mRNA-1273 vaccinated participants had reduced neutralizing titers against E484K or K417N/E484K/N50Y combination (Wang et al, 2021), however there has been no assessment of sera from mRNA-1273 clinical trial participants against the full constellation of S mutations found in the B.1.1.7 or B.1.351 variants.

Neutralization of sera from mRNA-1273 vaccinated Phase 1 clinical trial participants against recombinant VSV-based SARS-CoV-2 PsVN assay with S protein from the original USA-WA1/2020 isolate, D614G variant, the B.1.1.7 and B.1.351 variants, and variants that have previously emerged (20E, 20A.EU2, D614G-N439K, and mink cluster 5 variant) was examined (data discussed in the Examples). The effect of both single mutations and combinations of mutations present in the RBD region of the S protein was assessed. In addition, orthogonal assessments in VSV and pseudotyped lentiviral neutralization assays were performed on sera from NHPs that received the mRNA-1273 vaccine at two different dose levels, as this has been a useful pre-clinical model for vaccine induced immunogenicity and protection. Using both of these assays provided confirmatory data on pseudovirus neutralization. Overall, this comprehensive pseudovirus neutralization analysis in humans and non-human primates that received mRNA-1273 provides a critical demonstration necessary to elucidate how vaccines may be impacted by SARS-CoV-2 variants.

The invention pertains, inter alia, to vaccines comprising a nucleic acid encoding a SARS-CoV-2 antigen, which varies by at least one amino acid mutation from the SARS-CoV-2 2P spike antigen (encoded by mRNA-1273). Such a vaccine, optionally referred to herein as a variant vaccine, can be administered to seropositive or seronegative subjects. For example, a subject may be naïve and not have antibodies that react with SARS-CoV-2 or may have preexisting antibodies to SARS-CoV-2 because they have previously had an infection with SARS-CoV-2 or may have previously been administered a dose of a vaccine (e.g., an mRNA vaccine) that induces antibodies against SARS-CoV-2. A variant vaccine may be the only vaccine comprising a nucleic acid encoding a SARS-CoV-2 antigen that a subject receives. Alternatively, a variant vaccine may be administered in combination with other vaccines comprising a nucleic acid encoding a SARS-CoV-2 antigen, as a prime and/or boost dose.

Thus, the disclosure, in some aspects provides a method comprising administering to a subject a vaccine comprising a nucleic acid encoding a SARS-CoV-2 spike antigen, optionally a 2P stabilized spike antigen of a second circulating SARS-CoV-2 virus, wherein the subject has previously been administered a first vaccine comprising a nucleic acid encoding a first SARS-CoV-2 2P stabilized spike antigen of a first circulating SARS-CoV-2 virus, and wherein each of the first and second 2P stabilized spike antigens are administered in an effective amount to induce an immune response specific for the first antigen and the second antigen, wherein the second circulating SARS-CoV-2 virus has a spike protein having an amino acid sequence with at least one amino acid mutation with respect to a spike protein amino acid sequence of the first circulating SARS-CoV-2 virus, and wherein the mutation is an amino acid substitution, deletion or insertion.

In some aspects, the disclosure provides a method comprising administering to a subject a first vaccine comprising a nucleic acid encoding a first SARS-CoV-2 2P stabilized spike antigen and administering to the subject a second vaccine comprising a nucleic acid encoding a second SARS-CoV-2 2P spike antigen, optionally a 2P stabilized spike antigen, wherein each of the nucleic acids encoding the first and second stabilized spike antigens are administered in an effective amount to induce an immune response specific for the respective encoded antigens, wherein the second encoded SARS-CoV-2 spike antigen has an amino acid sequence with at least one amino acid mutation with respect to the first encoded spike protein amino acid sequence, and wherein the mutation is an amino acid substitution, deletion, or insertion.

In another aspect, the disclosure provides a method comprising administering to a subject a first vaccine comprising a nucleic acid encoding a first SARS-CoV-2 2P stabilized spike antigen and administering to the subject a second vaccine comprising a nucleic acid encoding a second SARS-CoV-2 2P spike antigen, optionally a 2P stabilized spike antigen, wherein each of the nucleic acids encoding the first and second stabilized spike antigens are administered in an effective amount to induce an immune response specific for the respective encoded antigens, wherein the second encoded SARS-CoV-2 spike antigen has an amino acid sequence with at least one amino acid mutation with respect to the first encoded spike protein amino acid sequence, wherein the mutation is an amino acid substitution, deletion, or insertion, and wherein the first encoded SARS-CoV-2 spike antigen is of a first circulating SARS-CoV-2 virus and wherein the second encoded SARS-CoV-2 spike antigen is of a second circulating SARS-CoV-2 virus.

In some aspects, the disclosure provides a method comprising administering to a subject a first vaccine comprising a nucleic acid encoding a first SARS-CoV-2 2P stabilized spike antigen and administering to the subject a second vaccine comprising a nucleic acid encoding a second SARS-CoV-2 2P spike antigen, optionally a 2P stabilized spike antigen, wherein each of the nucleic acids encoding the first and second stabilized spike antigens are administered in an effective amount to induce an immune response specific for the respective encoded antigens, wherein the second encoded SARS-CoV-2 spike antigen has an amino acid sequence with at least one amino acid mutation with respect to the first encoded spike protein amino acid sequence, wherein the mutation is an amino acid substitution, deletion, or insertion, and wherein the first encoded SARS-CoV-2 spike antigen is representative of a first circulating SARS-CoV-2 virus and wherein the second encoded SARS-CoV-2 spike antigen is representative of a second circulating SARS-CoV-2 virus.

In some aspects, the disclosure provides a method comprising administering to a subject a first vaccine comprising a nucleic acid encoding a first SARS-CoV-2 2P stabilized spike antigen and administering to the subject a second vaccine comprising a nucleic acid encoding a second SARS-CoV-2 2P spike antigen, optionally a 2P stabilized spike antigen, wherein each of the nucleic acids encoding the first and second stabilized spike antigens are administered in an effective amount to induce an immune response specific for the respective encoded antigens, wherein the second encoded SARS-CoV-2 spike antigen has an amino acid sequence with at least one amino acid mutation with respect to the first encoded spike protein amino acid sequence, wherein the mutation is an amino acid substitution, deletion, or insertion, and wherein the first encoded SARS-CoV-2 spike antigen is representative of a plurality of first circulating SARS-CoV-2 viruses and/or wherein the second encoded SARS-CoV-2 spike antigen is representative of a second plurality of circulating SARS-CoV-2 viruses.

In some embodiments, the second circulating SARS-CoV-2 virus is an immunodominant emerging strain detected during a period when the first circulating SARS-CoV-2 virus is present in a subject population. In some embodiments, the second circulating SARS-CoV-2 virus and the first circulating SARS-CoV-2 virus are detectable in a subject population within at least one year. In some embodiments, the second circulating SARS-CoV-2 virus and the first circulating SARS-CoV-2 virus are detectable in a subject population during a same season. In some embodiments, the second circulating SARS-CoV-2 virus and the first circulating SARS-CoV-2 virus are detectable in a subject population during a same pandemic or endemic.

In some embodiments, the first nucleic acid encoding the SARS-CoV-2 2P stabilized spike antigen is a first nucleic acid encoding the first SARS-CoV-2 2P stabilized spike antigen.

In some embodiments, the first nucleic acid is a DNA or RNA. In some embodiments, the RNA is a messenger RNA (mRNA). In some embodiments, the nucleic acid encoding a second SARS-CoV-2 2P stabilized spike antigen of a second circulating SARS-CoV-2 virus is s second nucleic acid and is a messenger RNA (mRNA).

In some embodiments, the vaccine comprises the nucleic acid encoding the first SARS-CoV-2 spike antigen in combination with one or more additional spike protein-encoding nucleic acids. In some embodiments, the vaccine comprises the nucleic acid encoding the first SARS-CoV-2 spike antigen in combination with one or more additional nucleic acids encoding one or more SARS-CoV-2 antigens that are not spike protein-encoding nucleic acids.

In some embodiments, the immune response is a neutralizing antibody response against SARS-CoV-2. In some embodiments, the immune response is a T cell response against SARS-CoV-2.

In some embodiments, the first encoded antigen is administered to the subject as a first vaccine comprised of one or more prime or priming immunization and the second encoded antigen is administered to the subject as a boost.

In some embodiments, the second encoded antigen is administered to the subject as first vaccine comprised of one or more prime or priming immunizations and the first encoded antigen is administered to the subject as a boost.

In some embodiments, the first and second encoded antigens are administered to the subject together as a boost.

In some embodiments, the first encoded antigen is administered to the subject as a prime or priming immunization and as a boost to complete a vaccination.

In some embodiments, the first encoded antigen is administered to the subject as a prime or priming immunization and as a boost in an initial vaccination and the second encoded antigen is administered to the subject as a boost more than 3 months after the initial vaccination. In some embodiments, the first encoded antigen is administered to the subject as a prime or priming immunization and as a boost in an initial vaccination and the second encoded antigen is administered to the subject as a boost more than 6 months after the initial vaccination. In some embodiments, the boost is a seasonal boost or a pandemic shift boost. In some embodiments, the boost dose is 50 μg.

In some embodiments, the first antigen is a mRNA encoding the first SARS-CoV-2 spike antigen and wherein the spike antigen has an amino acid sequence of SEQ ID NO: 20. In some embodiments, the second antigen is a mRNA encoding the second SARS-CoV-2 spike antigen, wherein the spike antigen has an amino acid sequence with at least one amino acid mutation with respect to a protein of SEQ ID NO: 20, and wherein the mutation is an amino acid substitution, deletion or insertion.

In some aspects, the disclosure provides a composition comprising: a first messenger ribonucleic acid (mRNA) encoding a first SARS-CoV-2 spike antigen of a first circulating SARS-CoV-2 virus wherein the first SARS-CoV-2 spike antigen has an amino acid sequence of SEQ ID NO: 20 or an amino acid sequence with at least one amino acid mutation with respect to a protein of SEQ ID NO: 20 and a second mRNA encoding a second SARS-CoV-2 spike antigen of a second circulating SARS-CoV-2 virus, wherein the second SARS-CoV-2 spike antigen has an amino acid sequence with at least one amino acid mutation with respect to a protein of SEQ ID NO: 20, wherein the wherein the mutation is an amino acid substitution, deletion or insertion, and wherein the first SARS-CoV-2 spike antigen and the second SARS-CoV-2 spike antigen are different from one another.

In some embodiments, the composition further comprises a third messenger ribonucleic acid (mRNA) encoding a third SARS-CoV-2 spike antigen of a third SARS-CoV-2 virus, wherein the third SARS-CoV-2 spike antigen has an amino acid sequence with at least one amino acid mutation with respect to a protein of SEQ ID NO: 20, and wherein the mutation is an amino acid substitution, deletion or insertion.

In some embodiments, the composition further comprises a fourth messenger ribonucleic acid (mRNA) encoding a fourth SARS-CoV-2 spike antigen of a fourth SARS-CoV-2 virus, wherein the fourth SARS-CoV-2 spike antigen has an amino acid sequence with at least one amino acid mutation with respect to a protein of SEQ ID NO: 20, and wherein the mutation is an amino acid substitution, deletion or insertion.

In some embodiments, the composition further comprises a fifth messenger ribonucleic acid (mRNA) encoding a fifth SARS-CoV-2 spike antigen of a fifth SARS-CoV-2 virus, wherein the fifth SARS-CoV-2 spike antigen has an amino acid sequence with at least one amino acid mutation with respect to a protein of SEQ ID NO: 20, and wherein the mutation is an amino acid substitution, deletion or insertion.

In some embodiments, the composition further comprises a sixth messenger ribonucleic acid (mRNA) encoding a sixth SARS-CoV-2 spike antigen of a sixth SARS-CoV-2 virus, wherein the sixth SARS-CoV-2 spike antigen has an amino acid sequence with at least one amino acid mutation with respect to a protein of SEQ ID NO: 20, and wherein the mutation is an amino acid substitution, deletion or insertion.

In some embodiments, the first and second virus strains, and optionally the third, fourth, fifth and sixth virus strains are spreading in the population for at least a portion of 1 year.

The disclosure, in some aspects, provides a messenger ribonucleic acid (mRNA) encoding a SARS-CoV-2 2P stabilized spike protein, wherein the 2P stabilized spike protein has an amino acid sequence with at least one amino acid mutation with respect to a protein of SEQ ID NO: 20, wherein the mutation is an amino acid substitution, deletion or insertion, and wherein the 2P stabilized spike protein is a 2P stabilized version of a spike protein from a second circulating SARS-CoV-2 virus strain, and wherein a first circulating SARS-CoV-2 virus strain comprises a spike protein of SEQ ID NO: 36.

In some aspects of the disclosure, an mRNA encoding a protein having at least 90% or 95% sequence identity to a protein of any one of SEQ ID NOs: 5, 8, 11, 14, 17, 30, 33, 36, 39, and 42 is provided.

In some aspects of the disclosure, an mRNA having at least 90% or 95% sequence identity to an RNA of any one of SEQ ID NOs: 1, 6, 9, 12, 15, 28, 31, 34, 37, 40, 43, and 45 is provided.

In some aspects of the disclosure, an mRNA having at least 98% sequence identity to an RNA of any one of SEQ ID NOs: 1, 6, 9, 12, 15, 28, 31, 34, 37, 40, 43, and 45 is provided.

In some aspects of the disclosure, an mRNA comprising any one of SEQ ID NOs: 1, 6, 9, 12, 15, 28, 31, 34, 37, 40, 43, and 45 is provided.

In some embodiments, the mRNA comprises a chemical modification. In some embodiments, the mRNA is fully modified. In some embodiments, the chemical modification is 1-methylpseudouridine.

In some embodiments, the mRNAs are in a lipid nanoparticle and wherein the lipid nanoparticle comprises an ionizable amino lipid, a sterol, a neutral lipid, and a polyethylene glycol (PEG)-modified lipid. In some embodiments, the lipid nanoparticle comprises 40-55 mol % ionizable amino lipid, 30-45 mol % sterol, 5-15 mol % neutral lipid, and 1-5 mol % PEG-modified lipid. In some embodiments, the lipid nanoparticle comprises 40-50 mol % ionizable amino lipid, 35-45 mol % sterol, 10-15 mol % neutral lipid, and 2-4 mol % PEG-modified lipid. In some embodiments, the lipid nanoparticle comprises 45 mol %, 46 mol %, 47 mol %, 48 mol %, 49 mol %, or 50 mol % ionizable amino lipid.

In some embodiments, the ionizable amino lipid has the structure of Compound 1:

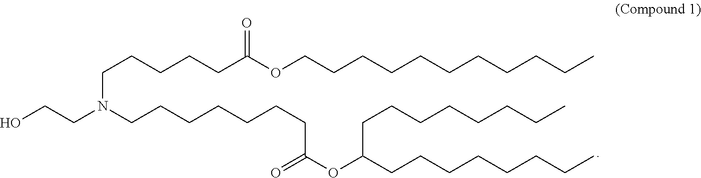 [SMILES: CCCCCCCCCCCOC(=O)CCCCCN(CCO)CCCCCCCC(=O)OC(CCCCCCCC)CCCCCCCC]

In some embodiments, the sterol is cholesterol or a derivative thereof. In some embodiments, the neutral lipid is 1,2 distearoyl-sn-glycero-3-phosphocholine (DSPC). In some embodiments, the PEG-modified lipid is 1,2 dimyristoyl-sn-glycerol, methoxypolyethyleneglycol (PEG2000 DMG).

The disclosure, in some aspects, provides a composition comprising: a first messenger ribonucleic acid (mRNA) encoding a first SARS-CoV-2 spike antigen of a first circulating SARS-CoV-2 virus wherein the first SARS-CoV-2 spike antigen has an amino acid sequence of SEQ ID NO: 20 and a second mRNA encoding a second SARS-CoV-2 spike antigen of a second circulating SARS-CoV-2 virus, wherein the second SARS-CoV-2 spike antigen has an amino acid sequence with at least one amino acid mutation with respect to a protein of SEQ ID NO: 20, wherein the mutation is an amino acid substitution, deletion or insertion, and wherein the first SARS-CoV-2 spike antigen and the second SARS-CoV-2 spike antigen are different from one another.

In some embodiments, the wherein the first SARS-CoV-2 spike antigen has an amino acid sequence with at least one amino acid mutation with respect to a protein of SEQ ID NO: 20, wherein the mutation is an amino acid substitution, deletion, or insertion.

The disclosure, in some aspects, provides a method comprising administering to a subject a vaccine comprising a first nucleic acid encoding a SARS-CoV-2 2P stabilized spike antigen, wherein the spike antigen has an amino acid sequence with at least one amino acid mutation with respect to a protein of SEQ ID NO: 20, and wherein the mutation is an amino acid substitution, deletion or insertion, wherein the subject is seropositive for a SARS-CoV-2 antigen of SEQ ID NO. 21 or 20.

Another aspect of the disclosure provides a method comprising administering to a subject a vaccine comprising a first nucleic acid encoding a SARS-CoV-2 2P stabilized spike antigen, wherein the spike antigen has an amino acid sequence with at least one amino acid mutation with respect to a protein of SEQ ID NO: 20, and wherein the mutation is an amino acid substitution, deletion or insertion, wherein the subject is seronegative for a SARS-CoV-2 antigen of SEQ ID NO. 21 or 20.

In some embodiments, the subject is administered a second dose of the vaccine between 2 weeks and 1 year after the first dose of vaccine is administered. In some embodiments, the subject is administered a second vaccine between 2 weeks and 1 year after the vaccine is administered, wherein the second vaccine comprises a second nucleic acid encoding a SARS-CoV-2 2P stabilized spike antigen of SEQ ID NO: 20. In some embodiments, the second vaccine comprises a mixture of the first and second nucleic acids, wherein the first nucleic acid and the second nucleic acid are present in the second vaccine at a ratio of 1:1.

In some embodiments, 50 μg of the vaccine comprising a nucleic acid encoding a SARS-CoV-2 spike antigen, optionally, a 2P stabilized spike antigen of a third circulating SARS-CoV-2 virus is administered to the subject. In some embodiments, the subject is administered an effective dose of the vaccine. In some embodiments, effective dose is 20 μg-50 μg. In some embodiments, the effective dose is 20 μg. In some embodiments, the effective dose is 25 μg. In some embodiments, the effective dose is 30 μg. In some embodiments, the effective dose is 40 μg. In some embodiments, the effective dose is 50 μg.

In some embodiments, the vaccine comprises a nucleic acid encoding a SARS-CoV-2 spike antigen having at least 95% sequence identity to SEQ ID NO: 11. In some embodiments, the vaccine comprises a nucleic acid having at least 95% sequence identity to SEQ ID NO: 9. In some embodiments, the vaccine comprises a nucleic acid comprising SEQ ID NO: 9.

In some embodiments, the vaccine comprises a nucleic acid encoding a SARS-CoV-2 spike antigen having at least 95% sequence identity to SEQ ID NO: 30. In some embodiments, the vaccine comprises a nucleic acid having at least 95% sequence identity to SEQ ID NO: 28. In some embodiments, the vaccine comprises a nucleic acid comprising SEQ ID NO: 28.

In some embodiments, the vaccine comprises a nucleic acid encoding a SARS-CoV-2 spike antigen having at least 95% sequence identity to SEQ ID NO: 26. In some embodiments, the vaccine comprises a nucleic acid having at least 95% sequence identity to SEQ ID NO: 24. In some embodiments, the vaccine comprises a nucleic acid comprising SEQ ID NO: 24.

In some embodiments, the first vaccine or the second vaccine comprises: (a) a nucleic acid encoding a SARS-CoV-2 spike antigen comprising two proline substitutions; and (b) a nucleic acid encoding a SARS-CoV-2 spike antigen comprising SEQ ID NO: 11. In some embodiments, the first vaccine or the second vaccine comprises: (a) a nucleic acid encoding a SARS-CoV-2 spike antigen comprising two proline substitutions; and (b) a nucleic acid encoding a SARS-CoV-2 spike antigen comprising SEQ ID NO: 26. In some embodiments, the first vaccine or the second vaccine comprises: (a) a nucleic acid encoding a SARS-CoV-2 spike antigen comprising two proline substitutions; and (b) a nucleic acid encoding a SARS-CoV-2 spike antigen comprising SEQ ID NO: 30.

In some embodiments, the ratio of (a) to (b) is 1:1, 1:2, 1:3, 1:4, 2:1, 3:1, or 4:1.

In some embodiments, the second vaccine comprises: (a) the nucleic acid encoding a first SARS-CoV-2 2P spike antigen of a first circulating SARS-CoV-2 virus; and (b) the nucleic acid encoding a second SARS-CoV-2 2P spike antigen of a second circulating SARS-CoV-2 virus. In some embodiments, the ratio of (a) to (b) is 1:1, 1:2, 1:3, 1:4, 2:1, 3:1, or 4:1.

The disclosure, in some aspects provides a composition comprising 50 μg-250 μg of a first messenger ribonucleic acid (mRNA) comprising a first open reading frame (ORF) that encodes a first SARS-CoV-2 prefusion stabilized spike(S) protein and a second mRNA comprising a second ORF that encodes a second SARS-CoV-2 prefusion stabilized spike(S) protein; and a lipid nanoparticle comprising a mixture of lipids that comprises an ionizable amino lipid, a non-cationic lipid, a sterol, and a PEG-modified lipid.

In some embodiments, the composition comprises 50 μg of mRNA in total. In some embodiments, the ratio of the first mRNA to the second mRNA is 1:1.

In some embodiments, the mRNA comprises a chemical modification. In some embodiments, the mRNA is fully modified. In some embodiments, the chemical modification is 1-methylpseudouridine.

In some embodiments, the mRNA further comprises a 5′ cap analog, optionally a 7mG(5′)ppp(5′)NlmpNp cap. In some embodiments, the mRNA further comprises a poly(A) tail, optionally having a length of 50 to 150 nucleotides (e.g., 100 nucleotides).

In some embodiments, the lipid nanoparticle comprises 40-55 mol % ionizable amino lipid, 30-45 mol % sterol, 5-15 mol % neutral lipid, and 1-5 mol % PEG-modified lipid. In some embodiments, the lipid nanoparticle comprises 40-50 mol % ionizable amino lipid, 35-45 mol % sterol, 10-15 mol % neutral lipid, and 2-4 mol % PEG-modified lipid. In some embodiments, the lipid nanoparticle comprises 45 mol %, 46 mol %, 47 mol %, 48 mol %, 49 mol %, or 50 mol % ionizable amino lipid. In some embodiments, the ionizable amino lipid has the structure of Compound 1:

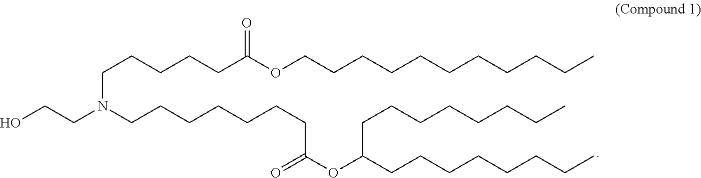 [SMILES: CCCCCCCCCCCOC(=O)CCCCCN(CCO)CCCCCCCC(=O)OC(CCCCCCCC)CCCCCCCC]

In some embodiments, the sterol is cholesterol or a derivative thereof. In some embodiments, the neutral lipid is 1,2 distearoyl-sn-glycero-3-phosphocholine (DSPC). In some embodiments, the PEG-modified lipid is 1,2 dimyristoyl-sn-glycerol, methoxypolyethyleneglycol (PEG2000 DMG).

In some embodiments, the composition further comprises Tris buffer, sucrose, and sodium acetate, or any combination thereof. In some embodiments, the composition further comprises 30-40 mM Tris buffer, 80-95 mg/mL sucrose, and 5-15 mM sodium acetate. In some embodiments, the composition has a pH value of 6-8, optionally 7.5.

In some embodiments, the disclosure provides a method comprising administering to a human subject, the composition described herein.

In some embodiments, the composition is administered intramuscularly, optionally into a deltoid region of the human subject.

In some embodiments, the human subject has previously been administered a SARS-CoV-2 vaccine. In some embodiments, the SARS-CoV-2 vaccine comprises an mRNA vaccine. In some embodiments, the mRNA vaccine comprises an mRNA comprising an ORF encoding a SARS-CoV-2 prefusion stabilized S protein. In some embodiments, the SARS-CoV-2 prefusion stabilized S protein comprises the first SARS-CoV-2 prefusion stabilized S protein. In some embodiments, the human subject has previously been administered at least one dose of the SARS-CoV-2 vaccine. In some embodiments, the human subject has previously been administered two doses of the SARS-CoV-2 vaccine.

In some embodiments, the method comprises administering the composition to the human subject at least six months after the most recent administration of the SARS-CoV-2 vaccine.

In some embodiments, the composition induces neutralizing antibody titers in the human subject. In some embodiments, the percentage of subjects with seroconversion after a single dose at Day 29 is at least 80%, at least 90%, at least 95% or 100%. In some embodiments, the percentage of subjects with seroconversion after a single dose at Day 29 is 100%.

A further aspect of the disclosure provides a method comprising administering to a subject a booster vaccine comprising a nucleic acid encoding a first SARS-CoV-2 antigen from a first SARS-CoV-2 virus, wherein the subject has previously been administered at least one prime dose of a first vaccine comprising a first nucleic acid encoding the SARS-CoV-2 antigen of the first the SARS-CoV-2 virus, wherein the booster vaccine is administered in an effective amount to induce a neutralizing immune response against a second SARS-CoV-2 virus, wherein the second SARS-CoV-2 virus comprises a second SARS-CoV-2 antigen, wherein the second SARS-CoV-2 antigen has an amino acid sequence with at least one amino acid mutation with respect to a corresponding protein antigen of the first SARS-CoV-2 virus, wherein the booster vaccine is administered in a dosage of 25-100 μg at least 6 months after a first dose of the first vaccine, and wherein the first antigen is a full length stabilized spike protein having a 2P mutation.

In some embodiments, the booster vaccine is administered in a dosage of 50 μg.

In some embodiments, the booster vaccine is administered at least about 6 months after a second dose of the first vaccine. In some embodiments, the booster vaccine is administered 6-12 months after a second dose of the first vaccine. In some embodiments, the booster vaccine is administered at least about 8 months after a second dose of the first vaccine.

In some embodiments, the boost dose is a seasonal boost or a pandemic shift boost to provide a neutralizing immune response against a plurality of variants of concern.

In some embodiments, the first vaccine or the second vaccine comprises: a nucleic acid encoding a SARS-CoV-2 spike antigen comprising two proline substitutions; and a nucleic acid encoding a SARS-CoV-2 spike antigen comprising SEQ ID NO: 33.

In some embodiments, the first vaccine or the second vaccine comprises: a nucleic acid encoding a SARS-CoV-2 spike antigen comprising two proline substitutions; and a nucleic acid encoding a SARS-CoV-2 spike antigen comprising SEQ ID NO: 36.

In some embodiments, the first vaccine or the second vaccine comprises: a nucleic acid encoding a SARS-CoV-2 spike antigen comprising two proline substitutions; and a nucleic acid encoding a SARS-CoV-2 spike antigen comprising SEQ ID NO: 39.

In some embodiments, the first vaccine or the second vaccine comprises: a nucleic acid encoding a SARS-CoV-2 spike antigen comprising two proline substitutions; and a nucleic acid encoding a SARS-CoV-2 spike antigen comprising SEQ ID NO: 42. In some embodiments, the vaccine comprises a nucleic acid having at least 95% sequence identity to SEQ ID NO: 40. In some embodiments, the vaccine comprises a nucleic acid comprising SEQ ID NO: 40. In some embodiments, the vaccine comprises a nucleic acid having at least 95% sequence identity to SEQ ID NO: 43. In some embodiments, the vaccine comprises a nucleic acid comprising SEQ ID NO: 43. In some embodiments, the vaccine comprises a nucleic acid having at least 95% sequence identity to SEQ ID NO: 45. In some embodiments, the vaccine comprises a nucleic acid comprising SEQ ID NO: 45.

### BRIEF DESCRIPTION OF THE DRAWINGS

FIGS. 1A-1B show neutralization of SARS-CoV-2 pseudoviruses by serum from immunized non-human primates (NHP) or Phase 1 participants. FIG. 1A shows the data from sera collected four weeks post-boost from Rhesus macaques (NHPs) immunized with 30 μg mRNA encoding Spike protein with two proline substitutions on a prime-boost schedule. FIG. 1B shows the data from sera collected one week post-boost from Phase 1 trial participants immunized with 100 μg mRNA encoding Spike protein with two proline substitutions on a prime-boost schedule. Neutralization was measured by a recombinant VSV-based SARS-CoV-2 pseudovirus neutralization assay incorporating full-length Spike protein of the USA-WA1/2020 isolate (D614) or the indicated Spike variants (from left to right: D614G, A222V-D614G, S477N-D614G, N439K-D614G, mink cluster 5 variant). The horizontal dotted lines indicate the lower limit of quantification (LLOQ). D=D614 of USA-WA1/2020 isolate, G=D614G variant.

FIGS. 2A-2B show neutralizing antibodies in NHPs against the original (D614), D614G, and Spike variants. Rhesus macaques (NHPs) were immunized with 100 or 30 μg mRNA encoding Spike protein with two proline substitutions on a prime-boost schedule, and sera were collected 4 weeks post the boost. Neutralization was measured by a recombinant VSV-based pseudovirus neutralization assay (FIGS. 2A-2B). The assays incorporated full-length Spike protein of the original D614 (D), D614G (G), or the indicated Spike variants present in the B.1.1.7 variant (FIG. 2A) or B.1.351 variant (FIG. 2B). The horizontal dotted lines indicate the lower limit of quantification (LLOQ). D=D614 of USA-WA1/2020 isolate, G=D614G variant.

FIGS. 3A-3C show neutralization curves of NHP samples in the VSV-based pseudovirus neutralization assay. FIGS. 3A-3B show the data from the 100 μg mRNA encoding Spike protein with two proline substitutions dose and FIG. 3C shows the data from the 30 μg mRNA encoding Spike protein with two proline substitutions dose. Individual animal numbers from the study are indicated above each graph. G=D614G.

FIGS. 4A-4D show the results of neutralization assays against B.1.1.7 and B.1.351 from human sera collected from humans after administration of mRNA encoding Spike protein with two proline substitutions. The Phase 1 trial participant sera from administration of mRNA encoding Spike protein with two proline substitutions were collected seven days after the boosting, on day 36. Neutralization was measured by a recombinant VSV-based pseudovirus neutralization assay that incorporated D614G (G) or the indicated Spike mutations present in the B.1.1.7 variant (FIGS. 4A, 4C) or B.1.351 variant (FIGS. 4B, 4D). Results from individual participant sera are represented as dots on each figure, with lines connecting the D614G and variant neutralization titers (FIGS. 4C, 4D). The horizontal dotted lines indicate the lower limit of quantification (LLOQ). D=D614 USA-WA1/2020 isolate, G=D614G variant.

FIGS. 5A-5B show neutralization curves of human serum samples in the VSV-based pseudovirus neutralization assay. Serum from each clinical trial participant is represented as a separate graph. G=D614G variant.

FIG. 6 shows neutralization titers of murine serum samples. Serum was from mice administering 1 μg of mRNA encoding a Spike protein with two proline substitutions. G=D614 variant.

FIG. 7 shows neutralization titers of murine serum samples. Serum was from mice administered 1 μg of mRNA encoding a Spike protein having the D614G mutation. G=D614 variant.

FIGS. 8A-8G show neutralization titers of murine serum samples. Sera were obtained from mice 15 days after administration of PBS (negative control) or 10 μg of mRNA encoding SARS-CoV-2 Spike proteins. FIG. 8A shows neutralization titers of sera obtained from mice administered 10 μg of mRNA-1273 encoding a Spike protein with two proline substitutions. FIG. 8B shows neutralization titers of sera obtained from mice administered 10 μg of mRNA-1273.351 encoding a Spike protein of the SARS-CoV-2 B.1.351 (RSA) variant. FIG. 8C shows neutralization titers of sera obtained from mice administered a 1:1 mixture of mRNA-1273 and mRNA-1273.351 (5 μg of each mRNA, 10 μg total mRNA). FIG. 8D shows neutralization titers of sera obtained from mice administered PBS. FIG. 8E shows a comparison of neutralization titers towards B.1.351 variant Spike protein and D614G variant Spike protein, using sera obtained from mice administered 10 μg mRNA-1273. FIG. 8F shows a comparison of neutralization titers towards B.1.351 variant Spike protein and D614G variant Spike protein, using sera obtained from mice administered 10 μg mRNA-1273.351. FIG. 8G shows a comparison of neutralization titers towards B.1.351 variant Spike protein and D614G variant Spike protein, using sera obtained from mice administered a 1:1 mixture of mRNA-1273 and mRNA-1273.351 (5 μg of each mRNA, 10 μg total mRNA).

FIG. 9 shows titers of IgG specific to a SARS-CoV-2 Spike protein with two proline substitutions. Sera were obtained from mice administered PBS, 1 μg, or 10 μg mRNA encoding SARS-CoV-2 Spike protein with two proline substitutions (mRNA-1273), SARS-CoV-2 B.1.351 Spike protein (Mrna-1273.351), or a 1:1 mixture of mRNA-1273 and mRNA-1273.351 (0.5 μg or 5 μg of each mRNA, 1 μg or 10 μg total mRNA).

FIGS. 10A-10D show the ability of sera elicited by mice immunized with mRNA encoding SARS-CoV-2 Spike proteins to bind SARS-CoV-2 antigens. FIG. 10A shows binding towards SARS-CoV-2 Spike protein with two proline substitutions. FIG. 10B shows binding towards SARS-CoV-2 B.1.351 (RSA) variant Spike protein with two proline substitutions. FIG. 10C shows binding towards SARS-CoV-2 N protein. FIG. 10D shows binding towards the receptor-binding domain (RBD) of SARS-CoV-2 B.1.351 (RSA) variant Spike protein. Sera were obtained from mice administered PBS, 1 μg, or 10 μg mRNA encoding SARS-CoV-2 Spike protein with two proline substitutions (mRNA-1273), SARS-CoV-2 B.1.351 Spike protein (RSA-full), SARS-CoV-2 Spike protein with two proline substitutions and a D614G mutation (WH202020_NatSP_2P_001_D614G), or a 1:1 mixture of mRNA-1273 and mRNA-1273.351 (0.5 μg or 5 μg of each mRNA, 1 μg or 10 μg total mRNA).

FIG. 11 shows neutralizing antibody titers in mice following 1, 2, or 3 doses of vaccine. Balb/c mice were immunized with 1 or 0.1 μg mRNA-1273 and were boosted with 1 or 0.1 μg mRNA-1273.351 on study day 213. Results from individual mouse sera is represented as dots on each figure, and the line is the mean of each group. The horizontal dotted line indicates the limit of detection (LOD).

FIG. 12 shows neutralizing antibody titers in mice following 1, 2, or 3 doses of vaccine. BALB/c mice were immunized with 1 or 0.1 μg mRNA-1273 (dose 1 on day 1 and dose 2 on day 22) and were boosted with 1 or 0.1 μg mRNA-1273.351 on day 213. Results from individual mouse sera (n=5 per group) are represented as dots on each figure, and the line is the mean of each group. The horizontal dotted line indicates the LLOD for log 10 IgG titer at 1.602.

FIGS. 13A-13E show neutralizing antibody titers in humans following 2 doses of 100 μg mRNA-1273 (dose 1 on day 1 and dose 2 on day 22), and a 3rd dose (booster dose) with 50 μg of mRNA-1273 6.2 to 6.7 months after day 1. FIG. 13A shows neutralizing assay titers of sera obtained prior to administration of the booster dose, based on neutralization of VSV pseudoviruses expressing a Spike protein with a D614G mutation relative to wild-type sequence (left bar), a Spike protein having the mutations associated with the B.1.351 variant (middle bar), and a Spike protein having the mutations associated with the P.1 variant. FIG. 13B shows neutralizing assay titers of sera obtained 14 days after administration of the booster dose towards the same pseudoviruses expressing the same Spike proteins tested in FIG. 13A. In FIGS. 13A-13B, the geometric mean neutralizing antibody titer, shown above the bar, is denoted by the top of the bar and the 95% confidence intervals are shown by the error bars. FIGS. 13C-13E show the change in neutralizing antibody titers from day 1 (prior to administration of the booster dose) to day 15 (14 days after administration of the booster dose), towards VSV pseudoviruses expressing a D614G Spike protein (FIG. 13C), B.1.351 Spike protein (FIG. 13D), and P.1 Spike protein (FIG. 13E). The fold increases for day 15/day 1 are shown above the bars. ****=p<0.0001 by the Wilcoxon matched-pairs signed rank test. For all plots, the titers for individual participants are shown by the circles. The horizontal dotted lines indicate the lower limit of quantification (LLOQ).

FIGS. 14A-14E show neutralizing antibody titers in humans following 2 doses of 100 μg mRNA-1273 (dose 1 on day 1 and dose 2 on day 22), and a 3^{rd} dose (booster dose) with 50 μg of mRNA-1273.351 6.2 to 6.7 months after day 1. FIG. 14A shows neutralizing assay titers of sera obtained prior to administration of the booster dose, based on neutralization of VSV pseudoviruses expressing a Spike protein with a D614G mutation relative to wild-type sequence (left bar), a Spike protein having the mutations associated with the B.1.351 variant (middle bar), and a Spike protein having the mutations associated with the P.1 variant. FIG. 14B shows neutralizing assay titers of sera obtained 14 days after administration of the booster dose, towards the same pseudoviruses expressing the same Spike proteins tested in FIG. 14A. In FIGS. 14A-14B, the geometric mean neutralizing antibody titer, shown above the bar, is denoted by the top of the bar and the 95% confidence intervals are shown by the error bars. FIGS. 14C-14E show the change in neutralizing antibody titers from day 1 (prior to administration of the booster dose) to day 15 (14 days after administration of the booster dose), towards VSV pseudoviruses expressing a D614G Spike protein (FIG. 14C), B.1.351 Spike protein (FIG. 14D), and P.1 Spike protein (FIG. 14E). The fold increases for day 15/day 1 are shown above the bars. ****=p<0.0001 by the Wilcoxon matched-pairs signed rank test. For all plots, the titers for individual participants are shown by the circles. The horizontal dotted lines indicate the lower limit of quantification (LLOQ).

FIGS. 15A-15E show neutralizing antibody titers towards D614G and B.1.351 SARS-CoV-2 pseudoviruses, of sera from human participants administered two doses of mRNA-1273 and boosted with a 3rd dose of either mRNA-1273, or mRNA-1273.351 encoding a Spike protein with the mutations associated with the B.1.351 variant. FIGS. 15A-15B show the reduction in neutralizing antibody titers towards B.1.351 Spike protein relative to a D614G Spike protein in sera obtained prior to administration of a booster dose containing 50 μg mRNA-1273 (FIG. 15A), or on day 15, 14 days after booster dose administration (FIG. 15B). FIGS. 15C-15D show the reduction in neutralizing antibody titers towards B.1.351 Spike protein relative to a D614G Spike protein in sera obtained prior administration of a booster dose containing 50 μg mRNA-1273.351 (FIG. 15C), or on day 15, 14 days after booster dose administration (FIG. 15D). FIG. 15E shows neutralizing antibody titers towards pseudoviruses expressing a D614G Spike protein prior to administration of a booster dose (left of dashed line, squares), and neutralizing antibody titers towards a panel of pseudoviruses expressing a D614G, B.1.351, or P.1 Spike protein, 14 days after administration of 50 μg mRNA-1273 (circles) or 50 μg mRNA-1273.351 (diamonds). The horizontal dotted lines indicate the lower limit of quantification (LLOQ). For bar plots, geometric mean neutralizing antibody titer, shown above the bar, is denoted by the top of the box and the 95% confidence intervals are shown by the error bars.

FIGS. 16A-16C show neutralizing antibody titers towards pseudoviruses expressing variant SARS-CoV-2 Spike proteins, of sera obtained from human participants administered two doses of 100 μg mRNA-1273 (FIGS. 16A-16B), and optionally administered a booster dose of 50 μg mRNA-1273, 50 μg mRNA-1273.351 encoding a B.1.351 variant Spike protein, or 50 μg of mRNA-1273.211 (1:1 mixture of mRNA-1273 and mRNA-1273.351) (FIG. 16C). FIG. 16A shows neutralization titers of sera towards pseudoviruses expressing a D614G, B.1.617.1-v1, B.1.617.1-v2, or A. VOI-V2 Spike protein. FIG. 16B shows the relative reduction in neutralization towards B.1.617.1-v1, B.1.617.1-v2, or A. VOI-V2 Spike proteins, relative to D614G Spike protein. FIG. 16C shows neutralization titers towards B.1.617.1-v1 Spike protein, of sera obtained from human subjects that were administered two doses of 100 μg mRNA-1273 (left bar), followed by a booster dose of 50 μg mRNA-1273 (2^{nd} bar), 50 μg mRNA-1273.351 (3^{rd} bar), or 50 μg mRNA-1273.211 (4^{th} bar). Horizontal bars represent the increase in neutralization titers towards B.1.617.1-v1 after administration of a booster dose, relative to sera from subjects administered only two doses of 100 μg mRNA-1273. The horizontal dotted lines indicate the lower limit of quantification (LLOQ).

FIGS. 17A-17D show neutralization titers of murine serum samples. Sera was obtained from mice that were administered 1 μg of mRNA-1273 on day 1 (prime dose) and again on day 22 (booster dose). Mice were administered 1 μg of mRNA-1273.351, encoding a Spike protein with the mutations associated with the B.1.351 SARS-CoV-2 variant, on day 213 (3rd dose), and day 234 (4th dose). Samples were taken at day 212 (before administration of the 3rd dose), day 233 (before administration of the 4th dose), and day 248 (14 days after administration of the 4th dose). FIG. 17A shows the neutralizing antibody titer at each time point, based on neutralization of pseudoviruses comprising a SARS-CoV-2 Spike protein with a D614G mutation (left bars) or the mutations associated with the South Africa B.1.351 variant. FIG. 17B shows, for each Spike protein tested in FIG. 17A, the kinetics of neutralization titers from day 212 through day 248. FIG. 17C shows, at each time point, the relative change in neutralization titers towards a B.1.351 Spike protein compared to a Spike protein comprising only a D614G mutation. FIG. 17D shows reference neutralization titers of sera from day 36, two weeks after a second dose, towards D614G Spike protein. The horizontal dotted lines indicate the lower limit of quantification (LLOQ). For bar plots, geometric mean neutralizing antibody titer, shown above the bar, is denoted by the top of the box and the 95% confidence intervals are shown by the error bars.

FIGS. 18A-18D show neutralization titers of murine serum samples. Sera was obtained from mice that were administered 0.1 μg of mRNA-1273 on day 1 (prime dose) and again on day 22 (booster dose). Mice were administered 0.1 μg of mRNA-1273.351, encoding a Spike protein with the mutations associated with the B.1.351 SARS-CoV-2 variant, on day 213 (3rd dose), and day 234 (4th dose). Samples were taken at day 212 (before administration of the 3rd dose), day 233 (before administration of the 4th dose), and day 248 (14 days after administration of the 4th dose). FIG. 18A shows the neutralizing antibody titer at each time point, based on neutralization of pseudoviruses comprising a SARS-CoV-2 Spike protein with a D614G mutation (left bars) or the mutations associated with the South Africa B.1.351 variant. FIG. 18B shows, for each Spike protein tested in FIG. 18A, the kinetics of neutralization titers from day 212 through day 248. FIG. 18C shows, at each time point, the relative change in neutralization titers towards a B.1.351 Spike protein compared to a Spike protein comprising only a D614G mutation. FIG. 18D shows reference neutralization titers of sera from day 36, two weeks after a second dose, towards D614G Spike protein. The horizontal dotted lines indicate the lower limit of quantification (LLOQ). For bar plots, geometric mean neutralizing antibody titer, shown above the bar, is denoted by the top of the box and the 95% confidence intervals are shown by the error bars.

FIGS. 19A-19C show neutralization titers of murine serum samples. Sera was obtained from mice that were administered 1 μg of mRNA-1273 on day 1 (prime dose) and again on day 22 (booster dose). Mice were administered 1 μg of mRNA-1273.351, encoding a Spike protein with the mutations associated with the B.1.351 SARS-CoV-2 variant, on day 58 (3rd dose), and day 78 (4th dose). Samples were taken at day 57 (before administration of the 3rd dose), day 77 (before administration of the 4th dose), and day 92 (14 days after administration of the 4th dose). FIG. 19A shows the neutralizing antibody titer at each time point, based on neutralization of pseudoviruses comprising a SARS-CoV-2 Spike protein with a D614G mutation (left bars) or the mutations associated with the South Africa B.1.351 variant. FIG. 19B shows, for each Spike protein tested in FIG. 19A, the kinetics of neutralization titers from day 57 through day 92. FIG. 19C shows, at each time point, the relative change in neutralization titers towards a B.1.351 Spike protein compared to a Spike protein comprising only a D614G mutation. The horizontal dotted lines indicate the lower limit of quantification (LLOQ). For bar plots, geometric mean neutralizing antibody titer, shown above the bar, is denoted by the top of the box and the 95% confidence intervals are shown by the error bars.

FIGS. 20A-20C show neutralization titers of murine serum samples. Sera was obtained from mice that were administered 0.1 μg of mRNA-1273 on day 1 (prime dose) and again on day 22 (booster dose). Mice were administered 0.1 μg of mRNA-1273.351, encoding a Spike protein with the mutations associated with the B.1.351 SARS-CoV-2 variant, on day 58 (3rd dose), and day 78 (4th dose). Samples were taken at day 57 (before administration of the 3rd dose), day 77 (before administration of the 4th dose), and day 92 (14 days after administration of the 4th dose). FIG. 20A shows the neutralizing antibody titer at each time point, based on neutralization of pseudoviruses comprising a SARS-CoV-2 Spike protein with a D614G mutation (left bars) or the mutations associated with the South Africa B.1.351 variant. FIG. 20B shows, for each Spike protein tested in FIG. 20A, the kinetics of neutralization titers from day 57 through day 92. FIG. 20C shows, at each time point, the relative change in neutralization titers towards a B.1.351 Spike protein compared to a Spike protein comprising only a D614G mutation. The horizontal dotted lines indicate the lower limit of quantification (LLOQ). For bar plots, geometric mean neutralizing antibody titer, shown above the bar, is denoted by the top of the box and the 95% confidence intervals are shown by the error bars.

FIGS. 21A-21H show Spike protein-specific IgG and neutralization titers of murine serum samples. Sera were collected from mice administered 10, 1, or 0.1 μg mRNA encoding a 2P-stabilized from of the wild-type Spike protein (mRNA-1273), B.1.351 Spike protein (mRNA-1273.351), or B.1.1.7 Spike protein (mRNA-1273.117) on day 1 (prime dose), and again on day 22 (2nd dose). Sera were collected at day 36 (14 days after 2nd dose). FIG. 21A shows total IgG specific to wild-type 2P-stabilized SARS-CoV-2 Spike protein, as measured by ELISA. FIG. 21B shows neutralization titers of day 36 serum samples from mice administered two doses of 1 μg mRNA-1273, towards pseudoviruses containing one of a panel of Spike proteins, including 1) a Spike protein with a D614G mutation, 2) a Spike protein with the mutations associated with the B.1.351 variant, 3) a Spike protein with the mutations associated with the P.1 variant, 4) a Spike protein with the mutations associated with the B.1.1.7 variant, and 5) a Spike protein with the mutations associated with the B.1.1.7 variant as well as an E484K mutation. FIG. 21C shows the relative reduction in neutralization titers for sera against each of the viruses tested in FIG. 21B, relative to the baseline of neutralization titers towards D614G Spike protein. FIG. 21D shows neutralization titers of day 36 serum samples from mice administered two doses of 1 μg mRNA-1273.351, towards pseudoviruses containing one of a panel of Spike proteins, including 1) a Spike protein with a D614G mutation, 2) a Spike protein with the mutations associated with the B.1.351 variant. 3) a Spike protein with the mutations associated with the P.1 variant, and 4) a Spike protein with the mutations associated with the B.1.1.7 variant as well as an E484K mutation. FIG. 21E shows the relative reduction in neutralization titers for sera against each of the viruses tested in FIG. 21D, relative to the baseline of neutralization titers towards D614G Spike protein. FIG. 21F shows neutralization titers of day 36 serum samples from mice administered two doses of 1 μg mRNA-1273.117, towards pseudoviruses containing one of a panel of Spike proteins, including 1) a Spike protein with a D614G mutation, 2) a Spike protein with the mutations associated with the B.1.351 variant, 3) a Spike protein with the mutations associated with the P.1 variant, 4) a Spike protein with the mutations associated with the B.1.1.7 variant, and 5) a Spike protein with the mutations associated with the B. 1.1.7 variant as well as an E484K mutation. FIG. 21G shows the relative reduction in neutralization titers for sera against each of the viruses tested in FIG. 21F, relative to the baseline of neutralization titers towards D614G Spike protein. FIG. 21H shows reference neutralizing antibody titers of mice administered two doses of either mRNA-1273, mRNA-1273.351, or mRNA-1273.211 (1:1 mixture of 0.50 μg each mRNA-1273 and mRNA-1273.351) on days 1 and 22, with sera collected on day 36. Sera from each group of mice were tested against pseudoviruses containing one of a panel of Spike proteins, including 1) a Spike protein with a D614G mutation, 2) a Spike protein with the mutations associated with the B.1.351 variant, 3) a Spike protein with the mutations associated with the CAL.20C variant, 4) a Spike protein with the mutations associated with the P.1 variant. The horizontal dotted lines indicate the lower limit of quantification (LLOQ). For bar plots, geometric mean neutralizing antibody titer, shown above the bar, is denoted by the top of the box and the 95% confidence intervals are shown by the error bars.

FIG. 22 is a graph depicting the neutralizing antibody titers (log 10) elicited against a panel of SARS-CoV-2 variants.

FIGS. 23A-23B show neutralization titers of serum samples taken from human Phase 1 participants. FIG. 23A shows neutralization titers of human Phase 1 participant sera against D614G, B.1.617.1-v1, B.1.617.1-v2, and B.1.617.2. FIG. 23B shows the relative reduction in neutralization titers for sera against each of the viruses tested in FIG. 23A.

FIGS. 24A-24B show neutralization titers of serum samples taken two weeks after a third dose (booster) of vaccine comprising mRNA-1273, mRNA-1273.351, or mRNA-1273.211. The serum samples were tested against D614G and variants B.1.351, P.1, B.1.427/1.429, B.1.526, A. VOI.V2, B.1.617.1, and B.1.617.2. Neutralization titers are shown in FIG. 24A and FIG. 24B shows the fold-reduction in neutralization titer against different variants relative to the neutralization titer of the D614G spike protein.

FIGS. 25A-25E show D614 and B.1.351 neutralization data in non-human primates (NHPs) using a lentiviral pseudovirus neutralization assay. Temporal studies for D614G (FIG. 25A) and B.1.351 (FIG. 25B) are shown. In addition, the reduction in neutralizing antibodies was analyzed for each group using lentiviral pseudovirus neutralization (FIG. 25C), VSV pseudovirus neutralization (FIG. 25D), and live virus neutralization (FIG. 25E).

FIGS. 26A-26B show the results from 2020 NHP challenge studies following two doses of mRNA-1273 and challenge with USA-WA1/20. The graphs show the number of genome copies of the virus in lung samples (bronchoalveolar lavage, BAL, fluid; FIG. 26A) and from nasal swabs (FIG. 26B).

FIGS. 27A-27B show the results from 2021 NHP challenge studies following two doses of mRNA-1273 and challenge with USA-WA1/20. The graphs show the number of RNA genome copies of the virus in lung samples (bronchoalveolar lavage, BAL, fluid; FIG. 27A) and from nasal swabs (FIG. 27B).

FIGS. 28A-28B show the results from 2021 NHP challenge studies following two doses of mRNA-1273 and challenge with D614G or B.1.351. FIG. 28A shows a reduction in neutralization titers between the two viruses using live virus neutralization and FIG. 28B shows a reduction in neutralization titers between eh two viruses using pseudovirus neutralization.

FIGS. 29A-29B show the results from 2021 NHP challenge studies following two doses of mRNA-1273 and challenge with D614G or B.1.351. The graphs show the number of RNA genome copies of the virus in lung samples (bronchoalveolar lavage, BAL, fluid; FIG. 29A) and from nasal swabs (FIG. 29B) at two time points.

FIGS. 30A-30B show the results from 2021 NHP challenge studies following two doses of mRNA-1273 and challenge with USA-WA1/20. The graphs show the number of RNA genome copies of the virus in lung samples (bronchoalveolar lavage, BAL, fluid; FIG. 30A) and from nasal swabs (FIG. 30B) at three different time points.

FIGS. 31A-31B show the neutralization of recombinant SARS-CoV-2 VSV-based pseudoviruses (D614G, B.1.351 and P.1) by serum from participants 1 month after the primary series (two doses of mRNA-1273) and 6-8 months later. The geometric mean neutralizing antibody titer is denoted by the top of the box, and 95% confidence intervals are shown by the brackets. The titers for individual participants are shown by the circles. The horizontal dotted lines indicate the lower limit of quantification (LLOQ).

FIG. 32 includes three graphs showing that administration of a booster shot increases neutralizing antibody titers against wild-type (D614) and variants of concern (B.1.351 and P.1). Three boosting strategies were tested: mRNA-1273, mRNA-1273.351, and mRNA-1273.211.

FIGS. 33A-33C show neutralization titers after administration of a booster shot. Three different boosting strategies were tested: mRNA-1273 (FIG. 33A), mRNA-1273.351 (FIG. 33B), and mRNA-1273.211 (FIG. 33C). The variants of concern/interest surveyed were: B.1.351, P.1, B.1.427/B.1.429, B.1.526, A. VOI.V2, B.1.617.1, and B.1.617.2.

FIG. 34 is a cohort comparison between different boosting strategies with respect to two variants of concern: B.1.351 and P.1. D614G is shown as a comparator.

FIGS. 35A-35B show the neutralization of wild-type (FIG. 35A) and B.1.351 (FIG. 35B) lentivirus-based pseudoviruses by participant serum immediately before and after boosters using a clinically validated recombinant lentivirus-based SARS-CoV-2 pseudovirus assay (D614G and B.1.351). Sera was collected immediately prior to receiving a booster (D1) and on day 15 (D15) and 29 (D29) after the booster dose of 50 μg of mRNA-1273, 50 or 20 μg of mRNA-1273.351, or 50 μg mRNA-1273.211. The geometric mean neutralizing antibody titers with 95% confidence intervals are denoted. The titers for individual participants are shown by the circles. The fold increase versus titers measured versus samples collected prior to the boost are shown. The horizontal dotted lines indicate the lower limit of quantification (LLOQ). N=20 participants per booster cohort.

FIGS. 36A-36B show correlation of results from clinically validated pseudovirus assay and VSV-based pseudovirus assay used for exploratory analysis of SARS-CoV-2 variants. Neutralizing Ab titers from participant sera in the clinically validated recombinant lentivirus-based SARS-CoV-2 pseudovirus assay was correlated to a VSV-based SARS-CoV-2 pseudoviruses assay used for exploratory analysis of SARS-CoV-2 variants (D614G (FIG. 36A) and B.1.351 (FIG. 36B)). Sera was collected immediately prior to receiving a booster (D1) and 2 weeks after (D15) the booster dose of 50 μg of mRNA-1273, mRNA-1273.351, or mRNA-1273.211. The titers for individual participants are shown by the circles. For wild-type assay, the available Part B (D1) and Part C (D1 and D15) data points were used for correlation analysis. For B.1.351 assay, the available Part C (D1 and D15) data points were used. Spearman nonparametric analysis was performed for correlation analysis of both assays.

FIGS. 37A-37D show an exploratory analysis of neutralization of wild-type and variants VSV-based pseudoviruses by participant serum. Neutralization of recombinant VSV-based SARS-CoV-2 pseudoviruses (D614G, B.1.351 and P.1) by serum from participants 1 month after the primary series vaccination with 100 μg mRNA-1273, immediately prior to receiving a booster, and 2 weeks after the booster dose of 50 μg of mRNA-1273 (FIG. 37A), 50 μg of mRNA-1273.351 (FIG. 37B), 50 μg of mRNA-1273.211 (FIG. 37C), or 20 μg of mRNA-1273.351 (FIG. 37D). The groups, from left to right, in each group are D614G, B.1.351, and P.1 (as shown in FIG. 37C). The geometric mean neutralizing antibody titers with 95% confidence intervals are denoted. The titers for individual participants are shown by the circles. The GMT fold change versus the peak titers against the wild-type D614G virus after the primary vaccination series are shown. The horizontal dotted lines indicate the lower limit of quantification (LLOQ). N=20 participants per booster cohort.

FIGS. 38A-38D show the effect of B.1.617 on neutralization and wild-type D614G and VOCs neutralization post-boost versus peak D614G titers after the primary series. Sera was collected from trial participants 1 month after the primary series vaccination with 100 μg mRNA-1273, immediately prior to the booster dose, and two weeks after the 50 μg boosters. FIG. 38A shows the neutralization of D614G, B.1.617.1, and B.1.617.2 from sera collected from 11 Part B individuals immediately prior to the booster. Sera collected after the primary series or 2-weeks after the boost with mRNA-1273 (FIG. 38B), mRNA-1273.351 (FIG. 38C), or mRNA-1273.211 (FIG. 38D) were analyzed in PsVN assays. The GMT titers against the wild-type virus or variants measured in booster trial participants 2 weeks after the booster were evaluated versus peak titers measured against the wild-type virus after the primary series vaccination, and the fold change for each virus are shown. GMTs for each variant virus are listed above each graph. The D57 GMT titers is indicated by the grey line. Results from individual participants are represented as dots on each figure. N=20 for D614G, B.1.351, and P.1 assays. N=11 for B.1.427/B.1.429, B.1.526, A. VOI. V2, B.1.617.1, and B. 1.617.2, with shaded dots indicating these participants.

FIGS. 39A-39B show wild-type D614G and VOCs GMT neutralization after boosting versus COVE study GMT benchmark. Sera from trial participants two weeks after the 50 μg boost with mRNA-1273, mRNA-1273.351, or mRNA-1273.211 were analyzed for neutralizing antibody using the recombinant VSV-based SARS-CoV-2 pseudovirus assay. FIG. 39A shows GMT neutralization titers against the wild-type virus (D614G) or variants (B.1.351, P.1, B.1.427/B.1.429, B.1.526, A. VOI.2, B.1.617.1, B.1.617.2). The GMT of 1 of the wild-type D614G virus COVE study benchmark measured at D57 in the pivotal P301 mRNA-1273 efficacy trial (n=59) is indicated by the black dotted line. FIG. 39B shows GMT ratio versus the Phase 3 GMT titer comparator, measured at D57 in the pivotal P301 mRNA-1273 efficacy trial. The GMT titers against the wild-type or variant viruses measured in booster trial participants 2 weeks after the booster were evaluated versus peak titers measured against the wild-type D614G virus after the primary series vaccination. The GMT of 1 is indicated by the dotted line. N=20 for D614G, B.1.351, and P.1. N=11 for B.1.427/B.1.429, B.1.526, A. VOI. V2, B. 1.617.1, and B.1.617.2.

FIG. 40 is a graph showing that administration of a booster shot (mRNA-1273, 50 μg) increases neutralizing antibody titers against wild-type (D614) and variants of concern (B.1.351 (beta), P.1 (gamma), and B.1.617.2 (delta)) relative to two time points after the second administration of the vaccine.

FIG. 41 is a graph showing neutralizing antibodies elicited by different vaccine formulations in mice before and after administration of a booster (third) vaccine formulation.

FIGS. 42A-42B are graphs showing neutralizing antibodies elicited by different vaccine formulations in mice after administration of two doses of vaccine on day 56 (before administration of a booster dose).

FIGS. 43A-43B are graphs showing neutralizing antibodies elicited by different vaccine formulations in mice on day 77, following administration of two doses of vaccine and administration of a booster dose.

FIGS. 44A-44B are graphs showing neutralizing antibodies elicited by different vaccine formulations in mice on day 77, following administration of two doses of vaccine and administration of a booster dose. In each group, the three doses administered were the same vaccine formulation (dosage and mRNA encoding the antigen).

### DETAILED DESCRIPTION

Severe acute respiratory syndrome coronavirus 2 (SARS-CoV-2) is a newly emerging respiratory virus with high morbidity and mortality. SARS-CoV-2 has rapidly spread around the world compared with SARS-CoV, which appeared in 2002, and Middle East respiratory syndrome coronavirus (MERS-CoV), which emerged in 2012. The World Health Organization (WHO) reports that, as of March 2021, the current outbreak of COVID-19 has had over 120 million confirmed cases worldwide with more than 2.65 million deaths. New cases of COVID-19 infection are on the rise and are still increasing rapidly. It is thus crucial that a variety of safe and effective vaccines and drugs be developed to prevent and treat COVID-19 and reduce the serious impact that COVID-19 is having across the world. Vaccines and drugs made using a variety of modalities, and vaccines having improved safety and efficacy, are imperative. There remains a need to accelerate the advanced design and development of vaccines and therapeutic drugs against coronavirus disease and in particular coronavirus 2019 (COVID-19).

On Jan. 7, 2020, severe acute respiratory syndrome coronavirus 2 (SARS-CoV-2) was identified as the etiological agent of a novel pneumonia that emerged in December 2019, in Wuhan City, Hubei province in China (Lu H. et al. (2020) J Med Virol. April; 92(4): 401-402.). Soon after, the virus caused an outbreak in China and has spread to the world. According to the analysis of genomic structure of SARS-CoV-2, it belongs to β-coronaviruses (CoVs) (Chan et al. 2020 Emerg Microbes Infect.; 9(1): 221-236).

Subsequently, a number of SARS-CoV-2 variant strains have emerged and have predominated in particular initial geographic areas. However, some variants that quickly predominate in one geographic area can spread rapidly around the globe. These variants are known as variants of concern (VOC). Two main variants have been found since the fall of 2020, including one in the United Kingdom (20B/501Y.V1, VOC 202012/01, or B.1.1.7 lineage, or alpha variant) and one in South Africa (20C/501Y.V2 or B.1.351 lineage, or beta variant). The two variants emerged separately from one another, but appear to have improved transmissibility relative to the USA-WA1/2020 isolate. Further, there are concerns that these variants as well as other circulating strains and any future variants may further mutate to avoid neutralization by existing vaccines and therapeutic modalities such as antibodies. In this way, the SARS-CoV-2 variants, and any other emerging mutant SARS-CoV-2 strains, are an international health concern.

The threat of emerging mutant strains of viruses presents a significant challenge to vaccine development. The compositions disclosed herein provide a significant advance in combatting the emerging viral strains that pose a global health concern. Disclosed herein are vaccines and vaccine protocols with broad viral neutralization capabilities that reduce the threat of infection from more than one strain of virus, through single or multiple administrations of the same or different combinations of antigens from different strains. For instance, the vaccination strategies disclosed herein, in some embodiments, comprise “primary series” of vaccinations and subsequent boost(s) of SARS-CoV-2 2P stabilized spike protein antigen. The primary series (also referred to herein as initial, original or first vaccine, vaccination) involves the administration of one or more vaccines (e.g. two vaccine administrations) of the SARS-CoV-2 2P stabilized spike protein antigen from the originally identified strain of SARS-CoV-2. The primary series of vaccine may be an mRNA vaccine encoding an antigen having an amino acid sequence of SEQ ID NO: 20. A subsequent booster or booster series of vaccines is then administered, for instance, shortly after the original vaccine or at a significantly later time in the vaccination protocol (e.g., after neutralizing Ab titers have dropped or after approval of a new strain vaccine.

In aspects disclosed herein, emerging SARS-CoV-2 variant strains are used to design mRNA “boost” as a supplement to prior administered SARS-CoV-2 vaccines and includes traditional boosts, seasonal boosts and pandemic shift boosts. A boost, as used herein refers to any subsequent dose. A traditional boost is a second dose of an antigen administered to a subject following a period of time, such as 21-28 days or even 2 weeks to 6 months. The traditional boost involves the administration of the same antigen representing the same virus strain to the subject in order to generate a robust immune response against that viral strain and optionally other variant strains.

During a pandemic or endemic, emerging viral strains may develop which are not effectively susceptible to neutralization with a vaccine designed against the original strain. In particular, SARS-CoV-2 emerging viral strains appear to arise through radial evolution; that is, with a variety of different mutations, as compared to linear evolution, in which mutations accumulate upon one another as the virus evolves. In such instances, a pandemic shift boost may be used to provide immune protection against emerging viral strains. A pandemic shift boost is a subsequent vaccine which is administered to a subject following a complete course of a first vaccine. The complete course of the first vaccine may comprise one or more administrations of the first vaccine. The pandemic shift boost is comprised of a vaccine that includes an antigen which is derived from a variant viral strain that has emerged during a pandemic or endemic of the viral infection. The pandemic shift boost may be administered at any time following the administration of the first vaccine. The first vaccine may be a vaccine against the originally detected strain of the virus, a combination of the original strain of the virus and variant strain(s) of the virus, or variant strains of the virus, as long as the pandemic shift boost comprises a vaccine against a different variant strain of the virus from the first vaccine.

Additionally variant viral strains of SARS-CoV-2 may emerge at times outside of a pandemic or endemic. These strains may emerge, for instance, seasonally. Such variant strains may be used to design seasonal SARS-CoV2 vaccines which as delivered as a seasonal boost. A seasonal boost is a subsequent vaccine which is administered to a subject following a complete course of a first vaccine which happens outside of a pandemic or endemic, as variant strains arise. Viral surveillance methods are used in the design of traditional vaccines. However, due to the slow development time of traditional vaccines, the antigen design decisions are often made so far in advance that the vaccine does not match the viral strains circulating when the vaccines are administered. During the development period the viruses may mutate, or other strains may become more prevalent, such that the traditional vaccines become less effective. The traditional vaccines cannot adapt because they are already in production, and it would take additional time to design and manufacture a new vaccine. In contrast, the mRNA vaccines described herein are able to overcome these challenges. They can be produced in a matter of weeks, so that they can be designed against the coronaviruses circulating closer to the inoculation date. For instance, a seasonal or annual coronavirus vaccination program can be developed that rapidly develops a coronavirus vaccine in response to viral strains circulating at the time of vaccination. That is, it is thought that prediction of the viruses closer to a coronavirus season or other outbreak will be more accurate than predictions from several months before the season or the outbreak begins, and therefore the mRNA vaccines described herein will also be more effective because they are designed to target circulating viruses closer to the coronavirus season or scheduled inoculations. Thus, in exemplary aspects, the vaccines of the disclosure may be designed to combat seasonal coronavirus strains, and as such are vaccines for use in an upcoming or forthcoming Northern hemisphere season or Southern hemisphere season. Based on an understanding of circulating coronaviruses at a given point in time, the vaccines are designed to combat such viruses as they are predicted to be those that will be circulating or prevalent in the upcoming or forthcoming virus season. The mRNA vaccines can be designed in a matter of days and a recent vaccine developed by applicant preceded from design to manufactured vaccine in just over 5 weeks. Data can be captured and analyzed as to what viruses are circulating and with what prevalence, much closer to the start of an inoculation program such as seasonal vaccination.

A key protein on the surface of coronavirus, including the SARS-CoV-2 and mutant strains described herein, is the Spike(S) protein. A stabilized version of the spike protein having a two proline mutation relative to wild type SARS-CoV-2 has been developed and has an amino acid sequence of SEQ ID NO: 20. The 2P stabilized spike antigen is a full length spike protein including the 2Ps. The vaccination protocols described herein comprise various vaccines of full length 2P stabilized spike protein from the original SARS-CoV-2 strain and/or emerging variant SARS-CoV-2 strains, wherein each antigen includes the 2P mutation.

A variety of mRNA constructs have been designed and are disclosed herein. When formulated in appropriate delivery vehicles mRNA encoding a 2P stabilized version of the spike antigen of emerging variant strains are capable of inducing a strong immune response against SARS-CoV-2, thus producing effective and potent mRNA vaccines/boosters to provide the diversity essential to eradicating the original virus as well as subsequent strains. Intramuscular administration of the mRNA encoding various Spike protein antigens in an LNP, in particular, Spike protein subunit and domain antigens, results in delivery of the mRNA to immune tissues and cells of the immune system where it is rapidly translated into proteins antigens. Other immune cells, for example, B cells and T cells, are then able to recognize and mount an immune response against the encoded protein and ultimately create a long-lasting protective response against the coronavirus. Low immunogenicity, a drawback in protein vaccine development due to poor presentation to the immune system or incorrect folding of the antigens, is avoided through the use of the highly effective mRNA vaccines encoding spike protein, subunits and domains thereof disclosed herein.

Due to the constant evolving nature of viruses, scientists continuously monitor the sequences and strains of viruses circulating in humans. These various circulating strains may be used as boosts or individual vaccines as disclosed herein, or additionally to design multivalent mRNA vaccines. Viral surveillance can be used to provide annual or seasonal (or other scheduled) information to select the precise virus strains to be used as the basis of mRNA vaccines. Once circulating strains are identified, the composition of a vaccine that targets two or three (or more) most representative virus types in circulation can be developed based on those strains. This exercise of adding antigens from new strains to the vaccine can be repeated on an annual basis or other time frame as required to maintain viral immunity in the population. As used herein, “population” or “subject population” refers to the global population, a regional population, or a national population. For example, a regional population may refer to a geographically distinct population (e.g., hemisphere, continent) or a region of a country, as some new strains may be more prevalent in certain regions of the world, continents, or countries. In some embodiments, the subject population is a national population (e.g., the population of the United States). The mRNA vaccines described herein, in some embodiments, encode multiple antigens from multiple circulating strains in a single lipid nanoparticle (LNP). The mRNA vaccines comprise, in some embodiments, a combination of at least two antigens, each derived from a unique strain of coronavirus.

Thus, the present disclosure provides compositions (e.g., mRNA vaccines) that elicit potent neutralizing antibodies against coronavirus antigens in subjects. Such a composition can be administered to seropositive or seronegative subjects. A seropositive subject may be naïve and not have antibodies that react with SARS-CoV-2. A seronegative subject may have preexisting antibodies to SARS-CoV-2 because they have previously had an infection with SARS-CoV-2 or may have previously been administered a dose of a vaccine (e.g., an mRNA vaccine) that induces antibodies against SARS-CoV-2. In some embodiments, a composition includes mRNA encoding at least one (e.g., one, two, or more) coronavirus antigens, such as SARS-CoV-2 antigens from different SARS-CoV-2 mutant strains (also referred to herein as variants). In some embodiments, the mRNA vaccine comprises multiple mRNAs encoding SARS-CoV-2 antigens from different variants in a single lipid nanoparticle. In some embodiments, the mRNA vaccine comprises an mRNA encoding a SARS-CoV-2 antigen comprising one or more mutations from at least two different SARS-CoV-2 variants (e.g., encoding a combination of the mutations and/or deletions found in the B.1.1.7 and 5021. V2 variants).

At least four groups of SARS-CoV-2 mutants are currently of concern due to increasing prevalence, higher hACE2 binding affinity, or reported escape from mAb and convalescent sera. One exemplary circulating strain (UK) of coronavirus is N501Y-UK, or B.1.1.7 (alpha variant), which has the following mutations: ΔH69-ΔV70-ΔY144-N501Y-A570D-P681H-T716I-S982A-D1118H. This strain has been observed to spread quickly through a region. N501Y causes increased binding affinity to hACE2, making viral uptake more likely. ΔH69-ΔV70 has been shown to have reduced sensitivity to convalescent sera and P681H locates immediately adjacent to furin cleavage site.

Another strain (South Africa), N501Y-SA (B.1.351) (beta variant) with a K417N-E484K-N501Y mutation has also shown fast regional spread and higher viral load in patients. E484K has been shown to have reduced sensitivity to convalescent sera. Both N501Y and E484K are located in the receptor binding domain (RBD) and these mutations increase RBD binding affinity to hACE2.

An additional strain, identified in Japan from four people traveling from Brazil, P.1 (B1.1.248; 201/501Y.V1) (gamma variant) has emerged. This variant contains 12 mutations in its spike protein, including N501U and E484K. It is thought that the further mutations that may affect its ability to be recognized by antibodies and it is thought to be more transmissible than the wild-type virus (USA-WA1/2020 isolate).

An additional subclade of B.1.1.248 has emerged in Amazonas state, Brazil to cause concern over re-infection of people previously infected. The two subclades of B.1.1.28 are designated P.2 (alias of B.1.1.28.2) and P.1 (alias of B.1.1.28.1). To be clear, the variant of concern (VOC) is the subclade designated P.1 (alias of B.1.1.28.1) that has caused a noted re-infection of a woman previously infected and who previously had recovered. The reinfection may be the result of limited or transitory immunity induced in the initial infection or it may reflect a superior ability of the new strain to evade previous immune responses. This new strain contains 12 spike protein mutations including 3 in the RBD (K417T, E484K, N501Y) and one new N-glycosylation site at T20N. The S protein mutations include the following 12 mutations: L18F, T20N, P26S, D138Y, R190S, K417T, E484K, N501Y, D614G, H655Y, T1027I, V1176F (see Naveca et al., SARS-CoV-2 reinfection by the new Variant of Concern (VOC) P.1 in Amazonas, Brazil, 2021). The reinfection caused similar moderate symptoms as the initial infection, but recorded a higher viral load in nasopharyngal and pharyngeal samples. The reinfection may be the result of the E484K mutation in the spike protein and its ability to facilitate evasion of SARS-CoV-2 neutralizing antibodies.

The B.1.429 (also called CAL.20C or 542R. V1) strain (epsilon variant) was found at Cedars-Sinai Medical Center in Los Angeles. The variant contains five mutations: I4205V (ORF1a), D1183Y (ORF1b), and S13I, W152C, and L452R (spike protein) (Zhang et al., medRxiv preprint, Jan. 20, 2021). The L452R mutation is located within the RBD and has been found to be resistant to certain monoclonal antibodies against the spike protein.

In Germany, a new variant has been detected in 35 out of 73 new patients in Garmisch-Partenkirchen. The variant is currently being sequenced, although at least one point mutation has been detected in the spike protein.

In India, two related variants, both belonging to the B.1.617.1 subclade, have emerged. The genome of one B.1.617.1 variant, referred to as v1, or B.1.617.1 v1 (kappa variant), encodes a Spike protein having the following 8 substitutions: T95I, G142D, E154K, L452R, E484Q, D614G, P681R, and Q1071H. The genome of the other B.1.617.1 variant, referred to as v2, or B.1.617.1 v2, encodes a Spike protein with the following 8 substitutions: G142D, E154K, L452R, E484Q, D614G, P681R, Q1071H, and H1101D.

Also in India, another variant, belonging to the B.1.617.2 subclade, has emerged. The genome of the B.1.617.2 (Delta) variant encodes a Spike protein having the following ten substitutions: T19R, G142D, E156G, F157, R158, L452R, T478K, D614G, P681R, D950N, in addition to two deletions: F157del and R158del.

In Angola, a new variant, referred to as A. VOI.V2, with multiple Spike protein mutations has been detected through genomic surveillance. The genome of the A. VOI.V2 variant encodes a Spike protein having the following 15 mutations, including 10 substitutions and 5 amino acid deletions: D80Y, ΔY144, ΔI210, D215G, ΔR246, ΔS247, ΔY248, L249M, W258L, R346K, T478R, E484K, H655Y, P681H, and Q957H.

A variant of interest (VOI), lambda (C.37) has been investigated. The variant was first documented in Peru and is most frequently found in South America. It has relatively high risk scores, due mainly to its high number of deletions in the N-terminal domain (NTD) and it is possible that its RSYLTPGD246-253N mutation may increase its ability to evade neutralizing antibodies.

Another variant of interest (VOI), mu (B.1621) has been reported and first documented in Colombia. The variant comprises an insertion, 146N, and several amino acid substitutions in the Spike protein (Y144T, Y145S, R346K, E484K, N501Y and P681H).

Two additional related variants, B.1.243 and B.1.243.1 have been found primarily in North America (Arizona). The B.1.243.1 variant has the E484K mutation, in addition to a further Spike protein mutation (V213G), which may render it more resistant to neutralizing antibodies.

A variant of concern, Omicron (B.1.1.529), having multiple Spike protein mutations was detected initially in Botswana. The mutations observed in the variant include those found in the Delta variant that are believed to increase transmissibility and mutations, and those seen in the Beta and Delta variants that are believed to promote immune escape. In particular, the genome of the Omicron variant encodes a Spike protein having the following mutations: A67V, Δ69-70, T95I, G142D/Δ143-145, Δ211/L212I, ins214EPE, G339D, S371L, S373P, S375F, K417N, N440K, G446S, S477N, T478K, E484A, Q493K, G496S, Q498R, N501Y, Y505H, T547K, D614G, H655Y, N679K, P681H, N764K, D796Y, N856K, Q954H, N969K, and L981F.

These exemplary strains and other newly emerging strains are candidates for the methods and formulations disclosed herein. mRNA encoding antigens from these and other coronavirus strains have been designed for mRNA vaccines.

In some embodiments, the mRNA vaccines described herein may be administered as a prime or priming immunization (e.g., the first administration of a coronavirus vaccine to a subject). In some embodiments, the mRNA vaccines described herein may be administered as a booster, that is, a dose administered after the prime or priming immunization, as described herein. In some embodiments, the booster and the prime or priming immunization comprise the same mRNA or mRNAs. In other embodiments, the booster and the prime or priming immunization comprise different mRNA or mRNAs. In other embodiments multiple mRNA vaccines encoding different antigens (each directed at a strain or multiple strains) may be administered together or in tandem to provide a wide spectrum neutralization platform against multiple coronavirus strains. Combinations of mRNAs have been demonstrated to be particularly effective in vivo and, quite surprisingly, even producing robust immune responses against variant strains that are not part of the vaccine. For instance, it was shown that when a multivalent mRNA-vaccine was administered as a booster it elicited robust and comparable neutralizing titers against both variant strains of the viruses not included in the prime or boost.

The genome of SARS-CoV-2 is a single-stranded positive-sense RNA (+ssRNA) with the size of 29.8-30 kb encoding about 9860 amino acids (Chan et al. 2000, supra; Kim et al. 2020 Cell, May 14; 181(4): 914-921.e10.). SARS-CoV-2 is a polycistronic mRNA with 5′-cap and 3′-poly-A tail. The SARS-CoV-2 genome is organized into specific genes encoding structural proteins and nonstructural proteins (Nsps). The order of the structural proteins in the genome is 5′-replicase (open reading frame (ORF) 1/ab)-structural proteins [Spike(S)-Envelope (E)-Membrane (M)-Nucleocapsid (N)]-3′. The genome of coronaviruses includes a variable number of open reading frames that encode accessory proteins, nonstructural proteins, and structural proteins (Song et al. 2019 Viruses; 11(1): p. 59). Most of the antigenic peptides are located in the structural proteins (Cui et al. 2019 Nat. Rev. Microbiol.; 17(3): 181-192). Spike surface glycoprotein(S), a small envelope protein (E), matrix protein (M), and nucleocapsid protein (N) are four main structural proteins. Since S-protein contributes to cell tropism and virus entry and also it is capable to induce neutralizing antibodies (NAb) and protective immunity, it can be considered one of the most important targets in coronavirus vaccine development among all other structural proteins. Moreover, amino acid sequence analysis has shown that S-protein contains conserved regions among the coronaviruses, which may be the basis for universal vaccine development

The compositions of the invention, e.g., vaccine compositions, feature nucleic acids, in particular, mRNAs, designed to encode an antigen of interest, e.g., an antigen derived from a betacoronavirus structural protein, in particular, antigens derived from SARS-CoV-2 Spike protein. The compositions of the invention, e.g., vaccine compositions, do not comprise antigens per se, but rather comprise nucleic acids, in particular, mRNA(s) that encode antigens or antigenic sequences once delivered to a cell, tissue or subject. Delivery of nucleic acids, in particular mRNA(s) is achieved by formulating said nucleic acids in appropriate carriers or delivery vehicles (e.g., lipid nanoparticles) such that upon administration to cells, tissues or subjects, nucleic acid is taken up by cells which, in turn, express protein(s) encoded by the nucleic acids, e.g., mRNAs.

Antigens, as used herein, are proteins capable of inducing an immune response (e.g., causing an immune system to produce antibodies against the antigens). The vaccines of the present disclosure provide a unique advantage over traditional protein-based vaccination approaches in which protein antigens are purified or produced in vitro, e.g., recombinant protein production technologies. The vaccines of the present disclosure feature mRNA encoding the desired antigens, which when introduced into the body, i.e., administered to a mammalian subject (for example a human) in vivo, cause the cells of the body to express the desired antigens. In order to facilitate delivery of the mRNAs of the present disclosure to the cells of the body, the mRNAs are encapsulated in lipid nanoparticles (LNPs). Upon delivery and uptake by cells of the body, the mRNAs are translated in the cytosol and protein antigens are generated by the host cell machinery. The protein antigens are presented and elicit an adaptive humoral and cellular immune response. Neutralizing antibodies are directed against the expressed protein antigens and hence the protein antigens are considered relevant target antigens for vaccine development. Herein, use of the term “antigen” encompasses immunogenic proteins and immunogenic fragments (an immunogenic fragment that induces (or is capable of inducing) an immune response to a (at least one) SARS-CoV-2 variant), unless otherwise stated. It should be understood that the term “protein” encompasses peptides and the term “antigen” encompasses antigenic fragments. Other molecules may be antigenic such as bacterial polysaccharides or combinations of protein and polysaccharide structures, but for the viral vaccines included herein, viral proteins, fragments of viral proteins and designed and or mutated proteins derived from SARS-CoV-2 are the antigens provided herein.

Many proteins have a quaternary or three-dimensional structure, which consists of more than one polypeptide or several polypeptide chains that associate into an oligomeric molecule. As used herein the term “subunit” refers to a single protein molecule, for example, a polypeptide or polypeptide chain resulting from processing of a nascent protein molecule, which subunit assembles (or “coassembles”) with other protein molecules (e.g., subunits or chains) to form a protein complex. Proteins can have a relatively small number of subunits and therefore be described as “oligomeric” or can consist of a large number of subunits and therefore be described as “multimeric”. The subunits of an oligomeric or multimeric protein may be identical, homologous or totally dissimilar and dedicated to disparate tasks.

Proteins or protein subunits can further comprise domains. As used herein, the term “domain” refers to a distinct functional and/or structural unit within a protein. Typically, a “domain” is responsible for a particular function or interaction, contributing to the overall role of a protein. Domains can exist in a variety of biological contexts. Similar domains (i.e., domains sharing structural, functional and/or sequence homology) can exist within a single protein or can exist within distinct proteins having similar or different functions. A protein domain is often a conserved part of a given protein tertiary structure or sequence that can function and exist independently of the rest of the protein or subunit thereof.

In structural and molecular biology, identical, homologous or similar subunits or domains can help to classify newly identified or novel proteins, as was done immediately upon publication of the SARS-CoV-2 viral genomic sequence.

As used herein, the term antigen is distinct from the term “epitope” which is a substructure of an antigen, e.g., a polypeptide, such as 7-10 amino acids, or carbohydrate structure, which may be recognized by an antigen binding site but is insufficient to induce an immune response. The art describes protein antigens that are delivered to subjects or immune cells in isolated form, e.g., isolated protein, polypeptide or peptide antigens, however, the design, testing, validation, and production of protein antigens can be costly and time-consuming, especially when producing proteins at large scale. By contrast, mRNA technology is amenable to rapid design and testing of mRNA constructs encoding a variety of antigens. Moreover, rapid production of mRNA coupled with formulation in appropriate delivery vehicles (e.g., lipid nanoparticles), can proceed quickly and can rapidly produce mRNA vaccines at large scale. Potential benefit also arises from the fact that antigens encoded by the mRNAs of the invention are expressed by the cells of the subject, e.g., are expressed by the human body, and thus the subject, e.g., the human body, serves as the “factory” to produce the antigens which, in turn, elicits the desired immune response.

The compositions, as provided herein, may include an RNA or multiple RNAs encoding two or more antigens of the same or different viral strains. Also provided herein are combination vaccines that include RNA encoding one or more coronavirus antigens and one or more antigen(s) of a different organism. Thus, the vaccines of the present disclosure may be combination vaccines that target one or more antigens of the same strain/species, or one or more antigens of different strains/species, e.g., antigens which induce immunity to organisms which are found in the same geographic areas where the risk of coronavirus infection is high or organisms to which an individual is likely to be exposed to when exposed to a coronavirus (e.g., COVID-19).

In some embodiments, the second or subsequent circulating SARS-CoV-2 virus is an immunodominant antigen from an emerging strain. An immunodominant antigen of an emerging strain is assessed with respect to the strain from which the antigen is derived, relative to a different strain of the virus, such as the original strain or other variant thereof. An immunodominant antigen of the emerging strain induces a stronger immune response against the emerging strain than against the different strain. In some embodiments, an immunodominant antigen of the emerging strain is more infective than a different strain of the virus, such as the original strain or other variant thereof.

### Encoded Coronavirus Spike(S) Protein Antigens

The envelope spike(S) proteins of known betacoronaviruses determine the virus host tropism and entry into host cells. Coronavirus spike(S) protein is a choice antigen for the vaccine design as it can induce neutralizing antibodies and protective immunity. S protein is critical for SARS-CoV-2 infection. The organization of the S protein is similar among betacoronaviruses, such as SARS-CoV-2, SARS-CoV, MERS-CoV, HKU1-CoV, MHV-CoV and NL63-CoV.

As used herein, the term “Spike protein” refers to a glycoprotein that that forms homotrimers protruding from the envelope (viral surface) of viruses including betacoronaviruses. Trimerized Spike protein facilitates entry of the virion into a host cell by binding to a receptor on the surface of a host cell followed by fusion of the viral and host cell membranes. The S protein is a highly glycosylated and large type I transmembrane fusion protein that is made up of 1,160 to 1,400 amino acids, depending upon the type of virus. Betacoronavirus Spike proteins comprise between about 1100 to 1500 amino acids.

SARS-CoV-2 spike(S) protein is a choice antigen for the vaccine design as it can induce neutralizing antibodies and protective immunity. mRNAs of the invention are designed to produce SARS-CoV-2 Spike proteins (i.e., encode Spike proteins such that Spike protein is expressed when the mRNA is delivered to a cell or tissue, for example a cell or tissue in a subject), as well as antigenic variants thereof. The skilled artisan will understand that, while an essentially full length or complete Spike protein may be necessary for a virus, e.g., a betacoronavirus, to perform its intended function of facilitating virus entry into a host cell, a certain amount of variation in Spike protein structure and/or sequence is tolerated when seeking primarily to elicit an immune response against Spike protein. For example, minor truncation, e.g., of one to a few, possibly up to 5 or up to 10 amino acids from the N- or C-terminus of the encoded Spike protein, e.g., encoded Spike protein antigen, may be tolerated without changing the antigenic properties of the protein. Likewise, variation (e.g., conservative substitution) of one to a few, possibly up to 5 or up to 10 amino acids (or more) of the encoded Spike protein, e.g., encoded Spike protein antigen, may be tolerated without changing the antigenic properties of the protein. In some embodiments, the Spike protein is a stabilized Spike protein, for example, the Spike protein is stabilized by two proline substitutions (a 2P mutation).

In some embodiments, the Spike protein is from a different virus strain. A strain is a genetic variant of a microorganism (e.g., a virus). New viral strains can be created due to mutation or swapping of genetic components when two or more viruses infect the same cell in nature, for example, by antigenic drift or antigenic shift.

Antigenic drift is a kind of genetic variation in viruses, arising by the accumulation of mutations in the virus genes that code for virus-surface proteins that host antibodies recognize. This results in a new strain of virus particles that is not effectively inhibited by the antibodies that prevented infection by previous strains. This makes it easier for the changed virus to spread throughout a partially immune population.

Antigenic shift is the process by which two or more different strains of a virus, or strains of two or more different viruses, combine to form a new subtype having a mixture of the surface antigens of the two or more original strains. The term is often applied specifically to influenza, as that is the best-known example, but the process is also known to occur with other viruses. Antigenic shift is a specific case of reassortment or viral shift that confers a phenotypic change. Antigenic shift is contrasted with antigenic drift, which is the natural mutation over time of known strains of a virus which may lead to a loss of immunity, or in vaccine mismatch. Antigenic shift is often associated with a major reorganization of viral surface antigens, resulting in a reassortment change the virus's phenotype drastically.

A virus strain as used herein is a genetic variant or of a virus that is characterized by a differing isoform of one or more surface proteins of the virus. In the case of SARS-CoV-2, for example, a different amino acid sequence in the SARS-CoV-2 spike protein where the immune response in an individual to the new strain is less effective than to the strain used to immunize or first infect the individual. A new virus strain may arise from natural mutation or a combination of natural mutation and immune selection due to an ongoing immune response in an immunized or previously infected individual. A new virus strain can differ by one, two, three or more amino acid mutations in regions of the spike protein responsible for a viral function such as receptor binding or viral fusion with a target cell. A spike protein from a new strain may differ from the parental strain by as much as 90%, 91%, 92%, 93%, 94%, 95%, 96%, 97%, 98%, or 99% identity at the amino acid level.

A natural virus strain is a variant of a given virus that is recognizable because it possesses some “unique phenotypic characteristics” that remain stable (e.g., stable and heritable biological, serological, and/or molecular characters) under natural conditions. Such “unique phenotypic characteristics” are biological properties different from the compared reference virus, such as unique antigenic properties, host range (e.g., infecting a different kind of host), symptoms of disease caused by the strain, different type of disease caused by the strain (e.g., transmitted by different means), etc. A “unique phenotypic characteristic” can be detected clinically (e.g., clinical manifestations detected in a host infected with the strain) or within a comparative animal experiment in which a researcher skilled in the art of virology can distinguish between the reference control virus-infected animal and the animal infected with the alleged new strain, without knowing which animal received which virus and without having any information about the differences between the two viruses. Importantly, a virus variant with a simple difference in genome sequence is not a separate strain if there is no recognizable distinct viral phenotype. The extent of genomic sequence variation is irrelevant for the classification of a variant as a strain since a distinct phenotype sometimes arises from few mutations.

As an example, in some embodiments, the mRNA encodes an antigen from at least one virus strain variant or comprises mutations from at least one virus strain that is not wild-type SARS-CoV-2. In some embodiments, the vaccine comprises mRNA encoding a Spike protein associated with the B. 1.1.7 lineage (UK) variant (20B/501Y.V1 VOC 202012/01). The B.1.1.7 lineage variant has a mutation in the receptor binding domain (RBD) of the Spike protein at position 501, where amino acid asparagine (N) has been replaced with tyrosine (Y); an N501Y mutation. Further, the variant has a 69/70 deletion, which occurs spontaneously numerous times, leading to conformation changes in the Spike protein, a P681H mutation near the S1/S2 furin cleavage site, and a ORF8 stop codon (Q27 stop) caused by a mutation in ORF8. The 501. V2 (South Africa, SA) variant comprises multiple mutations in the Spike protein, including N501Y, and E484K, but does not have a deletion at 69/70. The E484K mutation is considered to be an “escape” mutation relative to at least one form of monoclonal antibody against SARS-CoV-2, such that it may change the antigenicity of the virus. Other mutations that have been discovered include the D614G mutation, which is thought to increase the transmission rate of the virus, and the N543Y mutation (emerged from mink farms in the Netherlands and Denmark). In some embodiments, the Spike protein comprises mutations from more than one variant (e.g., a combination of mutations found in the B.1.1.7 and 502Y.V2 variants). Table 2, below, presents examples of Spike protein mutations in SARS-CoV-2 variants.

TABLE 2
Spike mutations in SARS-CoV-2 variants

| Variant Name | Amino Acid Changes in Spike |
|---|---|
| 20A.EU1 | A222V-D614G |
| 20A.EU2 | S477N-D614G |
| N439K-D614G | N439K-D614G |
| Mink Cluster 5 Variant | ΔH69ΔV70-Y453F-D614G-I692V-M1229I |
| B.1.1.7 | ΔH69ΔV70-ΔY144-N501Y- |
| | A570D-D614G-P681H-T716I- |
| (a.k.a., 20I/501Y.V1, VOC | S982A-D1118H |
| 202012/01) | |
| B.1.1.7-E484K | ΔH69ΔV70-ΔY144-E484K- |
| | N501Y-A570D-D614G-P681H- |
| | T716I-S982A-D1118H |
| B.1.351 | L18F-D80A-D215G- |
| (a.k.a., 20H/501Y.V2) | ΔL242ΔA243ΔL244-R246I-K417N- |
| | E484K-N501Y-D614G-A701V |
| P.1 | L18F-T20N-P26S-D138Y- |
| | R190S-K417T-E484K-N501Y- |
| | D614G-H655Y-T1027I |
| B.1.429 (a.k.a., CAL.20C) | S13I, W152C, L452R, D614G |
| B.1.617.1 v1 (a.k.a., India | T95I-G142D-E154K-L452R- |
| Variant v1) | E484Q-D614G-P681R-Q1071H |
| B.1.617.1 v2 (a.k.a., India | G142D-E154K-L452R-E484Q- |
| Variant v2) | D614G-P681R-Q1071H- |
| | H1101D |
| B.1.617.2 (Delta variant; | T19R-G142D-E156G-F157del- |
| Indian) | R158del-L452R-T478K- |
| | D614G-P681R-D950N |
| A.VOI.V2 (a.k.a. Angola | D80Y-ΔY144-ΔI210-D215G- |
| Variant) | ΔR246-ΔS247-ΔY248-L249M- |
| | W258L-R346K-T478R-E484K- |
| | H655Y-P681H-Q957H |
| B.1.1.529 | A67V, Δ69-70, T95I, G142D/ |
| (Omicron variant; | Δ143-145, Δ211/L212I, |
| Botswana) | ins214EPE, G339D, S371L, |
| | S373P, S375F, K417N, N440K, |
| | G446S, S477N, T478K, E484A, |
| | Q493K, G496S, Q498R, |
| | N501Y, Y505H, T547K, D614G, |
| | H655Y, N679K, P681H, |
| | N764K, D796Y, N856K, Q954H, |
| | N969K, L981F |

In exemplary embodiments, a Spike protein, e.g., an encoded Spike protein antigen, has the amino acid sequence set forth in any one of SEQ ID NOs: 5, 8, 11, 14, 17, 23, 26, 30, 33, 36, 39, and 42. In other embodiments, a Spike protein, e.g., an encoded Spike protein antigen, has no greater than 100, no greater than 90, no greater than 80, no greater than 70, no greater than 60, no greater than 50, no greater than 40, no greater than 30, no greater than 20, no greater than 10, or no greater than 5 amino acid substitutions and/or deletions as compared to (when aligned with) a Spike protein having the amino acid sequence as set forth in any one of SEQ ID NOs: 5, 8, 11, 14, 17, 23, 26, 30, 33, 36, 39, and 42. Where minor variations are made in encoded Spike protein sequences, the variant preferably has the same activity as the reference Spike protein sequence and/or has the same immune specificity as the reference Spike protein, as determined for example, in immunoassays (e.g., enzyme-linked immunosorbent assays (ELISA assays).

S proteins of coronaviruses can be divided into two important functional subunits, of which include the N-terminal S1 subunit, which forms of the globular head of the S protein, and the C-terminal S2 region that forms the stalk of the protein and is directly embedded into the viral envelope. Upon interaction with a potential host cell, the S1 subunit will recognize and bind to receptors on the host cell, specifically angiotensin-converting enzyme 2 (ACE2) receptors, whereas the S2 subunit, which is the most conserved component of the S protein, will be responsible for fusing the envelope of the virus with the host cell membrane. (See e.g., Shang et al., PLoS Pathog. 2020 March; 16(3): e1008392.). Each monomer of trimeric S protein trimer contains the two subunits, S1 and S2, mediating attachment and membrane fusion, respectively. As part of the infection process in vivo, the two subunits are separated from each other by an enzymatic cleavage process. S protein is first cleaved by furin-mediated cleavage at the S1/S2 site in infected cells. In vivo, a subsequent serine protease-mediated cleavage event occurs at the S2′ site within S1. In SARS-CoV2, the S1/S2 cleavage site is at amino acids 676-TQTNSPRRAR/SVA-688 (SEQ ID NO: 47). The S2′ cleavage site is at amino acids 811-KPSKR/SFI-818 (SEQ ID NO: 48).

As used herein, for example in the context of designing SARS-CoV-2 S protein antigens encoded by the nucleic acids, e.g., mRNAs, of the invention, the term “S1 subunit” (e.g., S1 subunit antigen) refers to the N-terminal subunit of the Spike protein beginning at the S protein N-terminus and ending at the S1/S2 cleavage site whereas the term “S2 subunit” (e.g., S2 subunit antigen) refers to the C-terminal subunit of the Spike protein beginning at the S1/S2 cleavage site and ending at the C-terminus of the Spike protein. As described supra, the skilled artisan will understand that, while an essentially full length or complete Spike protein S1 or S2 subunit may be necessary for receptor binding or membrane fusion, respectively, a certain amount of variation in S1 or S2 structure and/or sequence is tolerated when seeking primarily to elicit an immune response against Spike protein subunits. For example, minor truncation, e.g., of one to a few, possibly up to 4, 5, 6, 7, 8, 9 or 10 amino acids from the N- or C-terminus of the encoded subunit, e.g., encoded S1 or S2 protein antigens, may be tolerated without changing the antigenic properties of the protein. Likewise, variation (e.g., conservative substitution) of one to a few, possibly up to 4, 5, 6, 7, 8, 9 or 10 amino acids (or more) of the encoded Spike protein subunits, e.g., encoded S1 or S2 protein antigen, may be tolerated without changing the antigenic properties of the protein(s). In exemplary embodiments, a Spike protein, e.g., an encoded Spike protein antigen, has the amino acid sequence set forth in any one of SEQ ID NOs: 5, 8, 11, 14, 17, 20, 23, 26, 29, 30, 32, 33, 36, 39, and 42.

In some embodiments, the mRNA vaccine encodes an antigen having at least one of the following mutations relative to the USA-WA1/2020 isolate: E484K, D614G, K417N, N501Y, L18F, D80A, D215G, R246I, A701V, A570D, P861H, T716I, S982A, D1118H. In some embodiments, the mRNA vaccine encodes an antigen having at least one of the following mutations relative to the SARS-CoV-2 S protein of SEQ ID NO: 20 (2P mutation version of WT): E484K, D614G, K417N, N501Y, L18F, D80A, D215G, R246I, A701V, A570D, P861H, T716I, S982A, D1118H. In some embodiments, the mRNA encodes an antigen having 2, 3, 4, 5, 6, 7, 8, 9, 10, 11, 12, 13, or all 14 of the mutations listed. In some embodiments, the mRNA encodes an antigen that has one or more deletions relative to the SARS-CoV-2 S protein of SEQ ID NO: 20. Exemplary deletions include, but are not limited to, positions, 69, 70, 144, and 242-244. In some embodiments, the mRNA encodes an antigen having 1, 2, 3, 4, 5, or 6 deletions. In some embodiments, the mRNA encoding an antigen has 1, 2, 3, 4, 5, or 6 deletions, and 1, 2, 3, 4, 5, 6, 7, 8, 9, 10, 11, 12, 13, or 14 mutations or any combination thereof.

In some embodiments, the mRNA vaccine comprises 1, 2, 3, 4, 5, or 6 mRNAs encoding different antigens, wherein each antigen comprises at least one mutation and/or at least one deletion. In some embodiments, the mRNA vaccine further comprises an mRNA encoding a wild-type SARS-CoV-2 S protein antigen or the antigenic fragment thereof. The mRNA vaccine, in some embodiments, is in a lipid nanoparticle (that is, the lipid nanoparticle comprises 1, 2, 3, 4, 5, or 6 mRNAs encoding different antigens).

In some aspects, compositions of the disclosure comprise at least: a first mRNA encoding a first SARS-CoV-2 spike antigen of a first SARS-CoV-2 virus wherein the first SARS-CoV-2 spike antigen has an amino acid sequence of SEQ ID NO: 20 or an amino acid sequence with at least one amino acid mutation, deletion, or both amino acid mutation and deletion with respect to a protein of SEQ ID NO: 20 and a second mRNA encoding a second SARS-CoV-2 spike antigen of a second SARS-CoV-2 virus wherein the second SARS-CoV-2 spike antigen has an amino acid sequence with at least one amino acid mutation, deletion, or both amino acid mutation and deletion with respect to a protein of SEQ ID NO: 20. In some embodiments, the first SARS-CoV-2 virus is a first circulating SARS-CoV-2 virus. In some embodiments, the second SARS-CoV-2 virus is a second circulating SARS-CoV-2 virus. In some embodiments, the first and second mRNAs are present in the composition in a 1:1, 1:2, 1:3, or 1:4 ratio. In another embodiment, the first and second mRNAs are present in the composition in a 2:1, 3:1 or 4:1 ratio. In some embodiments, the first and second mRNA are present in the composition in a 1:1 ratio. In one embodiment, the first mRNA encodes SEQ ID NO: 20 (2P mutation version of WT) and the second mRNA encodes SEQ ID NO: 11 (mRNA-1273.351; WH2020_NatSP_2P_L18F_D80A_D215G_L242_244del_R246I_K417N_E484K_N5 01Y_D614G_A701V). In one embodiment, the first mRNA encodes SEQ ID NO: 20 (2P mutation version of WT) and the second mRNA encodes SEQ ID NO: 26 (S2P_IN_B.1.617.2; now known as the Delta variant and comprising the following mutations: T19R, G142D, E156G, F157del, R158del, L452R, T478K, D614G, P681R, and D950N). In one embodiment, the first mRNA encodes SEQ ID NO: 20 (2P mutation version of WT) and the second mRNA encodes SEQ ID NO: 30 (S2P_IN_B.1.617.2; comprising the following mutations: T19R, T95I, G142D, E156G, F157del, R158del, L452R, T478K, D614G, P681R, D950N). “Circulating viruses”, as used herein, refers to viruses that have been in circulation for 1 month, 2 months, 3 months, 4 months, 5 months, 6 months, 7 months, 8 months, 9 months, 10 months, 11 months, a portion of a year, 1 year, 1.5 years, 2 years, 3 years, or longer.

In some embodiments, the composition further comprises a third mRNA encoding a third SARS-CoV-2 spike antigen of a third SARS-CoV-2 virus, wherein the third SARS-CoV-2 spike antigen has an amino acid sequence with at least one amino acid mutation with respect to a protein sequence of SEQ ID NO: 20.

In some embodiments, the composition further comprises a fourth mRNA encoding a fourth SARS-CoV-2 spike antigen of a fourth SARS-CoV-2 virus, wherein the fourth SARS-CoV-2 spike antigen has an amino acid sequence with at least one amino acid mutation with respect to a protein of SEQ ID NO: 20.

In some embodiments, the composition further comprises a fifth mRNA encoding a fifth SARS-CoV-2 spike antigen of a fifth SARS-CoV-2 virus, wherein the fifth SARS-CoV-2 spike antigen has an amino acid sequence with at least one amino acid mutation with respect to a protein of SEQ ID NO: 20.

In some embodiments, the composition further comprises a sixth mRNA encoding a sixth SARS-CoV-2 spike antigen of a sixth SARS-CoV-2 virus, wherein the sixth SARS-CoV-2 spike antigen has an amino acid sequence with at least one amino acid mutation with respect to a protein of SEQ ID NO: 20.

In some embodiments, the first and second antigens are antigens of the spike protein. In some embodiments, the third, fourth, fifth, and/or sixth antigens are antigens of the spike protein.

In some embodiments, the mRNAs are present in the composition in an equal amount (e.g., a 1:1 weight/weight ratio or a 1:1 molar ratio), for example, a ratio of 1:1 (:1:1:1:1) of mRNA encoding distinct coronavirus antigens. As used herein, a “weight/weight ratio” or wt/wt ratio or wt:wt ratio refers to the ratio between the weights (masses) of the different components. A “molar ratio” refers to the ratio between different components (e.g., the number of mRNA encoding each antigen). In some embodiments, the ratio is 1:1, 1:2, 1:3, 1:4, 2:1, 3:1, or 4:1. In each embodiment or aspect of the invention, it is understood that the featured vaccines include the mRNAs encapsulated within LNPs. While it is possible to encapsulate each unique mRNA in its own LNP, the mRNA vaccine technology enjoys the significant technological advantage of being able to encapsulate several mRNAs in a single LNP product. In other embodiments the vaccines are separate vaccines that are not co-formulated, but may be admixed separately before administration or simply administered separately.

Exemplary sequences of the coronavirus antigens and the RNA encoding the coronavirus antigens of the compositions of the present disclosure (e.g., SARS-CoV-2 variant antigens) are provided in Table 1. In some embodiments, the mRNA vaccines comprise a sequence that has at least 90%, 91%, 92%, 93%, 94%, 95%, 96%, 97%, 98%, or 99% identity to a sequence selected from SEQ ID NOs: 1, 6, 9, 12, 15, 21, 24, 28, 31, 34, 37, 40, 43, and 45. In some embodiments, the mRNA vaccines encode a polypeptide that is 90%, 91%, 92%, 93%, 94%, 95%, 96%, 97%, 98%, 99% or identical to a sequence selected from SEQ ID NOs: 5, 8, 11, 14, 17, 23, 26, 30, 33, 36, 39, and 42.

### Nucleic Acids

The compositions of the present disclosure comprise a (at least one) messenger RNA (mRNA) having an open reading frame (ORF) encoding a coronavirus antigen. In some embodiments, the mRNA further comprises a 5′ UTR, 3′ UTR, a poly(A) tail and/or a 5′ cap analog.

It should also be understood that the coronavirus vaccine of the present disclosure may include any 5′ untranslated region (UTR) and/or any 3′ UTR. Exemplary UTR sequences include SEQ ID NOs: 2, 4, 50, and 51; however, other UTR sequences may be used or exchanged for any of the UTR sequences described herein. In some embodiments, a 5′ UTR of the present disclosure comprises a sequence selected from SEQ ID NO: 50 (GGGAAAUA AGAGAGAAAAGAAGAGUAAGAAGAAAUAUAAGAGCCACC) and SEQ ID NO: 2 (GGGAAAUAAGAGAGAAAAGAAGAGUAAGAAGAAAUAUAAGACCCCGGCGCCGC CACC). In some embodiments, a 3′ UTR of the present disclosure comprises a sequence selected from SEQ ID NO: 51 (UGAUAAUAGGCUGGAGCCUCGGUGGCCAUGCUU CUUGCCCCUUGGGCCUCCCCCCAGCCCCUCCUCCCCUUCCUGCACCCGUACCCCCG UGGUCUUUGAAUAAAGUCUGAGUGGGCGGC) and SEQ ID NO: 4 (UGAUAA UAGGCUGGAGCCUCGGUGGCCUAGCUUCUUGCCCCUUGGGCCUCCCCCCAGCCCC UCCUCCCCUUCCUGCACCCGUACCCCCGUGGUCUUUGAAUAAAGUCUGAGUGGGC GGC). UTRs may also be omitted from the RNA polynucleotides provided herein.

Nucleic acids comprise a polymer of nucleotides (nucleotide monomers). Thus, nucleic acids are also referred to as polynucleotides. Nucleic acids may be or may include, for example, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) and/or chimeras and/or combinations thereof.

Messenger RNA (mRNA) is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents mRNA, the “T”'s would be substituted for “U”s. Thus, any of the DNAs disclosed and identified by a particular sequence identification number herein also disclose the corresponding mRNA sequence complementary to the DNA, where each “T” of the DNA sequence is substituted with “U.”

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in an RNA polynucleotide of the present disclosure.

### Variants

In some embodiments, the compositions of the present disclosure include RNA that encodes a SARS-CoV-2 antigen variant. Antigen variants or other polypeptide variants refers to molecules that differ in their amino acid sequence from a wild-type, native, or reference sequence. The antigen/polypeptide variants may possess substitutions, deletions, and/or insertions at certain positions within the amino acid sequence, as compared to a native or reference sequence. Examples of SARS-CoV-2 antigen variants are provided in Table 1. Ordinarily, variants possess at least 50% identity to a wild-type, native or reference sequence. In some embodiments, variants share at least 90% identity with a wild-type, native, or reference sequence. In some embodiments, the nucleic acid vaccines described herein encode SARS-CoV-2 variants comprising 1, 2, 3, 4, or more mutations relative to a reference sequence. In some embodiments, the nucleic acid vaccines described herein encode SARS-CoV-2 variants comprising less than 20, 18, 15, 12, or 10 mutations relative to a reference sequence. In some embodiments, the nucleic acid vaccines described herein encode SARS-CoV-2 variants having 1-50 1-40, 1-30, 1-25, 1-20, 1-15, 1-10, 5-50, 5-40, 5-30, 5-25, 5-20, 5-15, 5-10, 10-50, 10-40, 10-30, 10-25, 10-20, 10-15, 20-50, 20-40, 20-30, 20-25, 25-50, 25-40, 25-30, 30-50, 30-40, 40-50 mutations (e.g., substitutions). As used herein, “mutation” refers to an amino acid substitution, insertion, or deletion. A reference sequence refers to a naturally-occurring strain, for example, a naturally-occurring circulating strain of SARS-CoV-2.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

In some embodiments, a composition comprises an RNA or an RNA ORF that comprises a nucleotide sequence of any one of the sequences provided herein, or comprises a nucleotide sequence at least 90%, at least 95%, at least 96%, at least 97%, at least 98%, or at least 99% identical to a nucleotide sequence of any one of the sequences provided herein.

The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding peptides or polypeptides containing substitutions, insertions and/or additions, deletions and covalent modifications with respect to reference sequences, in particular the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an mRNA vaccine.

As recognized by those skilled in the art, protein fragments, functional protein domains, and homologous proteins are also considered to be within the scope of coronavirus antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to the coronavirus. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

### Stabilizing Elements

Naturally-occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, a composition includes an RNA polynucleotide having an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′)G [the ARCA cap]; G (5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, a composition includes a stabilizing element. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, an mRNA includes a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine: guanine phosphoribosyl transferase (GPT)).

In some embodiments, an mRNA includes the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence.

In some embodiments, an mRNA does not include a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

An mRNA may or may not contain an enhancer and/or promoter sequence, which may be modified or unmodified or which may be activated or inactivated. In some embodiments, the histone stem-loop is generally derived from histone genes and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, an mRNA has one or more AU-rich sequences removed. These sequences, sometimes referred to as AURES are destabilizing sequences found in the 3′UTR. The AURES may be removed from the RNA vaccines. Alternatively, the AURES may remain in the RNA vaccine.

### Signal Peptides

In some embodiments, a composition comprises an mRNA having an ORF that encodes a signal peptide fused to the coronavirus antigen. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than coronavirus antigens in nature) are known in the art and can be tested for desired properties and then incorporated into a nucleic acid of the disclosure.

### Scaffold Moieties

The mRNA vaccines as provided herein, in some embodiments, encode fusion proteins that comprise coronavirus antigens linked to one another or scaffold moieties. In some embodiments, such scaffold moieties impart desired properties to an antigen encoded by a nucleic acid of the disclosure. For example, scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In some embodiments, viral proteins or virus-like particles can be used to form stable nanoparticle structures. Examples of such viral proteins are known in the art. For example, in some embodiments, the scaffold moiety is a hepatitis B surface antigen (HBsAg). HBsAg forms spherical particles with an average diameter of ˜22 nm and which lacked nucleic acid and hence are non-infectious (Lopez-Sagaseta, J. et al. Computational and Structural Biotechnology Journal 14 (2016) 58-68). In some embodiments, the scaffold moiety is a hepatitis B core antigen (HBcAg) self-assembles into particles of 24-31 nm diameter, which resembled the viral cores obtained from HBV-infected human liver. HBcAg produced in self-assembles into two classes of differently sized nanoparticles of 300 Å and 360 Å diameter, corresponding to 180 or 240 protomers. In some embodiments, the coronavirus antigen is fused to HBsAG or HBcAG to facilitate self-assembly of nanoparticles displaying the coronavirus antigen.

In some embodiments, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K.J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Lawson D. M. et al. Nature. 1991; 349:541-544). Ferritin self-assembles into nanoparticles with robust thermal and chemical stability. Thus, the ferritin nanoparticle is well-suited to carry and expose antigens.

Lumazine synthase (LS) is also well-suited as a nanoparticle platform for antigen display. LS, which is responsible for the penultimate catalytic step in the biosynthesis of riboflavin, is an enzyme present in a broad variety of organisms, including archaea, bacteria, fungi, plants, and eubacteria (Weber S.E. Flavins and Flavoproteins. Methods and Protocols, Series: Methods in Molecular Biology. 2014). The LS monomer is 150 amino acids long and consists of beta-sheets along with tandem alpha-helices flanking its sides. A number of different quaternary structures have been reported for LS, illustrating its morphological versatility: from homopentamers up to symmetrical assemblies of 12 pentamers forming capsids of 150 Å diameter. Even LS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15:939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280:2097-2104).

In some embodiments, an RNA of the present disclosure encodes a coronavirus antigen fused to a foldon domain. The foldon domain may be, for example, obtained from bacteriophage T4 fibritin (see, e.g., Tao Y, et al. Structure. 1997 Jun. 15; 5(6): 789-98).

### Linkers and Cleavable Peptides

In some embodiments, the mRNAs of the disclosure encode more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLoS ONE 6: e18556). In some embodiments, the linker is an F2A linker. In some embodiments, the linker is a GGGS (SEQ ID NO: 49) linker. In some embodiments, the fusion protein contains three domains with intervening linkers, having the structure: domain-linker-domain-linker-domain.

Cleavable linkers known in the art may be used in connection with the disclosure. Exemplary such linkers include: F2A linkers, T2A linkers, P2A linkers, E2A linkers (See, e.g., WO2017127750). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one antigen/polypeptide separately within the same molecule) may be suitable for use as provided herein.

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art-non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding a coronavirus antigen). In some embodiments, a codon optimized sequence shares less than 90% sequence identity to a naturally-occurring or wild-type sequence (e.g., a naturally-occurring or wild-type mRNA sequence encoding a coronavirus antigen). In some embodiments, a codon optimized sequence shares less than 85% sequence identity to a naturally-occurring or wild-type sequence (e.g., a naturally-occurring or wild-type mRNA sequence encoding a coronavirus antigen). In some embodiments, a codon optimized sequence shares less than 80% sequence identity to a naturally-occurring or wild-type sequence (e.g., a naturally-occurring or wild-type mRNA sequence encoding a coronavirus antigen). In some embodiments, a codon optimized sequence shares less than 75% sequence identity to a naturally-occurring or wild-type sequence (e.g., a naturally-occurring or wild-type mRNA sequence encoding a coronavirus antigen).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a coronavirus antigen). In some embodiments, a codon optimized sequence shares between 65% and 75% or about 80% sequence identity to a naturally-occurring or wild-type sequence (e.g., a naturally-occurring or wild-type mRNA sequence encoding a coronavirus antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a coronavirus antigen encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, an mRNA is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemical Modifications

The compositions of the present disclosure comprise, in some embodiments, an RNA having an open reading frame encoding a coronavirus antigen, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367 all of which are incorporated by reference herein.

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a mRNA of the disclosure comprises 1-methyl-pseudouridine (mlv) substitutions at one or more or all uridine positions of the nucleic acid.

In some embodiments, a mRNA of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a mRNA of the disclosure comprises pseudouridine (ψ) substitutions at one or more or all uridine positions of the nucleic acid.

In some embodiments, a mRNA of the disclosure comprises pseudouridine (ψ) substitutions at one or more or all uridine positions of the nucleic acid and 5-methyl cytidine substitutions at one or more or all cytidine positions of the nucleic acid.

In some embodiments, a mRNA of the disclosure comprises uridine at one or more or all uridine positions of the nucleic acid.

In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure or can be replaced by a plurality of compounds having different structures (e.g., 2, 3, 4 or more unique structures).

### Untranslated Regions (UTRs)

The mRNAs of the present disclosure may comprise one or more regions or parts which act or function as an untranslated region. Where mRNAs are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR (A/G)CCAUGG (SEQ ID NO: 52), where R is a purine (adenine or guanine) three bases upstream of the start codon (AUG), which is followed by another ‘G’0.5′UTR also have been known to form secondary structures which are involved in elongation factor binding.

In some embodiments of the disclosure, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (IE1) gene (US20140206753, WO2013/185069), the sequence GGGAUCCUACC (SEQ ID NO: 24) (WO2014144196) may also be used. In another embodiment, 5′ UTR of a TOP gene is a 5′ UTR of a TOP gene lacking the 5′ TOP motif (the oligopyrimidine tract) (e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738), 5′ UTR element derived from the 5′UTR of an hydroxysteroid (17-β) dehydrogenase 4 gene (HSD17B4) (WO2015024667), or a 5′ UTR element derived from the 5′ UTR of ATP5A1 (WO2015024667) can be used. In some embodiments, an internal ribosome entry site (IRES) is used instead of a 5′ UTR.

In some embodiments, a 5′ UTR of the present disclosure comprises a sequence selected from SEQ ID NO: 2 and SEQ ID NO: 21.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA (U/A) (U/A) nonamers. Molecules containing this type of AREs include GM-CSF and TNF-α. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids (e.g., RNA) of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

Those of ordinary skill in the art will understand that 5′UTRs that are heterologous or synthetic may be used with any desired 3′ UTR sequence. For example, a heterologous 5′UTR may be used with a synthetic 3′UTR with a heterologous 3′UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo (dT) sequence for templated addition of a poly-A tail. 5′ UTR may comprise a first polynucleotide fragment and a second polynucleotide fragment from the same and/or different genes such as the 5′ UTRs described in US Patent Application Publication No. 20100293625 and PCT/US2014/069155, herein incorporated by reference in its entirety.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in U.S. application No. 20090226470, herein incorporated by reference in its entirety, and those known in the art.

### Non-Coding Sequences

Aspects of the disclosure relate to multivalent RNA compositions which comprise mRNAs, e.g., 2-15 mRNA polynucleotides each comprising a distinct open reading frame (ORF) encoding a coronavirus virus antigenic polypeptide, wherein each mRNA polynucleotide comprises one or more non-coding sequences in an untranslated region (UTR) having unique identifier sequences (non-coding sequences). In some embodiments the non-coding sequence is a unique non-coding sequence. In some embodiments, each mRNA in a multivalent vaccine composition comprises its own unique non-coding sequence. As used herein, “non-coding sequence” refers to a sequence of a biological molecule (e.g., nucleic acid, protein, etc.) that when combined with the sequence another biological molecule serves to identify the other biological molecule. Typically, a non-coding sequence is a heterologous sequence that is incorporated within or appended to a sequence of a target biological molecule and utilized as a reference in order to identify a target molecule of interest. In some embodiments, a non-coding sequence is a sequence of a nucleic acid (e.g., a heterologous or synthetic nucleic acid) that is incorporated within or appended to a target nucleic acid and utilized as a reference in order to identify the target nucleic acid. In some embodiments, a non-coding sequence is of the formula (N) n. In some embodiments, n is an integer in the range of 5 to 20, 5 to 10, 10 to 20, 7 to 20, or 7 to 30. In some embodiments, n is 2, 3, 4, 5, 6, 7, 8, 9, 10, 11, 12, 13, 14, 15, 16, 17, 18, 19, 20, 21, 22, 23, 24, 25, 26, 27, 28, 29, 30 or more. In some embodiments, each Nis a nucleotide that is independently selected from A, G, T, U, and C, or analogues thereof. Thus, some embodiments comprise nucleic acids (e.g., mRNAs) that (i) have a target sequence of interest (e.g., a coding sequence (e.g., that encodes an antigen protein or antigenic polypeptide)); and (ii) comprises a unique non-coding sequence.

In some embodiments, one or more in vitro transcribed mRNAs comprise one or more non-coding sequences in an untranslated region (UTR), such as a 5′ UTR or 3′ UTR. Inclusion of a non-coding sequence in the UTR of an mRNA prevents non-coding sequence from being translated into a peptide. In some embodiments, a non-coding sequence is positioned in a 3′ UTR of an mRNA. In some embodiments, the non-coding sequence is positioned upstream of the poly A tail of the mRNA. In some embodiments, the non-coding sequence is positioned downstream of (e.g., after) the polyA tail of the mRNA. In some embodiments, the non-coding sequence is positioned between the last codon of the ORF of the mRNA and the first “A” of the poly A tail of the mRNA. In some embodiments, a polynucleotide non-coding sequence positioned in a UTR comprises between 1 and 10 nucleotides (e.g., 1, 2, 3, 4, 5, 6, 7, 8, 9, or 10 nucleotides). In some embodiments, UTR comprising a polynucleotide non-coding sequence further comprises one or more (e.g., 1, 2, 3, or more) RNAse cleavage sites, such as RNase H cleavage sites. In some embodiments, each different RNA of a multivalent RNA composition comprises a different (e.g., unique) non-coding sequence. In some embodiments, RNAs of a multivalent RNA composition are detected and/or purified according to the polynucleotide non-coding sequences of the RNAs. In some embodiments, the mRNA non-coding sequences are used to identify the presence of mRNA or determine a relative ratio of different mRNAs in a sample (e.g., a reaction product or a drug product). In some embodiments, the mRNA non-coding sequences are detected using one or more of deep sequencing, PCR, and Sanger sequencing. Exemplary non-coding sequences include: AACGUGAU; AAACAUCG; ATGCCUAA; AGUGGUCA; ACCACUGU; ACAUUGGC; CAGAUCUG; CAUCAAGU; CGCUGAUC; ACAAGCUA; CUGUAGCC; AGUACAAG; AACAACCA; AACCGAGA; AACGCUUA; AAGACGGA; AAGGUACA; ACACAGAA; ACAGCAGA; ACCUCCAA; ACGCUCGA; ACGUAUCA; ACUAUGCA; AGAGUCAA; AGAUCGCA; AGCAGGAA; AGUCACUA; AUCCUGUA; AUUGAGGA; CAACCACA; GACUAGUA; CAAUGGAA; CACUUCGA; CAGCGUUA; CAUACCAA; CCAGUUCA; CCGAAGUA; ACAGUG; CGAUGU; UUAGGC; AUCACG; and UGACCA.

In some embodiments the multivalent RNA composition is produced by a method comprising:
(a) combining a linearized first DNA molecule encoding the first mRNA polynucleotide, a linearized second DNA molecule encoding the second mRNA polynucleotide, and, optionally, a linearized third, fourth, fifth, sixth, seventh, eighth, ninth or tenth DNA molecule encoding the third, fourth, fifth, sixth, seventh, eighth, ninth or tenth mRNA polynucleotide into a single reaction vessel, wherein the first DNA molecule, the second DNA molecule, and the third, fourth, fifth, sixth, seventh, eighth, ninth or tenth DNA molecule are obtained from different sources; and
(b) simultaneously in vitro transcribing the linearized first DNA molecule, the linearized second DNA molecule and the optional linearized third, fourth, fifth, sixth, seventh, eighth, ninth or tenth DNA molecule to obtain a multivalent RNA composition. The different sources may be bacterial cell cultures which may not be co-cultured. In some embodiments the amounts of the first, second and third, fourth, fifth, sixth, seventh, eighth, ninth or tenth DNA molecules present in the reaction mixture prior to the start of the IVT have been normalized.

### In Vitro Transcription of RNA

cDNA encoding the polynucleotides described herein may be transcribed using an in vitro transcription (IVT) system. In vitro transcription of RNA is known in the art and is described in International Publication WO 2014/152027, which is incorporated by reference herein in its entirety. In some embodiments, the RNA of the present disclosure is prepared in accordance with any one or more of the methods described in WO 2018/053209 and WO 2019/036682, each of which is incorporated by reference herein.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of an RNA polynucleotide, for example, but not limited to coronavirus mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7 mG(5′)ppp(5′)NlmpNp.

### Chemical Synthesis

Solid-phase chemical synthesis. Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of nucleic acids of the present disclosure by the sequential addition of monomer building blocks may be carried out in a liquid phase.

Combination of Synthetic Methods. The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present disclosure may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Lipid Nanoparticles (LNPs)

In some embodiments, the mRNA of the disclosure is formulated in a lipid nanoparticle (LNP). It is to be understood that “a lipid nanoparticle,” as used herein refers to a single LNP or a population of LNPs. Lipid nanoparticles typically comprise ionizable amino (cationic) lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Vaccines of the present disclosure are typically formulated in lipid nanoparticle. In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises 20-60 mol % ionizable amino lipid. In some embodiments, the lipid nanoparticle comprises 20-55 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 40 mol %, 41 mol %, 42 mol %, 43 mol %, 44 mol % 45 mol %, 46 mol %, 47 mol %, 48 mol %, 49 mol %, 50 mol %, or 60 mol % ionizable amino lipid.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

In some embodiments, the lipid nanoparticle comprises 20-60 mol % ionizable amino lipid, 5-25 mol % non-cationic lipid, 25-55 mol % sterol, and 0.5-15 mol % PEG-modified lipid. In some embodiments, the lipid nanoparticle comprises 40-50 mol % ionizable amino lipid, 5-15 mol % neutral lipid, 20-40 mol % cholesterol, and 0.5-3 mol % PEG-modified lipid. In some embodiments, the lipid nanoparticle comprises 45-50 mol % ionizable amino lipid, 9-13 mol % neutral lipid, 35-45 mol % cholesterol, and 2-3 mol % PEG-modified lipid. In some embodiments, the lipid nanoparticle comprises 48 mol % ionizable amino lipid, 11 mol % neutral lipid, 68.5 mol % cholesterol, and 2.5 mol % PEG-modified lipid.

In some embodiments, an ionizable amino lipid of the disclosure comprises a compound of Formula (I):

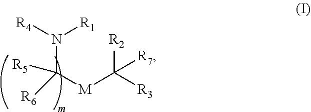

or a salt or isomer thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, a subset of compounds of Formula (I) includes those in which when R₄ is —(CH₂)ₙQ, —(CH₂)ₙCHQR, —CHQR, or —CQ(R)₂, then (i) Q is not-N(R)₂ when n is 1, 2, 3, 4 or 5, or (ii) Q is not 5, 6, or 7-membered heterocycloalkyl when n is 1 or 2. In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(—CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂),OR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂),CHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, a subset of compounds of Formula (I) includes those of Formula (IA):

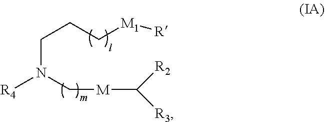

or a salt or isomer thereof, wherein 1 is selected from 1, 2, 3, 4, and 5; m is selected from 5, 6, 7, 8, and 9; Mi is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

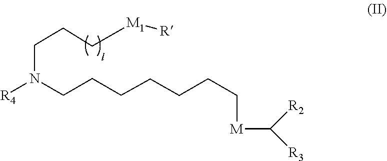

or a salt or isomer thereof, wherein 1 is selected from 1, 2, 3, 4, and 5; Mi is a bond or M′; R₄ is unsubstituted C₁₋₃ alkyl, or —(CH₂)ₙQ, in which n is 2, 3, or 4, and Q is
OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

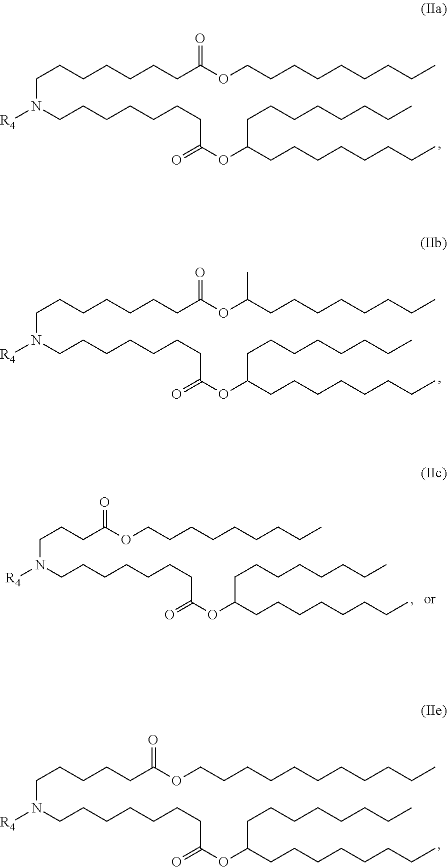

or a salt or isomer thereof, wherein R₄ is as described herein.

In some embodiments, a subset of compounds of Formula (I) includes those of Formula (IId):

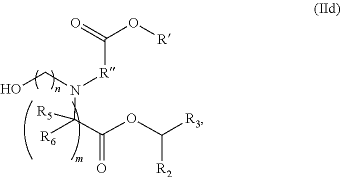

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid of the disclosure comprises a compound having structure:

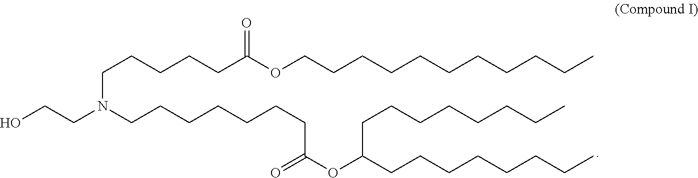 [SMILES: CCCCCCCCCCCOC(=O)CCCCCN(CCO)CCCCCCCC(=O)OC(CCCCCCCC)CCCCCCCC]

In some embodiments, an ionizable amino lipid of the disclosure comprises a compound having structure:

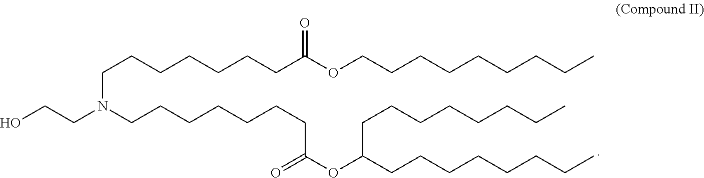 [SMILES: CCCCCCCCCOC(=O)CCCCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is DMG-PEG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable amino lipid of Compound 1, wherein the non-cationic lipid is DSPC, the structural lipid that is cholesterol, and the PEG lipid is DMG-PEG (e.g., PEG2000-DMG).

In some embodiments, the lipid nanoparticle comprises 45-55 mole percent (mol %) ionizable amino lipid (e.g., Compound 1). For example, lipid nanoparticle may comprise 45-47, 45-48, 45-49, 45-50, 45-52, 46-48, 46-49, 46-50, 46-52, 46-55, 47-48, 47-49, 47-50, 47-52, 47-55, 48-50, 48-52, 48-55, 49-50, 49-52, 49-55, or 50-55 mol % ionizable amino lipid (e.g., Compound 1). For example, lipid nanoparticle may comprise 45, 46, 47, 48, 49, 50, 51, 52, 53, 54, or 55 mol % ionizable amino lipid.

In some embodiments, the lipid nanoparticle comprises 5-15 mol % non-cationic (neutral) lipid (e.g., DSPC). For example, the lipid nanoparticle may comprise 5-6, 5-7, 5-8, 5-9, 5-10, 5-11, 5-12, 5-13, 5-14, 5-15, 6-7, 6-8, 6-9, 6-10, 6-11, 6-12, 6-13, 6-14, 6-15, 7-8, 7-9, 7-10, 7-11, 7-12, 7-13, 7-14, 7-15, 8-9, 8-10, 8-11, 8-12, 8-13, 8-14, 8-15, 9-10, 9-11, 9-12, 9-13, 9-14, 9-15, 10-11, 10-12, 10-13, 10-14, 10-15, 11-12, 11-13, 11-14, 11-15, 12-13, 12-14, 13-14, 13-15, or 14-15 mol % non-cationic (neutral) lipid (e.g., DSPC). For example, the lipid nanoparticle may comprise 5, 6, 7, 8, 9, 10, 11, 12, 13, 14, or 15 mol % DSPC.

In some embodiments, the lipid nanoparticle comprises 35-40 mol % sterol (e.g., cholesterol). For example, the lipid nanoparticle may comprise 35-36, 35-37, 35-38, 35-39, 35-40, 36-37, 36-38, 36-39, 36-40, 37-38, 37-39, 37-40, 38-39, 38-40, or 39-40 mol % cholesterol. For example, the lipid nanoparticle may comprise 35, 35.5, 36, 36.5, 37, 37.5, 38, 38.5, 39, 39.5, or 40 mol % cholesterol.

In some embodiments, the lipid nanoparticle comprises 1-3 mol % DMG-PEG. For example, the lipid nanoparticle may comprise 1-1.5, 1-2, 1-2.5, 1-3, 1.5-2, 1.5-2.5, 1.5-3, 2-2.5, 2-3, or 2.5-3. mol % DMG-PEG. For example, the lipid nanoparticle may comprise 1, 1.5, 2, 2.5, or 3 mol % DMG-PEG.

In some embodiments, the lipid nanoparticle comprises 50 mol % ionizable amino lipid, 10 mol % DSPC, 38.5 mol % cholesterol, and 1.5 mol % DMG-PEG. In some embodiments, the lipid nanoparticle comprises 48 mol % ionizable amino lipid, 11 mol % DSPC, 38.5 mol % cholesterol, and 2.5 mol % PEG2000-DMG.

In some embodiments, an LNP of the disclosure comprises an N: P ratio of from about 2:1 to about 30:1.

In some embodiments, an LNP of the disclosure comprises an N: P ratio of about 6:1.

In some embodiments, an LNP of the disclosure comprises an N: P ratio of about 3:1.

In some embodiments, an LNP of the disclosure comprises a wt/wt ratio of the ionizable amino lipid component to the RNA of from about 10:1 to about 100:1.

In some embodiments, an LNP of the disclosure comprises a wt/wt ratio of the ionizable amino lipid component to the RNA of about 20:1.

In some embodiments, an LNP of the disclosure comprises a wt/wt ratio of the ionizable amino lipid component to the RNA of about 10:1.

In some embodiments, an LNP of the disclosure has a mean diameter from about 50 nm to about 150 nm.

In some embodiments, an LNP of the disclosure has a mean diameter from about 70 nm to about 120 nm.

### Multivalent Vaccines

The compositions, as provided herein, may include RNA or multiple RNAs encoding two or more antigens of the same or different species. In some embodiments, composition includes an RNA or multiple RNAs encoding two or more coronavirus antigens. In some embodiments, the RNA may encode 1, 2, 3, 4, 5, 6, 7, 8, 9, or 10, or more coronavirus antigens.

In some embodiments, two or more different mRNA encoding antigens may be formulated in the same lipid nanoparticle. In other embodiments, two or more different RNA encoding antigens may be formulated in separate lipid nanoparticles (each RNA formulated in a single lipid nanoparticle). The lipid nanoparticles may then be combined and administered as a single vaccine composition (e.g., comprising multiple RNA encoding multiple antigens) or may be administered separately. In some embodiments, when the composition comprises two different RNA encoding antigens, the ratio of RNA encoding antigens is 1:1, 1:2, 1:4, 4:1, or 2:1.

### Initial or First Vaccine

In some embodiments the first or initial vaccine is an mRNA vaccine encoding a 2P stabilized spike protein. For instance, the initial or first vaccine may be an mRNA encoding a spike antigen having an amino acid sequence of SEQ ID NO: 20. In other embodiments the first vaccine may be any vaccine modality comprising a 2P stabilized spike protein. As a non-limiting example, the first vaccine composition may be a recombinant vaccine. As used herein, “recombinant vaccine” refers to a vaccine made by genetic engineering, the process and method of manipulating the genetic material of an organism. In most cases, a recombinant vaccine encompasses one or more nucleic acids encoding protein antigens that have either been produced and purified in a heterologous expression system (e.g., bacteria or yeast) or purified from large amounts of the pathogenic organism. Following administration, a vaccinated person produces antibodies to the one or more protein antigens, thus protecting him/her from disease. In some embodiments, the recombinant vaccine is a vectored vaccine. Viral vectored vaccines comprise a polynucleotide sequence not of viral origin (i.e., a polynucleotide heterologous to the virus), that encodes a peptide, polypeptide, or protein capable of eliciting an immune response in a host contacted with the vector. Expression of the polynucleotide results in the generation of a neutralizing antibody response and/or a cell mediated response, e.g., a cytotoxic T cell response. Examples of viral vectored vaccines include, but are not limited to, those developed by Oxford/AstraZeneca (COVID-19 Vaccine AstraZeneca), CanSino Biological Inc./Beijing Institute of Biotechnology, Gamaleya Research Institute, Zydus Cadila, Institut Pasteur/Themis/University of Pittsburgh Center for Vaccine Research, University of Hong Kong, and Altimmune (NasoVAX). In some embodiments, the recombinant vaccine is a nucleic acid-based (e.g., DNA, mRNA) coronavirus vaccine. Exemplary DNA vaccines include those being developed by Inovio Pharmaceuticals (INO-4800), Genexine Consortium (GX-19), OncoSec and the Cancer Institute (CORVax12 and TAVO™), Karolinska Institute/Cobra Biologics, Osaka University/Anges/Takara Bio, and Takis/Applied DNA Sciences/Evvivax. Exemplary mRNA vaccines include those being developed by BioNTech/Pfizer, Imperial College London, Curevac, and Walvax Biotech/People's Liberation Army (PLA) Academy of Military Science.

### Pharmaceutical Formulations

Provided herein are compositions (e.g., pharmaceutical compositions), methods, kits and reagents for prevention or treatment of coronavirus in humans and other mammals, for example. The compositions provided herein can be used as therapeutic or prophylactic agents. They may be used in medicine to prevent and/or treat a coronavirus infection.

In some embodiments, the SARS-CoV-2 vaccine containing RNA as described herein can be administered to a subject (e.g., a mammalian subject, such as a human subject), and the RNA polynucleotides are translated in vivo to produce an antigenic polypeptide (antigen).

An “effective amount” of a composition (e.g., comprising RNA) is based, at least in part, on the target tissue, target cell type, means of administration, physical characteristics of the RNA (e.g., length, nucleotide composition, and/or extent of modified nucleosides), other components of the vaccine, and other determinants, such as age, body weight, height, sex and general health of the subject. Typically, an effective amount of a composition provides an induced or boosted immune response as a function of antigen production in the cells of the subject. In some embodiments, an effective amount of the composition containing RNA polynucleotides having at least one chemical modifications are more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, the compositions (comprising polynucleotides and their encoded polypeptides) in accordance with the present disclosure may be used for treatment or prevention of a coronavirus infection. A composition may be administered prophylactically or therapeutically as part of an active immunization scheme to healthy individuals or early in infection during the incubation phase or during active infection after onset of symptoms. In some embodiments, the amount of RNA provided to a cell, a tissue or a subject may be an amount effective for immune prophylaxis.

A composition may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the vaccine composition and may include a traditional boost, seasonal boost or a pandemic shift boost. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, or 6 months, 7 months, 8 months, 9 months, 10 months, 11 months, one year, or more. In some embodiments, the time of administration between the initial administration of the prophylactic composition and the booster is at least 6 months. In exemplary embodiments, the time of administration between the initial administration of the prophylactic composition and the booster may be, but is not limited to, 1 week, 2 weeks, 3 weeks, 1 month, 2 months, 3 months, or 6 months. As is described herein, the booster may comprise the same or different mRNAs as compared to the earlier administration of the prophylactic composition. In some embodiments, the booster may comprise a combination of the same mRNA from the earlier administration of the prophylactic composition and at least one different mRNA. In some embodiments, the ratio of the mRNA from the earlier administration of the prophylactic composition and the at least one different mRNA is 1:1, 1:2, 1:4, 4:1, or 2:1. In one embodiment, the ratio is 1:1. In some embodiments, the booster may comprise different mRNAs as compared to the earlier administration of the prophylactic compositions. In some embodiments, such a booster may comprise 1, 2, 3, 4 or more mRNAs that were not present in the prophylactic composition. In some embodiments, the ratio of two mRNA polynucleotides (none of which were in the prophylactic composition) in the booster is 1:1, 1:2, 1:4, 4:1, or 2:1. In one embodiment, the ratio is 1:1. A boost or booster dose may be administered more than once, for example 2, 3, 4, 5, 6 or more times after the initial prophylactic (prime) dose. In some embodiments, a subsequent boost is administered within weeks, e.g., within 3-4 weeks of the first (or previous) boost. In some embodiments, a second boost is administered 1, 2, 3, 4, 5, 6, 7, 8, 9, 10, 11, 12, or more weeks after the first (or previous) boost. The booster, in some embodiments is monovalent (e.g., the mRNA encodes a single antigen). In some embodiments, the booster is multivalent (e.g., the mRNA encodes more than one antigen).

In some embodiments, the booster dose is 5 μg-30 μg, 5 μg-25 μg, 5 μg-20 μg, 5 μg-15 μg, 5 μg-10 μg, 10 μg-30 μg, 10 μg-25 μg, 10 μg-20 μg, 10 μg-15 μg, 15 μg-30 μg, 15 μg-25 μg, 15 μg-20 μg, 20 μg-30 μg, 25 μg-30 μg, or 25 μg-300 μg. In some embodiments, the booster dose is 10 μg-60 μg, 10 μg-55 μg, 10 μg-50 μg, 10 μg-45 μg, 10 μg-40 μg, 10 μg-35 μg, 10 μg-30 μg, 10 μg-25 μg, 10 μg-20 μg, 15 μg-60 μg, 15 μg-55 μg, 15 μg-50 μg, 15 μg-45 μg, 15 μg-40 μg, 15 μg-35 μg, 15 μg-30 μg, 15 μg-25 μg, 15 μg-20 μg, 20 μg-60 μg, 20 μg-55 μg, 20 μg-50 μg, 20 μg-45 μg, 20 μg-40 μg, 20 μg-35 μg, 20 μg-30 μg, 20 μg-25 μg, 25 μg-60 μg, 25 μg-55 μg, 25 μg-50 μg, 25 μg-45 μg, 25 μg-40 μg, 25 μg-35 μg, 25 μg-30 μg, 30 μg-60 μg, 30 μg-55 μg, 30 μg-50 μg, 30 μg-45 μg, 30 μg-40 μg, 30 μg-35 μg, 35 μg-60 μg, 35 μg-55 μg, 35 μg-50 μg, 35 μg-45 μg, 35 μg-40 μg, 40 μg-60 μg, 40 μg-55 μg, 40 μg-50 μg, 40 μg-45 μg, 45 μg-60 μg, 45 μg-55 μg, 45 μg-50 μg, 50 μg-60 μg, 50 μg-55 μg, or 55 μg-60 μg. In some embodiments, the booster dose is at least 10 μg and less than 25 μg of the composition. In some embodiments, the booster dose is at least 5 μg and less than 25 μg of the composition. For example, the booster dose is 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 250 μg, or 300 μg. In some embodiments, the booster dose is 50 μg.

In some embodiments, a composition may be administered intramuscularly, intranasally or intradermally, similarly to the administration of inactivated vaccines known in the art.

A composition may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers, better neutralizing immunity, produce more durable immune responses, and/or produce responses earlier than commercially available vaccines.

Provided herein are pharmaceutical compositions including RNA and/or complexes optionally in combination with one or more pharmaceutically acceptable excipients.

The RNA may be formulated or administered alone or in conjunction with one or more other components. For example, an immunizing composition may comprise other components including, but not limited to, adjuvants.

In some embodiments, an immunizing composition does not include an adjuvant (it is adjuvant free).

An RNA may be formulated or administered in combination with one or more pharmaceutically-acceptable excipients. In some embodiments, vaccine compositions comprise at least one additional active substances, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, an immunizing composition is administered to humans, human patients or subjects. For the purposes of the present disclosure, the phrase “active ingredient” generally refers to the RNA vaccines or the polynucleotides contained therein, for example, RNA polynucleotides (e.g., mRNA polynucleotides) encoding antigens.

Formulations of the vaccine compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, an RNA is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with the RNA (e.g., for transplantation into a subject), hyaluronidase, nanoparticle mimics and combinations thereof.

### Dosing/Administration

Provided herein are immunizing compositions (e.g., RNA vaccines), methods, kits and reagents for prevention and/or treatment of coronavirus infection in humans and other mammals. Immunizing compositions can be used as therapeutic or prophylactic agents. In some embodiments, immunizing compositions are used to provide prophylactic protection from coronavirus infection. In some embodiments, immunizing compositions are used to treat a coronavirus infection. In some embodiments, embodiments, immunizing compositions are used in the priming of immune effector cells, for example, to activate peripheral blood mononuclear cells (PBMCs) ex vivo, which are then infused (re-infused) into a subject.

A subject may be any mammal, including non-human primate and human subjects. Typically, a subject is a human subject.

In some embodiments, an immunizing composition (e.g., RNA vaccine) is administered to a subject (e.g., a mammalian subject, such as a human subject) in an effective amount to induce an antigen-specific immune response. The RNA encoding the coronavirus spike protein antigen is expressed and translated in vivo to produce the antigen, which then stimulates an immune response in the subject.

Prophylactic protection from a coronavirus can be achieved following administration of an immunizing composition (e.g., an RNA vaccine) of the present disclosure. Immunizing compositions can be administered once, twice, three times, four times or more but it is likely sufficient to administer the vaccine once (optionally followed by a single booster). It is possible, although less desirable, to administer an immunizing composition to an infected individual to achieve a therapeutic response. Dosing may need to be adjusted accordingly.

A method of eliciting an immune response in a subject against a coronavirus antigen (or multiple antigens) is provided in aspects of the present disclosure. In some embodiments, a method involves administering to the subject an immunizing composition comprising a mRNA having an open reading frame encoding a coronavirus antigen, thereby inducing in the subject an immune response specific to the coronavirus antigen, wherein anti-antigen antibody titer in the subject is increased following vaccination relative to anti-antigen antibody titer in a subject vaccinated with a prophylactically effective dose of a traditional vaccine against the antigen. An “anti-antigen antibody” is a serum antibody the binds specifically to the antigen.

A prophylactically effective dose is an effective dose that prevents infection with the virus at a clinically acceptable level. In some embodiments, the effective dose is a dose listed in a package insert for the vaccine. A traditional vaccine, as used herein, refers to a vaccine other than the mRNA vaccines of the present disclosure. For instance, a traditional vaccine includes, but is not limited, to live microorganism vaccines, killed microorganism vaccines, subunit vaccines, protein antigen vaccines, DNA vaccines, virus like particle (VLP) vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments, the anti-antigen antibody titer in the subject is increased 1 log to 10 log following vaccination relative to anti-antigen antibody titer in a subject vaccinated with a prophylactically effective dose of a traditional vaccine against the coronavirus or an unvaccinated subject. In some embodiments, the anti-antigen antibody titer in the subject is increased 1 log, 2 log, 3 log, 4 log, 5 log, or 10 log following vaccination relative to anti-antigen antibody titer in a subject vaccinated with a prophylactically effective dose of a traditional vaccine against the coronavirus or an unvaccinated subject.

A method of eliciting an immune response in a subject against a coronavirus is provided in other aspects of the disclosure. The method involves administering to the subject a composition comprising an mRNA comprising an open reading frame encoding a coronavirus antigen, thereby inducing in the subject an immune response specific to the coronavirus, wherein the immune response in the subject is equivalent to an immune response in a subject vaccinated with a traditional vaccine against the coronavirus at 2 times to 100 times the dosage level relative to the composition.

In some embodiments, the immune response in the subject is equivalent to an immune response in a subject vaccinated with a traditional vaccine at twice the dosage level relative to a composition of the present disclosure. In some embodiments, the immune response in the subject is equivalent to an immune response in a subject vaccinated with a traditional vaccine at three times the dosage level relative to a composition of the present disclosure. In some embodiments, the immune response in the subject is equivalent to an immune response in a subject vaccinated with a traditional vaccine at 4 times, 5 times, 10 times, 50 times, or 100 times the dosage level relative to a composition of the present disclosure. In some embodiments, the immune response in the subject is equivalent to an immune response in a subject vaccinated with a traditional vaccine at 10 times to 1000 times the dosage level relative to a composition of the present disclosure. In some embodiments, the immune response in the subject is equivalent to an immune response in a subject vaccinated with a traditional vaccine at 100 times to 1000 times the dosage level relative to a composition of the present disclosure.

In other embodiments, the immune response is assessed by determining [protein] antibody titer in the subject. In other embodiments, the ability of serum or antibody from an immunized subject is tested for its ability to neutralize viral uptake or reduce coronavirus transformation of human B lymphocytes. In other embodiments, the ability to promote a robust T cell response(s) is measured using art recognized techniques.

Other aspects the disclosure provide methods of eliciting an immune response in a subject against a coronavirus by administering to the subject composition comprising an mRNA having an open reading frame encoding a coronavirus antigen, thereby inducing in the subject an immune response specific to the coronavirus antigen, wherein the immune response in the subject is induced 2 days to 10 weeks earlier relative to an immune response induced in a subject vaccinated with a prophylactically effective dose of a traditional vaccine against the coronavirus. In some embodiments, the immune response in the subject is induced in a subject vaccinated with a prophylactically effective dose of a traditional vaccine at 2 times to 100 times the dosage level relative to a composition of the present disclosure.

In some embodiments, the immune response in the subject is induced 2 days, 3 days, 1 week, 2 weeks, 3 weeks, 5 weeks, or 10 weeks earlier relative to an immune response induced in a subject vaccinated with a prophylactically effective dose of a traditional vaccine.

Also provided herein are methods of eliciting an immune response in a subject against a coronavirus by administering to the subject an mRNA having an open reading frame encoding a first antigen, wherein the RNA does not include a stabilization element, and wherein an adjuvant is not co-formulated or co-administered with the vaccine.

A composition may be administered by any route that results in a therapeutically effective outcome. These include, but are not limited, to intradermal, intramuscular, intranasal, and/or subcutaneous administration. The present disclosure provides methods comprising administering RNA vaccines to a subject in need thereof. The exact amount required will vary from subject to subject, depending on the species, age, and general condition of the subject, the severity of the disease, the particular composition, its mode of administration, its mode of activity, and the like. The RNA is typically formulated in dosage unit form for ease of administration and uniformity of dosage. It will be understood, however, that the total daily usage of the RNA may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount (e.g., effective dose) of the RNA, as provided herein, may be as low as 20 μg, administered for example as a single dose or as two 10 μg doses. In some embodiments, the effective amount (e.g., effective dose) is a total dose of 20 μg-300 μg 5 μg-30 μg, 5 μg-25 μg, 5 μg-20 μg, 5 μg-15 μg, 5 μg-10 μg, 10 μg-30 μg, 10 μg-25 μg, 10 μg-20 μg, 10 μg-15 μg, 15 μg-30 μg, 15 μg-25 μg, 15 μg-20 μg, 20 μg-30 μg, 25 μg-30 μg, or 25 μg-300 μg. In some embodiments, the effective dose (e.g., effective amount) is at least 10 μg and less than 25 μg of the composition. In some embodiments, the effective dose (e.g., effective amount) is at least 5 μg and less than 25 μg of the composition. For example, the effective amount may be a total dose of 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 250 μg, or 300 μg. In some embodiments, the effective amount (e.g., effective dose) is a total dose of 10 μg. In some embodiments, the effective amount is a total dose of 20 μg (e.g., two 10 μg doses). In some embodiments, the effective amount is a total dose of 25 μg. In some embodiments, the effective amount is a total dose of 30 μg. In some embodiments, the effective amount is a total dose of 50 μg. In some embodiments, the effective amount is a total dose of 60 μg (e.g., two 30 μg doses). In some embodiments, the effective amount is a total dose of 75 μg. In some embodiments, the effective amount is a total dose of 100 μg. In some embodiments, the effective amount is a total dose of 150 μg. In some embodiments, the effective amount is a total dose of 200 μg. In some embodiments, the effective amount is a total dose of 250 μg. In some embodiments, the effective amount is a total dose of 300 μg. Any of the doses provided above may be an effective amount for a booster dose; for example, in some embodiments, the booster dose is a total dose of 50 μg. In some embodiments, the composition comprises two or more mRNA polynucleotides and effective amount is a total dose of 20 μg (e.g., 10 μg of a first mRNA and 10 μg of a second mRNA). In some embodiments, the composition comprises two or more mRNA polynucleotides and effective amount is a total dose of 50 μg (e.g., 25 μg of a first mRNA and 25 μg of a second mRNA). In some embodiments, the composition comprises two or more mRNA polynucleotides and effective amount is a total dose of 100 μg (e.g., 50 μg of a first mRNA and 50 μg of a second mRNA).

The RNA described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Vaccine Efficacy

Some aspects of the present disclosure provide formulations of the compositions (e.g., RNA vaccines), wherein the RNA is formulated in an effective amount to produce an antigen specific immune response in a subject (e.g., production of antibodies specific to a coronavirus antigen). “An effective amount” is a dose of the RNA effective to produce an antigen-specific immune response. Also provided herein are methods of inducing an antigen-specific immune response in a subject.

As used herein, an immune response to a vaccine or LNP of the present disclosure is the development in a subject of a humoral and/or a cellular immune response to a (one or more) coronavirus protein(s) present in the vaccine. For purposes of the present disclosure, a “humoral” immune response refers to an immune response mediated by antibody molecules, including, e.g., secretory (IgA) or IgG molecules, while a “cellular” immune response is one mediated by T-lymphocytes (e.g., CD4+ helper and/or CD8+ T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+ and CD8+ T-cells.

In some embodiments, the antigen-specific immune response is characterized by measuring an anti-coronavirus antigen antibody titer produced in a subject administered a composition as provided herein. An antibody titer is a measurement of the amount of antibodies within a subject, for example, antibodies that are specific to a particular antigen or epitope of an antigen. Antibody titer is typically expressed as the inverse of the greatest dilution that provides a positive result. Enzyme-linked immunosorbent assay (ELISA) is a common assay for determining antibody titers, for example.

A variety of serological tests can be used to measure antibody against encoded antigen of interest, for example, SAR-CoV-2 virus or SAR-CoV-2 viral antigen, e.g., SAR-CoV-2 spike or S protein, of domain thereof. These tests include the hemagglutination-inhibition test, complement fixation test, fluorescent antibody test, enzyme-linked immunosorbent assay (ELISA), and plaque reduction neutralization test (PRNT). Each of these tests measures different antibody activities. In exemplary embodiments, A plaque reduction neutralization test, or PRNT (e.g., PRNT50 or PRNT90) is used as a serological correlate of protection. PRNT measures the biological parameter of in vitro virus neutralization and is the most serologically virus-specific test among certain classes of viruses, correlating well to serum levels of protection from virus infection.

The basic design of the PRNT allows for virus-antibody interaction to occur in a test tube or microtiter plate, and then measuring antibody effects on viral infectivity by plating the mixture on virus-susceptible cells, preferably cells of mammalian origin. The cells are overlaid with a semi-solid media that restricts spread of progeny virus. Each virus that initiates a productive infection produces a localized area of infection (a plaque), that can be detected in a variety of ways. Plaques are counted and compared back to the starting concentration of virus to determine the percent reduction in total virus infectivity. In PRNT, the serum sample being tested is usually subjected to serial dilutions prior to mixing with a standardized amount of virus. The concentration of virus is held constant such that, when added to susceptible cells and overlaid with semi-solid media, individual plaques can be discerned and counted. In this way, PRNT end-point titers can be calculated for each serum sample at any selected percent reduction of virus activity.

In functional assays intended to assess vaccinal immunogenicity, the serum sample dilution series for antibody titration should ideally start below the “seroprotective” threshold titer. Regarding SARS-CoV-2 neutralizing antibodies, the “seroprotective” threshold titer remains unknown; but a seropositivity threshold of 1:10 can be considered a seroprotection threshold in certain embodiments.

In some embodiments a neutralizing immune response is an immune response that produces a level of antibodies that meet or exceed a seroprotection threshold.

PRNT end-point titers are expressed as the reciprocal of the last serum dilution showing the desired percent reduction in plaque counts. The PRNT titer can be calculated based on a 50% or greater reduction in plaque counts (PRNT50). A PRNT50 titer is preferred over titers using higher cut-offs (e.g., PRNT90) for vaccine sera, providing more accurate results from the linear portion of the titration curve.

There are several ways to calculate PRNT titers. The simplest and most widely used way to calculate titers is to count plaques and report the titer as the reciprocal of the last serum dilution to show >50% reduction of the input plaque count as based on the back-titration of input plaques. Use of curve fitting methods from several serum dilutions may permit calculation of a more precise result. There are a variety of computer analysis programs available for this (e.g., SPSS or GraphPad Prism)

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by a composition (e.g., RNA vaccine).

In some embodiments, an anti-coronavirus antigen antibody titer produced in a subject is increased by at least 1 log relative to a control. For example, anti-coronavirus antigen antibody titer produced in a subject may be increased by at least 1.5, at least 2, at least 2.5, or at least 3 log relative to a control. In some embodiments, the anti-coronavirus antigen antibody titer produced in the subject is increased by 1, 1.5, 2, 2.5 or 3 log relative to a control. In some embodiments, the anti-coronavirus antigen antibody titer produced in the subject is increased by 1-3 log relative to a control. For example, the anti-coronavirus antigen antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-coronavirus antigen antibody titer produced in a subject is increased at least 2 times relative to a control. For example, the anti-coronavirus antigen n antibody titer produced in a subject may be increased at least 3 times, at least 4 times, at least 5 times, at least 6 times, at least 7 times, at least 8 times, at least 9 times, or at least 10 times relative to a control. In some embodiments, the anti-coronavirus antigen antibody titer produced in the subject is increased 2, 3, 4, 5, 6, 7, 8, 9, or 10 times relative to a control. In some embodiments, the anti-coronavirus antigen antibody titer produced in a subject is increased 2-10 times relative to a control. For example, the anti-coronavirus antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

In some embodiments, an antigen-specific immune response is measured as a ratio of geometric mean titer (GMT), referred to as a geometric mean ratio (GMR), of serum neutralizing antibody titers to coronavirus. A geometric mean titer (GMT) is the average antibody titer for a group of subjects calculated by multiplying all values and taking the nth root of the number, where n is the number of subjects with available data.

A control, in some embodiments, is an anti-coronavirus antigen antibody titer produced in a subject who has not been administered a composition (e.g., RNA vaccine). In some embodiments, a control is an anti-coronavirus antigen antibody titer produced in a subject administered a recombinant or purified protein vaccine. Recombinant protein vaccines typically include protein antigens that either have been produced in a heterologous expression system (e.g., bacteria or yeast) or purified from large amounts of the pathogenic organism.

In some embodiments, the ability of a composition (e.g., RNA vaccine) to be effective is measured in a murine model. For example, a composition may be administered to a murine model and the murine model assayed for induction of neutralizing antibody titers. Viral challenge studies may also be used to assess the efficacy of a vaccine of the present disclosure. For example, a composition may be administered to a murine model, the murine model challenged with virus, and the murine model assayed for survival and/or immune response (e.g., neutralizing antibody response, T cell response (e.g., cytokine response)).

In some embodiments, an effective amount of a composition (e.g., RNA vaccine) is a dose that is reduced compared to the standard of care dose of a recombinant protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified protein vaccine, or a live attenuated or inactivated vaccine, or a VLP vaccine, that a physician/clinician or other medical professional would administer to a subject to treat or prevent coronavirus infection or a related condition, while following the standard of care guideline for treating or preventing coronavirus infection or a related condition.

In some embodiments, the anti-coronavirus antigen antibody titer produced in a subject administered an effective amount of an composition is equivalent to an anti-coronavirus antigen antibody titer produced in a control subject administered a standard of care dose of a recombinant or purified protein vaccine, or a live attenuated or inactivated vaccine, or a VLP vaccine.

Vaccine efficacy may be assessed using standard analyses (see, e.g., Weinberg et al., J Infect Dis. 2010 Jun. 1; 201(11): 1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

$$Efficacy = ( A R U - A RV ) / ARU \times 100 ; \\ and \\ Efficacy = ( 1 - RR ) \times 100.$$

Likewise, vaccine effectiveness may be assessed using standard analyses (see, e.g., Weinberg et al., J Infect Dis. 2010 Jun. 1; 201(11): 1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

$$Effectiveness = ( 1 - OR ) \times 100.$$

In some embodiments, efficacy of the composition (e.g., RNA vaccine) is at least 60% relative to unvaccinated control subjects. For example, efficacy of the composition may be at least 65%, at least 70%, at least 75%, at least 80%, at least 85%, at least 95%, at least 98%, or 100% relative to unvaccinated control subjects.

Sterilizing Immunity. Sterilizing immunity refers to a unique immune status that prevents effective pathogen infection into the host. In some embodiments, the effective amount of a composition of the present disclosure is sufficient to provide sterilizing immunity in the subject for at least 1 year. For example, the effective amount of a composition of the present disclosure is sufficient to provide sterilizing immunity in the subject for at least 2 years, at least 3 years, at least 4 years, or at least 5 years. In some embodiments, the effective amount of a composition of the present disclosure is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

Detectable Antigen. In some embodiments, the effective amount of a composition of the present disclosure is sufficient to produce detectable levels of coronavirus antigen as measured in serum of the subject at 1-72 hours post administration.

Titer. An antibody titer is a measurement of the number of antibodies within a subject, for example, antibodies that are specific to a particular antigen (e.g., an anti-coronavirus antigen). Antibody titer is typically expressed as the inverse of the greatest dilution that provides a positive result. Enzyme-linked immunosorbent assay (ELISA) is a common assay for determining antibody titers, for example.

A neutralizing immune response is an immune response that is a neutralizing antibody response and/or an effective neutralizing T cell response. In some embodiments a neutralizing antibody response produces a level of antibodies that meet or exceed a seroprotection threshold.

An effective T cell response is a response which produces a baseline level of viral activated or viral specific T cells including CD8+ and CD4+T helper type 1 cells. CD8+ cytotoxic T lymphocytes typically clear the intracellular virus compartment and CD4+ T cells exert various functions in the body such as helping B and other T cells, promoting memory generation and indirect or direct cytotoxic activity. In some embodiments the effective T cells comprises a high proportion of CD8+ T cells and/or CD4+ T cells, relative to a baseline level (in a naïve subject). In some embodiments these T cells are differentiated towards an early-differentiated memory phenotype with co-expression of CD27 and CD28.

In some embodiments, the effective amount of a composition of the present disclosure is sufficient to produce a 1,000-10,000 neutralizing antibody titer produced by neutralizing antibody against the coronavirus antigen as measured in serum of the subject at 1-72 hours post administration. In some embodiments, the effective amount is sufficient to produce a 1,000-5,000 neutralizing antibody titer produced by neutralizing antibody against the coronavirus antigen as measured in serum of the subject at 1-72 hours post administration. In some embodiments, the effective amount is sufficient to produce a 5,000-10,000 neutralizing antibody titer produced by neutralizing antibody against the coronavirus antigen as measured in serum of the subject at 1-72 hours post administration.

In some embodiments, the neutralizing antibody titer is at least 100 NT₅₀. For example, the neutralizing antibody titer may be at least 200, 300, 400, 500, 600, 700, 800, 900 or 1000 NT₅₀. In some embodiments, the neutralizing antibody titer is at least 10,000 NT₅₀. In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, an anti-coronavirus antigen antibody titer produced in the subject is increased by at least 1 log relative to a control. For example, an anti-coronavirus antigen antibody titer produced in the subject may be increased by at least 2, 3, 4, 5, 6, 7, 8, 9 or 10 log relative to a control.

In some embodiments, an anti-coronavirus antigen antibody titer produced in the subject is increased at least 2 times relative to a control. For example, an anti-coronavirus antigen antibody titer produced in the subject is increased by at least 3, 4, 5, 6, 7, 8, 9 or 10 times relative to a control.

In some embodiments, a geometric mean, which is the nth root of the product of n numbers, is generally used to describe proportional growth. Geometric mean, in some embodiments, is used to characterize antibody titer produced in a subject.

A control may be, for example, an unvaccinated subject, or a subject administered a live attenuated viral vaccine, an inactivated viral vaccine, or a protein subunit vaccine.

### ADDITIONAL EMBODIMENTS

1. A method comprising administering to a subject a vaccine comprising a nucleic acid encoding a SARS-CoV-2 spike antigen, optionally a 2P stabilized spike antigen of a second circulating SARS-CoV-2 virus, wherein the subject has previously been administered a first vaccine comprising a nucleic acid encoding a first SARS-CoV-2 2P stabilized spike antigen of a first circulating SARS-CoV-2 virus, and wherein each of the first and second 2P stabilized spike antigens are administered in an effective amount to induce an immune response specific for the first antigen and the second antigen, wherein the second circulating SARS-CoV-2 virus has a spike protein having an amino acid sequence with at least one amino acid mutation with respect to a spike protein amino acid sequence of the first circulating SARS-CoV-2 virus, and wherein the mutation is an amino acid substitution, deletion or insertion.
2. A method comprising administering to a subject a first vaccine comprising a nucleic acid encoding a first SARS-CoV-2 2P stabilized spike antigen and administering to the subject a second vaccine comprising a nucleic acid encoding a second SARS-CoV-2 spike antigen, optionally a 2P stabilized spike antigen, wherein each of the nucleic acids encoding the first and second stabilized spike antigens are administered in an effective amount to induce an immune response specific for the respective encoded antigens, wherein the second encoded SARS-CoV-2 spike antigen has an amino acid sequence with at least one amino acid mutation with respect to the first encoded spike protein amino acid sequence, and wherein the mutation is an amino acid substitution, deletion, or insertion.
3. A method comprising administering to a subject a first vaccine comprising a nucleic acid encoding a first SARS-CoV-2 2P stabilized spike antigen and administering to the subject a second vaccine comprising a nucleic acid encoding a second SARS-CoV-2 spike antigen, optionally a 2P stabilized spike antigen, wherein each of the nucleic acids encoding the first and second stabilized spike antigens are administered in an effective amount to induce an immune response specific for the respective encoded antigens, wherein the second encoded SARS-CoV-2 spike antigen has an amino acid sequence with at least one amino acid mutation with respect to the first encoded spike protein amino acid sequence, wherein the mutation is an amino acid substitution, deletion, or insertion, and wherein the first encoded SARS-CoV-2 spike antigen is of a first circulating SARS-CoV-2 virus and wherein the second encoded SARS-CoV-2 spike antigen is of a second circulating SARS-CoV-2 virus.
4. A method comprising administering to a subject a first vaccine comprising a nucleic acid encoding a first SARS-CoV-2 2P stabilized spike antigen and administering to the subject a second vaccine comprising a nucleic acid encoding a second SARS-CoV-2 spike antigen, optionally a 2P stabilized spike antigen, wherein each of the nucleic acids encoding the first and second stabilized spike antigens are administered in an effective amount to induce an immune response specific for the respective encoded antigens, wherein the second encoded SARS-CoV-2 spike antigen has an amino acid sequence with at least one amino acid mutation with respect to the first encoded spike protein amino acid sequence, wherein the mutation is an amino acid substitution, deletion, or insertion, and wherein the first encoded SARS-CoV-2 spike antigen is representative of a first circulating SARS-CoV-2 virus and wherein the second encoded SARS-CoV-2 spike antigen is representative of a second circulating SARS-CoV-2 virus.
5. A method comprising administering to a subject a first vaccine comprising a nucleic acid encoding a first SARS-CoV-2 2P stabilized spike antigen and administering to the subject a second vaccine comprising a nucleic acid encoding a second SARS-CoV-2 spike antigen, optionally a 2P stabilized spike antigen, wherein each of the nucleic acids encoding the first and second stabilized spike antigens are administered in an effective amount to induce an immune response specific for the respective encoded antigens, wherein the second encoded SARS-CoV-2 spike antigen has an amino acid sequence with at least one amino acid mutation with respect to the first encoded spike protein amino acid sequence, wherein the mutation is an amino acid substitution, deletion, or insertion, and wherein the first encoded SARS-CoV-2 spike antigen is representative of a plurality of first circulating SARS-CoV-2 viruses and/or wherein the second encoded SARS-CoV-2 spike antigen is representative of a second plurality of circulating SARS-CoV-2 viruses.
6. The method of any one of paragraphs 1-5, wherein the first antigen is a mRNA encoding the first SARS-CoV-2 spike antigen and wherein the spike antigen has an amino acid sequence of SEQ ID NO: 20.
7. The method of any one of paragraphs 1-6, wherein the second antigen is a mRNA encoding the second SARS-CoV-2 spike antigen, wherein the spike antigen has an amino acid sequence with at least one amino acid mutation with respect to a protein of SEQ ID NO: 20, and wherein the mutation is an amino acid substitution, deletion or insertion.
8. A composition comprising: a first messenger ribonucleic acid (mRNA) encoding a first SARS-CoV-2 spike antigen of a first circulating SARS-CoV-2 virus wherein the first SARS-CoV-2 spike antigen has an amino acid sequence of SEQ ID NO: 20 or an amino acid sequence with at least one amino acid mutation with respect to a protein of SEQ ID NO: 20 and a second mRNA encoding a second SARS-CoV-2 spike antigen of a second circulating SARS-CoV-2 virus, wherein the second SARS-CoV-2 spike antigen has an amino acid sequence with at least one amino acid mutation with respect to a protein of SEQ ID NO: 20, wherein the wherein the mutation is an amino acid substitution, deletion or insertion, and wherein the first SARS-CoV-2 spike antigen and the second SARS-CoV-2 spike antigen are different from one another.
9. The composition of paragraph 8, wherein the composition further comprises a third messenger ribonucleic acid (mRNA) encoding a third SARS-CoV-2 spike antigen of a third SARS-CoV-2 virus, wherein the third SARS-CoV-2 spike antigen has an amino acid sequence with at least one amino acid mutation with respect to a protein of SEQ ID NO: 20, and wherein the mutation is an amino acid substitution, deletion or insertion.
10. The composition of paragraph 9, wherein the composition further comprises a fourth messenger ribonucleic acid (mRNA) encoding a fourth SARS-CoV-2 spike antigen of a fourth SARS-CoV-2 virus, wherein the fourth SARS-CoV-2 spike antigen has an amino acid sequence with at least one amino acid mutation with respect to a protein of SEQ ID NO: 20, and wherein the mutation is an amino acid substitution, deletion or insertion.
11. The composition of paragraph 10, wherein the composition further comprises a fifth messenger ribonucleic acid (mRNA) encoding a fifth SARS-CoV-2 spike antigen of a fifth SARS-CoV-2 virus, wherein the fifth SARS-CoV-2 spike antigen has an amino acid sequence with at least one amino acid mutation with respect to a protein of SEQ ID NO: 20, and wherein the mutation is an amino acid substitution, deletion or insertion.
12. The composition of paragraph 11, wherein the composition further comprises a sixth messenger ribonucleic acid (mRNA) encoding a sixth SARS-CoV-2 spike antigen of a sixth SARS-CoV-2 virus, wherein the sixth SARS-CoV-2 spike antigen has an amino acid sequence with at least one amino acid mutation with respect to a protein of SEQ ID NO: 20, and wherein the mutation is an amino acid substitution, deletion or insertion.
13. The composition of any one of paragraphs 8-12, wherein the first and second virus strains, and optionally the third, fourth, fifth and sixth virus strains are spreading in the population for at least a portion of 1 year.
14. A messenger ribonucleic acid (mRNA) encoding a SARS-CoV-2 2P stabilized spike protein, wherein the 2P stabilized spike protein has an amino acid sequence with at least one amino acid mutation with respect to a protein of SEQ ID NO: 20, wherein the mutation is an amino acid substitution, deletion or insertion, and wherein the 2P stabilized spike protein is a 2P stabilized version of a spike protein from a second circulating SARS-CoV-2 virus strain, and wherein a first circulating SARS-CoV-2 virus strain comprises a spike protein of SEQ ID NO: 11.
15. An mRNA, wherein the mRNA encodes a protein having at least 95% sequence identity to a protein of any one of SEQ ID NOs: 5, 8, 11, 14, 17, 23, 36, 30, 33, 36, 39, and 42.
16. An mRNA, wherein the mRNA has at least 95% sequence identity to an RNA of any one of SEQ ID NOs: 1, 6, 9, 12, 15, 21, 24, 28, 31, 34, 37, 40, 43, and 45.
17. An mRNA, wherein the mRNA has at least 98% sequence identity to an RNA of any one of SEQ ID NOs: 1, 6, 9, 12, 15, 21, 24, 28, 31, 34, 37, 40, 43, and 45.
18. An mRNA, wherein the mRNA comprises an RNA of any one of SEQ ID NOs: 1, 6, 9, 12, 15, 21, 24, 28, 31, 34, 37, 40, 43, and 45.
19. The mRNA of any one of paragraphs 14-18, wherein the mRNA comprises a chemical modification.
20. The mRNA of paragraph 19, wherein the mRNA is fully modified.
21. The mRNA of paragraph 19 or 20, wherein the chemical modification is 1-methylpseudouridine.
22. The mRNA of any one of paragraphs 14-21, wherein the mRNAs are in a lipid nanoparticle and wherein the lipid nanoparticle comprises an ionizable amino lipid, a sterol, a neutral lipid, and a polyethylene glycol (PEG)-modified lipid.
23. The mRNA of paragraph 22, wherein the lipid nanoparticle comprises 40-55 mol % ionizable amino lipid, 30-45 mol % sterol, 5-15 mol % neutral lipid, and 1-5 mol % PEG-modified lipid.
24. The mRNA of any one of paragraphs 22-23, wherein the lipid nanoparticle comprises 40-50 mol % ionizable amino lipid, 35-45 mol % sterol, 10-15 mol % neutral lipid, and 2-4 mol % PEG-modified lipid.
25. The mRNA of any one of paragraphs 22-24, wherein the lipid nanoparticle comprises 45 mol %, 46 mol %, 47 mol %, 48 mol %, 49 mol %, or 50 mol % ionizable amino lipid.
26. The mRNA of any one of paragraphs 22-25, wherein the ionizable amino lipid has the structure of Compound 1:

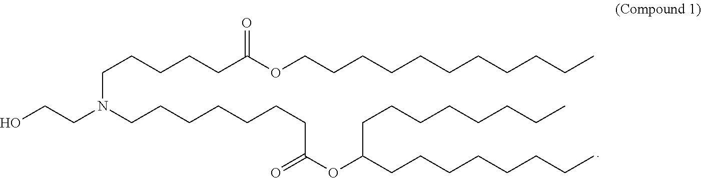 [SMILES: CCCCCCCCCCCOC(=O)CCCCCN(CCO)CCCCCCCC(=O)OC(CCCCCCCC)CCCCCCCC]

27. The mRNA of any one of any one of paragraphs 22-26, wherein the sterol is cholesterol or a derivative thereof.
28. The mRNA of any one of paragraphs 22-27, wherein the neutral lipid is 1,2 distearoyl-sn-glycero-3-phosphocholine (DSPC).
29. The mRNA of any one of paragraphs 22-28, wherein the PEG-modified lipid is 1,2 dimyristoyl-sn-glycerol, methoxypolyethyleneglycol (PEG2000 DMG).
30. A method comprising administering to a subject a vaccine comprising a first nucleic acid encoding a SARS-CoV-2 2P stabilized spike antigen, wherein the spike antigen has an amino acid sequence with at least one amino acid mutation with respect to a protein of SEQ ID NO: 20, and wherein the mutation is an amino acid substitution, deletion or insertion, wherein the subject is seropositive for a SARS-CoV-2 antigen of SEQ ID NO. 21 or 20.
31. A method comprising administering to a subject a vaccine comprising a first nucleic acid encoding a SARS-CoV-2 2P stabilized spike antigen, wherein the spike antigen has an amino acid sequence with at least one amino acid mutation with respect to a protein of SEQ ID NO: 20, and wherein the mutation is an amino acid substitution, deletion or insertion, wherein the subject is seronegative for a SARS-CoV-2 antigen of SEQ ID NO. 21 or 20.
32. The method of paragraph 30 or 31, wherein the subject is administered a second dose of the vaccine between 2 weeks and 1 year after the first dose of vaccine is administered.
33. The method of paragraph 31 or 32, wherein the subject is administered a second vaccine between 2 weeks and 1 year after the vaccine is administered, wherein the second vaccine comprises a second nucleic acid encoding a SARS-CoV-2 2P stabilized spike antigen of SEQ ID NO: 20.
34. The method of paragraph 33, wherein the second vaccine comprises a mixture of the first and second nucleic acids, wherein the first nucleic acid and the second nucleic acid are present in the second vaccine at a ratio of 1:1.
35. The method of any one of paragraphs 30-34, where 50 μg of the vaccine comprising a nucleic acid encoding a SARS-CoV-2 spike antigen, optionally, a 2P stabilized spike antigen of a third circulating SARS-CoV-2 virus is administered to the subject.
36. The method of any one of paragraphs 30-35, wherein the vaccine comprises a nucleic acid encoding a SARS-CoV-2 spike antigen having at least 95% sequence identity to SEQ ID NO: 11.
37. The method of any one of paragraphs 30-36, wherein the vaccine comprises a nucleic acid having at least 95% sequence identity to SEQ ID NO: 9.
38. The method of paragraph 37, wherein the vaccine comprises a nucleic acid comprising SEQ ID NO: 9.
39. The method of any one of paragraphs 1-7 and 30-38, wherein the first vaccine or the second vaccine comprises: (a) a nucleic acid encoding a SARS-CoV-2 spike antigen comprising two proline substitutions; and (b) a nucleic acid encoding a SARS-CoV-2 spike antigen comprising SEQ ID NO: 11.
40. The method of any one of paragraphs 1-7 and 30-38, wherein the first vaccine or the second vaccine comprises: (a) a nucleic acid encoding a SARS-CoV-2 spike antigen comprising two proline substitutions; and (b) a nucleic acid encoding a SARS-CoV-2 spike antigen comprising SEQ ID NO: 26.
41. The method of any one of paragraphs 1-7 and 30-38, wherein the first vaccine or the second vaccine comprises: (a) a nucleic acid encoding a SARS-CoV-2 spike antigen comprising two proline substitutions; and (b) a nucleic acid encoding a SARS-CoV-2 spike antigen comprising SEQ ID NO: 30.
42. The method of any one of paragraphs 39-41, wherein the ratio of (a) to (b) is 1:1, 1:2, 1:3, 1:4, 2:1, 3:1, or 4:1.
43. The method of any one of paragraphs 1-7 and 30-38, wherein the second vaccine comprises: (a) the nucleic acid encoding a first SARS-CoV-2 2P spike antigen of a first circulating SARS-CoV-2 virus; and (b) the nucleic acid encoding a second SARS-CoV-2 2P spike antigen of a second circulating SARS-CoV-2 virus.
44. The method of paragraph 43, wherein the ratio of (a) to (b) is 1:1, 1:2, 1:3, 1:4, 2:1, 3:1, or 4:1.
45. A method comprising: administering to a subject a booster vaccine comprising a nucleic acid encoding a first SARS-CoV-2 antigen from a first SARS-CoV-2 virus, wherein the subject has previously been administered at least one prime dose of a first vaccine comprising a first nucleic acid encoding the SARS-CoV-2 antigen of the first the SARS-CoV-2 virus, wherein the booster vaccine is administered in an effective amount to induce a neutralizing immune response against a second SARS-CoV-2 virus, wherein the second SARS-CoV-2 virus comprises a second SARS-CoV-2 antigen, wherein the second SARS-CoV-2 antigen has an amino acid sequence with at least one amino acid mutation with respect to a corresponding protein antigen of the first SARS-CoV-2 virus, wherein the booster vaccine is administered in a dosage of 25-100 μg at least 6 months after a first dose of the first vaccine, and wherein the first antigen is a full length stabilized spike protein having a 2P mutation.
46. The method of paragraph 45, wherein the booster vaccine is administered in a dosage of 50 μg.
47. The method of paragraph 45 or 46, wherein the booster vaccine is administered at least about 6 months after a second dose of the first vaccine.
48. The method of paragraph 45 or 46, wherein the booster vaccine is administered 6-12 months after a second dose of the first vaccine.
49. The method of paragraph 45 or 46, wherein the booster vaccine is administered at least about 8 months after a second dose of the first vaccine.
50. The method of any one of paragraphs 45-49, wherein the boost dose is a seasonal boost or a pandemic shift boost to provide a neutralizing immune response against a plurality of variants of concern.
51. The method of any one of paragraphs 1-7 and 30-38, wherein the first vaccine or the second vaccine comprises: (a) a nucleic acid encoding a SARS-CoV-2 spike antigen comprising two proline substitutions; and (b) a nucleic acid encoding a SARS-CoV-2 spike antigen comprising SEQ ID NO: 33.
52. The method of any one of paragraphs 1-7 and 30-38, wherein the first vaccine or the second vaccine comprises: (a) a nucleic acid encoding a SARS-CoV-2 spike antigen comprising two proline substitutions; and (b) a nucleic acid encoding a SARS-CoV-2 spike antigen comprising SEQ ID NO: 36.
53. The method of any one of paragraphs 1-7 and 30-38, wherein the first vaccine or the second vaccine comprises: (a) a nucleic acid encoding a SARS-CoV-2 spike antigen comprising two proline substitutions; and (b) a nucleic acid encoding a SARS-CoV-2 spike antigen comprising SEQ ID NO: 39.
54. The method of any one of paragraphs 1-7 and 30-38, wherein the first vaccine or the second vaccine comprises: (a) a nucleic acid encoding a SARS-CoV-2 spike antigen comprising two proline substitutions; and (b) a nucleic acid encoding a SARS-CoV-2 spike antigen comprising SEQ ID NO: 42.
55. The method of any one of paragraphs 1-7 and 30-54 and the composition of any one of paragraphs 8-29, wherein at least one mRNA further comprises one or more non-coding sequences in an untranslated region (UTR), optionally a 5′ UTR or 3′ UTR.
56. The method or composition of paragraph 55, wherein all of the mRNAs further comprise one or more non-coding sequences in an UTR, optionally a 5′ UTR or 3′ UTR.
57. The method or composition of paragraph 56, wherein the non-coding sequence is positioned in a 3′ UTR of an mRNA, upstream of the polyA tail of the mRNA.
58. The method or composition of paragraph 56, wherein the non-coding sequence is positioned in a 3′ UTR of an mRNA, downstream of the polyA tail of the mRNA.
59. The method or composition of paragraph 56, wherein the non-coding sequence is positioned in a 3′ UTR of an mRNA between the last codon of the ORF of the mRNA and the first “A” of the poly A tail of the mRNA.
60. The method or composition of paragraph 56, wherein the non-coding sequence comprises between 1 and 10 nucleotides.
61. The method or composition of any one of paragraphs 55-60, wherein the non-coding sequence comprises one or more RNAse cleavage sites.
62. The method or composition of claim 61, wherein the RNAse cleavage site comprises an RNase H cleavage site.

### EXAMPLES

### Example 1. MRNA Vaccine Induces Human Neutralizing Antibodies Against Spike Mutants from Global SARS-CoV-2 Variants

Severe acute respiratory syndrome coronavirus-2 (SARS-CoV-2) is the causative infection of a global pandemic that has led to more than 2 million deaths worldwide. The Moderna mRNA-1273 vaccine has demonstrated ˜94% efficacy in a Phase 3 study and has been approved under Emergency Use Authorization. The emergence of SARS-CoV-2 variants with mutations in the spike protein, most recently circulating isolates containing these substitutions from the United Kingdom (B.1.1.7), Republic of South Africa (B.1.351), Brazil (P.1 lineage), New York (B.1.526), and California (B.1.427/B.1.429 or CAL.20C lineage), has led to lower neutralization from convalescent serum by pseudovirus neutralization (PsVN) assays and resistance to certain monoclonal antibodies. Here, using two orthogonal VSV and lentivirus PsVN assays expressing spike variants of 20E. (EU1), 20A.EU2, D614G-N439, mink cluster 5, B.1.1.7 (UK) and B.1.351 (RSA) variants, the neutralizing capacity of sera from human subjects or non-human primates that received mRNA-1273 were assessed. No significant impact on neutralization against the B.1.1.7 variant was detected in either case; however, reduced neutralization was measured against the mutations present in B.1.351. Geometric mean titer (GMT) of human sera from clinical trial participants in VSV PsVN assay using D614G spike was 1/1852. VSV pseudoviruses with spike containing K417N-E484K-N501Y-D614G and full B.1.351 mutations resulted in 2.7 and 6.4-fold reduction, respectively, when compared to the D614G VSV pseudovirus. Importantly, the VSV PsVN GMT of these human sera to the full B.1.351 spike variant was still 1/290, with all sera evaluated able to fully neutralize. Similarly, sera from NHPs immunized with 30 or 100 μg of mRNA-1273 had VSV PsVN GMTs of ˜1/323 or 1/404, respectively, against the full B. 1.351 spike variant with a ˜5 to 10-fold reduction compared to D614G. Testing vaccine immune sera against a variety of pseudoviruses, the B.1.351 variant showed the greatest decrease in PsVN activity when compared to neutralizing activity against the D614G pseudovirus. Nevertheless, the GMT of VSV PsVN titers in human vaccine sera against the B.1.351 variant remained at ˜1/300. The studies are described in more detail below.

In this study, neutralization of sera from Phase 1 clinical trial participants vaccinated with mRNA encoding Spike protein with two proline substitutions (“mRNA vaccine”) against recombinant vesicular stomatitis virus (VSV)-based SARS-CoV-2 in a pseudovirus neutralization (PsVN) assay was assessed with Spike protein from the USA-WA1/2020 isolate, later arising variants including the D614G variant, the B.1.1.7 and B.1.1.351 variants, and variants that previously emerged (20E.EU1, 20A.EU2, D614G-N439K, and mink cluster 5 variant). The effect of both single mutations and combinations of mutations present in the receptor binding domain (RBD) region of the S protein were examined. Orthogonal assessments in VSV and pseudotyped lentiviral neutralization assays were also performed on sera from non-human primates (NHPs) that received the mRNA encoding Spike protein with two proline substitutions vaccine at two different doses.

To assess the ability of the mRNA vaccine to elicit potently neutralizing antibodies against this broad spectrum of SARS-CoV-2 variants, the sera of from mRNA-immunized NHPs that received 30 μg administered twice and participants in the Phase 1 clinical study immunized with the mRNA vaccine at the approved dose of 100 μg given twice were analyzed. Neutralization activity of immune sera was measured with SARS-CoV-2 full-length Spike pseudotyped recombinant VSV-AG-firefly luciferase virus, and in PsVN assays against homotypic SARS-CoV-2_D614, which contains the Spike protein of the USA-WA1/2020 isolate (D614), the D614G version, or Spike protein from 20A.EU1, 20A.EU2 and mink cluster 5 variants (Table 2).

TABLE 2
Spike mutations in SARS-CoV-2 variants

| Variant Name | Amino Acid Changes in Spike |
|---|---|
| 20A.EU1 | A222V-D614G |
| 20A.EU2 | S477N-D614G |
| N439K-D614G | N439K-D614G |
| Mink Cluster 5 Variant | ΔH69ΔV70-Y453F-D614G-I692V-M1229I |
| B.1.1.7 | ΔH69ΔV70-ΔY144-N501Y- |
| (a.k.a., 20I/501Y.V1, VOC | A570D-D614G-P681H-T716I- |
| 202012/01) | S982A-D1118H |
| B.1.351 | L18F-D80A-D215G- |
| (a.k.a., 20H/501Y.V2) | ΔL242ΔA243ΔL244-R246I-K417N- |
| | E484K-N501Y-D614G-A701V |

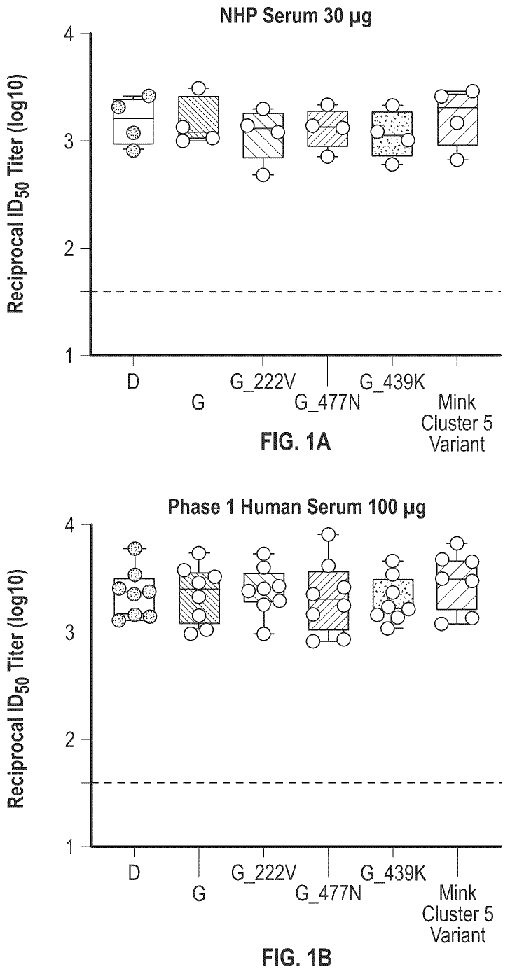

Results demonstrated that the antibody response elicited by the mRNA-1273 vaccine provides similar levels of neutralization against these SARS-CoV-2 Spike variants that emerged prior to the B.1.1.7 and B.1.351 lineages as against the USA-WA1/2020 (D614) strain. This observation includes the G614 variant that has been shown have higher neutralization titers in lentiviral pseudovirus neutralization assays (FIGS. 1A-1B). One of these Spike variants is from mink cluster 5 variant of Denmark, which contains 69-70 del in addition to other mutations (Y453F-1692V-M1229I).

Sera from NHPs vaccinated on day 1 and 29 with 30 or 100 μg mRNA vaccines were analyzed next. Neutralizing antibody responses were measured using orthogonal assessments with both the lentiviral and the VSV-based PsVN assays. Pseudoviruses in both assays incorporated the full-length Spike protein, conferring the USA-WA1/2020 (D614), G614, isolated, partial, or complete set of mutations that are present in the B.1.1.7 and B.1.351 lineages (Table 3).

TABLE 3
Spike variants evaluated the PsVN assay
assessment of mRNA vaccinated NHP sera

| | | Partial or full | |
|---|---|---|---|
| Pseudovirus | Strain | Spike mutants | Mutations |
| VSV | All | | D614G |
| | B.1.1.7 | Full | ΔH69ΔV70-ΔY144- |
| | | | N501Y-A570D-D614G- |
| | | | P681H-T716I-S982A-D1118H |
| | B.1.351 | Partial | K417N-E484K-N501Y-D614G |
| | B.1.351 | Full | L18F-D80A-D215G- |
| | | | ΔL242ΔA243ΔL244- |
| | | | R246I-K417N-E484K- |
| | | | N501Y-D614G- |
| | | | A701V |

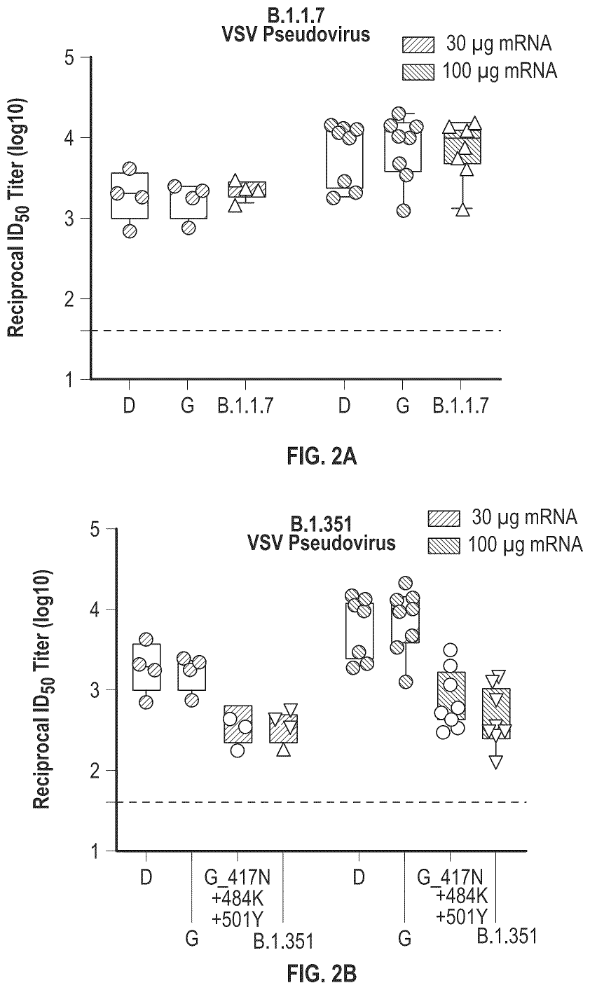

The mutations present in the B.1.1.7 variant, either the complete set of Spike mutations or specific mutations (Table 3) had minimal effect on neutralization in both the VSV and lentiviral neutralization assays. In the VSV assay, no difference was observed between the D614 and the G614 viruses, and there was potent neutralization measured against both (FIG. 2A). Moreover, no decrease in neutralization titers was measured from the B.1.1.7 mutations.

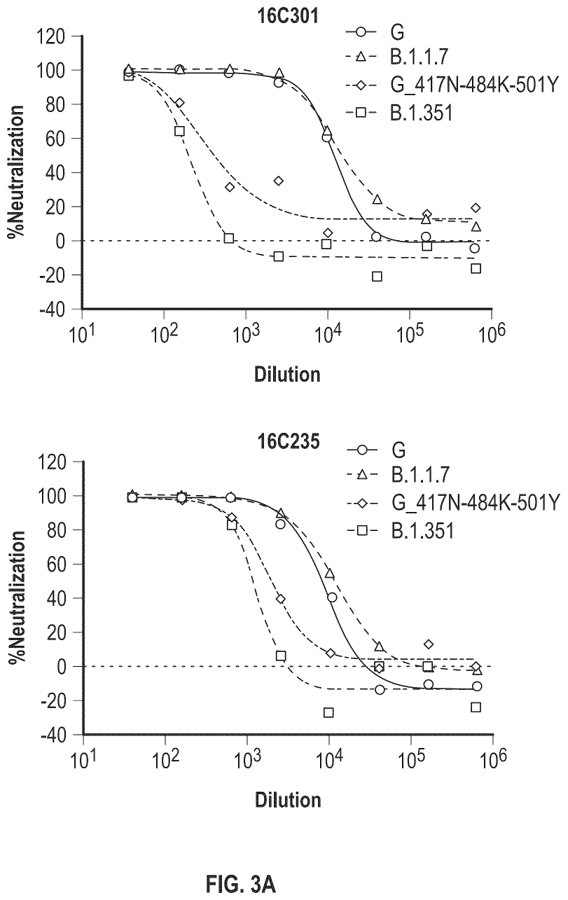

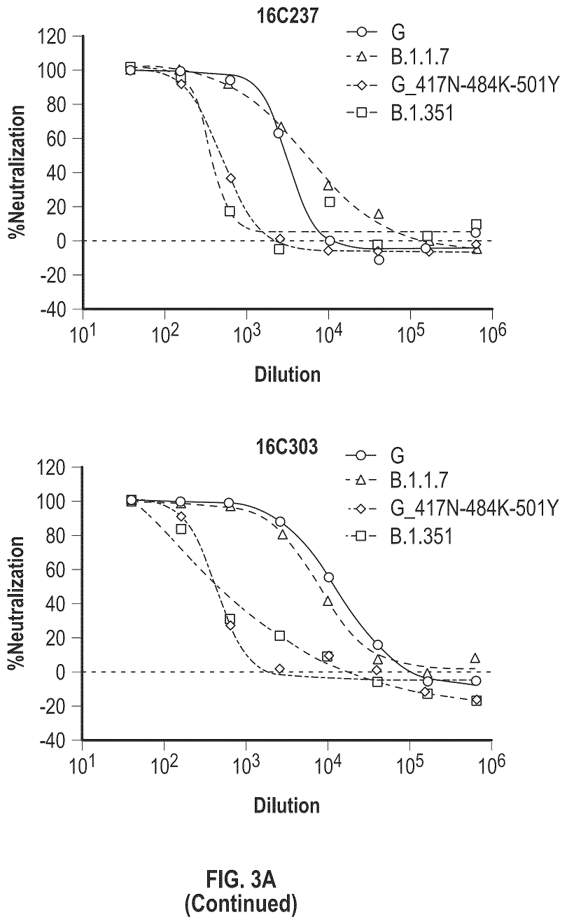

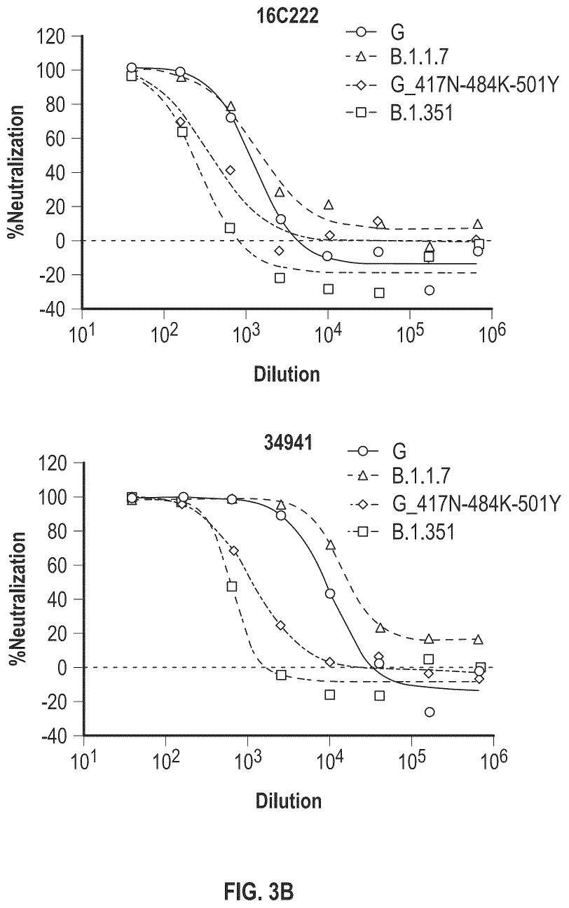

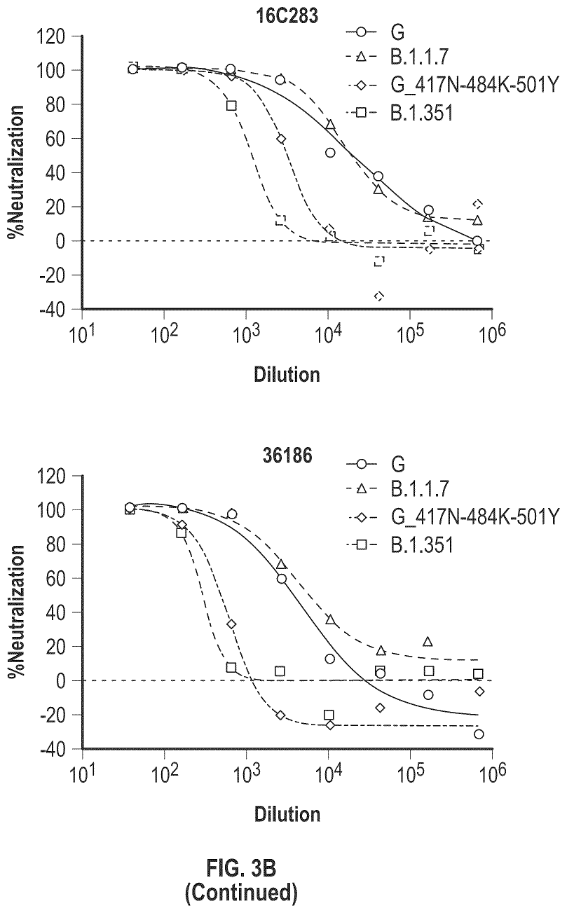

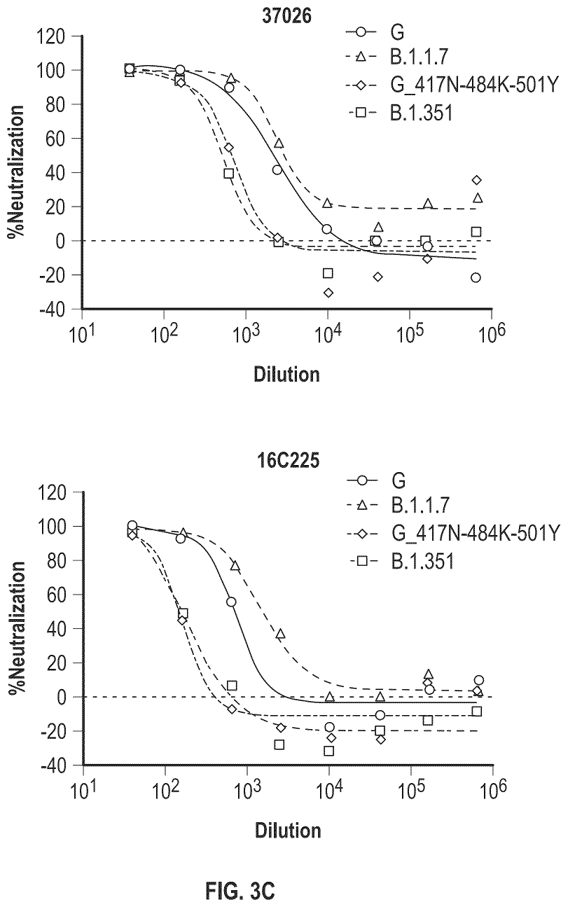

A significant drop in neutralization titers was measured against both the full panel of Spike mutations and specific mutations in the B.1.351 variant, listed in Table 3. In the VSV assay, a 4.3- and 4.8-fold drop in neutralization titers from sera collected from 30 μg dosed animals and a 9.6 and >10-fold drop in neutralization titers from sera from 100 μg dosed animals were measured against the partial or full panel of mutations, respectively (FIG. 2B). Neutralization titers remained high, at or above the level elicited by vaccination of NHPs with a 10 μg dose of mRNA vaccines that were protected from high-dose viral challenge with the WA strain (Corbett et al, 2020, Evaluation of the mRNA-1273 Vaccine against SARS-CoV-2 in Nonhuman Primates. N Engl J Med 383, 1544-1555). All samples were still able to fully neutralize the virus although at lower dilutions as shown by the neutralization curves from the assay (FIG. 3A-3C).

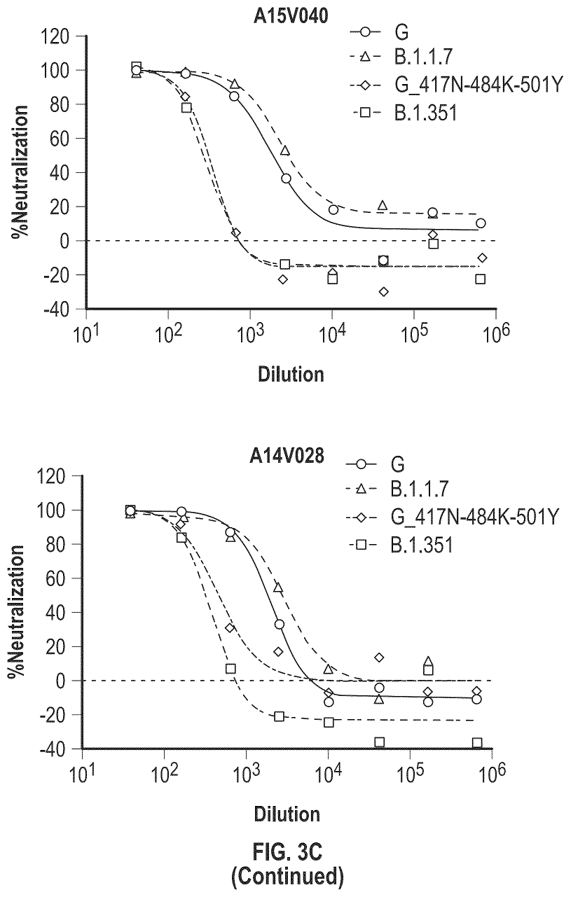

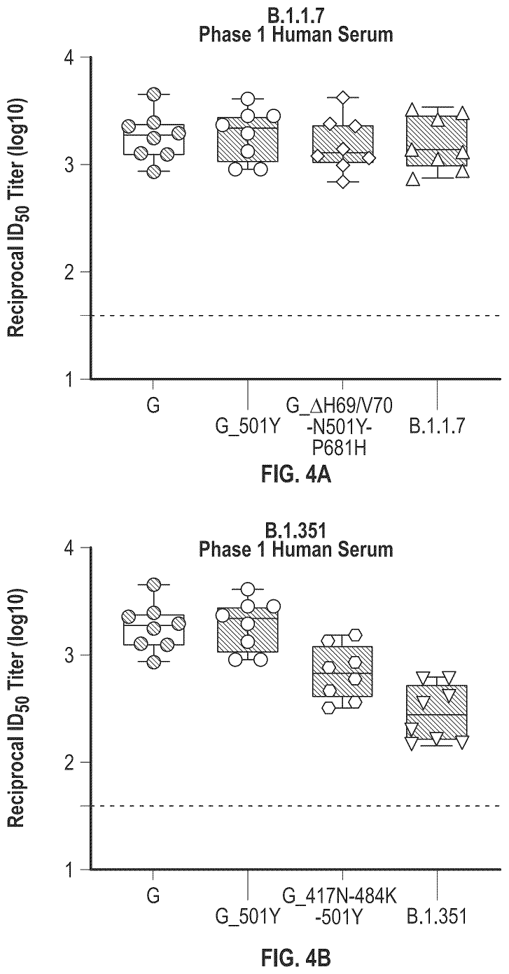

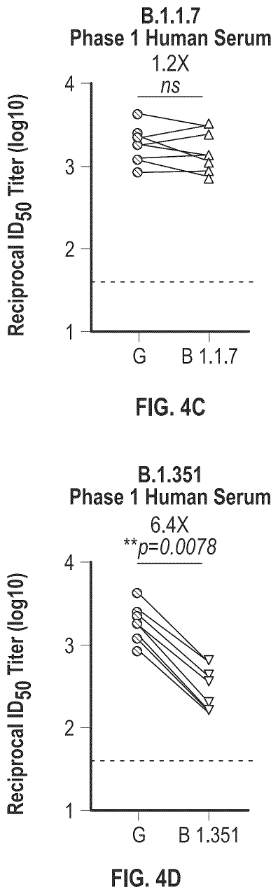

Next, mRNA Phase 1 human sera's neutralization against B.1.1.7 and B.1.351 was examined. The mutations present in the B.1.1.7 variant, either the full panel of Spike mutations or specific mutations (Table 3) had minimal effect on neutralization of human mRNA vaccine phase 1 participant sera (FIG. 4A). In the VSV assay, no significant decrease in neutralization titers were measured from the B.1.1.7 mutations, either partial set of mutations (D614G_N501Y, D614G_ΔH69ΔV70_N501Y_P681H) or the full B.1.1.7 variant (FIG. 4C).

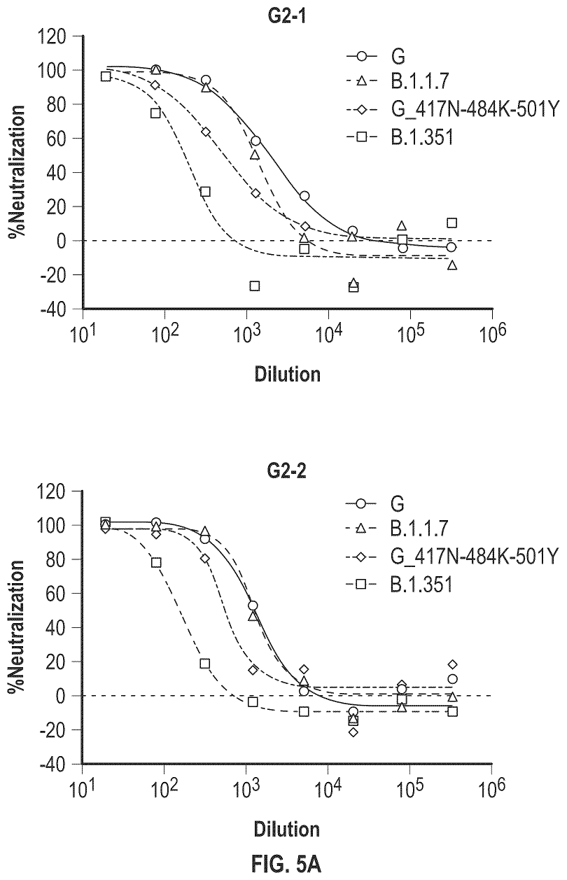

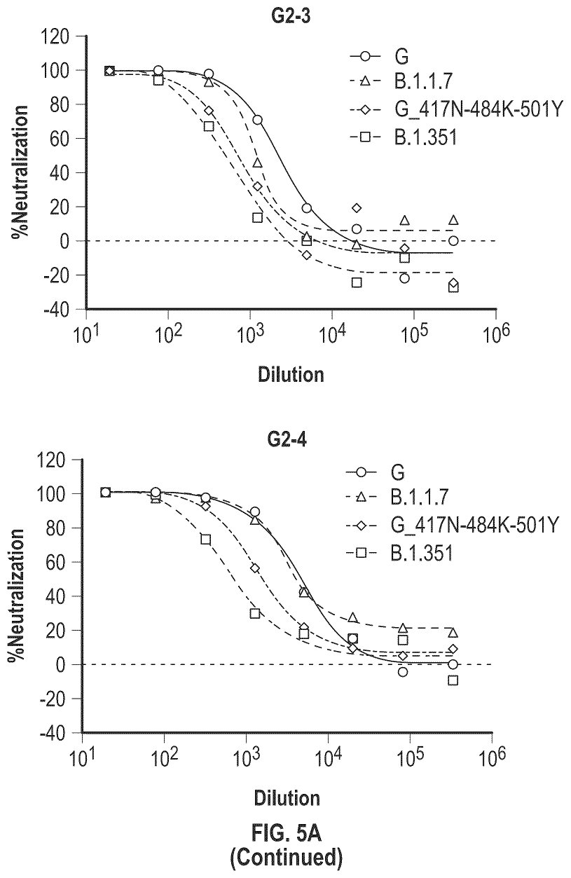

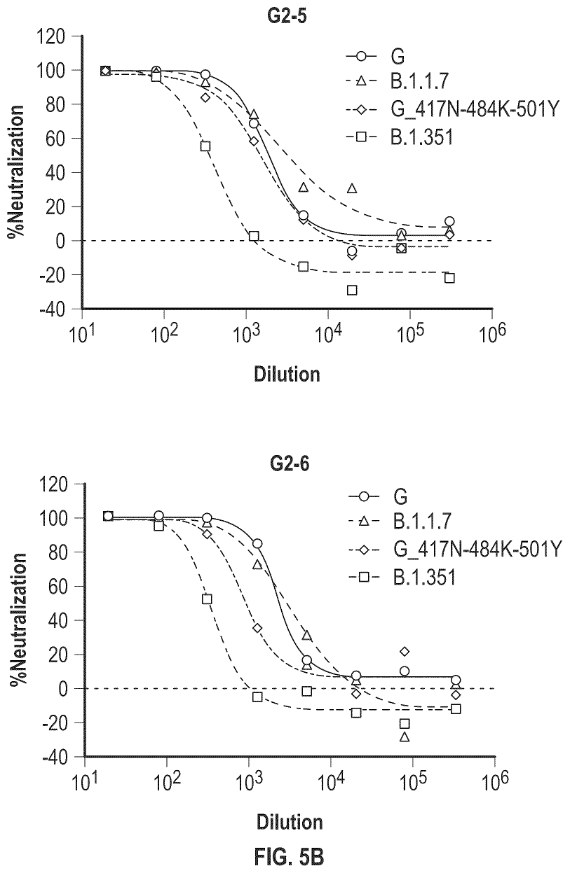

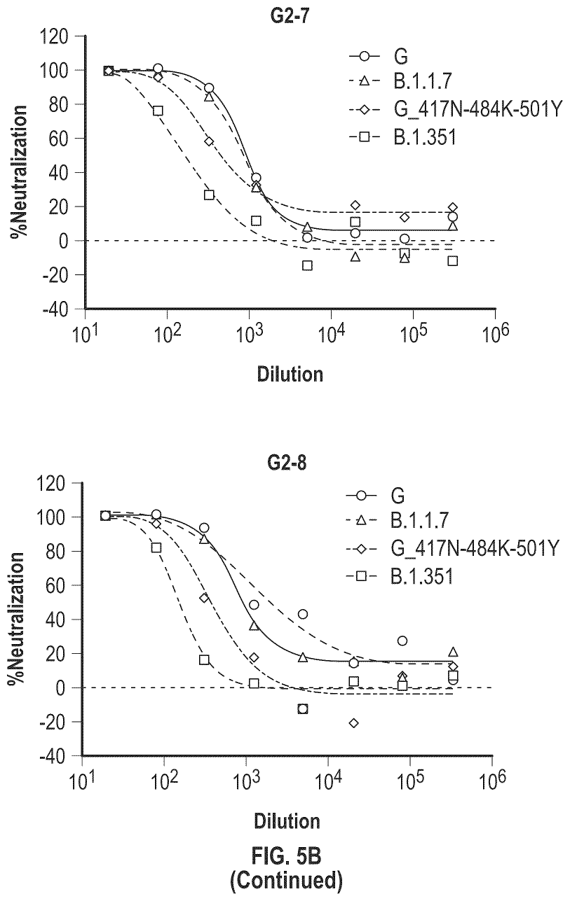

In contrast, a significant drop in neutralization titers were measured against both the full panel of Spike mutations and specific mutations in the B.1.351 variant, listed in Table 3. In the VSV assay, using Phase 1 one week post-boost clinical trial samples, a 2.7- and 6.4-fold reduction in neutralization titers against the partial or full panel of mutations, respectively, was detected (FIG. 4B). Despite diminished neutralizing responses against the B.1.351 variant, neutralization titers were still generally high, and all sera samples completely neutralized the VSV pseudovirus, albeit at lower dilutions as depicted by the neutralization assay curves (FIGS. 5A-5B). Individual animal numbers from the study are indicated above each group.

### Discussion

In this study, the neutralization capacity of sera from eight Phase 1 clinical trial participants (aged 18-55 years) who received two 100 μg doses of the mRNA vaccine, and NHPs immunized with two doses of 30 μg or 100 μg of the mRNA vaccine were assessed. Neutralization was measured against the original D614 Spike, the dominant D614G Spike variant, mutations in 20A.EU1, 20A.EU2, mink cluster 5 variant, N439K-D614G, and either the full panel or key mutations found in the B.1.1.7 and B.1.351 variants strains. The 30 μg dose in NHPs was selected, as it elicits similar neutralizing titers against both D614 and D614G VSV pseudoviruses to those of humans receiving two 100-μg doses of the mRNA vaccine. Assessing the 30-μg dose in NHPs may help elucidate any dose-dependent effects on neutralizing responses toward the new Spike variants.

Both single and combined mutations of interest found in the B.1.1.7 or B.1.351 variants were evaluated in vitro, utilizing a VSV-pseudovirus reporter system. In analyses of both human and NHP sera, the neutralizing responses against the original D614 and the D614G Spike variant were first determined to provide a baseline for comparison with the newer, more elusive variants. Consistent with prior analyses, all eight samples from Phase 1 participants demonstrated robust neutralizing responses against both D614 and D614G SARS-CoV-2 Spikes. Additionally, mRNA-immunized NHPs showed neutralizing antibody titers in line with established efficacy reports (Corbett et al. SARS-CoV-2 mRNA vaccine design enabled by prototype pathogen preparedness. Nature (2020), 586, 567-571).

No significant impact on neutralization was observed from either the full set of mutations found in the B.1.1.7 variant or the N501Y and the 69-70 deletion. Although these mutations have been reported to lessen neutralization from convalescent sera and to increase infectivity, sera from the Phase 1 participants and NHPs immunized with mRNA-1273 were able to neutralize the B. 1.1.7 variant to the same level as the D614G virus.

Consistent with other recent reports (Wang et al. (2021) mRNA vaccine-elicited antibodies to SARS-CoV-2 and circulating variants. BioRxiv 2021.01.15.426911), assessing neutralization of some of the mutations found in B.1.351, there was a 2.7-fold reduction in neutralization after incorporation of the 3 mutations found in the RBD (K417N-E484K-N501Y) and a 6.4-fold reduction in neutralization when the full panel of mutations was included. The VSV-based pseudovirus neutralization titer against the full panel of mutations remained at ˜ 1/300 and all samples were able to fully neutralize the mutant viruses. The data in NHP showed a >10 or 4.8-fold reduction from 100 and 30 μg dose groups, respectively, compared to D614G; however, the VSV-based PsVN titer against the full panel of mutations at both doses was ˜ 1/300. All samples from the clinical trial subjects and NHP fully neutralized the variant viruses at lower dilutions of sera, demonstrating that, despite the reduction in neutralization titers, the polyclonal sera were still able to fully neutralize the virus. A true ID50 shift was observed, meaning all sera showed a reduction in ID50 titers but none showed lack of neutralization.

The data from this sample set shows that, in humans and NHP, there is no reduction in PsVN assay pseudovirus neutralization titers against the B.1.1.7 variant and a ˜5-fold reduction against the B.1.351 variant. Nevertheless, the pseudovirus neutralization titer against the B.1.351 in both humans and NHP is still ˜1/300. Prior studies have shown that pseudovirus neutralization titers against D614G correlate with viral neutralization titers. The VSV pseudovirus neutralization titers in humans after two doses of mRNA-1273 was 1/1852 in the VSV PsVN assay, and protection was ˜95%.

Protection was also observed even after a single immunization with much lower neutralization titers, suggesting that the current mRNA vaccine regimen is inducing immune responses likely to be well above a protective threshold. This is also supported by NHP data in which vaccination of NHPs with only 10 μg mRNA protects animals from SARS-CoV-2 challenge (Corbett, NEJM 2020b), although immune response and neutralization titers are well below what is measured after administration of 30 or 100 μg of mRNA-1273 as shown herein.

It seems likely that neutralization titers against the E484K and B.1.351 by sera from NHP vaccinated twice with 30 μg of the mRNA vaccine or from human Phase 1 trial participants vaccinated with 100 μg mRNA vaccine would be sufficient to mediate protection against viral challenge with these variants. Together, these data indicate that, despite the decreased neutralization potency against the B.1.351 variant, vaccination with the mRNA vaccine is likely to protect from the B.1.351 variant. It is important to prepare for the emergence of any variants that may be able to escape from these antibody neutralization responses induced by the current vaccines and to be ready to induce broader neutralization capability to suppress further emerging variants.

### Methods

Animal Studies. Rhesus macaques (NHPs) were immunized with 10 or 30 μg mRNA encoding Spike protein with two proline substitutions (“mRNA vaccine”) on a prime-boost schedule, and sera was collected 4 weeks after the boosting dose (day 56).

Clinical Trial. Humans were immunized with 100 μg mRNA encoding Spike protein with two proline substitutions (“mRNA vaccine”) on a prime-boost schedule and sera was collected 1 week post the boost (day 36). Study protocols and results are reported in Jackson, et al. (2020). See An mRNA Vaccine against SARS-CoV-2-Preliminary Report. N Engl J Med 2020; 383:1920-1931.

Recombinant VSV-based Pseudovirus Neutralization. Codon-optimized full-length spike protein of the USA-WA1/2020 isolate (D614), D614G, or the indicated spike variants listed in Tables 2 and 3 were cloned into pCAGGS vector. To make SARS-CoV-2 full-length spike pseudotyped recombinant VSV-AG-firefly luciferase virus, BHK-21/WI-2 cells (Kerafast, EH1011) were transfected with the spike expression plasmid and subsequently infected with VSVΔG-firefly-luciferase as previously described (Whitt, 2010, Journal of Virological Methods 169, 365-374). For the neutralization assay, serially diluted serum samples were mixed with pseudovirus and incubated at 37° C. for 45 minutes. The virus-serum mix was subsequently used to infect A549-hACE2-TMPRSS2 cells for 18 hours at 37° C. before adding ONE-Glo reagent (Promega E6120) for measurement of luciferase signal (relative luminescence unit; RLU). The percentage of neutralization was calculated based on RLU of the virus only control, and subsequently analyzed using four-parameter logistic curve (Prism 8).

### Example 2: Coronavirus Strain Challenge

The instant study is designed to test the efficacy in hamsters, mice and/or rabbits of candidate coronavirus vaccines comprising an mRNA as disclosed herein encoding a coronavirus antigen (e.g., the spike(S) protein, the S1 subunit (S1) of the spike protein, or the S2 subunit (S2) of the spike protein, a domain etc), such as a SARS-CoV-2 antigen, against a lethal challenge with a coronavirus. Animals are challenged with a lethal dose (10×LD90; ˜ 100 plaque-forming units; PFU) of coronavirus.

The animals used are ˜6-8 week old animals in groups of ˜10. Animals are vaccinated on weeks 0 and 3 via an IM, ID or IV route of administration. Candidate vaccines are chemically modified or unmodified. Animal serum is tested for microneutralization. Animals are then challenged with ˜1 LD90 of coronavirus on week ˜6-8 via an IN, IM, ID or IV route of administration. Endpoint is day ˜13-15 post infection, death or euthanasia. Animals displaying severe illness as determined by >30% weight loss, extreme lethargy or paralysis are euthanized. Body temperature and weight are assessed and recorded daily.

### Example 3—In Vivo Expression of SARS-CoV-2 mRNA Vaccine Constructs

BALB/c mice, 6-8 weeks of age, are administered either 2 μg or 10 μg of a COVID-19 construct or Tris buffer (as a control) intramuscularly in each hind leg. The constructs comprise any of the mRNA encoding antigens disclosed herein in cationic (amino) lipid nanoparticles, 10.7 mM sodium acetate, 8.7% sucrose, 20 mM Tris (pH 7.5). One day later, spleens and lymph nodes are collected to detect protein expression using flow cytometry.

### Example 4—SARS-CoV-2 mRNA Protects Humanized Mice from Lethal Challenge

Humanized DPP4 288/330^{+/+} mice are immunized at weeks 0 and 3 weeks with 0.01, 0.1, or 1 μg of SARS-CoV-2 mRNA encoding antigens. Mock-immunized mice are immunized with PBS. Four weeks post-boost, mice are challenged with a lethal dose of mouse-adapted SARS-CoV. Following challenge, mice are monitored for weight loss and signs of viral infection. At days 3 and 5 post-challenge, lungs from 5 mice/group are harvested for analysis of viral titers and hemorrhage.

### Example 5—SARS-CoV-2 mRNA Strain Variant Immunogenicity

BALB/c mice, 6-8 weeks of age, were administered either 1 μg or 10 μg of mRNA encoding a SARS-CoV-2 antigen or PBS (as a control) intramuscularly in one hind leg (formulated as a 50 μL dose) on day 1 and day 22. Seventeen groups were tested (n=10 mice/group): PBS, and then both dosage levels of mRNA encoding Spike protein with two proline substitutions (SEQ ID NO: 18), WH2020_NatSP_2P_E484K_D614G (SEQ ID NO: 1), WH2020_NatSP_2P_K417N_E484K_N501Y_D614G (SEQ ID NO: 6), WH2020_NatSP_2P_L18F_D80A_D215G_L242_244del_R246I_K417N_E484K_N501Y D61 4G_A701V (SEQ ID NO: 9), WH2020_NatSP_2P_H69del_V70del_Y144del_N501Y_A570D_D614G_P681H_T716I S982 A_D1118h (SEQ ID NO: 12), a 1:1 mix of SEQ ID NO: 1 and SEQ ID NO: 6 (5 μg+5 μg, or 0.5 μg+0.5 μg), a 1:1 mix of SEQ ID NO: 1 and SEQ ID NO: 9 (5 μg+5 μg, or 0.5 μg+0.5 μg), and the D614G variant. In each vaccine, the mRNA was formulated in lipid nanoparticles (LNPs) including 0.5-15% PEG-modified lipid, 5-25% non-cationic lipid, 25-55% sterol, and 20-60% ionizable amino lipid. The PEG-modified lipid was 1,2 dimyristoyl-sn-glycerol, methoxypolyethyleneglycol (PEG2000 DMG), the non-cationic lipid was 1,2 distearoyl-sn-glycero-3-phosphocholine (DSPC), the sterol was cholesterol, and the ionizable amino lipid had the structure of Compound 1, for example. An overview of this dosing schedule is provided in Table 4.

TABLE 4
Administration schedule of SARS-CoV-2 mRNA variants.

| | | | Dose Mass | Dose | |
|---|---|---|---|---|---|
| | N= | TA | (μg/animal) | Regime | Read-out |
| 1 | 10 | PBS | | Two | ELISA |
| 2 | 10 | mRNA encoding Spike protein with | 10 | doses, | (S-2P) & |
| 3 | 10 | two proline substitutions | 1 | prime on | Neutralization |
| 8 | 10 | WH2020_NatSP_2P_L18F_D80A_D215G_L242_244del_ | 10 | Day 1 and | |
| 9 | 10 | R246I_K417N_E484K_N501Y_D614G_A701V | 1 | boost on | |
| | | (mRNA-1273.351) | | Day 22 | |
| 14 | 10 | 1:1 mix of mRNA-1273 & | 10 (5 + 5) | | |
| 15 | 10 | WH2020_NatSP_2P_L18F_D80A_D215G_L242_244del_ | 1 (0.5 + 0.5) | | |
| | | R246I_K417N_E484K_N501Y_D614G_A701V | | | |
| | | (mRNA-1273.351) | | | |
| 16 | 10 | WH2020_NatSP_2P_001_D614G | 10 | | |
| 17 | 10 | | 1 | | |

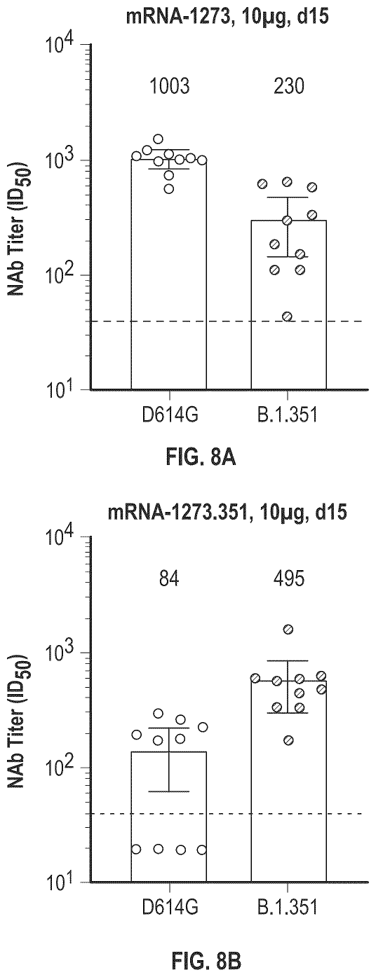

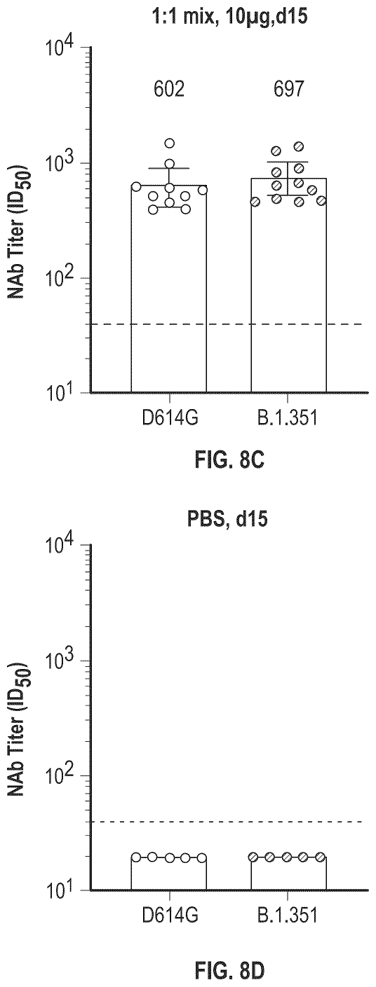

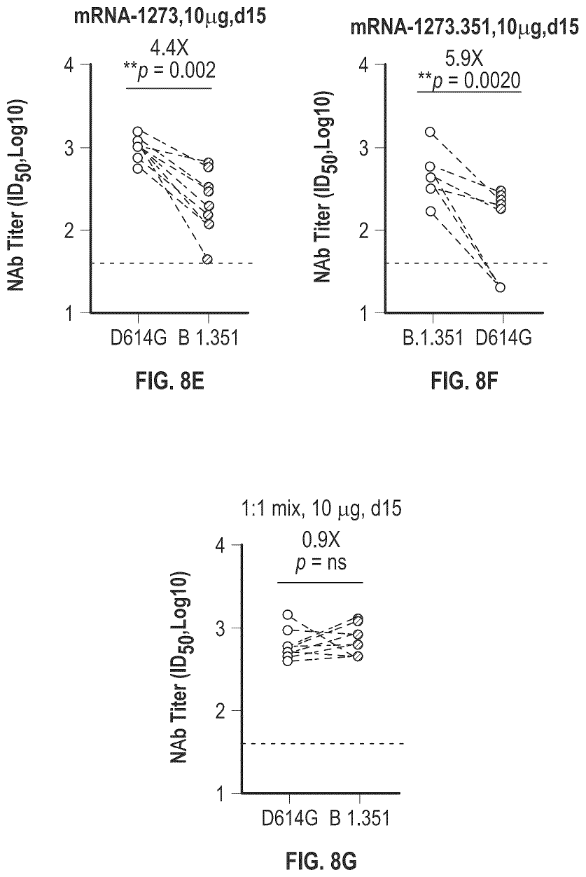

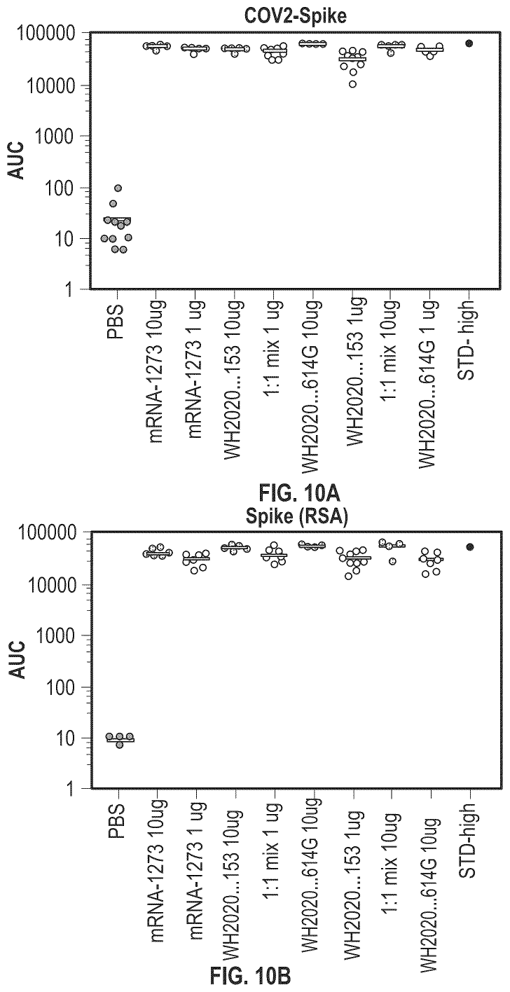

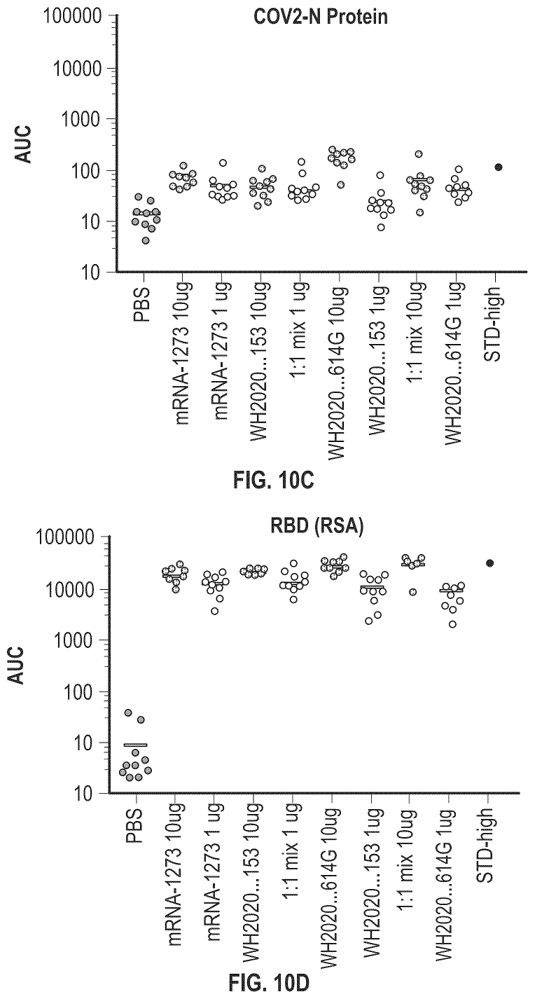

Blood samples were taken from the mice on day 15 and day 36 and analyzed by ELISA and neutralization assays as described herein. Briefly, codon-optimized full-length spike protein of the original SARS-CoV-2 isolate and the D614G mutation (Wild-type or D614G), or the Spike protein variants listed herein (e.g., B.1.351, P.1, CAL.20C) were cloned into pCAGGS vectors. To make SARS-CoV-2 full-length spike pseudotyped recombinant VSV-AG-firefly luciferase virus, BHK-21/WI-2 cells (Kerafast, EH1011) were transfected with the spike expression plasmid and subsequently infected with VSVΔG-firefly-luciferase as previously described (Whitt, 2010). For the neutralization assays, serially diluted serum samples were mixed with pseudovirus and incubated at 37 C for 45 minutes. The virus-serum mix was subsequently used to infect A549-hACE2-TMPRSS2 cells for 18 hours at 37C before adding ONE-Glo reagent (Promega E6120) for measurement of luciferase signal (relative luminescence unit; RLU). The percentage of neutralization was calculated based on RLU of the virus only control, and subsequently analyzed using four-parameter logistic curve (Prism 8). The results of these immunogenicity investigations are shown FIGS. 8A-10D.

Sera of mice immunized with the compositions listed in Table 4 were collected at day 15 post-administration of a first dose, and evaluated in a pseudovirus neutralization assay, as described above. mRNA-1273, encoding wild-type SARS-CoV-2 Spike protein stabilized with 2P substitutions, elicited 4.4× higher neutralization titers against the VSV PSV with the D614G Spike protein than the B.1.351 Spike protein (FIGS. 8A, 8E). Conversely, mRNA-1273.351, encoding SARS-CoV-2 Spike protein with 2P stabilizing substitutions and the variants listed in Table 4, elicited 5.9× higher neutralization titers against the B.1.351 Spike protein than against the D614G variant Spike protein (FIGS. 8B, 8F). Furthermore, a 1:1 mix of both mRNA-1273 and mRNA-1273.351 (5 μg of each mRNA) elicited similar neutralization titers against both Spike proteins, with no significant difference (FIGS. 8C, 8G).

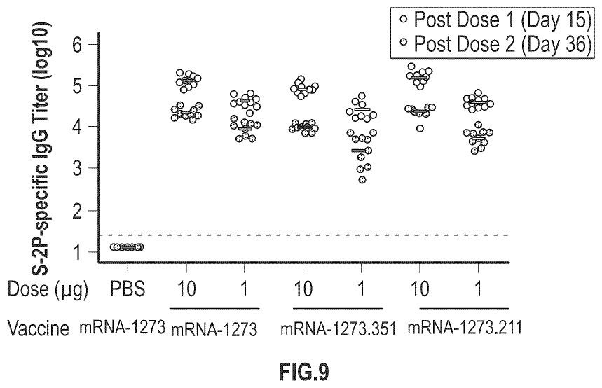

Sera of mice immunized with the compositions listed in Table 4 were collected at day 15 post-administration of a first dose, and day 36 post-administration of a second dose, and evaluated by ELISA to quantify titers of IgG specific to 2P-stabilized SARS-CoV-2 Spike protein (FIG. 9). Relative to mRNA-1273, mRNA-1273.351 elicited lower IgG titers specific to WA.1 S-2P protein, but a 1:1 mixture of mRNA-1273 and mRNA-1273.351 elicited similar titers to an equivalent dose, in terms of total RNA, of mRNA-1273 at the 15 day measurement (FIG. 9). Increased S-2P binding titers were measured after the second dose (day 36). These results demonstrated that both mRNA-1273.351 and mRNA-1273.211 were active and immunogenic. Slightly lower antibody levels were seen for mRNA-1273.351 versus mRNA-1273, potentially due to the coating S-2P protein used in the ELISA being homologous to mRNA-1273.

Sera of mice immunized with the 1 μg compositions listed in Table 4 were collected at day 36 post-administration of a second dose, and evaluated neutralizing antibody titers against the D614G variant Spike protein and against the B.1.351 Spike protein variant. The data is shown in Table 5 below, where mRNA-1273 is mRNA encoding Spike protein with two proline substitutions, mRNA-1273.351 is WH2020_NatSP_2P_L18F_D80A_D215G_L242_244del_R246I_K417N_E484K_N501Y_D61 4G_A701V and mRNA-1273.211 is a 1:1 ratio of mRNA-1273: mRNA-1273.351.

TABLE 5
Neutralization Titers (1 μg Dose)

| | | NAb Titer |
|---|---|---|
| Virus | Vaccine | (ID₅₀) |
| D614G | mRNA-1273 | 16749 |
| D614G | mRNA-1273.351 | 2733 |
| D614G | mRNA-1273.211 | 14832 |
| B.1.351 | mRNA-1273 | 7788 |
| B.1.351 | mRNA-1273.351 | 10948 |
| B.1.351 | mRNA-1273.211 | 19031 |

The data demonstrate that mRNA-1273.211 elicits a robust and comparable neutralizing titer against the D614G and B.1.351 Spike protein variants (1.3× change from D614G to B.1.351). Its response closely matched that of mRNA-1273 against the D614G variant. Similarly, mRNA-1273.351 elicited a robust neutralizing titer against the B.1.351 Spike protein variant; however, its neutralizing antibody titer was 1.6-fold lower than that of mRNA-1273. 211. The neutralizing activity of mRNA-1273.351 against D614G was found to be approximately 4-fold lower than that against B.1.351 Spike protein variant. mRNA-1273 was found to have a 2.1-fold lower neutralizing activity against the B.1.351 Spike protein variant as compared to the D614G Spike protein variant.

The experiment was also performed with a 10 μg dose. The results from the study are shown in Table 6 below.

TABLE 6
Neutralization Titers (10 μg Dose)

| | | NAb Titer |
|---|---|---|
| Virus | Vaccine | (ID₅₀) |
| D614G | mRNA-1273 | 358533 |
| D614G | mRNA-1273.351 | 111722 |
| D614G | mRNA-1273.211 | 220529 |
| B.1.351 | mRNA-1273 | 150199 |
| B.1.351 | mRNA-1273.351 | 216332 |
| B.1.351 | mRNA-1273.211 | 245387 |

The data largely follows the trend from the 1 μg dose group. The titer of the mRNA-1273.211 against the D614G spike protein variant is about 2-fold lower than that of mRNA-1273. The gap of mRNA-1273 between the two variants is also approximately 2-fold at both dose levels. Notably, the gap of mRNA-1273.351 neutralizing antibody titer levels against the two variants at the 10 μg dose becomes smaller (2-fold) than at the 1 μg dose (approximately 4-fold).

The sera of mice immunized with 1 μg compositions listed in Table 4 were collected at day 36 post-administration of a second dose, and evaluated with respect to neutralizing antibody titers against the D614G variant Spike protein, the CAL.20C Spike protein variant, and the P.1 Spike protein variant. The data is shown in Table 7 below.

TABLE 7
Neutralization Titers (1 μg Dose)

| | | NAb Titer |
|---|---|---|
| Virus | Vaccine | (ID₅₀) |
| D614G | mRNA-1273 | 16410 |
| D614G | mRNA-1273.351 | 2460 |
| D614G | mRNA-1273.211 | 12902 |
| CAL.20C | mRNA-1273 | 6847 |
| CAL.20C | mRNA-1273.351 | 1821 |
| CAL.20C | mRNA-1273.211 | 4089 |
| P.1 | mRNA-1273 | 8293 |
| P.1 | mRNA-1273.351 | 3230 |
| P.1 | mRNA-1273.211 | 10620 |

The data demonstrate that mRNA-1273.211 elicits a similar neutralization titer against D614G and the P.1 Spike protein variant. mRNA-1273.351 immunized mice were found to have a 4- to 6-fold drop of neutralizing antibody titer against the P.1, D614G, and CAL.20C Spike protein variants when compared to the neutralization titer to the B.1.351 Spike protein variant. Without wishing to be bound by theory, it is thought that the extensive N-terminal domain mutations in the B.1.351 Spike protein may drift its immunogenicity away from the D614G and P.1 variants. Both mRNA-1273 and mRNA-1273.211 has similar 3-fold drops in neutralization titer against CAL.20C (the relative titer change between the D614G variant and the CAL.20C variant was 2.4× for mRNA-1273 (p=0.0078), 1.4× for mRNA-1273.351, and 3.2× for mRNA-1273.211 (p=0.0078)). The relative titer change between the D614G variant and the P.1 variant was 2.0× for mRNA-1273, 0.8× for mRNA-1273.351, and 1.2× for mRNA-1273.211.

Additional spike variant antigen designs were similarly tested, including WH2020_NatSP_2P_E484K_D614G (SEQ ID NO: 1), WH2020_NatSP_2P_K417N_E484K_N501Y_D614G (SEQ ID NO: 6), WH2020_NatSP_2P_L18F_D80A_D215G_L242_244del_R246I_K417N_E484K_N501Y_D61 4G_A701V (SEQ ID NO: 10), WH2020_NatSP_2P_H69del_V70del_Y144del_N501Y_A570D_D614G_P681H_T716I_S982 A_D1118h (SEQ ID NO: 12), and a 1:1 mix of SEQ ID NO: 10 and SEQ ID NO: 6 (0.5 μg+0.5 μg). The sera of mice immunized with 1 μg of the mRNA vaccines described above were collected at day 36 post-administration of a second dose, and evaluated with respect to neutralizing antibody titers against the D614G variant Spike protein and the B.1.351 Spike protein variant. The data is shown in Table 8 below.

TABLE 8
Neutralization Titers (1 μg Dose)

| | | NAb |
|---|---|---|
| | | Titer |
| Virus | Vaccine | (ID₅₀) |
| D614G | WH2020_NatSP_2P_E484K_D614G (SEQ ID NO: 1) | 9691 |
| D614G | WH2020_NatSP_2P_H69del_V70del_Y144del_ | 6230 |
| | N501Y_A570D_D614G_P681H_T716I_S982A_D1118h | |
| | (SEQ ID NO: 12) | |
| D614G | WH2020_NatSP_2P_K417N_E484K_ | 16823 |
| | N501Y_D614G (SEQ ID NO: 6) | |
| D614G | 1:1 mix of SEQ ID NO: 10 and 6 | 23390 |
| | WH2020_NatSP_2P_L18F_D80A_D215G_L242_ | |
| | 244del_R246I_K417N_E484K_N501Y_D614G_A701V | |
| | (SEQ ID NO: 10) | |
| | WH2020_NatSP_2P_K417N_E484K_ | |
| | N501Y_D614G (SEQ ID NO: 6) | |
| B.1.351 | WH2020_NatSP_2P_E484K_D614G (SEQ ID NO: 1) | 7397 |
| B.1.351 | WH2020_NatSP_2P_H69del_V70del_Y144del_ | 2357 |
| | N501Y_A570D_D614G_P681H_T716I_S982A_D1118h | |
| | (SEQ ID NO: 12) | |
| B.1.351 | WH2020_NatSP_2P_K417N_E484K_ | 8566 |
| | N501Y_D614G (SEQ ID NO: 6) | |
| B.1.351 | 1:1 mix of SEQ ID NO: 10 and 6 | 16091 |
| | WH2020_NatSP_2P_L18F_D80A_D215G_L242_ | |
| | 244del_R246I_K417N_E484K_N501Y_D614G_A701V | |
| | (SEQ ID NO: 10) | |
| | WH2020_NatSP_2P_K417N_E484K_ | |
| | N501Y_D614G (SEQ ID NO: 6) | |

It was found that the S2P-E484K-D614G and S2P-K417N-E484K-N501Y-D614G vaccines neutralized the D614G variant virus well, and each had a modest drop (1.5- to 2-fold) against the B.1.351 virus. Similar to the results observed with mRNA-1273.211 (1:1 mix of mRNA-1273 and mRNA-1273.351), the 1:1 mixture of mRNA-1273 and S2P-K417N_E484K_N501Y_D614G had consistently high neutralizing titers. In contrast to the mRNA-1273.211 vaccine; however, the 1:1 mixture of mRNA-1273 and S2P-K417N_E484K_N501Y_D614G had a less robust response to the B.1.351 Spike protein variant.

The study was repeated with the S2P-D614G (SEQ ID NO: 18) mRNA vaccine (1 μg doses). On day 36 (after two doses), sera were assayed to determine neutralizing antibody titers against different virus variants, as shown in Table 9.

TABLE 9
Neutralization Titers (1 μg Dose)

| | | NAb Titer |
|---|---|---|
| Virus | Vaccine | (ID₅₀) |
| D614G | S2P-D614G | 23097 |
| B.1.351 | S2P-D614G | 13917 |
| CAL.20C | S2P-D614G | 19692 |
| P.1 | S2P-D614G | 21387 |
| B.1.1.7 + | S2P-D614G | 15596 |
| E484K | | |

The data demonstrates that vaccine S2P-D614G elicited higher neutralizing antibody titers against the variants compared to other mRNA vaccines tested. Moreover, it showed a more consistent antibody titer than the other mRNA vaccines tested. For example, the neutralizing antibody titer measured against the D614G variant was 1.7-fold greater than against the B.1.351 variant. The neutralizing titer against the D614G variant was 1.2 fold higher than against the CAL.20C variant; against the D614G variant and against the P.1 variant was 1.1-fold greater. Finally, the neutralizing titer against the D614G variant was 1.5 fold higher than against the B.1.1.7+E484K variant.

### Example 6—SARS-CoV-2 mRNA B.1.351 Variant Immunogenicity (10 Mutations Vs. 8 Mutations)

BALB/c mice, 6-8 weeks of age, are administered using either 0.1 μg, 1 μg, or 10 μg of mRNA encoding a SARS-CoV-2 Spike protein antigen or PBS (as a control) intramuscularly in one hind leg (formulated as a 50 μL dose) on day 1 and day 22. The mRNAs tested encode mRNA encoding Spike protein with two proline substitutions, WH2020_NatSP_2P, (SEQ ID NO: 18), WH2020_NatSP_2P_L18F_D80A_D215G_L242_244del_R246I_K417N_E484K_N501Y_D61 4G_A701V (SEQ ID NO: 9), or WH2020_NatSP_2P_H69del_V70del_Y144del_N501Y_A570D_D614G_P681H_T716I_S982 A_D1118h (SEQ ID NO: 12). In each vaccine, the mRNA is formulated in lipid nanoparticles (LNPs) including 0.5-15% PEG-modified lipid, 5-25% non-cationic lipid, 25-55% sterol, and 20-60% ionizable amino lipid. The PEG-modified lipid is 1,2 dimyristoyl-sn-glycerol, methoxypolyethyleneglycol (PEG2000 DMG), the non-cationic lipid is 1,2 distearoyl-sn-glycero-3-phosphocholine (DSPC), the sterol is cholesterol, and the ionizable amino lipid has the structure of Compound 1, for example.

Blood samples are taken from the mice on day 21 and day 36 and the serum analyzed by ELISA and neutralization assays as described herein. Lungs and spleen are removed and further analyzed.

### Example 7—SARS-CoV-2 mRNA B.1.351 Variant Third Dose Immunogenicity

BALB/c mice, 6-8 weeks of age, were administered either 0.1 μg or 1 μg of mRNA encoding a SARS-CoV-2 antigen or PBS (as a control) intramuscularly in one hind leg (formulated as a 50 μL dose) on day 1 and day 22. The mice were then administered a third dose at week 8 (day 57). In each vaccine, the mRNA was formulated in lipid nanoparticles (LNPs) including 0.5-15% PEG-modified lipid, 5-25% non-cationic lipid, 25-55% sterol, and 20-60% ionizable amino lipid. The PEG-modified lipid was 1,2 dimyristoyl-sn-glycerol, methoxypolyethyleneglycol (PEG2000 DMG), the non-cationic lipid was 1,2 distearoyl-sn-glycero-3-phosphocholine (DSPC), the sterol was cholesterol, and the ionizable amino lipid had the structure of Compound 1, for example. The administration schedule is provided in Table 10 below (note: mRNA-351 encodes wild-type SARS-CoV-2 with the following mutations: L18F-D80A-D215G-L242-244del-R246I-K417N-E484K-N501Y-D614G-A701V).

TABLE 10
Administration Schedule

| | | | Dose Mass | Prime/ | |
|---|---|---|---|---|---|
| GR | N= | TA | (ug/animal) | boost | 3^{rd} dose |
| 1 | 5 | PBS | — | PBS | PBS |
| 2 | 8 | mRNA encoding the wild-type | 1 | Prime on day 1 | mRNA encoding the wild-type |
| 3 | 8 | Spike protein with two proline | 0.1 | and boost on | Spike protein with two proline |
| | | substitutions | | day 22 with | substitutions (1 ug) 3^{rd} |
| | | | | 1273 (1; | dose on wk 8 |
| 4 | 8 | mRNA encoding the wild-type | 1 | 0.13 g) | mRNA-351 (1 ug) 3^{rd} dose |
| 5 | 8 | Spike protein with two proline | 0.1 | | on wk 8 |
| | | substitutions | | | 1:1 mix of mRNA encoding |
| | | | | | the wild-type Spike protein |
| 6 | 8 | mRNA encoding the wild-type | 1 | | with two proline substitutions + |
| 7 | 8 | Spike protein with two proline | 0.1 | | mRNA-351 (1 ug) |
| | | substitutions | | | 3^{rd} dose on wk 8 |
| 8 | 8 | mRNA encoding the wild-type | 1 | | 1:2 mix of mRNA encoding |
| 9 | 8 | Spike protein with two proline | 0.1 | | the wild-type Spike protein |
| | | substitutions | | | with two proline substitutions + |
| | | | | | mRNA-351 (1 ug) |
| | | | | | 3^{rd} dose on wk 8 |
| 10 | 8 | mRNA encoding the wild-type | 1 | | 1:3 mix of mRNA encoding |
| 11 | 8 | Spike protein with two proline | 0.1 | | the wild-type Spike protein |
| | | substitutions | | | with two proline substitutions + |
| | | | | | mRNA-351 (1 ug) |
| | | | | | 3^{rd} dose on wk 8 |
| 12 | 8 | mRNA-351 | 1 | | mRNA-351(1 ug ) 3^{rd} dose |
| 13 | 8 | | 0.1 | | on wk 8 |
| 14 | 8 | 1:1 mix of mRNA encoding the | 1 | | 1:1 mix of mRNA encoding |
| 15 | 8 | wild-type Spike protein with two | 0.1 | | the wild-type Spike protein |
| | | proline substitutions & mRNA-351 | | | with two proline substitutions + |
| | | | | | mRNA-351 (1 ug) |
| | | | | | 3^{rd} dose on wk 8 |
| 16 | 8 | 1:2 mix of mRNA encoding the | 1 | | 1:2 mix of mRNA encoding |
| 17 | 8 | wild-type Spike protein with two | 0.1 | | the wild-type Spike protein |
| | | proline substitutions & mRNA-351 | | | with two proline substitutions + |
| | | | | | mRNA-351 (1 ug) |
| | | | | | 3^{rd} dose on wk 8 |
| 18 | 8 | 1:3 mix of mRNA encoding the | 1 | | 1:3 mix of mRNA encoding |
| 17 | 8 | wild-type Spike protein with two | 0.1 | | the wild-type Spike protein |
| | | proline substitutions & mRNA-351 | | | with two proline substitutions + |
| | | | | | mRNA-351 (1 ug) |
| | | | | | 3^{rd} dose on wk8 |

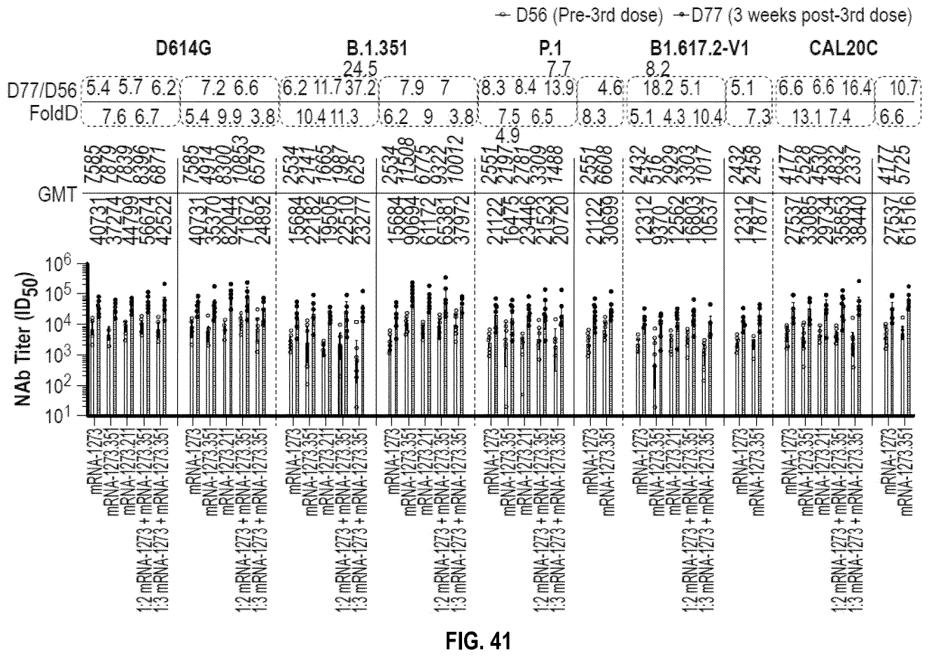

Blood samples were taken on days 21, 36, 56, and 77 and analyzed by ELISA and neutralization assays as described herein. The neutralizing titers are shown in FIG. 41, and demonstrate fold-increases between day 56 and day 77 of 4.6 to 37.2.

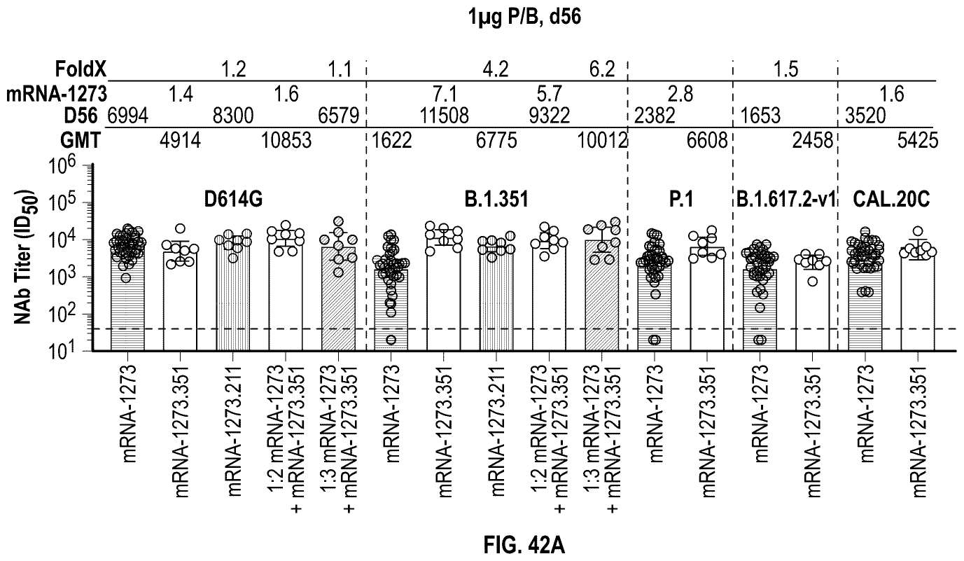

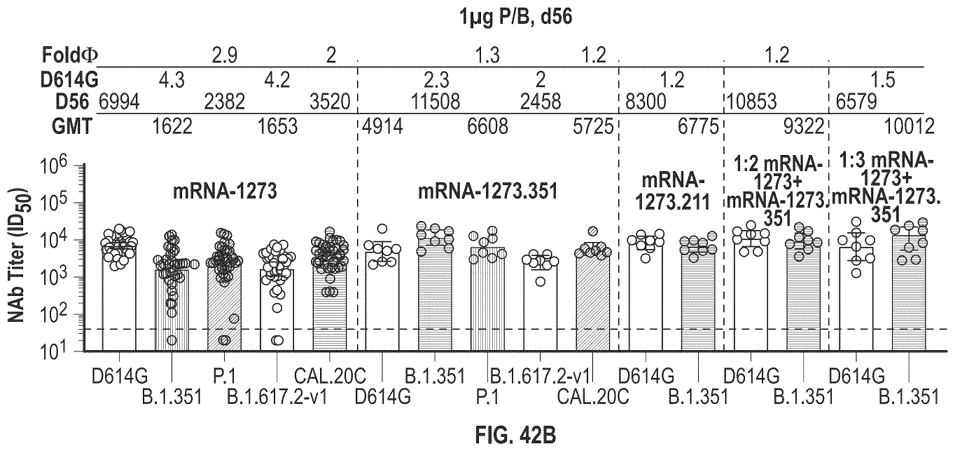

Overall, two doses of mRNA-1273 (n=40) consistently displayed a decrease in neutralizing antibody titer against B.1.351, P.1, B.1.617-2-v1, CAL.20C (FIGS. 42A-42B). In contrast, mRNA-1273.351 (n=8) had a 1.5-3-fold increase on neutralizing antibody titer against P.1, B.1.617.2-v1, CAL.20C (FIGS. 42A-42B). The different ratios tested, 1:2 and 1:3 mRNA-1273+mRNA-1273.351 (n=8), were found to perform equally well as mRNA-1273.351 against B.1.351; however, the 1:2 mRNA-1273+mRNA-1273.351 vaccine (n=8) was found to have the highest neutralizing antibody titer against D614G (FIGS. 42A-42B).

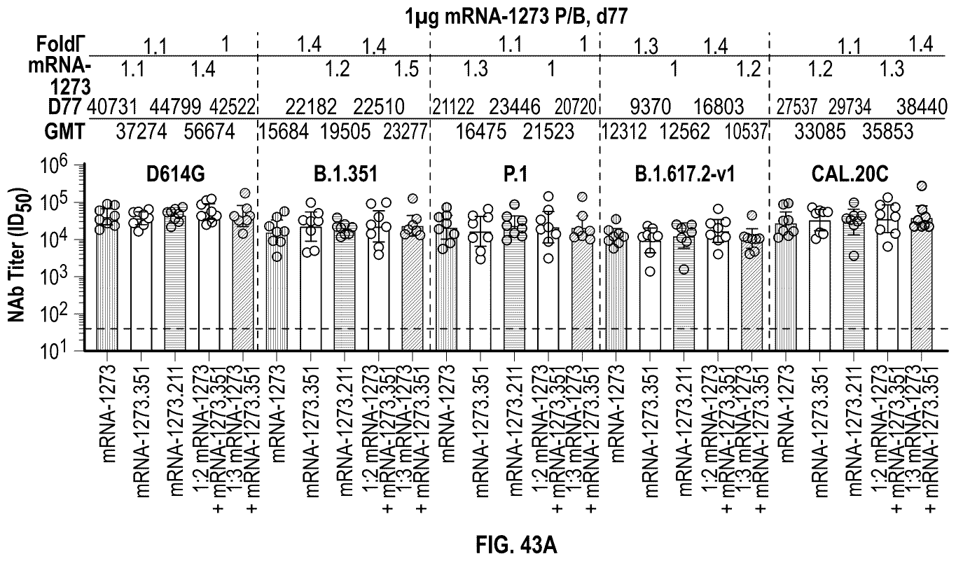

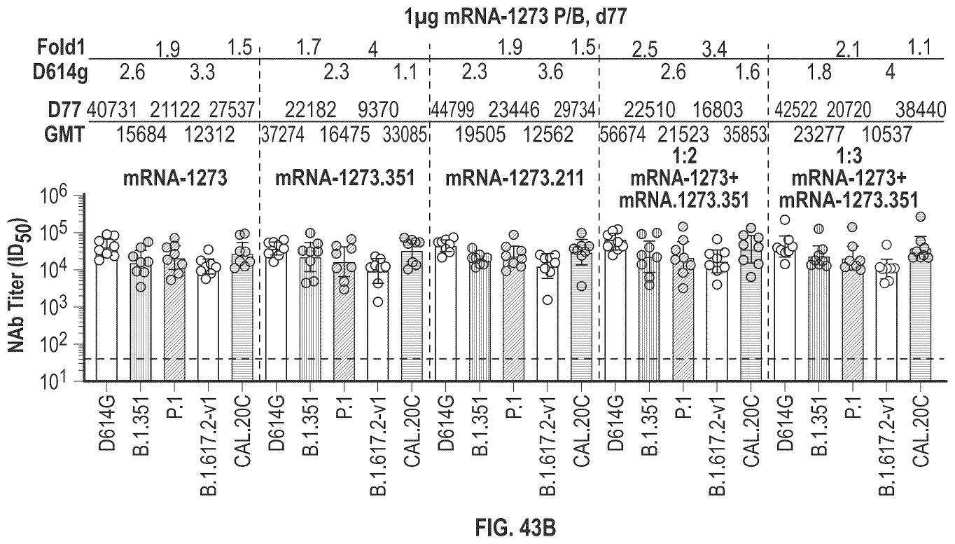

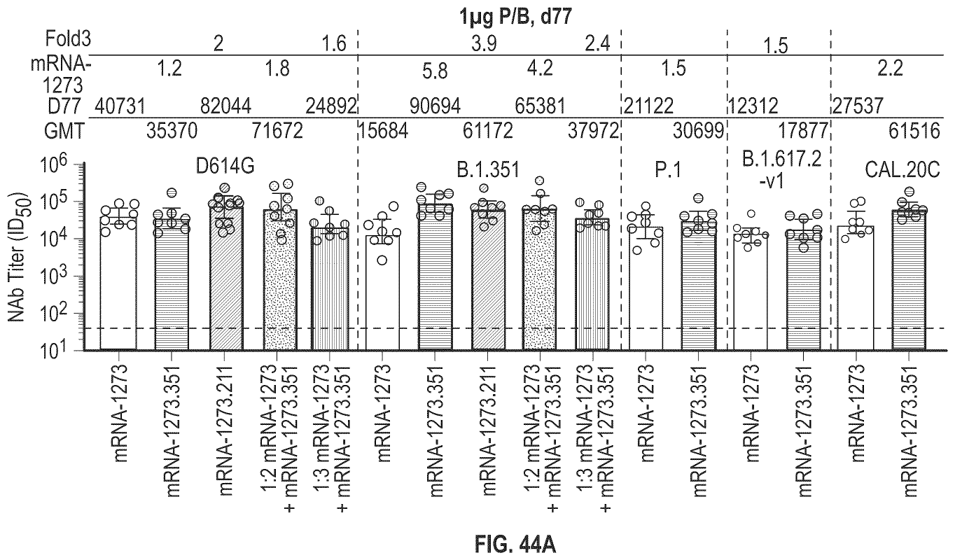

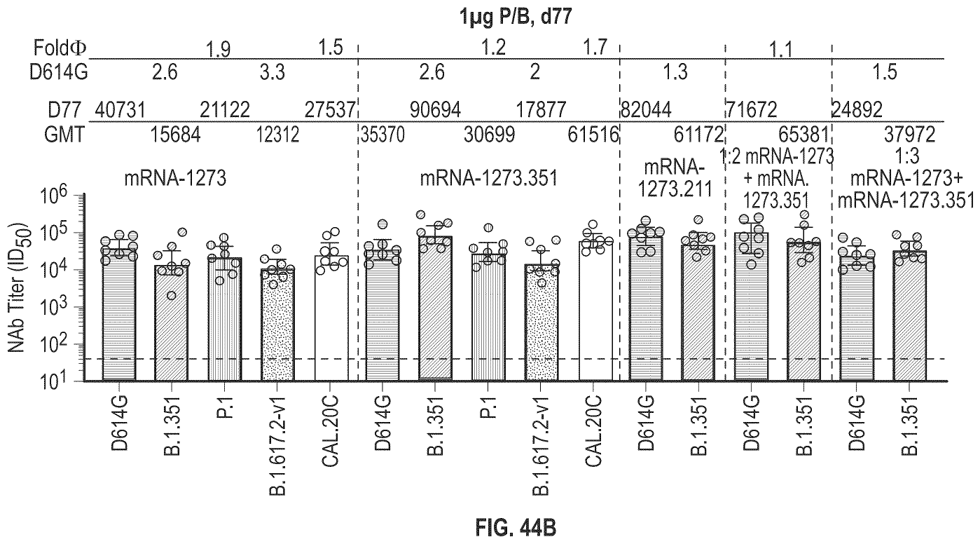

Three weeks after the third dose (booster), all vaccine formulations were found to have a reduction in neutralizing antibody titer compared to D614G, while all vaccine formulations seemed to behave equally well as mRNA-1273 across the full panel of viruses (FIGS. 43A-43B). In particular, mRNA-1273 was found to have a 1.5-3-fold reduction in neutralizing antibody titer against the full panel of viruses. In addition, mRNA-1273.211 and 1:2 mRNA-1273+mRNA-1273.351 performed equally well against D614G and B.1.351 but 1:3 mRNA-1273+mRNA-1273.351 seems to have a slightly lower neutralizing antibody titer (FIGS. 44A-44B). Ultimately, mRNA-1273.351 yielded the highest neutralizing antibody titer against B.1.351.

In summary, as a third dose (booster), all vaccine formulations appeared to perform equally well, although slightly better than the 1:3 mRNA-1273+mRNA-1273.351 formulation.

### Example 8—Immunogenicity of SARS-CoV-2 mRNA (3^{rd} and 4^{th} Doses)

BALB/c mice were immunized at days 1 and 22 with 1 μg of SEQ ID NO: 18 (WH2020_NatSP_2P) (n=8/group). The mice were subsequently administered a first booster dose (dose 3; 1 μg) on day 58 and a second booster dose (dose 4; 1 μg) on day 78 or a first booster dose (dose 3; 0.1 μg or 1 μg) on day 213 and a second booster dose (dose 4; 0.1 μg or 1 μg) on day 234. The first and second booster doses comprised mRNA encoding wild-type SARS-CoV-2 with the following mutations: L18F-D80A-D215G-L242-244del-R246I-K417N-E484K-N501Y-D614G-A701V. In each vaccine, the mRNA was formulated in lipid nanoparticles (LNPs) including 0.5-15% PEG-modified lipid, 5-25% non-cationic lipid, 25-55% sterol, and 20-60% ionizable amino lipid. The PEG-modified lipid was 1,2 dimyristoyl-sn-glycerol, methoxypolyethyleneglycol (PEG2000 DMG), the non-cationic lipid was 1,2 distearoyl-sn-glycero-3-phosphocholine (DSPC), the sterol was cholesterol, and the ionizable amino lipid had the structure of Compound 1, for example.

Blood was collected one day before the first booster dose, one day before the second booster dose, and two weeks following the second booster dose, and analyzed by ELISA and neutralization assays as described herein. Briefly, pseudoviruses with Spike mutation D614G (comparator variant) and B.1.351 (L18F, D80A, D215G, A242-244, R246I, K417N, E484K, N501Y, A701V) were constructed. Neutralization assays were performed using a validated lentivirus-based Spike-pseudotyped virus assay in 293T cells stably transduced to overexpress ACE2 described in Shen et al. (SARS-CoV-2 variant B.1.1.7 is susceptible to neutralizing antibodies elicited by ancestral Spike vaccines. Cell Host & Microbe, in press 2021).

With respect to the groups that received booster doses on Day 213 and Day 234, the neutralizing antibody titer was found to drop about 2-fold over the six-month period. That is, on day 36, the neutralizing titer was 9940 and on day 212 (the day before the first booster dose), the neutralizing antibody titer was 6729.

Neutralizing antibody titers against the D614G variant and the B.1.351 variant were measured on day 212 (before the third dose, 1 μg) and day 233 (after the third dose). The data is shown in Table 11 below.

TABLE 11
Neutralization Titers (1 μg Dose)

| | | NAb Titer |
|---|---|---|
| Virus | Day | (ID₅₀) |
| D614G | 212 | 6729 |
| D614G | 233 | 30386 |
| B.1.351 | 212 | 1015 |
| B.1.351 | 233 | 15524 |

As shown in Table 11, the neutralizing antibody titer was increased from day 212 to day 233 against the D614 variant Spike protein and more dramatically against the B.1.351 variant Spike protein. The relative titer change between day 212 and day 233 against the D614G variant was 4.5× and the relative titer change between day 212 and day 233 against the B.1.351 variant was 15×. The neutralizing antibody titer was 6.6× greater against the D614G variant than against the B.1.351 variant at day 212 (before administration of the booster); however, after the booster dose (day 233), the neutralization titers elicited against the D614G variant Spike protein were 2× those elicited by the B.1.351 variant Spike protein.

Similar trends were seen with the 0.1 μg dose. Neutralizing antibody titers against the D614G variant and the B.1.351 variant were measured on day 212 (before the third dose, 0.1 μg) and day 233 (after the third dose). The data is shown in Table 12 below.

TABLE 12
Neutralization Titers (0.1 μg Dose)

| | | NAb Titer |
|---|---|---|
| Virus | Day | (ID₅₀) |
| D614G | 212 | 548 |
| D614G | 233 | 1985 |
| B.1.351 | 212 | 66 |
| B.1.351 | 233 | 274 |

As shown in Table 12, the neutralizing antibody titer was increased from day 212 to day 233 against the D614 variant Spike protein and the B.1.351 variant Spike protein. The relative titer change between day 212 and day 233 against the D614G variant was 3.6× and the relative titer change between day 212 and day 233 against the B.1.351 variant was 4.2×. The neutralizing antibody titer was 8.3× greater against the D614G variant than against the B.1.351 variant at day 212 (before administration of the booster); however, after the booster dose (day 233), the neutralization titers elicited against the D614G variant Spike protein were 7.2× those elicited by the B.1.351 variant Spike protein.

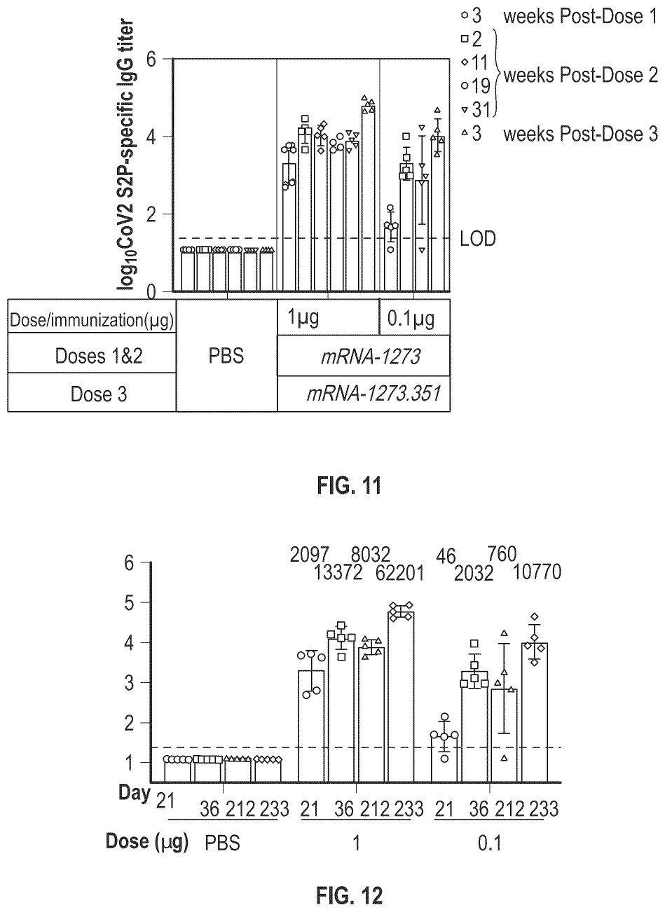

A summary of the study is presented in FIGS. 11-12.

These same trends were also observed in the groups that received booster doses on Day 58 and 78. BALB/c mice were immunized at days 1 and 22 with 1 μg of SEQ ID NO: 18 (WH2020_NatSP_2P) (n=8/group). The mice were subsequently administered a first booster dose (dose 3; 1 μg) on day 58. The first booster dose comprised mRNA encoding wild-type SARS-CoV-2 with the following mutations: L18F-D80A-D215G-L242-244del-R246I-K417N-E484K-N501Y-D614G-A701V. Serum samples collected on days 57 (before administration of the first booster dose) and on day 77 (after administration of the 1 μg booster dose). The data is shown in Table 13 below.

TABLE 13
Neutralization Titers (1 μg Dose)

| | | NAb Titer |
|---|---|---|
| Virus | Day | (ID₅₀) |
| D614G | 57 | 4955 |
| D614G | 77 | 24508 |
| B.1.351 | 57 | 1015 |
| B.1.351 | 77 | 16967 |

As shown in Table 13, the neutralizing antibody titer was increased from day 57 to day 77 against the D614G variant Spike protein and the B.1.351 variant Spike protein. The relative titer change between day 57 and day 77 against the D614G variant was 4.9× (p=0.0156) and the relative titer change between day 57 and day 77 against the B.1.351 variant was 17× (p=0.0156). The neutralizing antibody titer was 4.9× greater (p=0.0156) against the D614G variant than against the B.1.351 variant at day 57 (before administration of the booster); however, after the booster dose (day 77), the neutralization titers elicited against the D614G variant Spike protein were 1.8× those elicited by the B.1.351 variant Spike protein.

Similar trends were seen with the 0.1 μg dose. Neutralizing antibody titers against the D614G variant and the B.1.351 variant were measured on day 57 (before the third dose, 0.1 μg) and day 77 (after the third dose). The data is shown in Table 14 below.

TABLE 14
Neutralization Titers (0.1 μg Dose)

| | | NAb Titer |
|---|---|---|
| Virus | Day | (ID₅₀) |
| D614G | 57 | 72 |
| D614G | 77 | 376 |
| B.1.351 | 57 | 42 |
| B.1.351 | 77 | 290 |

As shown in Table 14, the neutralizing antibody titer was increased from day 57 to day 77 against the D614 variant Spike protein and the B.1.351 variant Spike protein. The relative titer change between day 57 and day 77 against the D614G variant was 5.2× (p=0.0313) and the relative titer change between day 57 and day 77 against the B.1.351 variant was 6.9× (p=0.0313). The neutralizing antibody titer was 1.7× greater against the D614G variant than against the B.1.351 variant at day 57 (before administration of the booster); however, after the booster dose (day 77), the neutralization titers elicited against the D614G variant Spike protein were 1.3× those elicited by the B.1.351 variant Spike protein.

### Discussion

In this Example, mRNA-1273.351 and mRNA-1273.211 were evaluated in mice as a booster in animals previously vaccinated with mRNA-1273. As a primary vaccination series, both vaccines were potently immunogenic after dose 1, with both S-2P binding and neutralizing antibody titers significantly increasing after the second dose. mRNA-1273.351 elicited robust neutralizing titers against B.1.351. However, neutralizing activity of mRNA-1273.351 against D614G virus was 4-fold lower than that against the B.1.351 virus, and 6.3-fold lower against D614G compared to mRNA-1273. In contrast, the multivalent mRNA-1273.211 vaccine elicited robust and comparable neutralizing titers against both the D614G and B.1.351 viruses. In addition, vaccination with mRNA-1273.211 elicited neutralizing titers against the B.1.351 variant closely matching those observed against the D614G virus after mRNA-1273 vaccination.

A boosting regimen, mRNA-1273.351 was evaluated in animals vaccinated with mRNA-1273 approximately 7 months previously. Despite concerns about the ability to further boost immunity driven by a primary series of mRNA-1273, the third dose of mRNA-1273.351 dramatically boosted both S-2P binding antibody titers and D614G and B.1.351 PSV neutralization titers. Neutralizing titers against B.1.351 PSV was increased to a level that is well above the peak neutralizing titer against D614G after the second dose of mRNA-1273, the latter of which is fully protective for the mouse-adapted USA-WA1/2020 isolate in mice. In addition, the boost also increased neutralizing titers against D614G, although the fold-increase was less than that against B.1.351, as expected. Overall, the boost with mRNA-1273.351 dramatically increased both D614G and B.1.351 neutralization titers, with titers much higher than the previous day 36 peak and with a decreased fold of reduction against B.1.351 relative to D614G virus, when compared to pre-3rd dose.

### Example 9: Protection and Durability of Coronavirus Vaccines in Non-Human Primates

Non-human primates (NHPs) were used to examine the durability and protection of different combinations of vaccines. In one study, NHPs were vaccinated with mRNA encoding Spike protein with two proline substitutions (“mRNA vaccine”) twice. At week 0, each NHP (n=8/group) was immunized with 100 μg or 30 μg of the mRNA vaccine. At week 4 or 5, the animals who were previously administered 100 μg of the mRNA vaccine are administered another 100 μg dose of the mRNA vaccine. Of the group that received a 30 μg dose of the vaccine, the animals are either administered another 30 μg dose or are not given a second dose. There is was a naïve control group that did not receive any mRNA vaccines. On week 12, the animals were challenged with 5×10⁵ PFU of virus (either D614G or B.1.351). Following challenge, blood samples were drawn on days 2, 4, 7, and 14; nasal washes were performed on days 2, 4, 7, and 14; nasal swabs were taken on days 2, 4, and 7; bronchoalveolar lavage (BAL) was sampled on days 2, 4, 7, and 14, and lung pathology was performed on days 8 and 14 (n=4/group/day).

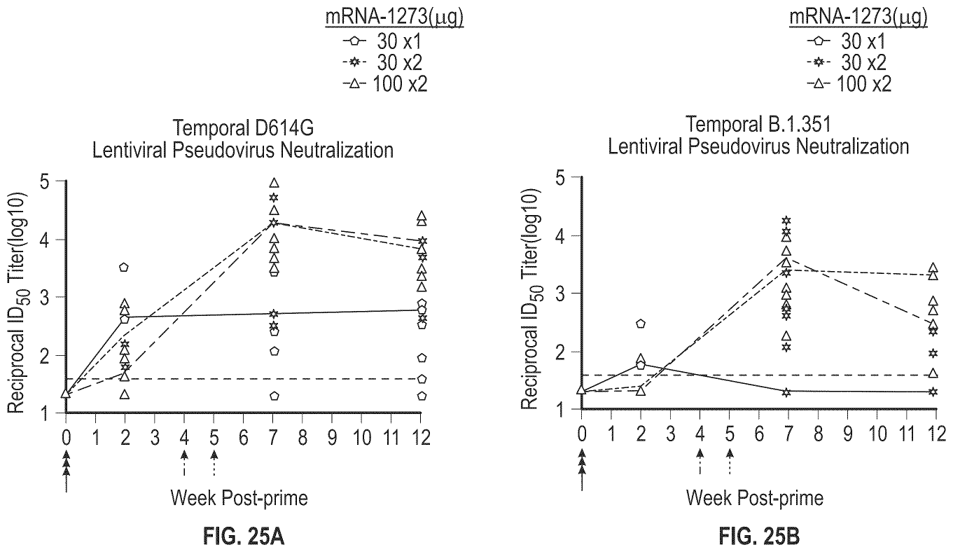

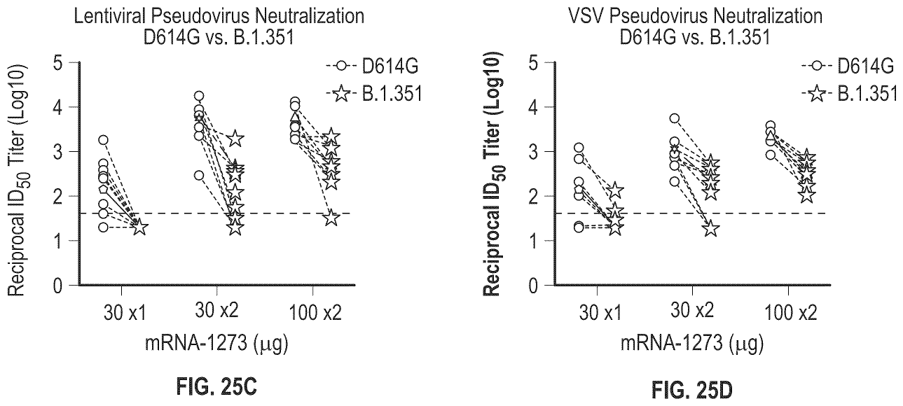

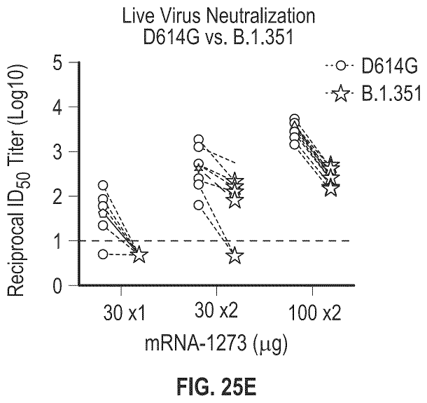

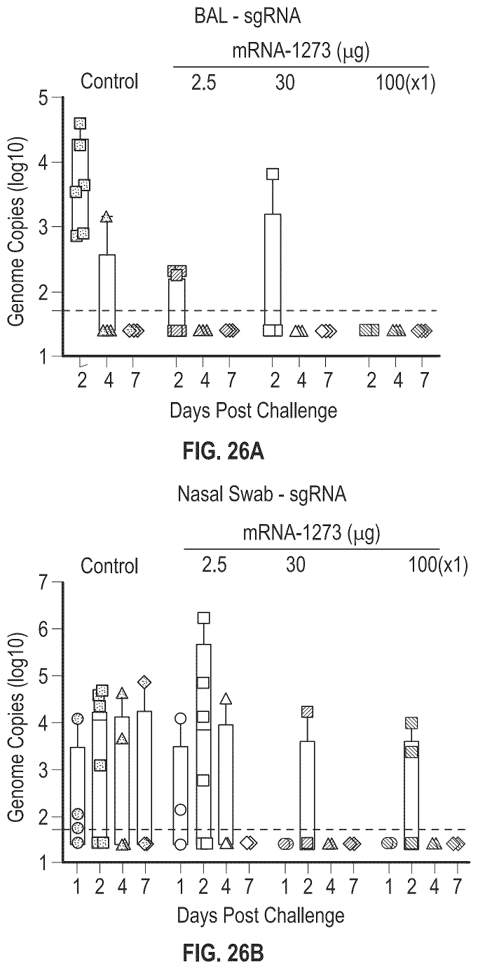

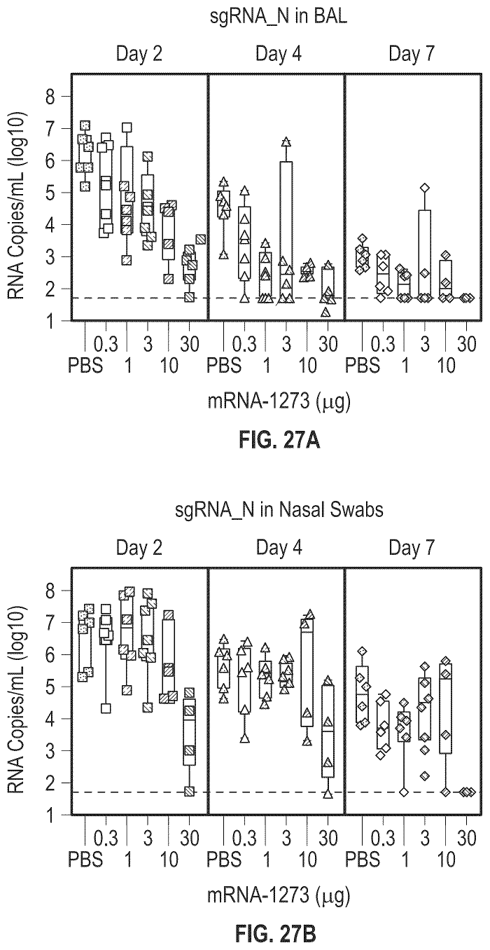

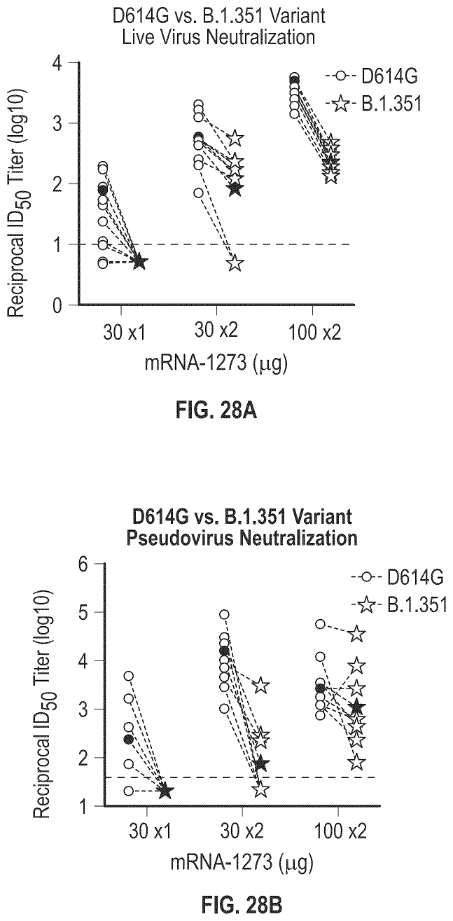

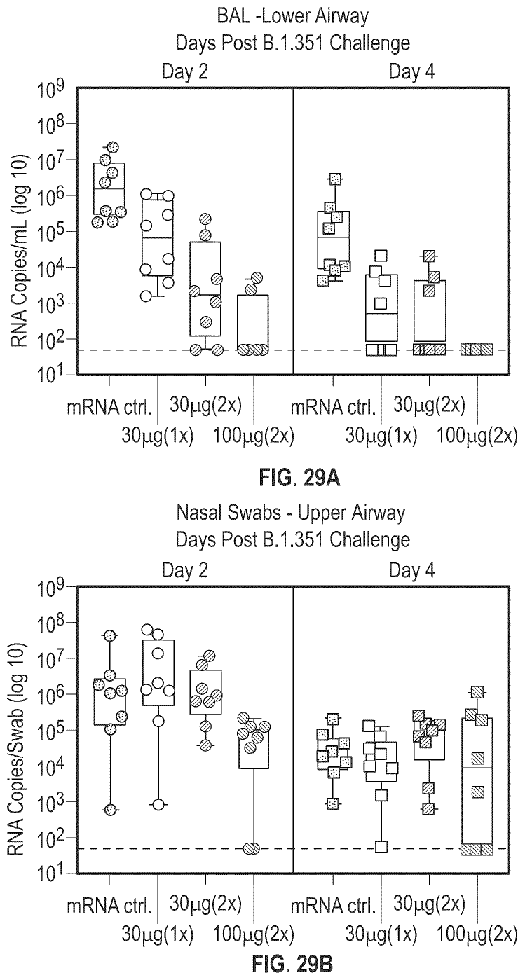

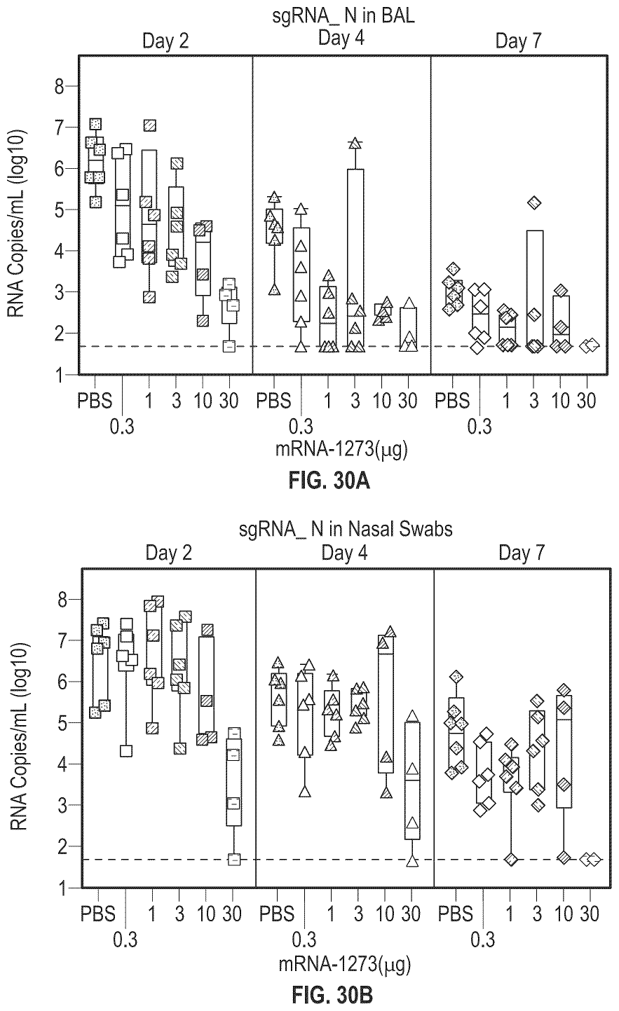

Neutralization titers were examined over time, as shown in FIGS. 25A and 25B and demonstrate a consistent titer that wanes slightly with time. Neutralizing antibody titers against B.1.351 were lower than those generated against D614G (FIGS. 25C-25E). As shown in FIGS. 26A-26B, the NHPs demonstrated full protection in nose and lungs following the 30 μg dose. In a later study, the 30 μg dose was found to be protective in the lower airway (BAL; FIG. 27A), but viral copies were detected in the nasal swab samples (FIG. 27B). Despite the robust challenge, the infection in the upper airway was rapidly controlled and the animals were considered to be protected, with the potential for transmission over a short time period. There was a reduction in neutralizing antibody titers against B.1.351 in animals vaccinated with mRNA-1273 (FIGS. 28A-28B) and viral replication was detected in the upper airway (FIG. 29B). The lower airway was protected (FIG. 29A). Moreover, protection was found against WA.1 and RSA (FIGS. 30A-30B), although viral replication in the upper airway suggests the potential for transmission.

In another study, animals are vaccinated with the mRNA vaccine twice (30 μg per dose) and then are administered a vaccine comprising mRNA encoding wild-type SARS-CoV-2 with the following mutations: L18F-D80A-D215G-L242-244del-R246I-K417N-E484K-N501Y-D614G-A701V (the B.1.351 Spike protein) or a combination of the mRNA vaccine and mRNA encoding the B.1.351 Spike protein. The animals are then challenged with the RSA isolate and examined to determine if the administration protocol boosts immunity and confer protection against the B.1.351 (RSA) isolate.

In a further study, animals are administered 10 μg, 30 μg, or 100 μg of an mRNA vaccine comprising mRNA encoding the B.1.351 Spike protein and then challenged with the RSA isolate to determine whether the vaccine mediates protection against RSA challenge and whether the dose of the vaccine influences the immunity and protection.

### Example 10: Protection and Durability of Coronavirus Vaccines in Hamster Models

Hamsters (Golden Syrian Hamsters) are used to examine the durability and protection of different combinations of vaccines. In one study, hamsters are vaccinated with mRNA encoding Spike protein with two proline substitutions (“mRNA vaccine”) twice or with mRNA encoding wild-type SARS-CoV-2 with the following mutations: L18F-D80A-D215G-L242-244del-R246I-K417N-E484K-N501Y-D614G-A701V (the B.1.351 Spike protein) twice. Each animal receives two doses of 25 μg, 5 μg, or 1 μg. The hamsters are then challenged with the RSA isolate to determine whether the titers generated are protective against the isolate.

In another study, hamsters are vaccinated with mRNA encoding the B.1.351 Spike protein twice. Each animal receives two doses of 25 μg, 5 μg, or 1 μg. The hamsters are then challenged with the RSA isolate or the WA1 isolate to determine whether the vaccine is protective against both isolates.

In a further study, animals are administered 25 μg, 5 μg, or 1 μg of an mRNA vaccine comprising mRNA encoding Spike protein with two proline substitutions in combination with mRNA encoding the B.1.351 Spike protein and then challenged with the WA1 isolate to determine whether the vaccine mediates protection against the challenge and whether the dose of the vaccine influences the immunity and protection.

In another study, animals are administered two doses of an mRNA vaccine comprising mRNA encoding Spike protein with two proline substitutions. The animals are then administered a 25 μg booster dose of the vaccine or 5 μg of a vaccine comprising mRNA encoding the B.1.351 Spike protein or a 1 μg of a vaccine comprising both an mRNA vaccine comprising mRNA encoding Spike protein with two proline substitutions (0.5 μg) and an mRNA encoding the B.1.351 Spike protein (0.5 μg).

### Example 12: In Vitro Neutralization Screening

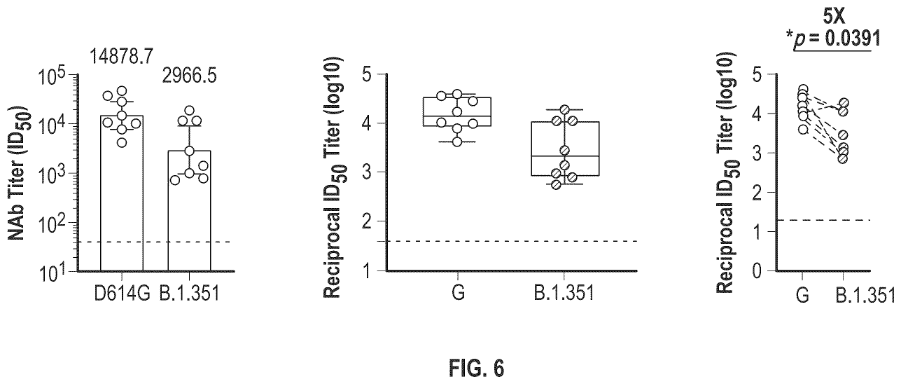

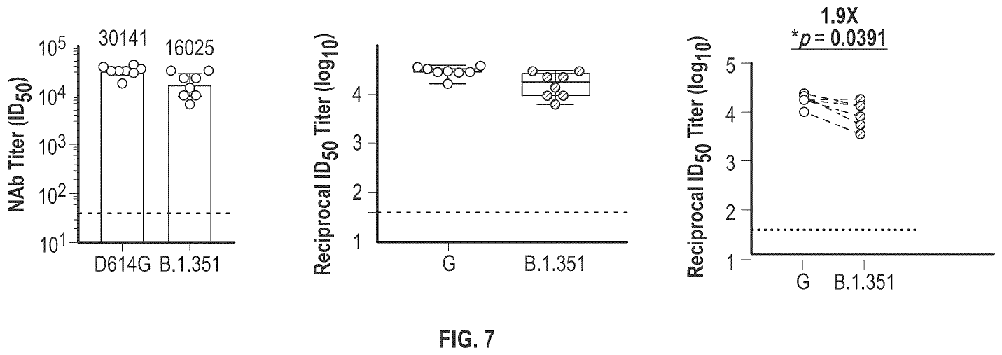

Codon-optimized full-length spike protein of the D614G variant or the spike protein of the B.1.351 variant were cloned into a pCAGGS vector. For the neutralization assay, serially diluted serum samples were mixed with pseudovirus and incubated at 37° C. for 45 minutes. The serum samples were from mice that had been administered 1 μg of mRNA encoding a Spike protein with two proline substitutions or mRNA encoding the D614G antigen. The virus-serum mix was subsequently used to infect A549-hACE2-TMPRSS2 cells for 18 hours at 37° C. before measuring neutralizing antibody titers. The results are shown in FIGS. 6 and 7 and demonstrate that the mRNA encoding a Spike protein with two proline substitutions had a 5-fold decrease in neutralization titer against the B.1.351 variant compared to the titer resulting from the D614G pseudovirus, while the mRNA encoding the D614G antigen resulted in a 1.9-fold decrease in neutralization titer between the D614 variant and the B.1.351 variant.

### Example 13: Immunization of Human Subjects with a Wild-Type or Strain-Matched Booster Doses

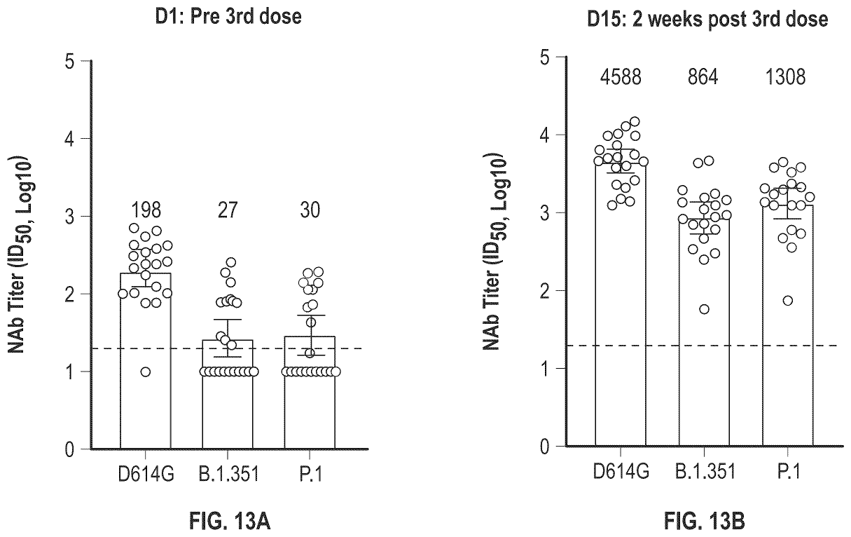

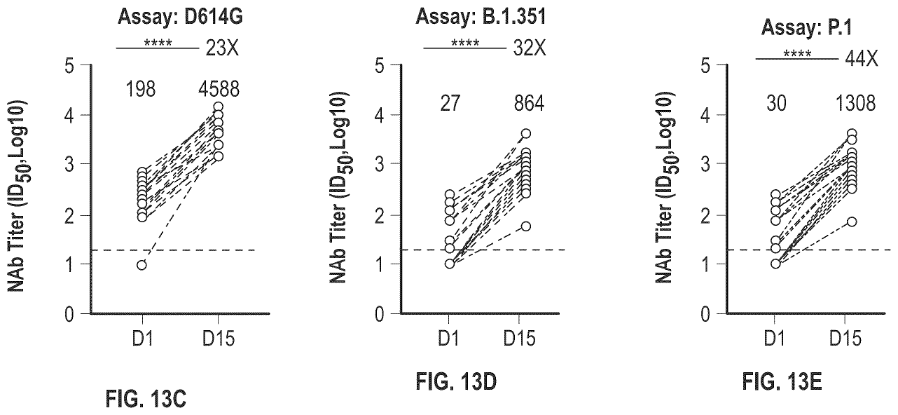

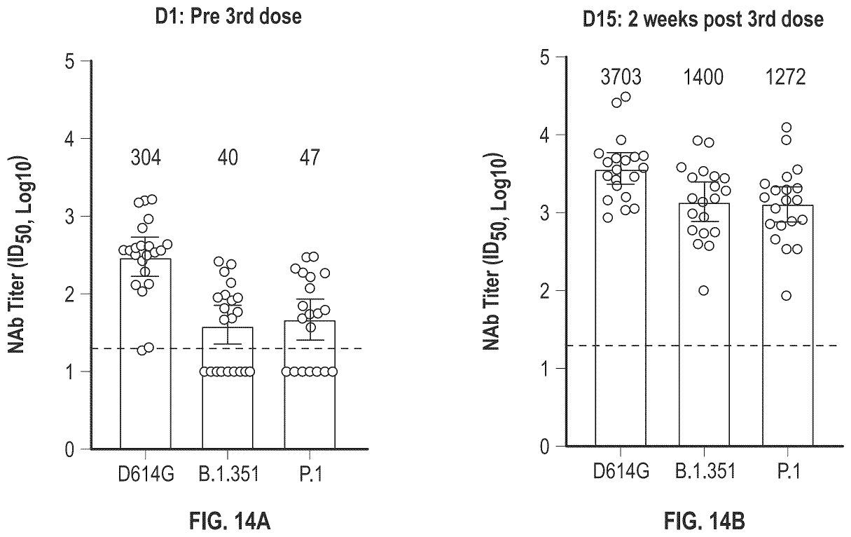

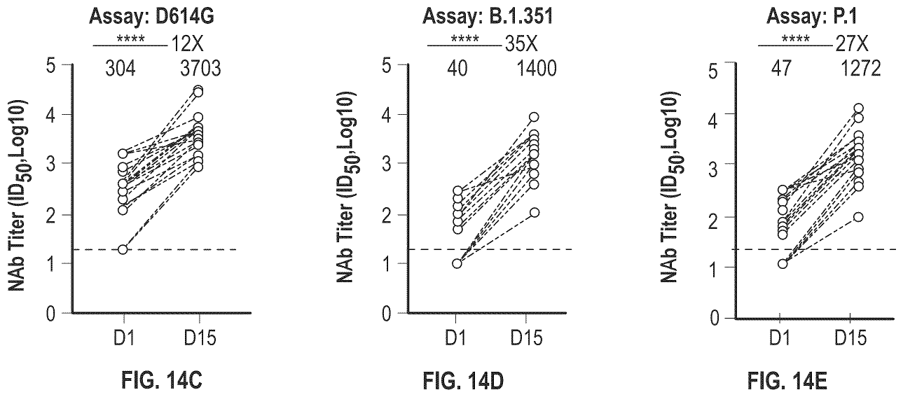

Human clinical trial participants were immunized by administration of two doses with 100 μg mRNA-1273 as described in Example 1. About 6 months after receiving the second dose of mRNA-1273, participants were administered a 3rd dose (booster dose) of either 50 μg mRNA-1273 (FIGS. 13A-13E), 50 μg mRNA-1273.351 (FIGS. 14A-14E), which encoded a 2P-stabilized SARS-CoV-2 Spike protein comprising the mutations associated with the B.1.351 SARS-CoV-2 variant. Sera were collected from participants immediately prior to administration of the booster dose (D1 or Day 1 in FIGS. 13A-15E), and again two weeks after administration of the booster dose (D15 or Day 15 in FIGS. 13A-15E). Sera from each time point were evaluated using a pseudovirus neutralization assay as described in Examples 1 and 12 to quantify neutralizing antibody titers towards pseudoviruses expressing one of 1) a D614G Spike protein, 2) a Spike protein with the mutations associated with the B.1.351 variant (B.1.351 Spike protein), or 3) a Spike protein with the mutations associated with the P.1 variant (P.1 Spike protein). The results of these neutralization assays, and the effects of each booster dose, are shown in FIGS. 13A-15E.

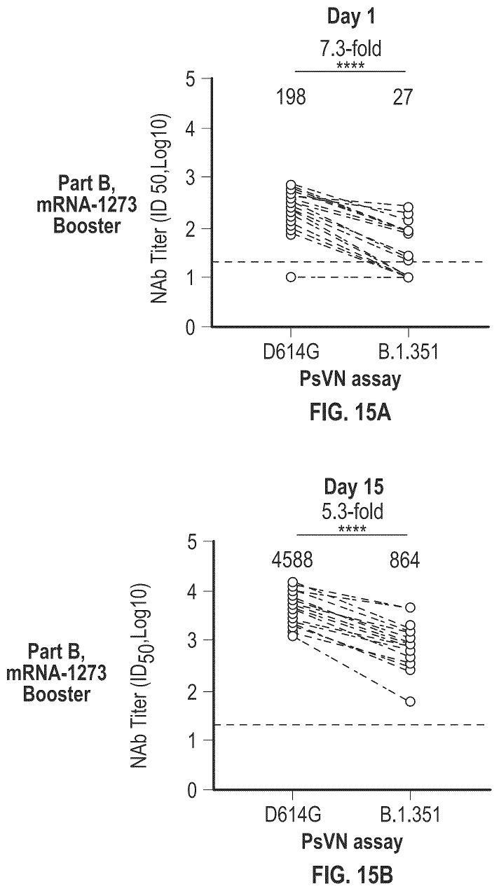

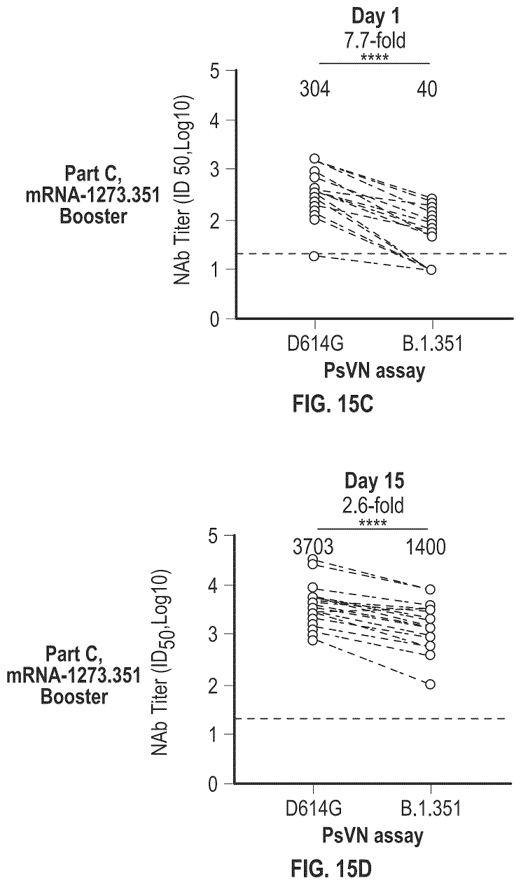

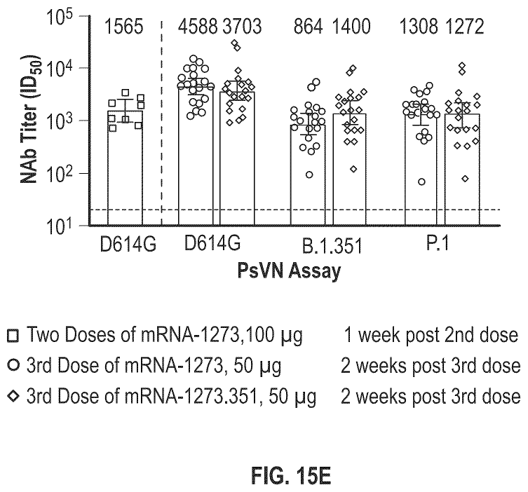

Six months after the second dose of mRNA-1273, and prior to the administration of booster doses, all groups still demonstrated neutralizing antibodies towards D614G Spike protein, but had minimal ability to neutralize B.1.351 or P.1 Spike proteins (FIGS. 13A, 14A). Administration of either the mRNA-1273 or mRNA-1273.351 booster dose (e.g., third total dose) markedly increased neutralizing antibody titers towards D614G, B.1.351, and P.1 Spike proteins, up to 44-fold compared to titers prior to booster dose administration (FIGS. 13C-13E, 14C-14E). Neutralizing antibody titers towards B.1.351 Spike protein were approximately 1.5 times greater in subjects administered the strain-matched mRNA-1273.351 booster dose (i.e., third total dose) instead of mRNA-1273, suggesting that strain-matched mRNA vaccines are effective at generating a robust antibody response towards the encoded protein (FIG. 13B, 14B). While mRNA-1273 elicited neutralizing antibodies towards D614G, B.1.351, and P.1 Spike proteins, neutralizing antibody titers towards B.1.351 Spike protein were uniformly lower than titers towards the D614G Spike protein (7.3-fold on day 1, 5.3-fold on day 15) (FIGS. 15A-15B). By contrast, administration of mRNA-1273.351 ameliorated this reduction in B.1.351-neutralizing titers relative to D614G-neutralizing titers, improving the difference from a 7.7-fold decrease prior to booster (third dose) administration, to a reduction of only 2.6-fold after mRNA-1273.351 booster (third dose) administration (FIGS. 15C-15D). Administration of either booster dose (third dose) elicited neutralizing antibodies towards B.1.351 and P.1 Spike proteins at titers that were comparable to those observed towards D614G Spike protein at 1 week after the second dose of mRNA-1273, suggesting mRNA-1273.351 is capable of eliciting a robust response to B.1.351 and other Spike proteins including the D614G strain (FIG. 15E). Surprisingly, both boosters increased neutralizing titers towards D614G Spike protein to titers that were 2- to 3-fold greater than those observed towards the same protein 1 week after the second dose of mRNA-1273, suggesting booster doses are useful for maintaining and even increasing neutralizing antibody responses to antigens encoded by previously administered mRNA vaccines.

Further reductions in the differences between the two assays may be found in samples collected from these participants at later timepoints, as the kinetics of the neutralizing antibody responses to the new epitopes in S-2P.351 protein encoded by mRNA-1273.351 may be different from the epitopes shared between the two immunogens. Strong homologous responses, both in terms of absolute titer and fold rise, assessed by the same strain in the assay used in the vaccine, were seen regardless of vaccine strain. Response to the wild-type virus was highest with boost of mRNA-1273 and response to B.1.351 was highest with mRNA-1273.351. In addition, heterologous responses, against variants when prototype vaccine was used to boost or against prototype after the mRNA-1273.351 booster were also seen. This supports the development of a variant vaccine as a booster dose to prevent infection caused by variant strains.

### Example 14: Neutralization of Variant Spike Proteins by Antibodies Elicited after Two Doses of mRNA Encoding 2P-Stabilized Spike Protein

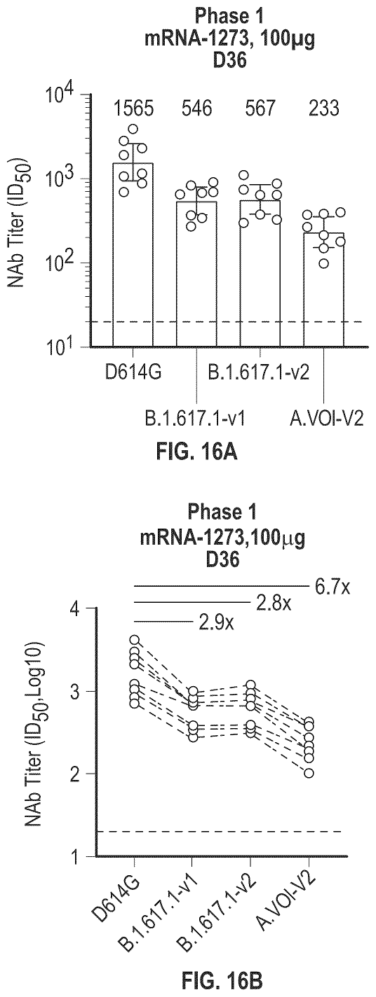

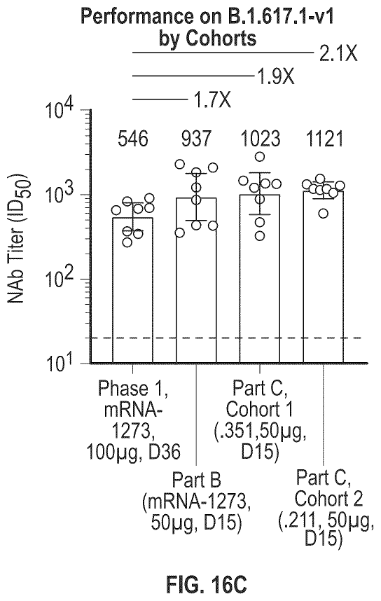

Human subjects were immunized with two doses of 100 μg mRNA-1273, as described in Example 13. Sera collected at day 36, two weeks after the 2^{nd} dose with mRNA-1273, and evaluated for neutralizing antibody titers towards variant Spike proteins of the B.1.617.1-v1, B.1.617.1-v2, and A. VOI-V2 in a pseudovirus neutralization assay. The results of these neutralization assays are shown in FIGS. 16A-16C. Neutralizing antibody titers towards either B.1.617 variant Spike protein were about 3-fold lower than titers towards the reference D614G Spike protein, and 6.7-fold lower towards the A. VOI-V2 Spike protein than to D614G (FIGS. 16A-16B).

The booster doses described in Example 13; of 50 μg of either mRNA-1273, mRNA-1273.351, or mRNA-1273.211 (1:1 mixture of mRNA-1273 and mRNA-1273.351, 25 μg each) were administered and sera were collected 15 days later and tested for neutralizing antibody titers towards the B.1.617-v1 Spike protein. Relative to day 36 sera from subjects immunized with two doses of 100 μg mRNA-1273, these 50 μg booster doses improved neutralizing antibody titers towards the B.1.617-v1 Spike protein by 1.7- to 2.1-fold (FIG. 16C). Booster doses containing mRNA-1273.351, either alone or in combination, resulted in greater increases than mRNA-1273 alone. These results indicate that while neutralizing antibody titers may be lower towards variant Spike proteins than the Spike protein encoded by an initial administered mRNA, booster doses are capable of eliciting antibodies towards variant Spike proteins to provide some protection against heterologous SARS-CoV-2 infections.

### Example 15: Immunization with 3^{rd} and 4^{th} Booster Doses of mRNAs Encoding Variant Spike Proteins at 6 Months

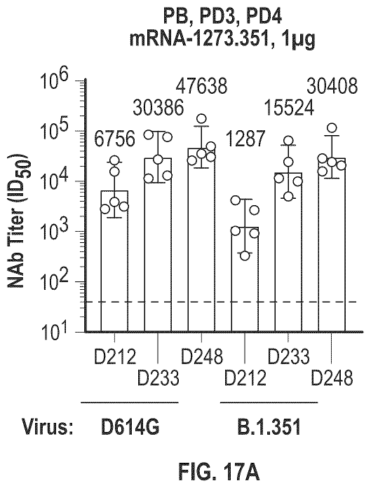

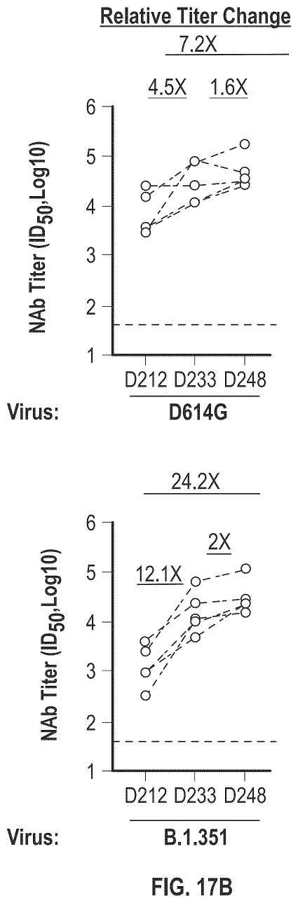

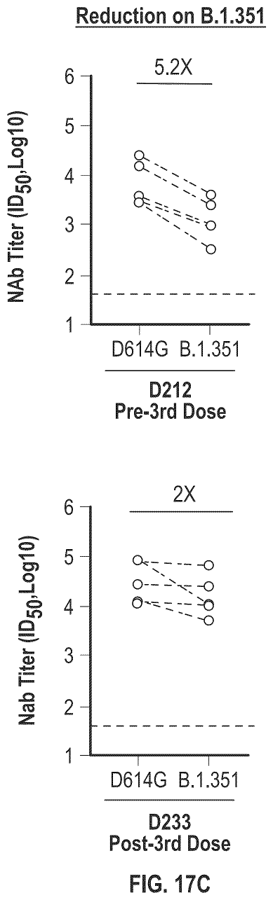

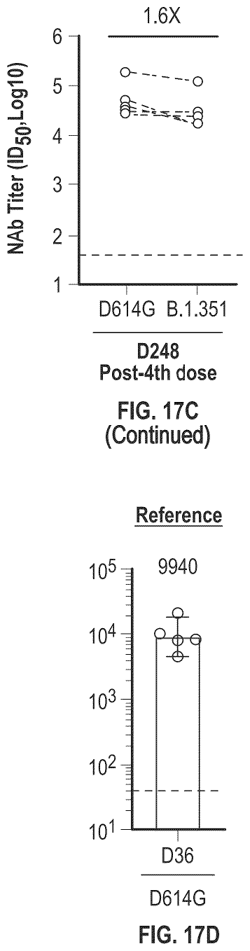

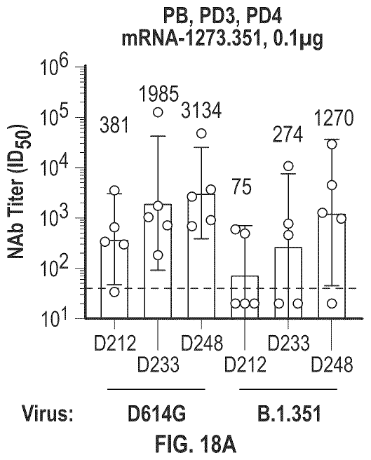

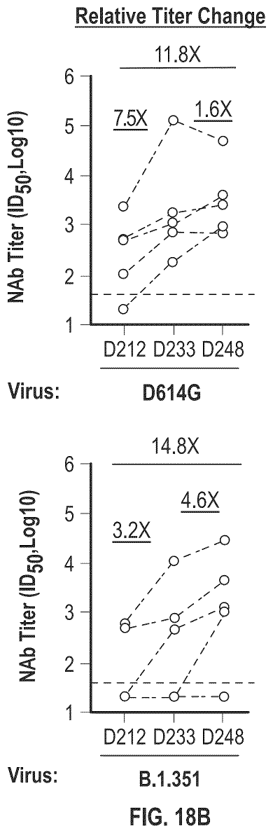

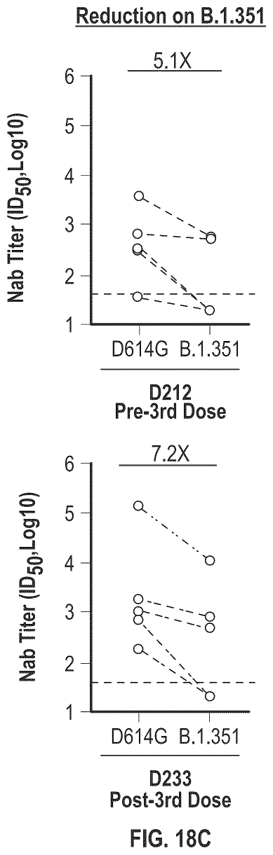

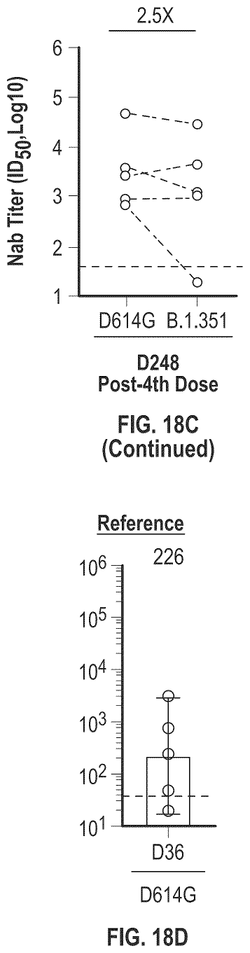

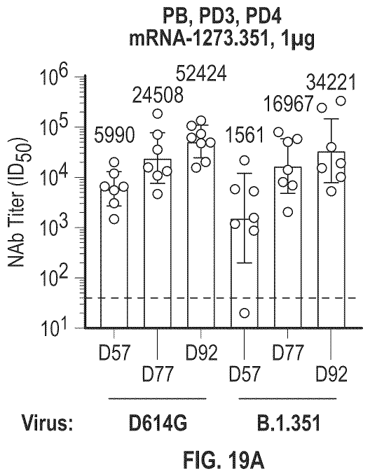

BALB/c mice, 6-8 weeks of age, were administered either 1 μg (FIGS. 17A-17D) or 0.1 μg (FIGS. 18A-18D) of mRNA-1273, or PBS (as a control) intramuscularly in one hind leg (formulated as a 50 μL dose) on day 1 (1st dose, prime) and day 22 (2nd dose, booster). At days 213 (3rd dose) and 234 (4th dose), mice were administered either 1 μg (FIGS. 17A-17D) or 0.1 μg (FIGS. 18A-18D) of mRNA-1273.351, which contained the mutations associated with the B.1.351 variant. In each vaccine, the mRNA was formulated in lipid nanoparticles (LNPs) including 0.5-15% PEG-modified lipid, 5-25% non-cationic lipid, 25-55% sterol, and 20-60% ionizable amino lipid. The PEG-modified lipid was 1,2 dimyristoyl-sn-glycerol, methoxypolyethyleneglycol (PEG2000 DMG), was non-cationic lipid is 1,2 distearoyl-sn-glycero-3-phosphocholine (DSPC), the sterol was cholesterol, and the ionizable amino lipid had the structure of Compound 1, for example. Sera were collected from mice on days 212 (prior to 3rd dose), 233 (21 days after 3rd dose, prior to 4th dose), and 248 (14 days after 4th dose), and analyzed for neutralization titers against VSV-based pseudoviruses expressing a SARS-CoV-2 Spike protein comprising either 1) a D614G mutation relative to the sequence of the WH2020 full-length Spike protein, or 2) mutations associated with the B.1.351 variant.

For mice administered 1 μg doses of mRNA in each vaccine, the results of these neutralization assays are shown in FIGS. 17A-17D. Each of the 3rd and 4th doses increased mean neutralization titers of sera against the D614G and B.1.351 Spike proteins (FIG. 17A). Neutralization titers towards each Spike protein increased over time, with this increase being more pronounced in neutralization titers towards the B.1.351 Spike protein (FIG. 17B). Prior to the 3rd dose, sera were approximately 5.2 times as effective at neutralizing the D614G Spike protein than the B.1.351 Spike protein (FIG. 17C). However, after each of the 3rd and 4th doses, the reduction was less pronounced, with sera neutralizing the D614G Spike protein only 2-fold as well as the B.1.351 Spike protein after the 3rd dose, and 1.6-fold as well after the 4th dose (FIG. 17C). Additionally, neutralization titers towards either Spike protein after the 4th dose were over 3-fold greater than reference neutralization titers towards the D614G Spike protein at day 36, 14 days after the 2nd dose (FIGS. 17A, 17D). These results indicate that the administration of mRNAs encoding variant Spike proteins effectively elicited neutralizing antibodies towards the variant Spike protein, and boost the antibody response to Spike proteins encoded by previously administered mRNAs.

For mice administered 0.1 μg doses of mRNA in each vaccine, the results of these neutralization assays are shown in FIGS. 18A-18D. Each of the 3rd and 4th doses increased mean neutralization titers of sera against the D614G and B.1.351 Spike proteins (FIG. 18A). Neutralization titers towards each Spike protein increased over time, with this increase being more pronounced in neutralization titers towards the B.1.351 Spike protein (FIG. 18B). Prior to the 3rd dose, sera were approximately 5.1 times as effective at neutralizing the D614G Spike protein than the B.1.351 Spike protein (FIG. 18C). However, after the 4th dose, this reduction was less pronounced, with sera neutralizing the D614G Spike protein only 2.5 times as well as the B.1.351 Spike protein (FIG. 18C). Additionally, neutralization titers towards either Spike protein after the 4th dose were over 5-fold greater than reference neutralization titers towards the D614G Spike protein at day 36, 14 days after the 2nd dose (FIGS. 18A, 18D). These results indicate that the administration of mRNAs encoding variant Spike proteins effectively elicited neutralizing antibodies towards the variant Spike protein, and boost the antibody response to Spike proteins encoded by previously administered mRNAs.

### Example 16: Immunization with 3^{rd} and 4^{th} Booster Doses of mRNAs Encoding Variant Spike Proteins at 2 Months

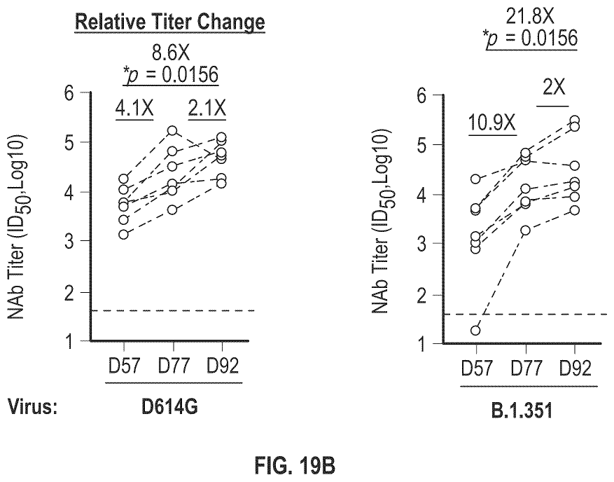

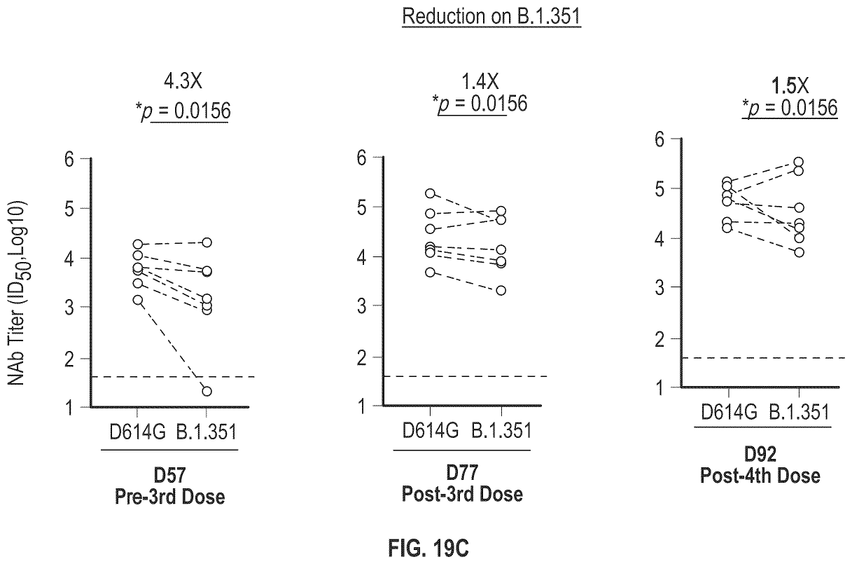

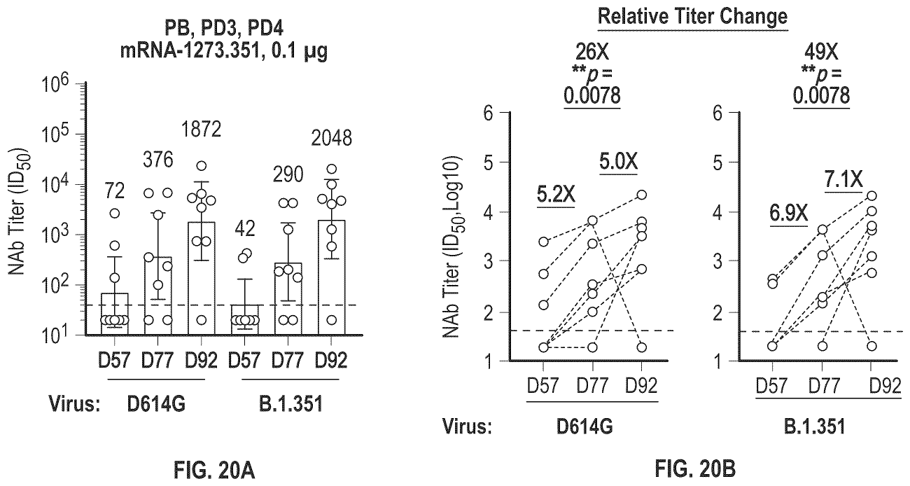

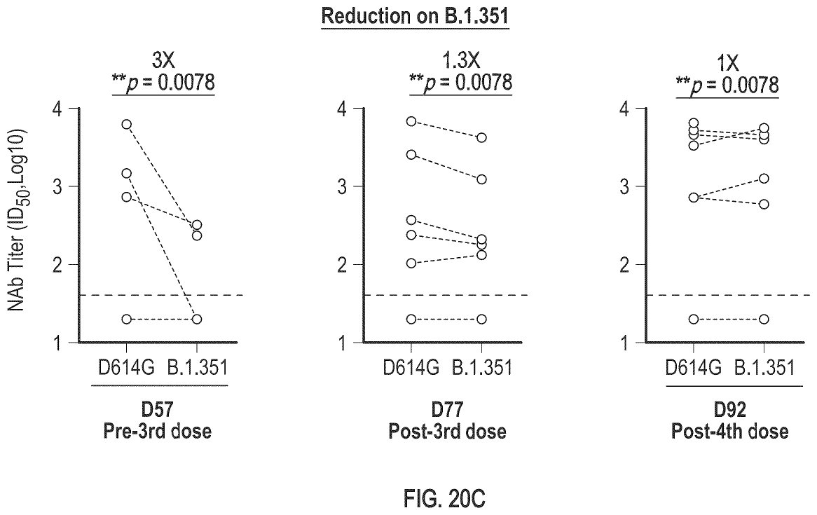

BALB/c mice, 6-8 weeks of age, were administered either 1 μg (FIGS. 19A-19C) or 0.1 μg (FIGS. 20A-20C) of mRNA-1273, or PBS (as a control) intramuscularly in one hind leg (formulated as a 50 μL dose) on day 1 (1st dose, prime) and day 22 (2nd dose, booster). At days 58 (3rd dose) and 78 (4th dose), mice were administered either 1 μg (FIGS. 19A-19D) or 0.1 μg (FIGS. 20A-20D) 1 μg of mRNA-1273.351, which contained the mutations associated with the B.1.351 variant. In each vaccine, the mRNA was formulated in lipid nanoparticles (LNPs) including 0.5-15% PEG-modified lipid, 5-25% non-cationic lipid, 25-55% sterol, and 20-60% ionizable amino lipid. The PEG-modified lipid was 1,2 dimyristoyl-sn-glycerol, methoxypolyethyleneglycol (PEG2000 DMG), was non-cationic lipid is 1,2 distearoyl-sn-glycero-3-phosphocholine (DSPC), the sterol was cholesterol, and the ionizable amino lipid had the structure of Compound 1, for example. Sera were collected from mice on days 57 (prior to 3rd dose), 77 (19 days after 3rd dose, prior to 4th dose), and 92 (14 days after 4th dose), and analyzed for neutralization titers against VSV-based pseudoviruses expressing a SARS-CoV-2 Spike protein comprising either 1) a D614G mutation relative to the sequence of the WH2020 full-length Spike protein, or 2) mutations associated with the B.1.351 variant.

For mice administered 1 μg doses of mRNA in each vaccine, the results of these neutralization assays are shown in FIGS. 19A-19C. Each of the 3rd and 4th doses increased mean neutralization titers of sera against the D614G and B.1.351 Spike proteins (FIG. 19A). Neutralization titers towards each Spike protein increased over time, with this increase being more pronounced in neutralization titers towards the B.1.351 Spike protein (FIG. 19B). Prior to the 3rd dose, sera were approximately 4.3 times as effective at neutralizing the D614G Spike protein than the B.1.351 Spike protein (FIG. 19C). However, after each of the 3rd and 4th doses, the reduction was less pronounced, with sera neutralizing the D614G Spike protein only 1.4 times as well as the B.1.351 Spike protein after the 3rd dose, and 1.5 times as well after the 4th dose (FIG. 19C). These results indicate that the administration of mRNAs encoding variant Spike proteins effectively elicit neutralizing antibodies towards the variant Spike protein, and boost the antibody response to Spike proteins encoded by previously administered mRNAs.

For mice administered 0.1 μg doses of mRNA in each vaccine, the results of these neutralization assays are shown in FIGS. 20A-20C. Each of the 3rd and 4th doses increased mean neutralization titers of sera against the D614G and B.1.351 Spike proteins (FIG. 20A). Neutralization titers towards each Spike protein increased over time, with this increase being more pronounced in neutralization titers towards the B.1.351 Spike protein (FIG. 20B). Prior to the 3rd dose, sera were approximately 3 times as effective at neutralizing the D614G Spike protein than the B.1.351 Spike protein (FIG. 20C). However, after each of the 3^{rd} and 4^{th} doses, this reduction was less pronounced, with sera neutralizing the D614G Spike protein only 1.3 times as well as the B.1.351 Spike protein after the third dose, and neutralization titers towards both Spike proteins were approximately equivalent after the 4^{th} dose (FIG. 20C). These results indicate that the administration of mRNAs encoding variant Spike proteins effectively elicit neutralizing antibodies towards the variant Spike protein, and boost the antibody response to Spike proteins encoded by previously administered mRNAs.

### Example 17: Immunization with Two Doses of mRNAs Encoding Variant Spike Proteins to Generate Neutralizing Antibodies to Variant Spike Proteins in Mice

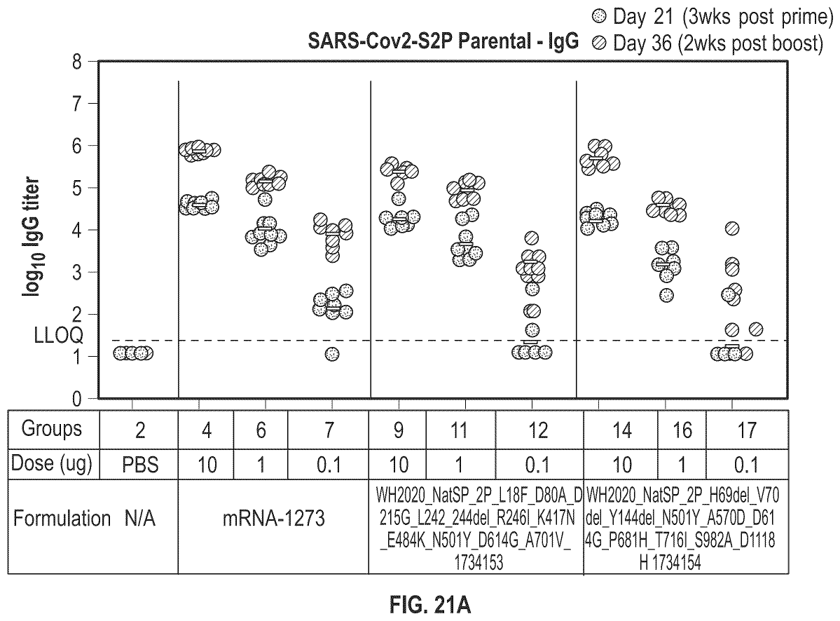

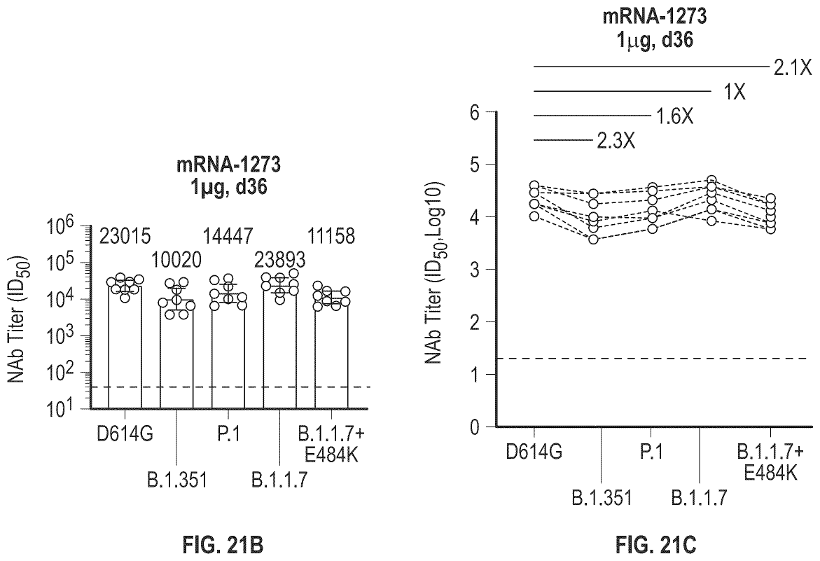

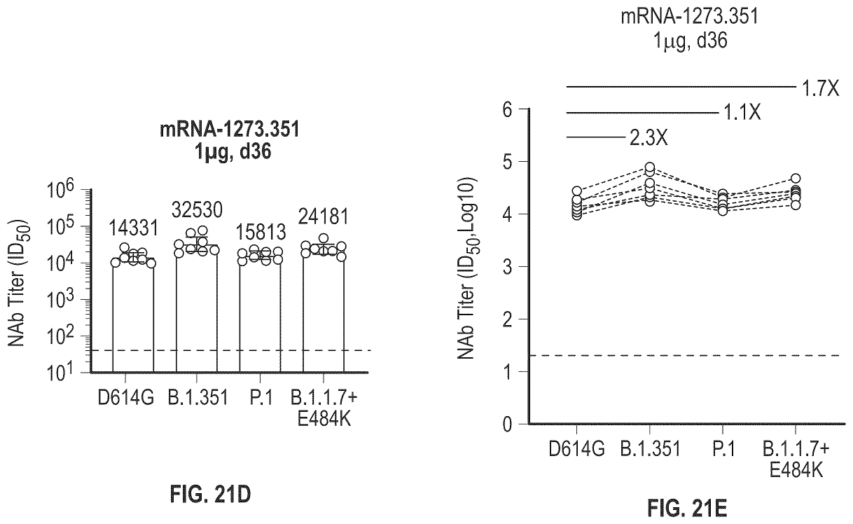

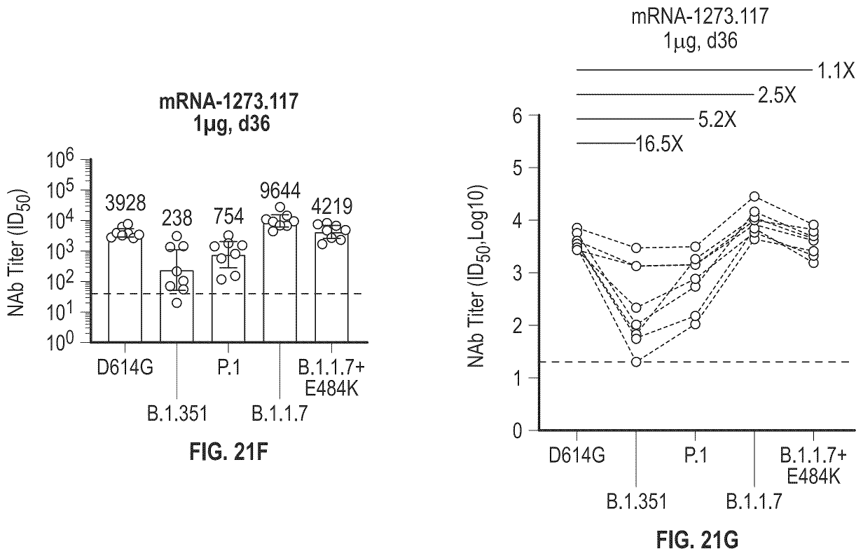

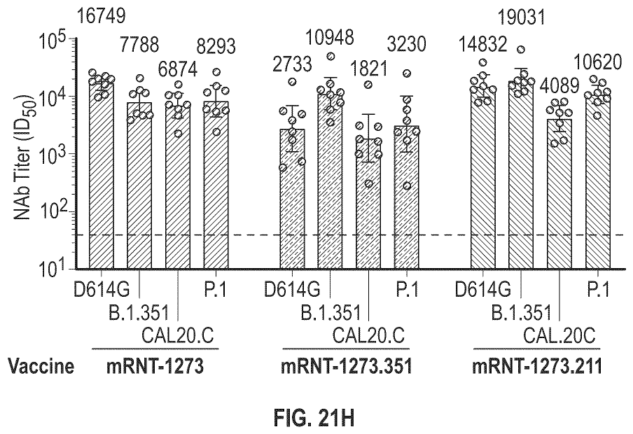

BALB/c mice, 6-8 weeks of age, were administered 0.1 μg, 1 μg, or 10 μg of mRNA encoding a SARS-CoV-2 antigen, specifically mRNA-1273, mRNA-1273.351, or mRNA-1273.117, encoding the Spike protein of the B.1.1.7 variant, or PBS (as a control) intramuscularly in one hind leg (formulated as a 50 μL dose) on day 1 (1^{st} dose, prime) and day 22 (2^{nd} dose, booster). Sera were collected from mice at days 21 (3 weeks after 1^{st} dose, before 2^{nd} dose), and 36 (2 weeks after 2^{nd} dose), and tested by ELISA to quantify total IgG specific to a parental SARS-CoV-2 Spike protein with the USA-WA1/2020 isolate amino acid sequence. Each 1^{st} dose of mRNA encoding a Spike protein antigen elicited a robust SARS-CoV-2 Spike protein-specific antibody response, with the 2^{nd} dose boosting IgG titers by 10- to 100-fold in each dose group (FIG. 21A).

Sera obtained at day 36 from mice vaccinated with two 1 μg doses of mRNA-1273, mRNA-1273.351, or mRNA-1273.117 were also evaluated for neutralization activity against a panel of VSV-based pseudoviruses, each expressing a different SARS-CoV-2 Spike protein. The panel of Spike proteins tested is shown in Table 15, and included a D614G Spike protein, a B.1.351 Spike protein, a P.1 Spike protein, a B.1.1.7 Spike protein, and a B.1.1.7 Spike protein comprising an E484K mutation. The results of these neutralization assays are shown in FIGS. 21B-21D. Two 1 μg doses of mRNA-1273 elicited robust neutralizing antibody responses towards each of the Spike proteins tested (FIG. 21B). Neutralization titers towards each of the variant Spike proteins, B.1.351, P.1, and B.1.1.7 with E484K mutation, were about 2-fold lower than neutralization titers towards the D614G reference Spike protein (FIGS. 21B-21C). However, mRNA-1273 elicited roughly equivalent neutralization titers towards both D614G and B.1.1.7 Spike proteins that did not contain an E484K mutation (FIGS. 21B-21C). Two 1 μg doses of mRNA-1273.351 elicited robust neutralizing antibody responses towards all Spike proteins tested, with neutralizing antibody titers towards variant Spike proteins that were at least equivalent to, or up to 2.3 times greater than, titers towards the reference D614G Spike protein (FIGS. 21D-21E). Two 1 μg doses of mRNA-1273.351 elicited robust neutralizing antibody responses towards all Spike proteins tested (FIG. 21F). While neutralizing antibody titers towards B.1.351 Spike protein were 16.5-fold lower than titers against D614G Spike protein, sera were 2.5 times more effective at neutralizing B.1.1.7 Spike protein-containing pseudoviruses than those containing the D614G Spike protein. The E484K mutation partially abrogated this increase, but titers against the B.1.1.7+E8484K Spike protein were roughly equivalent to, not markedly lower than, titers against the D614G Spike protein (FIG. 21G).

TABLE 15
Geometric mean neutralizing antibody titers towards variant Spike proteins
elicited by two doses of mRNA-1273, mRNA-1273.351, or mRNA-1273.117

| | NAb titer (ID₅₀) | NAb titer (ID₅₀) | NAb titer (ID₅₀) | |
|---|---|---|---|---|
| | after two | after two | after two | Neutralization |
| | 1 μg doses | 1 μg doses | 1 μg doses | titer elicited |
| Spike protein | mRNA-1273 | mRNA-1273.351 | mRNA-1273.117 | by PBS (ID₅₀) |
| D614G | 23015 | 14331 | 3928 | 20 (below L.O.D.) |
| B.1.351 | 10020 | 32530 | 238 | 20 (below L.O.D.) |
| P.1 | 14447 | 15813 | 754 | 20 (below L.O.D.) |
| B.1.1.7 | 23893 | (not tested) | 9644 | (not tested) |
| B.1.1.7 + E484K | 11158 | 24181 | 4219 | (not tested) |

In a separate experiment, BALB/c mice, 6-8 weeks of age, were administered 1 μg of mRNA encoding a SARS-CoV-2 antigen, specifically mRNA-1273, mRNA-1273.351, or mRNA-1273.211 (1:1 mixture of mRNA-1273 and mRNA-1273.351, 0.5 μg each), intramuscularly in one hind leg (formulated as a 50 μL dose) on day 1 (1^{st} dose, prime) and day 22 (2^{nd} dose, booster). Sera obtained at day 36 (14 days post-2^{nd} dose) and evaluated for neutralization activity against a panel of VSV-based pseudoviruses, each expressing a different SARS-CoV-2 Spike protein. The Spike proteins tested included 1) D614G Spike protein, 2) B.1.3.51 Spike protein, 3) CAL20.C Spike protein, and 4) P.1 Spike protein. The results of these neutralization assays are shown in FIG. 21H.

Each mRNA composition elicited robust neutralizing antibody responses towards each of the Spike proteins tested. Antibodies elicited by two doses of mRNA-1273 had the greatest neutralizing effect towards the reference D614G Spike protein, but neutralization titers towards variant (B.1.351, CAL20.C, and P.1) Spike proteins were only about 2-fold lower, and not severely reduced. mRNA-1273.351 elicited the most focused response towards the B. 1.351 Spike protein, with neutralization titers towards other (D614G, CAL20.C, and P.1) Spike proteins being 3- to 5-fold lower. However, mRNA-1273.211, a 1:1 mixture of both mRNA-1273 and mRNA-1273.351, elicited robust neutralizing antibody responses to both D614G and B.1.351 Spike proteins encoded by the mRNAs, as well as to the other Spike proteins tested. These results indicate that administration of mRNAs elicited robust neutralizing antibody response to encoded SARS-CoV-2 Spike proteins, and that multivalent mRNA compositions containing multiple mRNAs are useful for elicited broad responses to a diverse group of viral antigens.

### Example 18: Immunization with Two Doses of mRNAs Encoding Variant Spike Proteins to Generate Neutralizing Antibodies to Variant Spike Proteins in Mice

BALB/c mice, 6-8 weeks of age, were administered 1 μg of mRNA encoding a SARS-CoV-2 antigen, specifically mRNA-1273 or mRNA-1273.351, or PBS (as a control) intramuscularly in one hind leg (formulated as a 50 μL dose) on day 1 (1^{st} dose, prime) and day 22 (2^{nd} dose, booster).

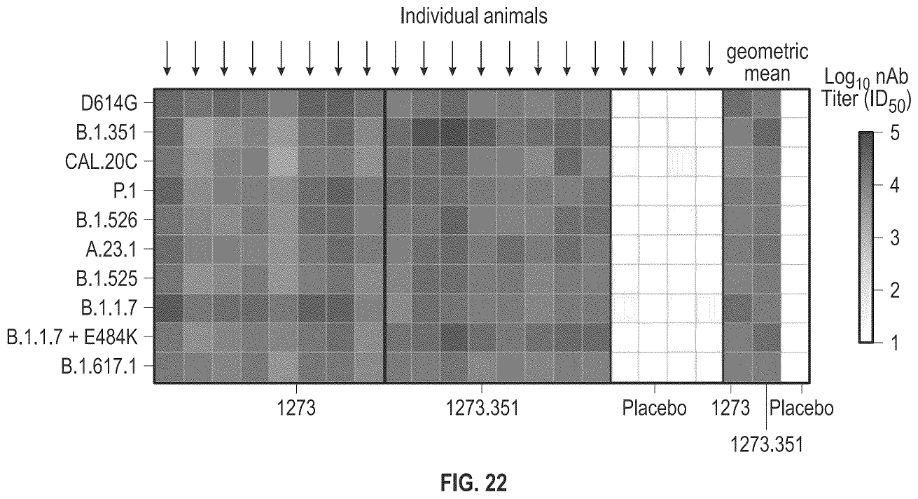

Sera obtained at day 36 from mice vaccinated with two 1 μg doses of mRNA-1273, mRNA-1273.351 were evaluated for neutralization activity against a panel of VSV-based pseudoviruses, each expressing a different SARS-CoV-2 Spike protein. The panel of Spike proteins tested included a D614G Spike protein, a B.1.351 Spike protein, a CAL.20C Spike protein, a P.1 Spike protein, a B.1.526 Spike protein, a A.23.1 Spike protein, a B.1.525 Spike protein, a B.1.1.7 Spike protein, a B.1.1.7 Spike protein comprising an E484K mutation, and a B.1.617.1 Spike protein. The results of these neutralization assays are shown in FIG. 22. Two 1 μg doses of mRNA-1273 or mRNA-1273.351 elicited robust neutralizing antibody responses towards each of the Spike proteins tested.

### Example 19—Immunogenicity of SARS-CoV-2 mRNA Variants (3^{rd} Doses)

BALB/c mice were immunized at days 1 and 22 with 1 μg of SEQ ID NO: 18 (WH2020_NatSP_2P) (n=8/group). The mice were subsequently administered a booster dose (dose 3; 1 μg) on day 57. The booster dose comprised mRNA-1273 (SEQ ID NO: 18), mRNA-1273.351 (SEQ ID NO: 9), or mRNA-1273.617.2 (SEQ ID NO: 28) (monovalent vaccines); or mRNA-1273+mRNA-1273.617.2, mRNA-1273+mRNA-1273.351, mRNA-1273+mRNA-1273.617.1, mRNA-1273+mRNA-1273.Angola, mRNA-1273.351+mRNA-1273.617.2, mRNA-1273.351+mRNA-1273.Angola, mRNA-1273+mRNA-1273.351+mRNA-1273.617.2, mRNA-1273+mRNA-1273.351+mRNA-1273.Angola, mRNA-1273+mRNA-1273.617.2+mRNA-1273.Angola, or mRNA-1273+mRNA-1273.351+mRNA-1273.617.2+mRNA-1273.Angola (multivalent vaccines at a 1:1 or 1:1:1 or 1:1:1:1 ratio). In each vaccine, the mRNA was formulated in lipid nanoparticles (LNPs) including 0.5-15% PEG-modified lipid, 5-25% non-cationic lipid, 25-55% sterol, and 20-60% ionizable amino lipid. The PEG-modified lipid was 1,2 dimyristoyl-sn-glycerol, methoxypolyethyleneglycol (PEG2000 DMG), the non-cationic lipid was 1,2 distearoyl-sn-glycero-3-phosphocholine (DSPC), the sterol was cholesterol, and the ionizable amino lipid had the structure of Compound 1, for example.

Blood was collected on days 36, 56, and 77 and analyzed by ELISA and neutralization assays as described herein. The results for days 36 (after the second dose) and 77 (after the booster dose) are shown in Table 16 below and demonstrate dramatically increased titers three weeks after administration of the booster dose compared to two weeks after the second dose of the vaccine (mRNA-1273) among the monovalent vaccines.

TABLE 16
Geometric Mean Titers (GMT) (1 μg Dose)-Monovalent Vaccines

| | Dose | | |
|---|---|---|---|
| Formulation | (ug) | Day 36-GMT | Day 77-GMT |
| PBS | 0 | 13 | 13 |
| mRNA-1273 | 1.0 | 79719 | 265697 |
| mRNA-1273.351 | 1.0 | 91875 | 317243 |
| mRNA-1273.617.2 | 1.0 | 71716 | 330799 |

Similar trends were seen with respect to the multivalent vaccines over the timepoints analyzed (Table 17).

TABLE 17
Geometric Mean Titers (GMT) (1 μg Dose)

| | Dose | Day 21- | Day 36- | Day 77- |
|---|---|---|---|---|
| Formulation | (ug) | GMT | GMT | GMT |
| PBS | 0 | 13 | 13 | 13 |
| | 0 | 13 | 13 | 13 |
| | 0 | 13 | 13 | 13 |
| mRNA-1273 | 1 | 11569 | 79719 | 265697 |
| mRNA-1273.351 | | 12107 | 91875 | 317243 |
| mRNA-1273.617.2 | | 6216 | 71716 | 330799 |
| mRNA-1273 + mRNA-1273.617.2 | | 8111 | 64622 | 289459 |
| mRNA-1273 + mRNA-1273.351 | | 10242 | 82166 | 282784 |
| mRNA-1273 + mRNA-1273.617.1 | | 12984 | 137997 | 359572 |
| mRNA-1273 + mRNA-1273.Angola | | 6698 | 81956 | 327273 |
| mRNA-1273.351 + mRNA-1273.617.2 | | 7907 | 88324 | 341445 |
| mRNA-1273.351 + mRNA-1273.Angola | | 11733 | 100648 | 425029 |
| mRNA-1273 + mRNA-1273.351 + mRNA-1273.617.2 | | 8548 | 88211 | 373908 |
| mRNA-1273 + mRNA-1273.351 + mRNA-1273.Angola | | 7871 | 97012 | 328856 |
| mRNA-1273 + mRNA-1273.617.2 + mRNA-1273.Angola | | 5469 | 88499 | 346611 |
| mRNA-1273 + mRNA-1273.351 + mRNA-1273.617.2 + mRNA-1273.Angola | | 5295 | 77195 | 346006 |

Neutralizing antibody titers against the D614G variant, the B.1.351 (beta) variant (L18F-D80A-D215G-ΔLAL242-244-R246I-K417N-E484K-N501Y-D614G-A701V), the P.1 (gamma) variant (L18F-T20N-P26S-D138Y-R190S-K417T-E484K-N501Y-D614G-H655Y-T1027I-V1176F), and the B.1.617.2 (delta) variant (T19R-G142D-E156G-F157del-R158del-L452R-T478K-D614G-P681R-D950N) were measured on day 56 (before the third dose) and day 77 using methods described above. The data for day 56 is shown in Table 18 below.

TABLE 18
Neutralization Titers (1 μg Dose)-Day 56 (all groups combined)

| | | NAb Titer | Fold Change |
|---|---|---|---|
| Virus | Day | (ID₅₀) | on D614G |
| D614G | 56 | 7104 | N/A |
| B.1.351 | 56 | 2625 | 2.7 |
| P.1 | 56 | 3128 | 2.3 |
| B.1.617.2 | 56 | 1821 | 3.9 |
| A.VOI.V2 | 56 | 567 | 12.5 |
| B.1.621 | 56 | 2286 | 3.1 |

Compared to what has been observed at two weeks post-vaccination, titers against D614G (WT) virus decreased one month after the mRNA-1273 primary series (at day 36, the D614G GMT was approximately 15,000). The highest reduction (12.5-fold) in neutralizing titers were observed for A. VOI.V2 when compared to D614G (WT).

Booster vaccination was observed to increase neutralizing titers, as shown in Tables 19-25 below.

TABLE 19
Neutralization Titers (1 μg Dose)-Day 56 vs. Day 77 (D614G GMT)

| | D614G GMT | | Fold increase |
|---|---|---|---|
| 3rd dose (Day 57) | D 56 | D 77 | on D 77 |
| mRNA-1273 | 5159 | 17605 | 3.4 |
| mRNA-1273.351 | 8605 | 34516 | 4.0 |
| mRNA-1273.617.2 | 6193 | 40399 | 6.5 |
| mRNA-1273.Angola | 6151 | 22317 | 3.6 |
| mRNA-1273 + mRNA-1273.351 (mRNA-1273.211) | 5392 | 49141 | 9.1 |
| mRNA-1273 + mRNA-1273.617.2 | 10033 | 60849 | 6.1 |
| mRNA-1273 + mRNA-1273.Angola | 4481 | 37835 | 8.4 |
| mRNA-1273.351 + mRNA-1273.617.2 | 9391 | 78162 | 8.3 |
| mRNA-1273.351 + mRNA-1273.Angola | 7519 | 35655 | 4.7 |
| mRNA-1273 + mRNA-1273.351 + mRNA-1273.617.2 | 7188 | 28891 | 4.0 |
| mRNA-1273 + mRNA-1273.351 + mRNA-1273.Angola | 10036 | 46016 | 4.6 |
| mRNA-1273 + mRNA-1273.617.2 + mRNA-1273.Angola | 7196 | 23300 | 3.2 |
| mRNA-1273 + mRNA-1273.351 + mRNA-1273.617.2 + mRNA-1273.Angola | 7813 | 50516 | 3.2 |

TABLE 20
Neutralization Titers (1 μg Dose)-Day 56 vs. Day 77 (B.1.351 GMT)

| | B.1.351 GMT | | Fold increase |
|---|---|---|---|
| 3rd dose (Day 57) | D 56 | D 77 | on D 77 |
| mRNA-1273 | 1793 | 7192 | 4.0 |
| mRNA-1273.351 | 3665 | 29008 | 7.9 |
| mRNA-1273.617.2 | 2217 | 22180 | 10.0 |
| mRNA-1273.Angola | 2850 | 9257 | 3.2 |
| mRNA-1273 + mRNA-1273.351 (mRNA-1273.211) | 2929 | 26766 | 9.1 |
| mRNA-1273 + mRNA-1273.617.2 | 3074 | 27674 | 9.0 |
| mRNA-1273 + mRNA-1273.Angola | 2702 | 16729 | 6.2 |
| mRNA-1273.351 + mRNA-1273.617.2 | 3076 | 49894 | 16.2 |
| mRNA-1273.351 + mRNA-1273.Angola | 2084 | 22926 | 11.0 |
| mRNA-1273 + mRNA-1273.351 + mRNA-1273.617.2 | 2363 | 18562 | 7.9 |
| mRNA-1273 + mRNA-1273.351 + mRNA-1273.Angola | 3534 | 22109 | 6.3 |
| mRNA-1273 + mRNA-1273.617.2 + mRNA-1273.Angola | 1961 | 10116 | 5.2 |
| mRNA-1273 + mRNA-1273.351 + mRNA-1273.617.2 + mRNA-1273.Angola | 2653 | 23235 | 8.8 |

TABLE 21
Neutralization Titers (1 μg Dose)-Day 56 vs. Day 77 (B.1.617.2 (delta) GMT)

| | B.1.617.2 GMT | | Fold increase |
|---|---|---|---|
| 3rd dose (Day 57) | D 56 | D 77 | on D 77 |
| mRNA-1273 | 879 | 4611 | 5.2 |
| mRNA-1273.351 | 3634 | 17822 | 4.9 |
| mRNA-1273.617.2 | 2423 | 21713 | 9.0 |
| mRNA-1273.Angola | 1380 | 9216 | 6.7 |
| mRNA-1273 + mRNA-1273.351 (mRNA-1273.211) | 1750 | 12064 | 6.9 |
| mRNA-1273 + mRNA-1273.617.2 | 3187 | 25999 | 8.2 |
| mRNA-1273 + mRNA-1273.Angola | 1336 | 13911 | 10.4 |
| mRNA-1273.351 + mRNA-1273.617.2 | 1807 | 22294 | 12.3 |
| mRNA-1273.351 + mRNA-1273.Angola | 1938 | 10752 | 5.5 |
| mRNA-1273 + mRNA-1273.351 + mRNA-1273.617.2 | 1217 | 10099 | 8.3 |
| mRNA-1273 + mRNA-1273.351 + mRNA-1273.Angola | 1880 | 8037 | 4.3 |
| mRNA-1273 + mRNA-1273.617.2 + mRNA-1273.Angola | 1886 | 9939 | 5.3 |
| mRNA-1273 + mRNA-1273.351 + mRNA-1273.617.2 + mRNA-1273.Angola | 2018 | 14229 | 7.1 |

TABLE 22
Neutralization Titers (1 μg Dose)-Day 56 vs. Day 77 (B.1.621 (mu) GMT)

| | B.1.621 GMT | | Fold increase |
|---|---|---|---|
| 3rd dose (Day 57) | D 56 | D 77 | on D 77 |
| mRNA-1273 | | | — |
| mRNA-1273.351 | | | — |
| mRNA-1273.617.2 | 1626 | 15183 | 9.3 |
| mRNA-1273.Angola | | | — |
| mRNA-1273 + mRNA-1273.351 (mRNA-1273.211) | 3149 | 19405 | 6.2 |
| mRNA-1273 + mRNA-1273.617.2 | 2400 | 13340 | 5.6 |
| mRNA-1273 + mRNA-1273.Angola | | | — |
| mRNA-1273.351 + mRNA-1273.617.2 | 2223 | 33417 | 15.0 |
| mRNA-1273.351 + mRNA-1273.Angola | | | — |
| mRNA-1273 + mRNA-1273.351 + mRNA-1273.617.2 | | | — |
| mRNA-1273 + mRNA-1273.351 + mRNA-1273.Angola | | | — |
| mRNA-1273 + mRNA-1273.617.2 + mRNA-1273.Angola | | | — |
| mRNA-1273 + mRNA-1273.351 + mRNA-1273.617.2 + mRNA-1273.Angola | | | — |

TABLE 23
Neutralization Titers (1 μg Dose)-Day 56 vs. Day 77 (P.1 (gamma) GMT)

| | P.1 GMT | | Fold increase |
|---|---|---|---|
| 3rd dose (Day 57) | D 56 | D 77 | on D 77 |
| mRNA-1273 | 2289 | 7340 | 3.2 |
| mRNA-1273.351 | 3221 | 28300 | 8.8 |
| mRNA-1273.617.2 | 2680 | 22183 | 8.3 |
| mRNA-1273.Angola | | | — |
| mRNA-1273 + mRNA-1273.351 (mRNA-1273.211) | 3474 | 29127 | 8.4 |
| mRNA-1273 + mRNA-1273.617.2 | 3481 | 26000 | 7.5 |
| mRNA-1273 + mRNA-1273.Angola | | | — |
| mRNA-1273.351 + mRNA-1273.617.2 | 4330 | 43279 | 10.0 |
| mRNA-1273.351 + mRNA-1273.Angola | | | |
| mRNA-1273 + mRNA-1273.351 + mRNA-1273.617.2 | 2831 | 23265 | 8.2 |
| mRNA-1273 + mRNA-1273.351 + mRNA-1273.Angola | | | — |
| mRNA-1273 + mRNA-1273.617.2 + mRNA-1273.Angola | | | — |
| mRNA-1273 + mRNA-1273.351 + mRNA-1273.617.2 + mRNA-1273.Angola | | | — |

TABLE 24
Neutralization Titers (1 μg Dose)-Day 56 vs. Day 77 (A.VOI.V2 GMT)

| | A.VOL.V2 GMT | | Fold increase |
|---|---|---|---|
| 3rd dose (Day 57) | D 56 | D 77 | on D 77 |
| mRNA-1273 | | | — |
| mRNA-1273.351 | | | — |
| mRNA-1273.617.2 | | | — |
| mRNA-1273.Angola | 536 | 4337 | 8.1 |
| mRNA-1273 + mRNA-1273.351 (mRNA-1273.211) | | | — |
| mRNA-1273 + mRNA-1273.617.2 | | | — |
| mRNA-1273 + mRNA-1273.Angola | 407 | 5569 | 13.7 |
| mRNA-1273.351 + mRNA-1273.617.2 | | | — |
| mRNA-1273.351 + mRNA-1273.Angola | 498 | 4296 | 8.6 |
| mRNA-1273 + mRNA-1273.351 + mRNA-1273.617.2 | | | — |
| mRNA-1273 + mRNA-1273.351 + mRNA-1273.Angola | 796 | 4201 | 5.3 |
| mRNA-1273 + mRNA-1273.617.2 + mRNA-1273.Angola | 736 | 2649 | 3.6 |
| mRNA-1273 + mRNA-1273.351 + mRNA-1273.617.2 + mRNA-1273.Angola | 521 | 5669 | 10.9 |

TABLE 25
Fold-Decrease on D614G

| | B.1.351 | | B.1.617.2 | | P.1 | | A.VOI.V2 | | B.1.621 | |
|---|---|---|---|---|---|---|---|---|---|---|
| | Fold | | Fold | | Fold | | Fold | | Fold | |
| | decrease on | | decrease on | | decrease on | | decrease on | | decrease on | |
| | D614G | | D614G | | D614G | | D614G | | D614G | |
| 3rd dose (Day 77) | D 56 | D 77 | D 56 | D 77 | D 56 | D 77 | D 56 | D 77 | D 56 | D 77 |
| mRNA-1273 | 2.9 | 2.4 | 5.9 | 3.8 | 2.3 | 2.4 | | | | |
| mRNA-1273.351 | 2.3 | 1.2 | 2.4 | 1.9 | 2.7 | 1.2 | | | | |
| mRNA-1273.617.2 | 2.8 | 1.8 | 2.6 | 1.9 | 2.3 | 1.8 | | | 3.8 | 2.7 |
| mRNA-1273.Angola | 2.2 | 2.4 | 4.5 | 2.4 | | | 11.5 | 5.1 | | |
| mRNA-1273 + | 1.8 | 1.8 | 3.1 | 4.1 | 1.6 | 1.7 | | | 1.7 | 2.5 |
| mRNA-1273.351 | | | | | | | | | | |
| (mRNA-1273.211) | | | | | | | | | | |
| mRNA-1273 + | 3.3 | 2.2 | 3.1 | 2.3 | 2.9 | 2.3 | | | 4.2 | 4.6 |
| mRNA-1273.617.2 | | | | | | | | | | |
| mRNA-1273 + | 1.7 | 2.3 | 3.4 | 2.7 | | | 11.0 | 6.8 | | |
| mRNA-1273.Angola | | | | | | | | | | |
| mRNA-1273.351 + | 3.1 | 1.6 | 5.2 | 3.5 | 2.2 | 1.8 | | | 4.2 | 2.3 |
| mRNA-1273.617.2 | | | | | | | | | | |
| mRNA-1273.351 + | 3.6 | 1.6 | 3.9 | 3.3 | | | 15.1 | 8.3 | | |
| mRNA-1273.Angola | | | | | | | | | | |
| mRNA-1273 + | 3.0 | 1.6 | 5.9 | 2.9 | 2.5 | 1.2 | | | | |
| mRNA-1273.351 + | | | | | | | | | | |
| mRNA-1273.617.2 | | | | | | | | | | |
| mRNA-1273 + | 2.8 | 2.1 | 5.3 | 5.7 | | | 12.6 | 11.0 | | |
| mRNA-1273.351 + | | | | | | | | | | |
| mRNA-1273.Angola | | | | | | | | | | |
| mRNA-1273 + | 3.7 | 2.3 | 3.8 | 2.3 | | | 9.8 | 8.8 | | |
| mRNA-1273.617.2 + | | | | | | | | | | |
| mRNA-1273.Angola | | | | | | | | | | |
| mRNA-1273 + | 2.9 | 2.2 | 3.9 | 3.6 | | | 15.0 | 8.9 | | |
| mRNA-1273.351 + | | | | | | | | | | |
| mRNA-1273.617.2 + | | | | | | | | | | |
| mRNA-1273.Angola | | | | | | | | | | |

As demonstrated above, neutralizing titers were lower one month post-primary series in mice than what is normally observed 2 weeks post vaccination (D36; D614G: ˜15000 GMT), and the highest fold-reduction was seen against the A. VOI. V2 variant after the primary series. About a 2.7-, 2.3-, and 3.9-fold reduction were observed on the beta (B.1.351), gamma (P.1), and delta (B.1.617.2) variants, respectively, when compared to D614G (WT). Against mu (B.1.621), about a 3.1-fold reduction was seen when compared to D614G.

After the third booster dose, all vaccine constructs produced an increase in neutralizing titers against all variants compared to what was observed on day 56. Variant vaccine constructs resulted in higher titers than the mRNA-1273 vaccine booster. Overall, the highest increase in titers against the variants were observed with the bivalent mRNA-1273.351+mRNA-1273.617 combination vaccine. Vaccination formulations that included mRNA-1273.617.2 gave high neutralizing titers against the matched B.1.617.2 pseudovirus in mice. Interference from combining different vaccine constructs was not observed in the neutralization titers when mice were administered the bi-, tri-, and quadrivalent vaccines.

In addition, the fold-decrease on D614G (WT) that was observed against variants after the primary series was less after the third booster dose. For example, the mRNA-1273+mRNA-1273.617.2 and mRNA-1273.351+mRNA-1273.617.2 formulations showed higher fold-reduction than other formulations; however, these two groups also produced the highest titer against each variant tested. Almost all booster vaccines were able to minimize the gap between the titers observed against the variant of interest or variant of concern and D614G (WT) pseudovirus. While some booster vaccinations resulted in higher fold-reduction than the results after one month after the primary series, the GMT after the booster dose were found to be still higher. Overall, administration of the mRNA-1273.351+mRNA-1273.617.2 vaccine in mice was found to decrease the fold-drop against the beta, delta, and mu variants.

The experiment was repeated with selected vaccine formulations and similar results were obtained. The results are shown below:

| | Dose | | | |
|---|---|---|---|---|
| Formulation/Material | (ug) | Day 21-GMT | Day 36-GMT | Day 78-GMT |
| PBS | | 13 | 13 | 13 |
| PBS | | 13 | 13 | 13 |
| PBS | | 13 | 13 | 13 |
| mRNA-1273 | 1 | 4831 | 164775 | 370729 |
| mRNA 1273.351 | 1 | 4296 | 126671 | 254320 |
| mRNA-1273.617.2-v2 | 1 | 3518 | 133100 | 332117 |
| mRNA-1273 + .1273.351 | 1 | 4892 | 113168 | 275097 |
| mRNA-1273 + .1273.617.2-v2 | 1 | 5886 | 185834 | 375110 |
| mRNA-1273 + 1273.351 + 1273.617.2-v2 | 1 | 6358 | 178206 | 371299 |
| mRNA1273.351 + 1273.617.2-v2 | 1 | 4705 | 118779 | 293289 |
| mRNA-1283 | 1 | 6179 | 135176 | 424079 |
| mRNA-1283.351 | 1 | 11888 | 100389 | 451348 |
| mRNA-1283.617.2 | 1 | 5287 | 130957 | 504622 |
| mRNA-1283 + 1283.351 | 1 | 5055 | 141217 | 429434 |
| mRNA-1283 + 1283.617.2 | 1 | 4763 | 115615 | 593690 |
| mRNA-1283 + 1283.351 + 1283.617.2 | 1 | 5398 | 98220 | 516556 |

### Example 20: Neutralizing Activity Against Variants (Two Doses of mRNA-1273)

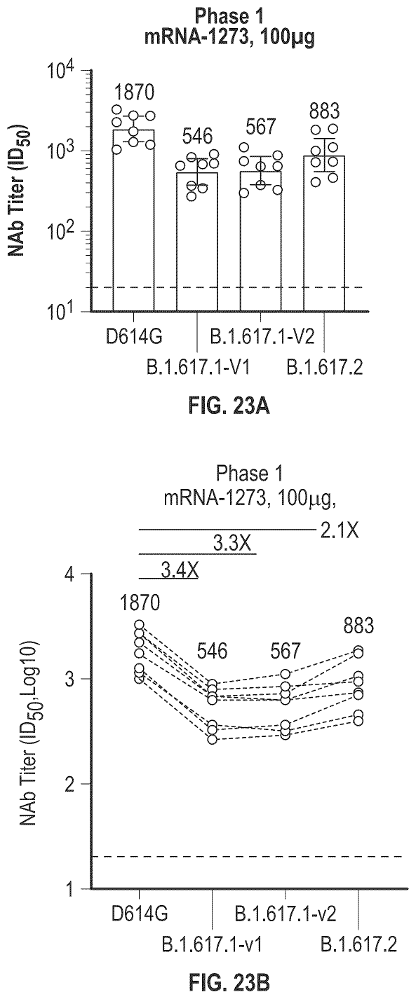

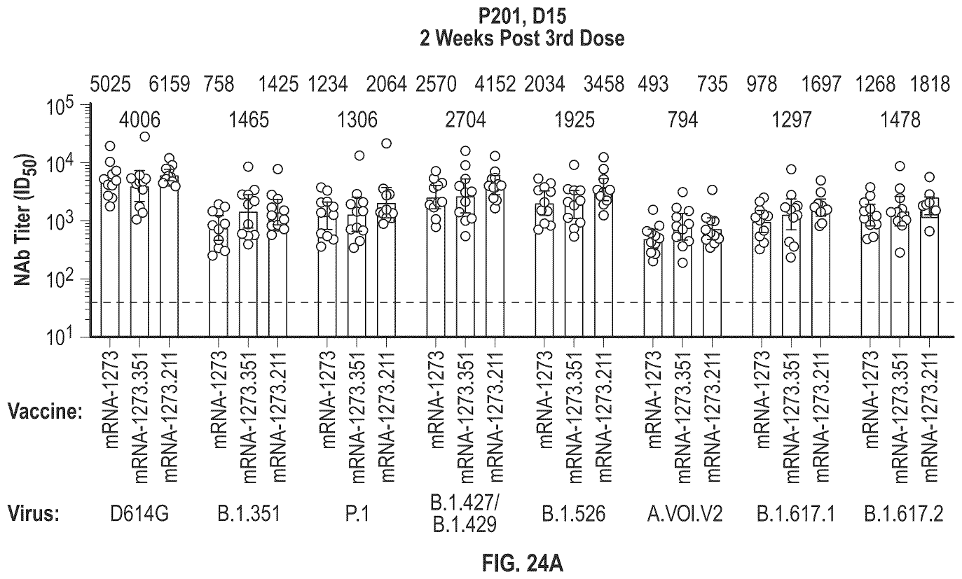

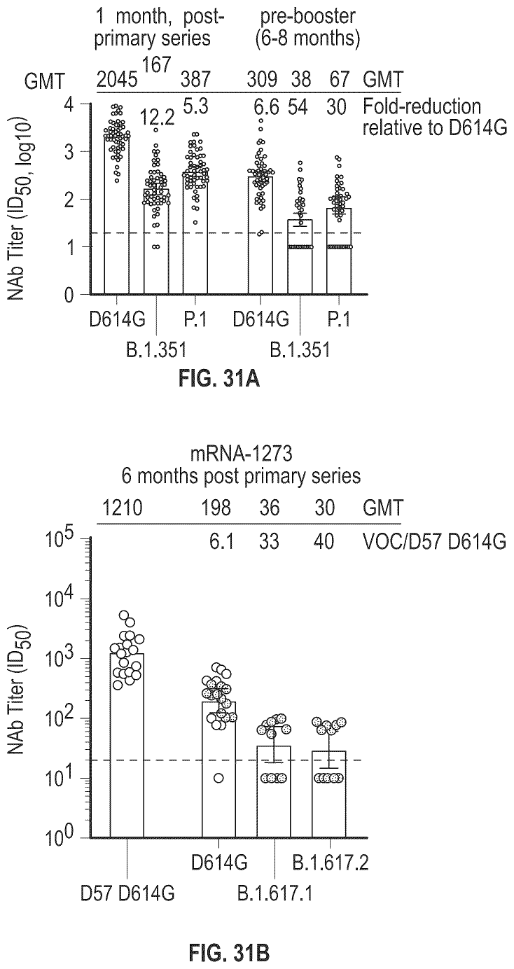

Human subjects were immunized with two doses of 100 μg mRNA-1273, as described in Example 13. FIG. 23A shows the neutralizing antibody titer of sera taken from participants against D614G, B.1.617.1-v1, B.1.617.1-v2, and B.1.617.2. FIG. 23B shows the relative decrease in neutralizing antibody titer compared to D614G. One month and 6-8 months after administration of the second dose, neutralizing antibodies against D614, B.1.351, P.1, and B.1.617 were measured. As can be seen in FIG. 31A, titers against the prototype strain were high after one month, with a significant reduction seen against both the B.1.351 variant (12.2 fold) and the P.1 variant (5.3 fold). Six to eight months after the primary series, neutralizing antibodies wane-they were 6.6 fold lower against the original D614G virus, and titers against B.1.351 and P.1 were 30-54-fold reduced versus peak titers measured after the primary series against the prototype strain virus (D614G) (FIG. 31B).

In a further study, neutralization activity of sera was measured against the following virus strains: D614G pseudovirus (predominant strain in 2020), B.1.1.7, B.1.1.7+E484K, B.1.427/B.1.429, P.1, B.1.351-v1, B.1.351-v2, B.1.351-v3, B.1.526, B.1.617.1-v1, B.1.617.1-v2, B.1.617.2, A.23.1-v1, A.23.1-v2, B.1.525, and A. VOI. V2. Sera from the phase 1 mRNA-1273 clinical trial (8 participants, 1 week following Dose 2) were evaluated against each variant. Results showed minimal effects on neutralization titers against B.1.1.7 and A.23.1-v1 compared to D614G (data not shown). In contrast, all other variants examined showed decreased neutralization titers compared with D614G, although all remained completely susceptible to mRNA-1273-elicited serum neutralization, but with reduced titers. Reductions in neutralization titers for these variants ranged from a factor of 2.1 to 8.4 compared with D614G, with the greatest effect on neutralization observed for A. VOI. V2 and B.1.351-v3 (8.0-fold and 8.4-fold reductions compared with D614G, respectively).

### Example 21. Neutralization Against Variants Post Third Dose (Booster)

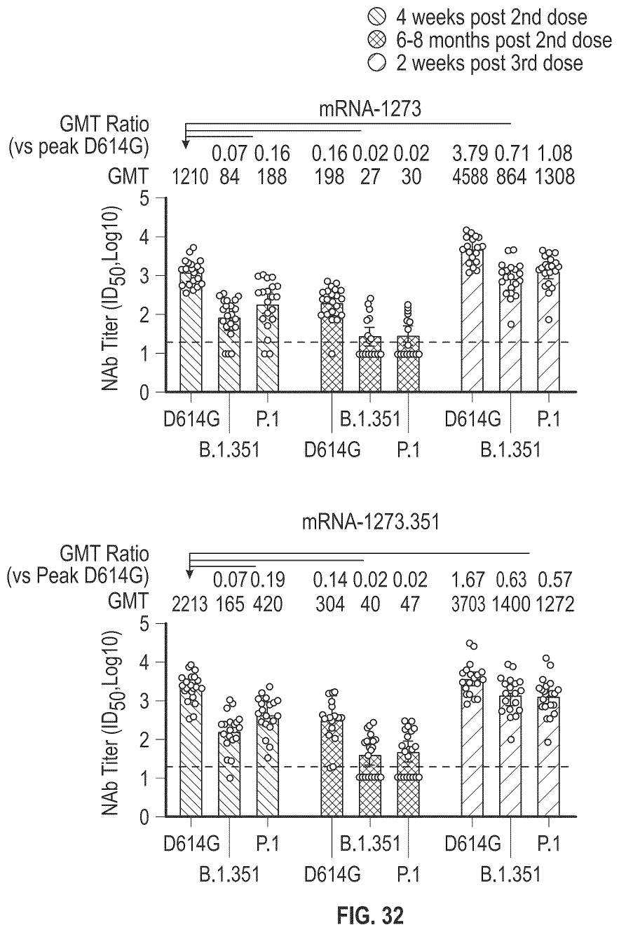

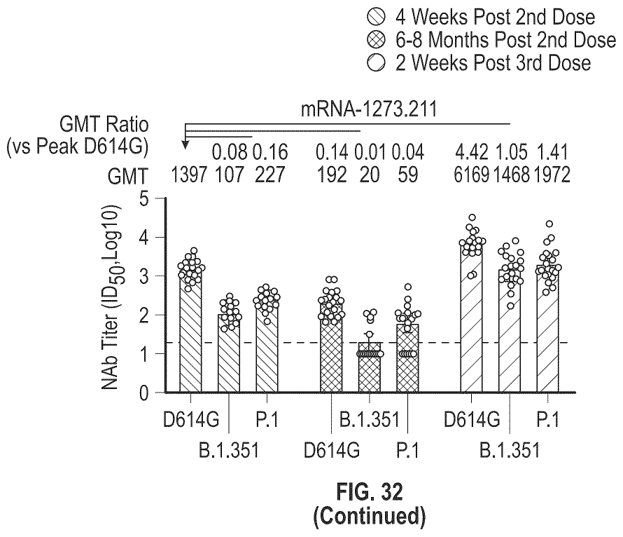

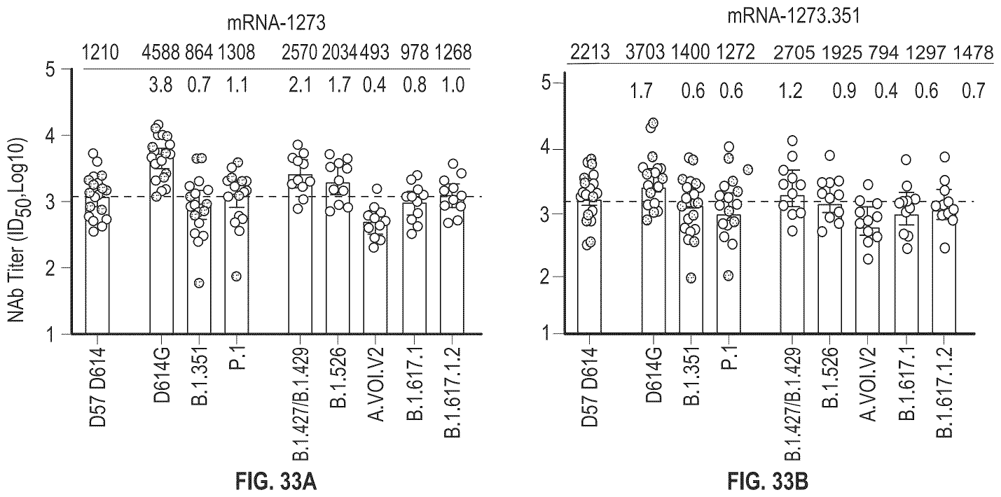

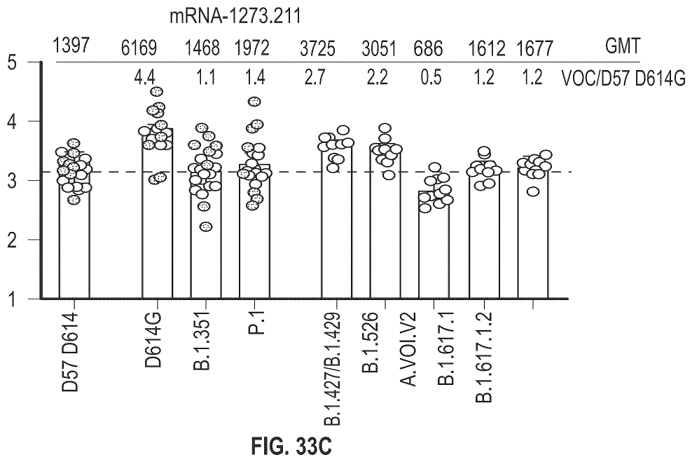

Human subjects were immunized with two doses of 100 μg mRNA-1273, as described in Example 13. A third dose was administered 6-8 months after the primary vaccination was complete. Neutralizing titers against B.1.351 and P.1 were measured over time. The results are shown in FIG. 32. The booster dose (mRNA-1273, mRNA-1273.351, or mRNA-1273.211) was found to increase neutralizing antibody in a surprising manner against both the wild type (D614G) and two variants tested (B.1.351 and P.1).

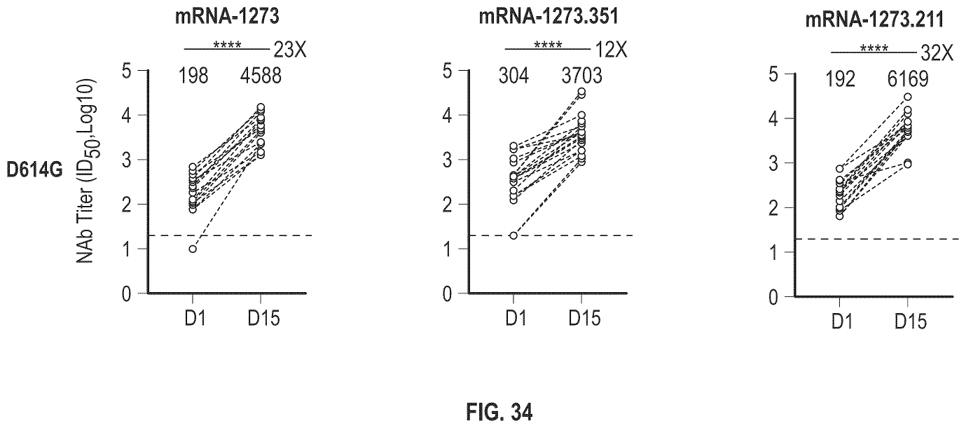

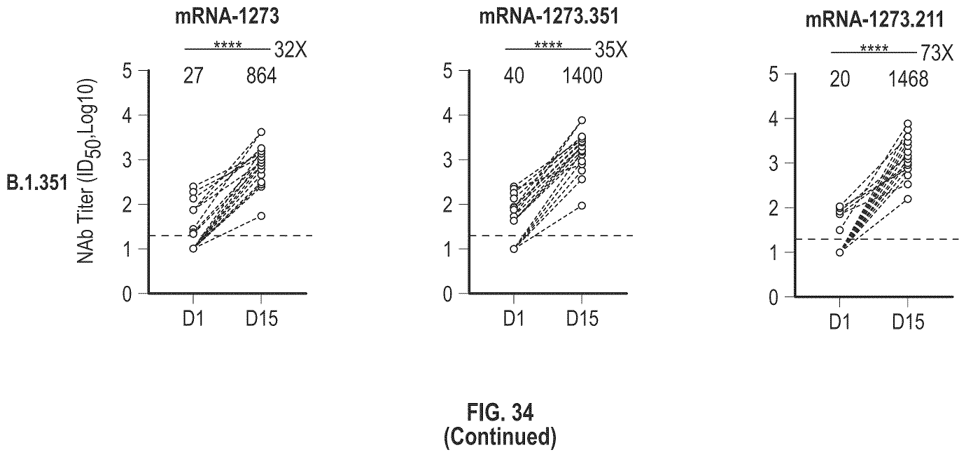

Further variants of concern and variants of interest were screened. As can be seen in FIGS. 33A-33C, the booster shots were found to neutralize each variant as well. A comparison of the neutralizing titers resulting from administration of one of the three booster shots is shown in FIG. 34. mRNA-1273.211 was found to outperform the two other booster shot strategies against all variants tested.

### Example 22. Safety and Immunogenicity of SARS-CoV-2 Variant mRNA Booster in Adults

mRNA-1273, a lipid nanoparticle-encapsulated messenger RNA vaccine encoding a prefusion stabilized S protein of the WA1/2020 isolate, demonstrated anti-SARS-CoV-2 immune responses in phase 1 (NCT04283461) and phase 2 (NCT04405076) trials in adults, an acceptable safety profile, and 94% efficacy against symptomatic Covid-19 disease in the phase 3 Coronavirus Efficacy (COVE) (NCT04470427) trial in over 30,000 participants. The vaccine received authorization from several global regulatory bodies including the U.S. Food and Drug Administration. Although the vaccine is highly effective in reducing the symptoms and severe complications of Covid-19, several viral variants with changes in the S protein have arisen, some of which have been identified as variants of concern (VOCs): Alpha [B.1.1.7], Beta [B.1.351], Gamma [P.1], and Delta [B.1.617.2]. Reduction in efficacy has been reported against some Covid-19 vaccines versus B.1.351 and more recently B.1.617.2.

Described herein are the preliminary safety and immunogenicity of single booster doses of mRNA-1273 (50 μg), modified mRNA-1273.351 (20 or 50 μg) encoding the spike protein of B.1.351 (beta variant), and multivalent mRNA-1273.211 (a 1:1 mix of mRNA-1273 [25 μg] and mRNA-1273.351 [25 μg]) in a Phase 2 trial.

### Results

### Trial Population

The Phase 2 trial consisted of a total of 660 participants. Upon study unblinding and implementation of Part B, 20 of the 186 participants who had originally received 2 priming doses of 100 μg mRNA-1273 were randomly selected based on visit assessments completed and sample availability to receive a single booster dose of 50 μg mRNA-1273. Sixty of the 14,711 participants, 12 who received two priming doses of 100 μg mRNA-1273, were selected to receive single booster doses of 50 μg of mRNA-1273.351 (Part C, cohort 1) or mRNA-1273.211 (Part C, cohort 2) or 20 μg of mRNA-1273.351 (Part C, cohort 3).

The baseline demographic characteristics of the 4 groups of participants who received booster doses of the prototype or the modified mRNA-1273 vaccines were generally similar (Table 26). Most of the participants were White and not Hispanic or Latino. The mean age of the participants who received boosters of mRNA-1273 (50 μg), mRNA-1273.351 (20 μg), mRNA-1273.351 (50 μg) or mRNA-1273.211 was 63.8, 47.5, 53.9 and 55.6 years, respectively. The duration (mean [SD]) between the second dose of mRNA-1273 in the primary series and the booster for mRNA-1273 (50 μg), mRNA-1273.351 (20 μg), mRNA-1273.351 (50 μg) or mRNA-1273.211 (50 μg) was 6.7 [0.5], 6.2 [0.3], 6.2 [0.3] and 6.2 [0.4] months, respectively.

TABLE 26
Demographics and Characteristics of Subjects

| | mRNA- | | mRNA- | | mRNA- | | mRNA- | |
|---|---|---|---|---|---|---|---|---|
| | 1273 | | 1273.351 | | 1273.351 | | 1273.211 | |
| | (50 μg) | | (20 μg) | | (50 μg) | | (50 μg) | |
| Characteristic n (%) | N = 20 | | N = 20 | | N = 20 | | N = 20 | |
| Age (years) | | | | | | | | |
| Mean (range) yr. | 63.8 | (38-76) | 47.5 | (26-67) | 53.9 | (27-70) | 55.6 | (28-79) |
| Gender | | | | | | | | |
| Male | 8 | (40) | 5 | (25) | 11 | (55) | 12 | (60) |
| Female | 12 | (60) | 15 | (75) | 9 | (45) | 8 | (40) |
| Race | | | | | | | | |
| White | 20 | (100) | 20 | (100) | 19 | (95) | 19 | (95) |
| Black or African-American | 0 | | 0 | | 0 | | 0 | |
| Asian | 0 | | 0 | | 1 | (5) | 0 | |
| American Indian or Alaska Native | 0 | | 0 | | 0 | | 1 | (5) |
| Native Hawaiian or other Pacific | 0 | | 0 | | 0 | | 0 | |
| Islander, Multiracial, Other, Not | | | | | | | | |
| reported, Unknown | | | | | | | | |
| Ethnicity | | | | | | | | |
| Hispanic or Latino | 0 | | 1 | (5) | 0 | | 1 | (5) |
| Not Hispanic or Latino | 20 | (100) | 19 | (95) | 20 | (100) | 19 | (95) |
| Not reported or Unknown | 0 | | 0 | | 0 | | 0 | |
| Time interval between second dose of | | | | | | | | |
| mRNA-1273 during the primary series | | | | | | | | |
| and the booster dose | | | | | | | | |
| Mean (SD) (months^{†}) | 6.7 | (0.5) | 6.2 | (0.3) | 6.2 | (0.3) | 6.2 | (0.4) |
| Range (months) | 5.9-7.5 | | 5.5-6.6 | | 5.6-6.6 | | 5.4-6.8 | |
| Body Mass Index (kg/m²) | | | | | | | | |
| Mean (SD) | 26.2 | (2.1) | 33.3 | (6.6) | 30.3 | (6.5)* | 33.0 | (7.5) |

Legend:
SD = standard deviation.
*Missing data for 1 participant.
^{†}Calculated with 30 days/month.

Neutralizing Responses to Wild-Type D614G and B.1.351 Immediately Prior to and after the Booster Dose

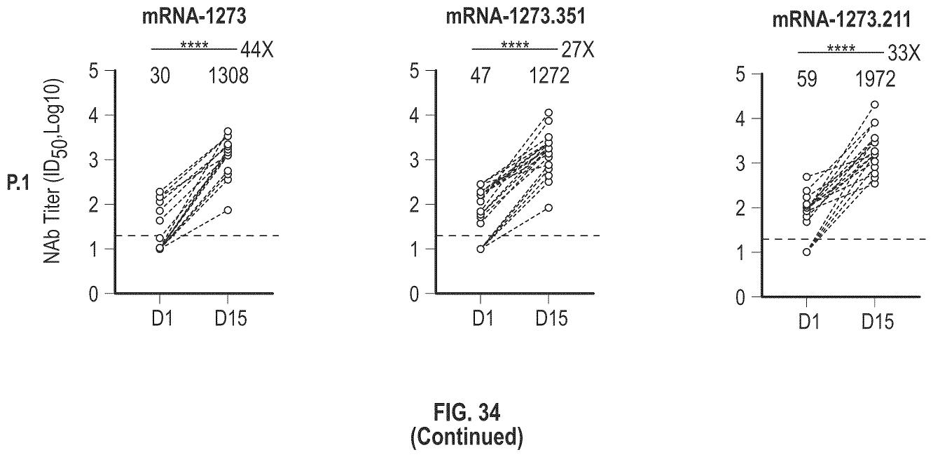

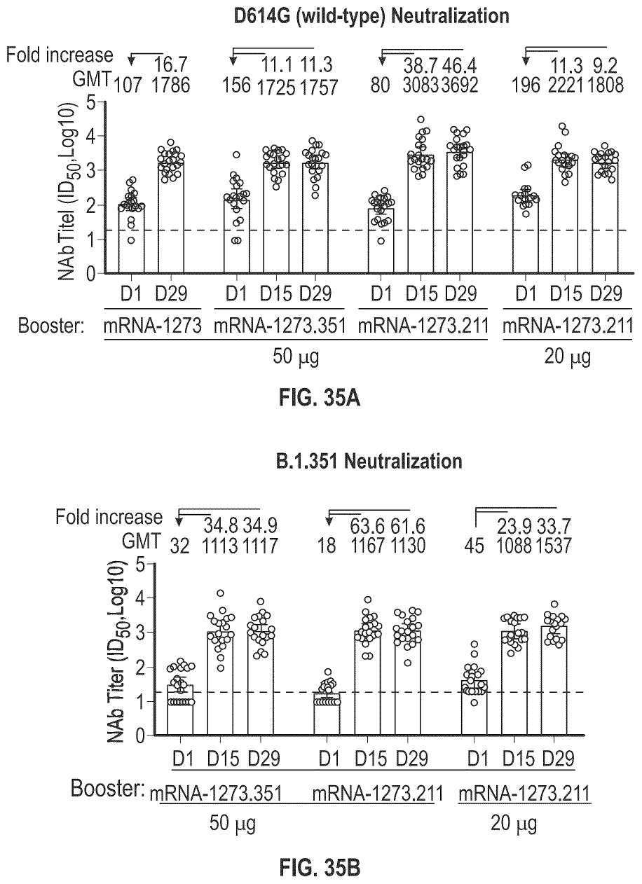

Wild-type D614G and B.1.351 neutralization was measured in samples collected ˜6 months after the primary series of mRNA-1273, but immediately before the booster dose (D1), and in samples collected on day 15 (D15) or 29 (D29) after the booster dose in a clinically validated Lentivirus PsVN assay. The wild-type D614G virus was neutralized by samples collected prior to the booster from participants in Part B and Part C cohorts 1, 2, and 3 (FIG. 35A), while neutralization of B.1.351 was low or nondetectable prior to the boost from participants in Part C cohorts 1, 2, and 3 (FIG. 35B).

After the booster dose, participant sera were collected on day 29 from Part B participants (mRNA-1273 booster) or on day 15 and 29 from Part C cohorts 1, 2, and 3 (50 μg mRNA-1273.351, 50 μg mRNA-1273.211, and 20 μg mRNA-1273.351 boosters, respectively). Neutralization of the wild-type D614G and B.1.351 viruses significantly increased after each booster dose. Against the wild-type D614G virus, 16.7, 11.3, 46.4, and 9.2-fold higher GMTs were measured in the mRNA-1273 (50 μg), mRNA-1273.351 (50 μg), mRNA-1273.211 (50 μg), and mRNA-1273.351 (20 μg) cohorts, respectively, on D29. Against the B.1.351 variant, 34.9, 61.6, and 33.7-fold higher GMTs were measured in the mRNA-1273.351 (50 μg), mRNA-1273.211 (50 μg), and mRNA-1273.351 (20 μg) cohorts, respectively, on D29. In addition, participants who did not have measurable titers against the wild-type D614G or B.1.351 virus ˜6 months after the primary series, prior to the boost, all had regained significant titers after the boost.

Correlation of the Clinically Validated Lentivirus PsVN Assay with a Research-Grade VSV-Based PsVN Assay

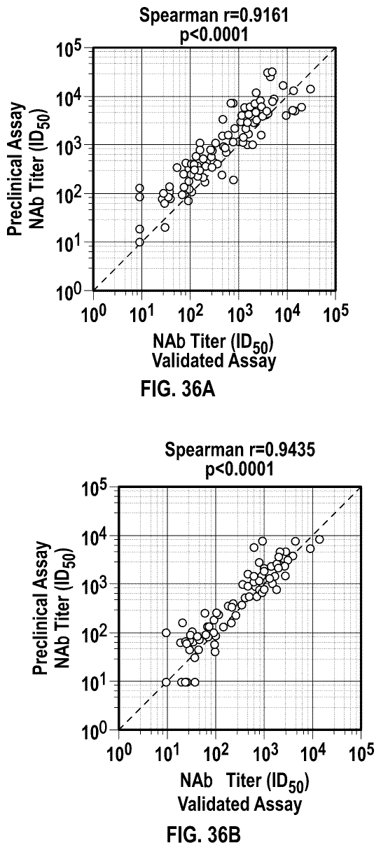

In order to support exploratory analysis of the Part B and Part C Cohort 1 and 2 clinical samples against SARS-CoV-2 variants, participant samples were analyzed in a research-grade VSV-based PsVN assay that has previously been used to evaluate the impact of SARS-CoV-2 variants on mRNA-1273 neutralization. Analysis of the results from the clinically validated and research-grade wild-type D614G and B.1.351 PsVN assays demonstrates significant correlation, with r=0.9160 against wild-type D614G and 0.9411 against B.1.351 in an analysis of results from D1 and D15 samples (FIGS. 36A-36B).

### Exploratory Analysis of the Kinetics of Neutralizing Responses to Wild-Type D614G and VOCs Post-Primary Series Vaccination

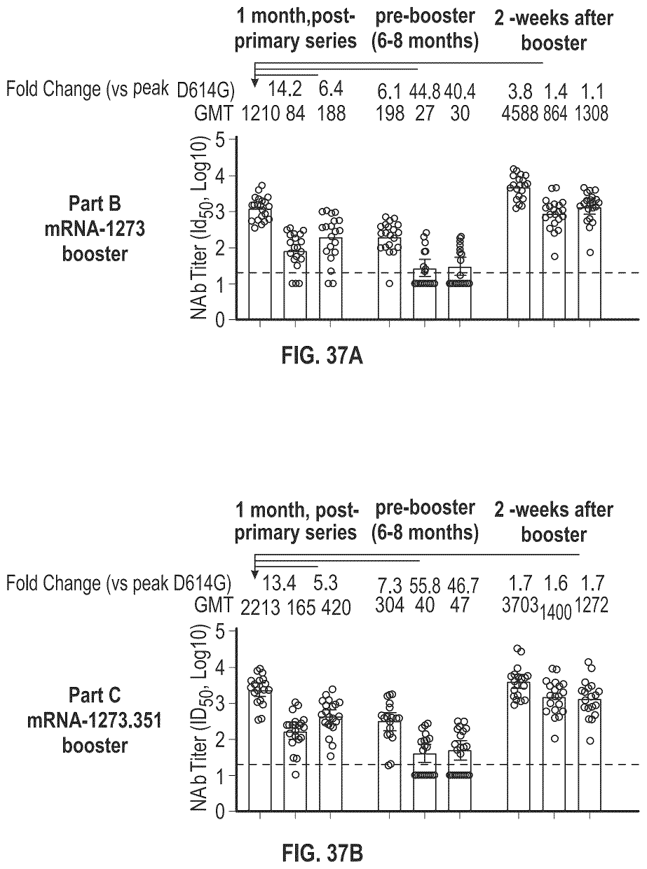

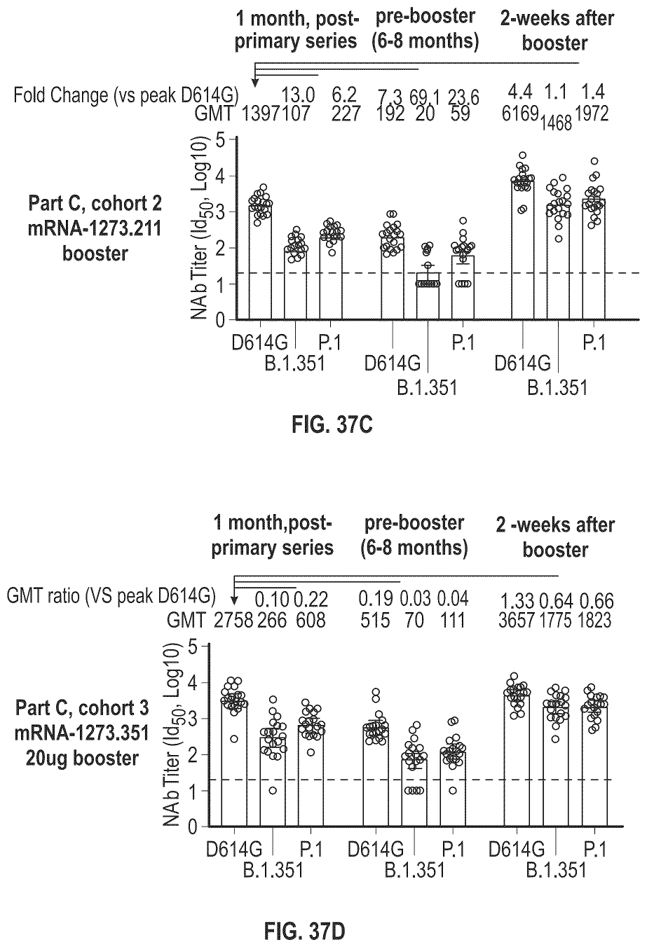

Exploratory analysis of samples collected after the primary mRNA-1273 vaccination series was performed using the VSV-based PsVN assay. Twenty-eight days after the primary 2-dose series, wild-type D614G neutralizing antibody GMTs were 1210 in the mRNA-1273, 2213 in the mRNA-1273.351 and 1397 in the mRNA-1273.211 Cohorts (FIGS. 37A-37D), with significant reductions seen against both B.1.351 (13-17-fold) and P.1 variants (5-7-fold). Approximately 6 months after the second dose of mRNA-1273, neutralizing antibody GMTs further decreased in comparison to peak titers measured against the D614G virus 1 month after the primary series. Titers against the wild-type D614G were 6 to 7-fold lower, while titers against the B.1.351 and P.1 variants were 24 to 69-fold lower. Approximately 44% and 30% of combined samples from Part B and C cohorts 1 and 2 were below the assay LLOQ against B.1.351 and P.1 viral variants.

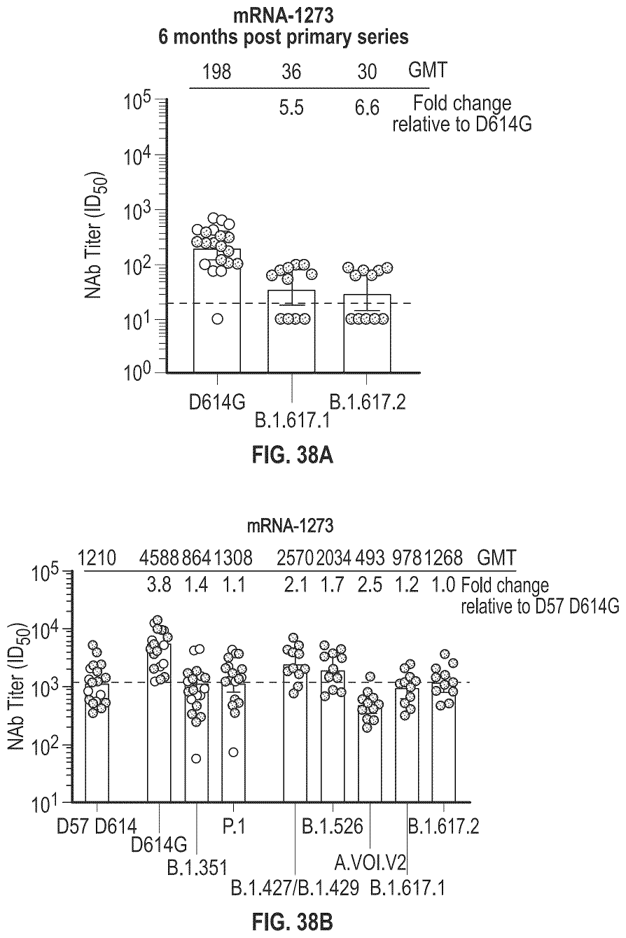

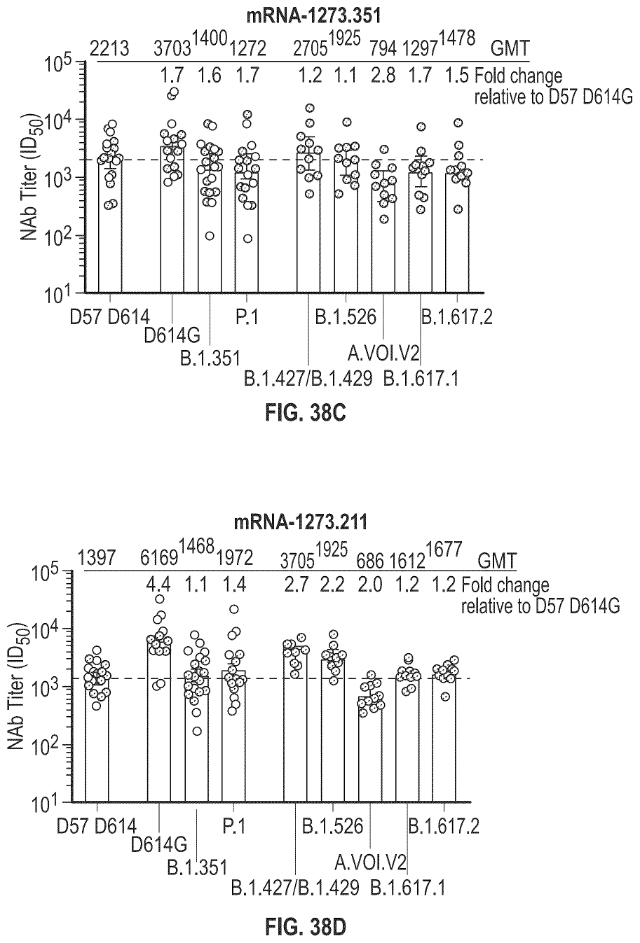

Similar to B.1.351 and P.1, neutralization of the B.1.617.2 variant, was considerably reduced 6 months after completion of the primary series. Sera from a random subset of the Part B cohort collected prior to the booster dose of mRNA-1273 showed a 5-6-fold reduction against B.1.617.1 and B.1.617.2 versus the wild-type D614G neutralizing titers measured at the same timepoint, with neutralization in 5 of 11 samples falling below the assay LLOQ against B.1.617.2 (FIG. 38A).

Exploratory Analysis of Neutralizing Responses Against Wild-Type D614G Virus and VOCs from Boosters

Wild-type D614G virus neutralizing titers were measured with the VSV-based PsVN assay in order to compare titers from samples collected 15 days after the booster dose versus peak titers measured from samples collected 28 days after the second dose of the mRNA-1273 primary series. After the boost, wild-type virus neutralization GMTs were 3.8, 1.7, and 4.4-fold higher from the mRNA-1273, mRNA-1273.351, and mRNA-1273.211 boosters, respectively, relative to the peak titers 28 days after the primary series vaccination (FIGS. 37A-37D). Against VOCs or VOIs, each of the booster strategies significantly increased variant-specific neutralization titers relative to those measured after the primary series. mRNA-1273.211 increased both B.1.351 and P.1 neutralization titers above the GMT level against the D614G strain measured after the primary series, with GMT titers increasing to 1468 against B.1.351 and 1973 against P.1 in the Part C cohort 2 participants 2-weeks after the booster dose.

### Exploratory Analysis of Booster Response to VOC Compared to a Primary Series GMT Benchmark

The VSV PsVN assay was used to assess COVE study samples collected 28 days after the primary series to establish a GMT benchmark. This benchmark was used to determine whether the boosters reached the same neutralization level shown in the pivotal study where efficacy was demonstrated, i.e., to levels seen in the D614G assay where 94% efficacy was measured, indicated by a GMT ratio (GMTr) ≥1.12 In the Part C, day 57 primary series participant samples (n=59) from the COVE study, a GMT of 2045 was established as the D614G neutralization benchmark, with GMTs per cohort that ranged from 1397-2758 (FIGS. 37A-37D).

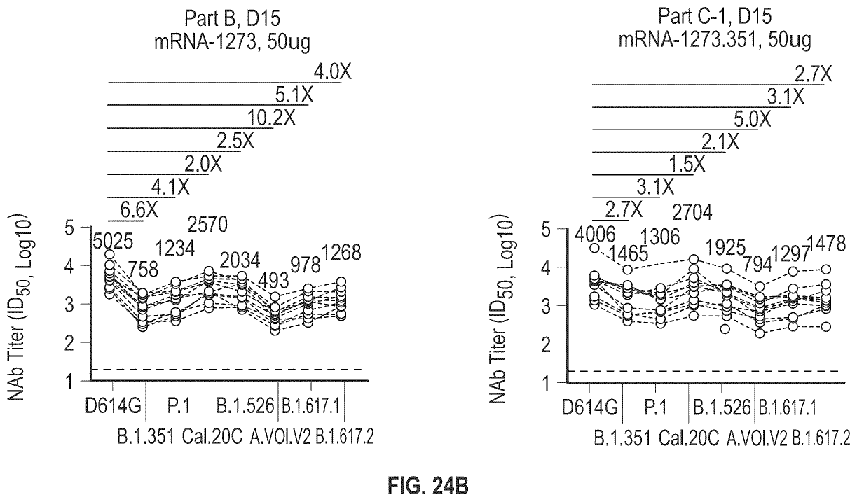

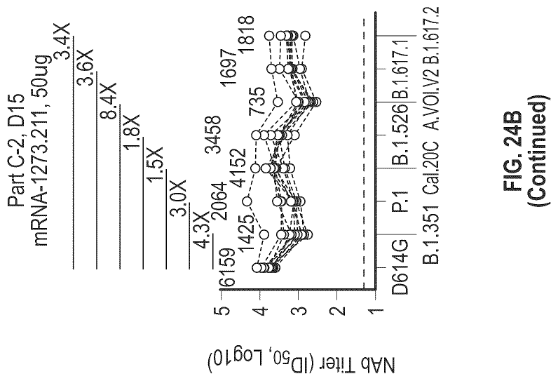

When samples collected 2-weeks after the respective booster dose were assessed against a panel of variants (FIGS. 24A-24B; 38B-38D), each mRNA booster significantly increased neutralization against all variants assessed, including B.1.617.2 and P.1, with neutralization against some of the variants approaching or exceeding the COVE study wild-type D614G GMT benchmarks. Of the three booster vaccines assessed, the multivalent mRNA-1273.211 booster showed the greatest increase in GMTs against the majority of VOCs (FIGS. 24A-24B; 38A-38D).

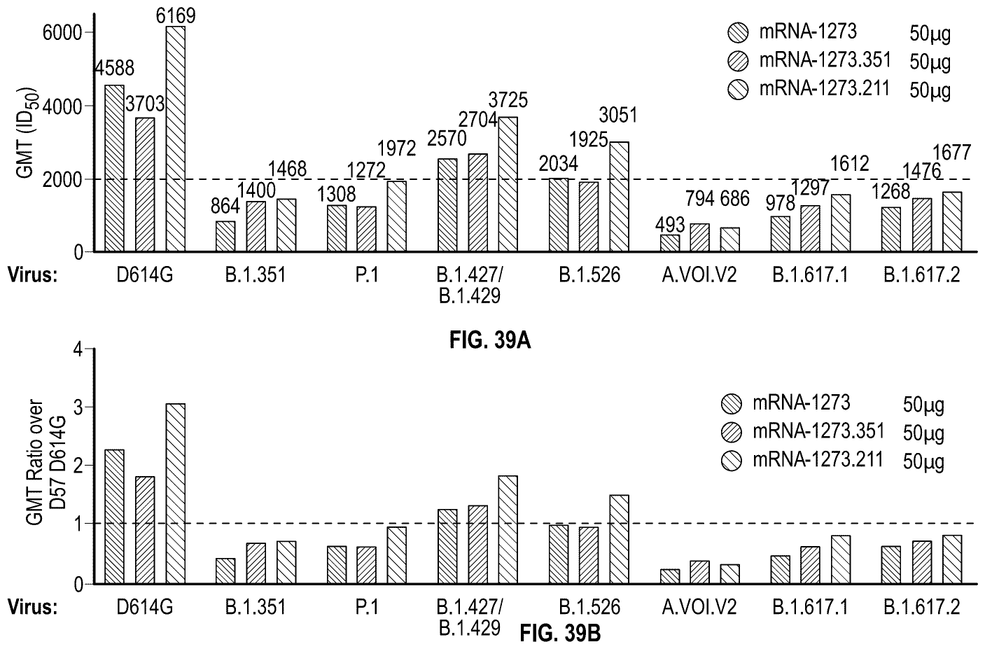

Compared to the COVE study D614G benchmark, the booster vaccines yielded higher GMTs against the wild-type D614G virus and several VOCs or VOIs, including B.1.617.2, based on a GMTr rise ≥1 (FIGS. 38B-38D). However, only the multivalent variant vaccine mRNA-1273.211 achieved a GMTr rise of >1 against all VOCs assessed. Of the three boosters, the multivalent mRNA-1273.211 50 μg booster also yielded higher variant GMTs versus the D57 D614G benchmark and against the largest number of variants including B.1.351, P.1, B.1.427/B.1.429, B.1.526, B.1.617.1, and B.1.617.2. A comparison of the GMT versus the overall COVE GMT D614G benchmark is shown in FIGS. 39A-39B.

### Discussion

This preliminary evaluation describes the antibody persistence of mRNA-1273 and administration of booster doses of mRNA-1273, mRNA-1273.351 and mRNA-1273.211 in a subset of 80 participants who had been vaccinated ˜6 months previously with the authorized dose and schedule of mRNA-1273. Antibody titers against the wild-type D614G peaked one month after the second dose of the primary series, and subsequently declined over the next 5 months prior to the delivery of the booster dose. These results are consistent with those reported in a lentiviral PsVN assay, where monitoring of neutralizing antibody levels was performed up to 6 months after the second dose of mRNA-1273. Reduction of neutralizing antibody was evident 28 days after the primary series vaccination against B.1.351 and P.1 to greater levels than measured against samples collected 7 days after the primary series, likely due to further affinity maturation of B-cells and alteration of the available antibody repertoire. Additional reduction or complete loss of detectible levels of neutralizing antibody ˜6 months after the primary vaccination was evident against B.1.351, P.1, and B.1.617.2.

The safety profiles following single injections of 50 μg mRNA-1273, 20 or 50 μg mRNA-1273.351, and 50 μg mRNA-1273.211 boosters were generally similar to those observed after a second dose of mRNA-1273 in the previously reported phase 2 and 3 studies.

Booster vaccination with mRNA-1273, mRNA-1273.351, and mRNA-1273.211 induced robust anamnestic responses, confirming that the robust B-cell memory generated by mRNA vaccines can be quickly and potently boosted. High neutralizing titers were measured against the wild-type D614G strain after a booster dose which were up to 4.4-fold higher than peak titers after the primary series. Increased VSV PsVN titers were measured against variant viruses including the key VOCs, B.1.351, P.1, and B.1.617.2, with titers against several variants approaching or exceeding those measured after the primary series against the wild-type D614G virus, particularly after boosting with mRNA-1273.211 (FIG. 37C). Increased titers against the VOCs suggest that further maturation of antibodies is feasible after a two-dose primary series of mRNA-1273, regardless of the composition of the booster dose. Additionally, boosting with mRNA-1273.351 and mRNA-1273.211 appeared to be more effective at increasing neutralization against the B.1.351 variant than with mRNA-1273.

For comparison to GMT titers measured in the Phase 3 COVE study where efficacy was established, 59 COVE participant samples were evaluated in the VSV PsVN assay with the wild-type D614G assay titers used to support additional analyses. The mRNA boosters each significantly increased neutralization against all variants assessed, including B.1.617.2 and P.1, with neutralization against some of the variants approaching or exceeding the COVE study benchmark. The multivalent mRNA-1273.211 50 μg booster yielded a GMTr rise ≥1 against all VOCs (FIG. 38D), indicating that variant neutralization GMTs after the booster exceeded peak wild-type virus GMTs after the primary series, potentially increasing breadth of coverage against VOCs or VOIs.

### Methods

### Study Design

The phase 2 mRNA-1273 P201 study (NCT04405076) enrolled adults at 8 sites in the U.S. Preliminary safety and immunogenicity results following two doses of 50 or 100 μg of mRNA-1273 have been previously reported. Once the primary efficacy endpoint for mRNA-1273 against Covid-19 was met in the phase 3 COVE trial and EUA was granted, both the phase 2 and 3 trial protocols were amended to transition the studies to open-label phases. The phase 2 study offered participants previously primed with two doses (50 or 100 μg) of mRNA-1273 in Part A an option to receive a single booster of 50 μg mRNA-1273 in Part B, however only 20 individuals primed with two doses of 100 μg of mRNA-1273 were included in this analysis. Part C was added to the phase 2 study, and participants at a single site from the phase 3 COVE trial who completed a two-dose series of 100 μg of mRNA-1273 were enrolled to receive a single booster of either 20 or 50 μg doses of mRNA-1273.351 or 50 μg of the multivalent mRNA-1273.211.

### Trial Participants

Eligible participants were adults, ≥18 years of age, considered by the investigator to be healthy at screening and were enrolled at one of the 8 participating study sites. Twenty Part B participants who received a single booster dose of mRNA-1273 50 μg in Part B were randomly selected for this sub-study analysis. For Part C, participants must have been previously enrolled in the mRNA-1273 phase 3 COVE study and received two doses of mRNA-1273 in Part A of that study, with a second dose at least 6 months prior to enrollment in Part C of the P201 study. Sixty participants were sequentially enrolled to receive mRNA-1273.351 50 g, mRNA-1273-211 50 μg, or mRNA-1273.351 20 μg (20/group).

### Trial Vaccines

The mRNA-1273.351 vaccine, like mRNA-1273, encodes the S protein of SARS-CoV-2 B.1.351 variant. mRNA-1273.211 was a 1:1 mix of 25 μg of mRNA-1273 and 25 μg of mRNA-1273.351, for a total dose of 50 μg of mRNA. All vaccines were formulated in lipid nanoparticles as previously described.

### Safety Assessment

Participants completed an electronic diary for 7 days post-booster to record solicited systemic and local adverse reactions, daily oral body temperatures, injection site erythema and swelling/induration. Trained site personnel called participants to assess safety every 4 weeks.

### Immunogenicity Assessments

For this sub-study analysis of 80 participants, samples were collected 28 days post-primary vaccination series, immediately prior to the booster vaccination (Day 1), and at days 8, 15, 29, 57, and 181 post-booster vaccination. In this preliminary analysis, neutralization results of sera collected 28 days after the primary series, immediately prior to the booster dose, and 15 and 29 days after the booster are provided. A clinically validated lentivirus PsVN assay, used to test the samples from phase 2 and 3 (COVE) trials, was used to analyze samples collected immediately prior to the booster (D1) and at D15 and D29 respectively. To enable exploratory analysis across a panel of SARS-CoV-2 variants, sera were analyzed for neutralizing antibody titers using a research-grade recombinant vesicular stomatitis virus (VSV)-based pseudovirus assay previously used to assess the impact of neutralization from variants against sera collected 7 days after the second dose of mRNA-1273. In this assay, the S protein of the prototype WA1/2020 isolate with the D614G mutation (wild-type D614G) or the S proteins from variants are encoded. The VSV PsVN neutralization assay demonstrates strong correlation and concordance with the clinically validated Lentivirus PsVN assay (FIGS. 36A-36B).

### Statistical Analysis

Geometric mean titer (GMT) and geometric mean fold rise (GMFR) were calculated based on log-transformed titers, and 95% confidence intervals (CI) based on the t-distribution of the log-transformed titers or the difference in the log-transformed titers for GMT and GMFR, respectively, then back transformed to the original scale. Analysis of the COVE study participant sera collected 28 days after the primary series was used to establish a GMT benchmark further used to derive GMT ratios after boosting. Wilcoxon matched-pairs signed rank test was used to compare results. Spearman nonparametric correlation was used for assay correlation.

### Example 23—Variant Booster Study

Subjects were administered two doses of mRNA-1273 as described in Example 13 (either two doses of 50 μg or two doses of 100 μg each). Booster doses were administered at least 6 months later. The booster doses tested include: mRNA-1273.211 (50 μg), mRNA-1273.211 (100 μg), mRNA-1273 (100 μg), mRNA-1273.617, and mRNA-1273.213 (100 μg dose: 50 μg mRNA-1273 and 50 μg 1273.617; SEQ ID NO: 28). The safety and immunogenicity of the booster dose were examined. In particular, the results from day 29 (after the booster dose) were compared to the results described in Example 22. The results relative to subjects who only received two 100 μg doses of mRNA-1273 (P301) are shown in Table 27 below and demonstrate that the booster dose increased geometric mean titer (GMT) levels relative to the subjects who only received two 100 μg doses of mRNA-1273 (P301). The booster dose also induced neutralizing antibodies that are significantly higher than at day 29 after dose 2 (e.g., the booster resulted in a 17-fold increase over pre-booster titers). The booster titers were comparable between the younger and older adult cohorts.

TABLE 27
Immune response after Booster Dose (50 μg) vs. Two Doses of 100 μg mRNA-1273

| | Pseudovirus Neutralizing | | Pseudovirus Neutralizing | | Pseudovirus Neutralizing | |
|---|---|---|---|---|---|---|
| | Antibody ID50 | | Antibody ID50 | | Antibody ID50 | |
| | All ages | | Ages 18-<65 | | Ages 65+ | |
| | P201 Part B | P301 | P201 Part B | P301 | P201 Part B | P301 |
| Baseline GMT | 125.7 | 9.6 | 145.6 | 9.8 | 82.5 | 9.4 |
| GMT | 1892.7 | 1081.1 | 1940.4 | 1206.6 | 1761.8 | 871.2 |
| (observed) | | | | | | |
| Ratio of GMT | 1.75 | | 1.61 | | 2.02 | |
| (P201 Part B | | | | | | |
| vs. P301) | | | | | | |
| Seroresponse | 90.1 (86.1, 93.3) | 98.4 (97.4, 99.1) | 89.0 (84.1, 92.8) | 98.4 (97.2, 99.2) | 93.4 (85.3, 97.8) | 98.3 (96.3, 99.4) |
| Rate (95% CI) | | | | | | |
| Difference in | −8.2 (−12.2, −5.2) | | −9.4 | | −4.9 | |
| SSR (95% CI) | | | | | | |

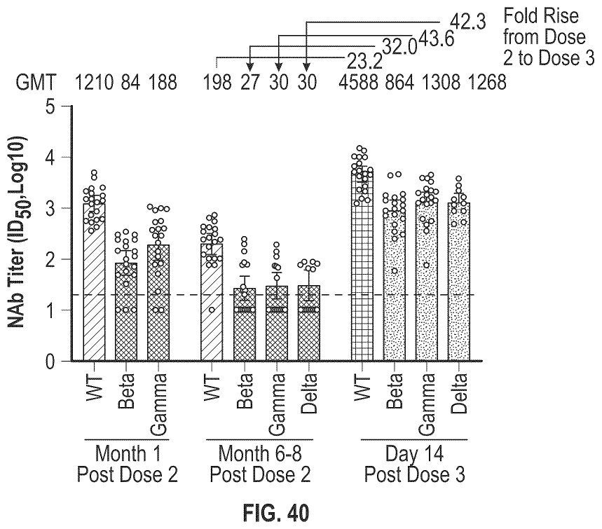

The samples were further analyzed by examining neutralizing titers against broader variants of concern over time using a VSV assay, as described above. The results are shown in FIG. 40. The booster dose (mRNA-1273, 50 μg) was found to increase neutralizing antibody against the wild type (D614G) and three variants tested (B.1.351 (beta), P.1 (gamma), and B.1.617.2 (delta)). While neutralizing titers against the ancestral strain (“WT” in FIG. 40) remained above the GMT, the GMTs waned substantially by 6 months post-dose 2 against the variants of concern. The booster increased the GMTs for all viruses tested, and the fold-rise from dose 2 to the booster dose (dose 3) ranged from 23.2-fold against the D614G (wild-type) virus to 43.6-fold against the gamma variant (FIG. 40).

### Example 24—Phase 2/3 Variant Booster Study

Subjects are administered two doses of mRNA-1273 as described in Example 13. Booster doses are administered 6-8 months later. The booster doses tested include: mRNA-1273.211 (50 μg), mRNA-1273.211 (100 μg), mRNA-1273 (100 μg), mRNA-1273.617, and mRNA-1273.213 (100 μg dose: 50 μg mRNA-1273 and 50 μg 1273.617; SEQ ID NO: 28). In some studies, three different doses of mRNA-1273.213 are tested: 25 μg (12.5 μg mRNA-1273 +12.5 μg 1273.617), 50 μg (25 μg mRNA-1273+25 μg 1273.617) and 100 μg (50 μg mRNA-1273+50 μg 1273.617). The endpoints are immunogenicity and safety of the dosages.

### Example 25—Immunogenicity of Omicron-Related Vaccines

BALB/c mice were immunized at days 1 and 22 with 1 μg or 0.1 μg of mRNA-1273 (SEQ ID NO: 18), mRNA-1273.529 (m-mu) (SEQ ID NO: 40), mRNA-1273.529 (PBSko_match) (SEQ ID NO: 37), mRNA-1273.529.IDR14A (SEQ ID NO: 43), or mRNA.529.IDR14B (SEQ ID NO: 45) (n=8/group). In each vaccine, the mRNA was formulated in lipid nanoparticles (LNPs) including 0.5-15% PEG-modified lipid, 5-25% non-cationic lipid, 25-55% sterol, and 20-60% ionizable amino lipid. The PEG-modified lipid was 1,2 dimyristoyl-sn-glycerol, methoxypolyethyleneglycol (PEG2000 DMG), the non-cationic lipid was 1,2 distearoyl-sn-glycero-3-phosphocholine (DSPC), the sterol was cholesterol, and the ionizable amino lipid had the structure of Compound 1, for example.

Blood was collected one day before the second dose (day 21), and serum and spleen samples will be taken on day 36 and analyzed by ELISA and neutralization assays as described herein. ELISAs were performed to quantify total IgG specific to a parental SARS-CoV-2 Spike protein with the USA-WA1/2020 isolate amino acid sequence and IgG specific to B.1.1.529 (the omicron variant) from the samples collected on day 21.

The antibody titers with respect to the parental SARS-CoV-2 spike protein and B.1.529 are shown in Table 28 below.

TABLE 28
Log10 SP2/SP2.529-specific IgG Titer (Day 21)

| | | | | IgG |
|---|---|---|---|---|
| Virus | Vaccine | Dose | | Titer |
| SP2 | mRNA-1273 | 1 | μg | 818 |
| B.1.1.529 | mRNA-1273 | 1 | μg | 75 |
| SP2 | mRNA-1273.529 (m-mu) | 1 | μg | 260 |
| B.1.1.529 | mRNA-1273.529 (m-mu) | 1 | μg | 406 |
| SP2 | mRNA-1273.529 (PBSko_match) | 1 | μg | 309 |
| B.1.1.529 | mRNA-1273.529 (PBSko_match) | 1 | μg | 228 |
| SP2 | mRNA-1273.529.IDR14A | 1 | μg | 104 |
| B.1.1.529 | mRNA-1273.529.IDR14A | 1 | μg | 212 |
| SP2 | mRNA-1273.529.IDR14B | 1 | μg | 1082 |
| B.1.1.529 | mRNA-1273.529.IDR14B | 1 | μg | 1292 |
| SP2 | mRNA-1273 | 0.1 | μg | 14 |
| B.1.1.529 | mRNA-1273 | 0.1 | μg | 7 |
| SP2 | mRNA-1273.529 (m-mu) | 0.1 | μg | 8 |
| B.1.1.529 | mRNA-1273.529 (m-mu) | 0.1 | μg | 11 |
| SP2 | mRNA-1273.529 (PBSko_match) | 0.1 | μg | 28 |
| B.1.1.529 | mRNA-1273.529 (PBSko_match) | 0.1 | μg | 15 |
| SP2 | mRNA-1273.529.IDR14A | 0.1 | μg | 7 |
| B.1.1.529 | mRNA-1273.529.IDR14A | 0.1 | μg | 10 |
| SP2 | mRNA-1273.529.IDR14B | 0.1 | μg | 145 |
| B.1.1.529 | mRNA-1273.529.IDR14B | 0.1 | μg | 80 |

As shown in Table 28, the B.1.529 IgG antibody titer was increased in the groups administered the B.1.529-specific vaccines compared to the mRNA-1273 group at both dosage levels.

### REFERENCES

1 Zhou, P. et al. A pneumonia outbreak associated with a new coronavirus of probable bat origin. Nature 579, 270-273, doi: 10.1038/s41586-020-2012-7 (2020).
2 Coronaviridae Study Group of the International Committee on Taxonomy of, V. The species Severe acute respiratory syndrome-related coronavirus: classifying 2019-nCOV and naming it SARS-CoV-2. Nat Microbiol 5, 536-544, doi: 10.1038/s41564-020-0695-z (2020).
3 Wrapp, D. et al. Cryo-EM structure of the 2019-nCOV spike in the prefusion conformation. Science 367, 1260-1263, doi: 10.1126/science.abb2507 (2020).
4 Jackson, L. A. et al. An mRNA Vaccine against SARS-CoV-2-Preliminary Report. N Engl J Med 383, 1920-1931, doi: 10.1056/NEJMoa2022483 (2020).
5 Baden, L. R. et al. Safety and immunogenicity of two heterologous HIV vaccine regimens in healthy, HIV-uninfected adults (TRAVERSE): a randomised, parallel-group, placebo-controlled, double-blind, phase 1/2a study. Lancet HIV 7, e688-e698, doi: 10.1016/S2352-3018 (20) 30229-0 (2020).
6 FDA (2021) COVID-19 Vaccines. https://www.fda.gov/emergency-preparedness-and-response/coronavirus-disease-2019-covid-19/covid-19-vaccines.
7 Korber, B. et al. Tracking Changes in SARS-CoV-2 Spike: Evidence that D614G Increases Infectivity of the COVID-19 Virus. Cell 182, 812-827 e819, doi: 10.1016/j.cell.2020.06.043 (2020).
8 Plante, J. A. et al. Spike mutation D614G alters SARS-CoV-2 fitness. Nature 592, 116-121, doi: 10.1038/s41586-020-2895-3 (2021).
9 Volz, E. et al. Evaluating the Effects of SARS-CoV-2 Spike Mutation D614G on Transmissibility and Pathogenicity. Cell 184, 64-75 e11, doi: 10.1016/j.cell.2020.11.020 (2021).
10 Yurkovetskiy, L. et al. Structural and Functional Analysis of the D614G SARS-CoV-2

Spike Protein Variant. Cell 183, 739-751 e738, doi: 10.1016/j.cell.2020.09.032 (2020).
11 Walls, A. C. et al. Structure, Function, and Antigenicity of the SARS-CoV-2 Spike Glycoprotein. Cell 181, 281-292 e286, doi: 10.1016/j.cell.2020.02.058 (2020).
12 Letko, M., Marzi, A. & Munster, V. Functional assessment of cell entry and receptor usage for SARS-CoV-2 and other lineage B betacoronaviruses. Nat Microbiol 5, 562-569, doi: 10.1038/s41564-020-0688-y (2020).
13 Wang, Q. et al. Structural and Functional Basis of SARS-CoV-2 Entry by Using Human ACE2. Cell 181, 894-904 e899, doi: 10.1016/j.cell.2020.03.045 (2020).
14 Shang, J. et al. Cell entry mechanisms of SARS-CoV-2. Proc Natl Acad Sci USA 117, 11727-11734, doi: 10.1073/pnas.2003138117 (2020).
15 Keech, C. et al. Phase 1-2 Trial of a SARS-CoV-2 Recombinant Spike Protein Nanoparticle Vaccine. N Engl J Med 383, 2320-2332, doi: 10.1056/NEJMoa2026920 (2020).
16 Walsh, E. E. et al. Safety and Immunogenicity of Two RNA-Based Covid-19 Vaccine Candidates. N Engl J Med 383, 2439-2450, doi: 10.1056/NEJMoa2027906 (2020).
17 Anderson, E. J. et al. Safety and Immunogenicity of SARS-CoV-2 mRNA-1273 Vaccine in Older Adults. N Engl J Med 383, 2427-2438, doi: 10.1056/NEJMoa2028436 (2020).
18 McCallum, M. et al. N-terminal domain antigenic mapping reveals a site of vulnerability for SARS-CoV-2. Cell, doi: 10.1016/j.cell.2021.03.028 (2021).
19 Li, Q. et al. The Impact of Mutations in SARS-CoV-2 Spike on Viral Infectivity and Antigenicity. Cell 182, 1284-1294 e1289, doi: 10.1016/j.cell.2020.07.012 (2020).
20 Tegally, H. et al. Detection of a SARS-CoV-2 variant of concern in South Africa. Nature, doi: 10.1038/s41586-021-03402-9 (2021).
21 Shen, X. et al. SARS-CoV-2 variant B.1.1.7 is susceptible to neutralizing antibodies elicited by ancestral spike vaccines. Cell Host Microbe, doi: 10.1016/j.chom.2021.03.002 (2021).
22 Wibmer, C. K. et al. SARS-CoV-2 501Y.V2 escapes neutralization by South African

COVID-19 donor plasma. Nat Med, doi: 10.1038/s41591-021-01285-x (2021).
23 Voysey, M. et al. Safety and efficacy of the ChAdOx1 nCOV-19 vaccine (AZD1222) against SARS-CoV-2: an interim analysis of four randomised controlled trials in Brazil, South Africa, and the UK. Lancet 397, 99-111, doi: 10.1016/S0140-6736 (20) 32661-1 (2021).
24 Voysey, M. et al. Single-dose administration and the influence of the timing of the booster dose on immunogenicity and efficacy of ChAdOx1 nCOV-19 (AZD1222) vaccine: a pooled analysis of four randomised trials. Lancet 397, 881-891, doi: 10.1016/S0140-6736 (21) 00432-3 (2021).
25 Andreano, E. et al. SARS-CoV-2 escape in vitro from a highly neutralizing COVID-19 convalescent plasma. bioRxiv, doi: 10.1101/2020.12.28.424451 (2020).
26 Hoffmann, M. et al. SARS-CoV-2 variants B.1.351 and P.1 escape from neutralizing antibodies. Cell, doi: 10.1016/j.cell.2021.03.036 (2021).
27 Garcia-Beltran, W. F. et al. Multiple SARS-CoV-2 variants escape neutralization by vaccine-induced humoral immunity. Cell, doi: 10.1016/j.cell.2021.03.013 (2021).
28 Starr, T. N. et al. Deep Mutational Scanning of SARS-CoV-2 Receptor Binding Domain Reveals Constraints on Folding and ACE2 Binding. Cell 182, 1295-1310 e1220, doi: 10.1016/j.cell.2020.08.012 (2020).
29 Starr, T. N. et al. Deep mutational scanning of SARS-CoV-2 receptor binding domain reveals constraints on folding and ACE2 binding. bioRxiv, doi: 10.1101/2020.06.17.157982 (2020).
30 Tada, T. et al. Decreased neutralization of SARS-CoV-2 global variants by therapeutic anti-spike protein monoclonal antibodies. bioRxiv, doi: 10.1101/2021.02.18.431897 (2021).
31 Tada, T. et al. Neutralization of viruses with European, South African, and United States SARS-CoV-2 variant spike proteins by convalescent sera and BNT162b2 mRNA vaccine-elicited antibodies. bioRxiv, doi: 10.1101/2021.02.05.430003 (2021).
32 Deng, X. et al. Transmission, infectivity, and antibody neutralization of an emerging SARS-CoV-2 variant in California carrying a L452R spike protein mutation. medRxiv, doi: 10.1101/2021.03.07.21252647 (2021).
33 Greaney, A. J. et al. Comprehensive mapping of mutations in the SARS-CoV-2 receptor-binding domain that affect recognition by polyclonal human plasma antibodies. Cell Host Microbe 29, 463-476 e466, doi: 10.1016/j.chom.2021.02.003 (2021).
34 Wang, P. et al. Antibody resistance of SARS-CoV-2 variants B.1.351 and B.1.1.7. Nature, doi: 10.1038/s41586-021-03398-2 (2021).
35 Zhou, H. et al. B.1.526 SARS-CoV-2 variants identified in New York City are neutralized by vaccine-elicited and therapeutic monoclonal antibodies. bioRxiv, doi: 10.1101/2021.03.24.436620 (2021).
36 Wu, K. et al. Serum Neutralizing Activity Elicited by mRNA-1273 Vaccine. N Engl J Med, doi: 10.1056/NEJMc2102179 (2021).
37 Novavax (2021). Novavax COVID-19 Vaccine Demonstrates 89.3% Efficacy in UK Phase 3 Trial. Press Release Jan. 28, 2021: https://ir.novavax.com/node/15506/pdf
38 K, E. et al. Efficacy of ChAdOx1 nCOV-19 (AZD1222) vaccineagainst SARS-CoV-2 variant of concern 202012/01 (B.1.1.7): an exploratory analysis of a randomisedcontrolled trial. Lancet, doi: 10.1016/S0140-6736 (21) 00628-0 (2021).
39 Pfizer (2021) Pfizer and BioNTech confirm high efficacy and no serious safety concerns through up to six months following second dose in updated topline analysis of landmark COVID-19 vaccine study. Press Release Apr. 1, 2021: https://www.pfizer.com/news/press-release/press-release-detail/pfizer-and-biontech-confirm-high-efficacy-and-no-serious.
40 Corbett, K. S. et al. SARS-CoV-2 mRNA vaccine design enabled by prototype pathogen preparedness. Nature 586, 567-571, doi: 10.1038/s41586-020-2622-0 (2020).
41 Ho, D. et al. Increased Resistance of SARS-CoV-2 Variants B.1.351 and B.1.1.7 to Antibody Neutralization. Res Sq, doi: 10.21203/rs.3.rs-155394/v1 (2021).
42 Wrobel, A. G. et al. SARS-CoV-2 and bat RaTG13 spike glycoprotein structures inform on virus evolution and furin-cleavage effects. Nat Struct Mol Biol 27, 763-767, doi: 10.1038/s41594-020-0468-7 (2020).
43 Lan, J. et al. Structure of the SARS-CoV-2 spike receptor-binding domain bound to the ACE2 receptor. Nature 581, 215-220, doi: 10.1038/s41586-020-2180-5 (2020).
44 Nelson, J. et al. Impact of mRNA chemistry and manufacturing process on innate immune activation. Sci Adv 6, eaaz6893, doi: 10.1126/sciadv.aaz6893 (2020).
45 Hassett, K. J. et al. Optimization of Lipid Nanoparticles for Intramuscular Administration of mRNA Vaccines. Mol Ther Nucleic Acids 15, 1-11, doi: 10.1016/j.omtn.2019.01.013 (2019).
46 John, S. et al. Multi-antigenic human cytomegalovirus mRNA vaccines that elicit potent humoral and cell-mediated immunity. Vaccine 36, 1689-1699, doi: 10.1016/j.vaccine.2018.01.029 (2018).
47 Bahl, K. et al. Preclinical and Clinical Demonstration of Immunogenicity by mRNA Vaccines against H10N8 and H7N9 Influenza Viruses. Mol Ther 25, 1316-1327, doi: 10.1016/j.ymthe.2017.03.035 (2017).
48 Vogel, A. B. et al. Self-Amplifying RNA Vaccines Give Equivalent Protection against Influenza to mRNA Vaccines but at Much Lower Doses. Mol Ther 26, 446-455, doi: 10.1016/j.ymthe.2017.11.017 (2018).
49 Whitt, M. A. Generation of VSV pseudotypes using recombinant DeltaG-VSV for studies on virus entry, identification of entry inhibitors, and immune responses to vaccines. J Virol Methods 169, 365-374, doi: 10.1016/j.jviromet.2010.08.006 (2010).

### ADDITIONAL SEQUENCES

It should be understood that any of the mRNA sequences described herein may include a 5′ UTR and/or a 3′ UTR. The UTR sequences may be selected from the following sequences, or other known UTR sequences may be used. It should also be understood that any of the mRNA constructs described herein may further comprise a poly(A) tail and/or cap (e.g., 7 mG(5′)ppp(5′)NlmpNp). Further, while many of the mRNAs and encoded antigen sequences described herein include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

5′ UTR:
(SEQ ID NO: 50)
GGGAAAUAAGAGAGAAAAGAAGAGUAAGAAGAAAUAUAAGAGCCACC
5′ UTR:
(SEQ ID NO: 2)
GGGAAAUAAGAGAGAAAAGAAGAGUAAGAAGAAAUAUAAGACCCCGGCG
CCGCCACC
3′ UTR:
(SEQ ID NO: 51)
UGAUAAUAGGCUGGAGCCUCGGUGGCCAUGCUUCUUGCCCCUUGGGCCU
CCCCCCAGCCCCUCCUCCCCUUCCUGCACCCGUACCCCCGUGGUCUUUG
AAUAAAGUCUGAGUGGGCGGC
3′ UTR:
(SEQ ID NO: 4)
UGAUAAUAGGCUGGAGCCUCGGUGGCCUAGCUUCUUGCCCCUUGGGCCU
CCCCCCAGCCCCUCCUCCCCUUCCUGCACCCGUACCCCCGUGGUCU
UUGAAUAAAGUCUGAGUGGGCGGC

TABLE 1
Sequence Listing
WH2020_NatSP_2P_E484K_D614G

| SEQ ID NO: 1 consists of from 5′ end to 3′ end: 5′ UTR SEQ ID NO: 2, | | 1 |
|---|---|---|
| mRNA ORF SEQ ID NO: 3, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGACCCCGGCGCCGCCACC | |
| ORF of mRNA | AUGUUCGUGUUCCUGGUGCUGCUGCCCCUGGUGAGCAGC | 3 |
| Construct | CAGUGCGUGAACCUGACCACCCGGACCCAGCUGCCACCAG | |
| (excluding the stop | CCUACACCAACAGCUUCACCCGGGGCGUCUACUACCCCGA | |
| codon) | CAAGGUGUUCCGGAGCAGCGUCCUGCACAGCACCCAGGA | |
| | CCUGUUCCUGCCCUUCUUCAGCAACGUGACCUGGUUCCAC | |
| | GCCAUCCACGUGAGCGGCACCAACGGCACCAAGCGGUUCG | |
| | ACAACCCCGUGCUGCCCUUCAACGACGGCGUGUACUUCGC | |
| | CAGCACCGAGAAGAGCAACAUCAUCCGGGGCUGGAUCUU | |
| | CGGCACCACCCUGGACAGCAAGACCCAGAGCCUGCUGAUC | |
| | GUGAAUAACGCCACCAACGUGGUGAUCAAGGUGUGCGAG | |
| | UUCCAGUUCUGCAACGACCCCUUCCUGGGCGUGUACUACC | |
| | ACAAGAACAACAAGAGCUGGAUGGAGAGCGAGUUCCGGG | |
| | UGUACAGCAGCGCCAACAACUGCACCUUCGAGUACGUGA | |
| | GCCAGCCCUUCCUGAUGGACCUGGAGGGCAAGCAGGGCA | |
| | ACUUCAAGAACCUGCGGGAGUUCGUGUUCAAGAACAUCG | |
| | ACGGCUACUUCAAGAUCUACAGCAAGCACACCCCAAUCA | |
| | ACCUGGUGCGGGAUCUGCCCCAGGGCUUCUCAGCCCUGG | |
| | AGCCCCUGGUGGACCUGCCCAUCGGCAUCAACAUCACCCG | |
| | GUUCCAGACCCUGCUGGCCCUGCACCGGAGCUACCUGACC | |
| | CCAGGCGACAGCAGCAGCGGGUGGACAGCAGGCGCGGCU | |
| | GCUUACUACGUGGGCUACCUGCAGCCCCGGACCUUCCUGC | |
| | UGAAGUACAACGAGAACGGCACCAUCACCGACGCCGUGG | |
| | ACUGCGCCCUGGACCCUCUGAGCGAGACCAAGUGCACCCU | |
| | GAAGAGCUUCACCGUGGAGAAGGGCAUCUACCAGACCAG | |
| | CAACUUCCGGGUGCAGCCCACCGAGAGCAUCGUGCGGUU | |
| | CCCCAACAUCACCAACCUGUGCCCCUUCGGCGAGGUGUUC | |
| | AACGCCACCCGGUUCGCCAGCGUGUACGCCUGGAACCGGA | |
| | AGCGGAUCAGCAACUGCGUGGCCGACUACAGCGUGCUGU | |
| | ACAACAGCGCCAGCUUCAGCACCUUCAAGUGCUACGGCG | |
| | UGAGCCCCACCAAGCUGAACGACCUGUGCUUCACCAACGU | |
| | GUACGCCGACAGCUUCGUGAUCCGUGGCGACGAGGUGCG | |
| | GCAGAUCGCACCCGGCCAGACAGGCAAGAUCGCCGACUAC | |
| | AACUACAAGCUGCCCGACGACUUCACCGGCUGCGUGAUC | |
| | GCCUGGAACAGCAACAACCUCGACAGCAAGGUGGGCGGC | |
| | AACUACAACUACCUGUACCGGCUGUUCCGGAAGAGCAAC | |
| | CUGAAGCCCUUCGAGCGGGACAUCAGCACCGAGAUCUAC | |
| | CAAGCCGGCUCCACCCCUUGCAACGGCGUGAAGGGCUUCA | |
| | ACUGCUACUUCCCUCUGCAGAGCUACGGCUUCCAGCCCAC | |
| | CAACGGCGUGGGCUACCAGCCCUACCGGGUGGUGGUGCU | |
| | GAGCUUCGAGCUGCUGCACGCCCCAGCCACCGUGUGUGGC | |
| | CCCAAGAAGAGCACCAACCUGGUGAAGAACAAGUGCGUG | |
| | AACUUCAACUUCAACGGCCUUACCGGCACCGGCGUGCUG | |
| | ACCGAGAGCAACAAGAAAUUCCUGCCCUUUCAGCAGUUC | |
| | GGCCGGGACAUCGCCGACACCACCGACGCUGUGCGGGAUC | |
| | CCCAGACCCUGGAGAUCCUGGACAUCACCCCUUGCAGCUU | |
| | CGGCGGCGUGAGCGUGAUCACCCCAGGCACCAACACCAGC | |
| | AACCAGGUGGCCGUGCUGUACCAGGGCGUGAACUGCACC | |
| | GAGGUGCCCGUGGCCAUCCACGCCGACCAGCUGACACCCA | |
| | CCUGGCGGGUCUACAGCACCGGCAGCAACGUGUUCCAGA | |
| | CCCGGGCCGGUUGCCUGAUCGGCGCCGAGCACGUGAACA | |
| | ACAGCUACGAGUGCGACAUCCCCAUCGGCGCCGGCAUCUG | |
| | UGCCAGCUACCAGACCCAGACCAAUUCACCCCGGAGGGCA | |
| | AGGAGCGUGGCCAGCCAGAGCAUCAUCGCCUACACCAUG | |
| | AGCCUGGGCGCCGAGAACAGCGUGGCCUACAGCAACAAC | |
| | AGCAUCGCCAUCCCCACCAACUUCACCAUCAGCGUGACCA | |
| | CCGAGAUUCUGCCCGUGAGCAUGACCAAGACCAGCGUGG | |
| | ACUGCACCAUGUACAUCUGCGGCGACAGCACCGAGUGCA | |
| | GCAACCUGCUGCUGCAGUACGGCAGCUUCUGCACCCAGCU | |
| | GAACCGGGCCCUGACCGGCAUCGCCGUGGAGCAGGACAA | |
| | GAACACCCAGGAGGUGUUCGCCCAGGUGAAGCAGAUCUA | |
| | CAAGACCCCUCCCAUCAAGGACUUCGGCGGCUUCAACUUC | |
| | AGCCAGAUCCUGCCCGACCCCAGCAAGCCCAGCAAGCGGA | |
| | GCUUCAUCGAGGACCUGCUGUUCAACAAGGUGACCCUAG | |
| | CCGACGCCGGCUUCAUCAAGCAGUACGGCGACUGCCUCGG | |
| | CGACAUAGCCGCCCGGGACCUGAUCUGCGCCCAGAAGUUC | |
| | AACGGCCUGACCGUGCUGCCUCCCCUGCUGACCGACGAGA | |
| | UGAUCGCCCAGUACACCAGCGCCCUGUUAGCCGGAACCAU | |
| | CACCAGCGGCUGGACUUUCGGCGCUGGAGCCGCUCUGCA | |
| | GAUCCCCUUCGCCAUGCAGAUGGCCUACCGGUUCAACGGC | |
| | AUCGGCGUGACCCAGAACGUGCUGUACGAGAACCAGAAG | |
| | CUGAUCGCCAACCAGUUCAACAGCGCCAUCGGCAAGAUCC | |
| | AGGACAGCCUGAGCAGCACCGCUAGCGCCCUGGGCAAGC | |
| | UGCAGGACGUGGUGAACCAGAACGCCCAGGCCCUGAACA | |
| | CCCUGGUGAAGCAGCUGAGCAGCAACUUCGGCGCCAUCA | |
| | GCAGCGUGCUGAACGACAUCCUGAGCCGGCUGGACCCUCC | |
| | CGAGGCCGAGGUGCAGAUCGACCGGCUGAUCACUGGCCG | |
| | GCUGCAGAGCCUGCAGACCUACGUGACCCAGCAGCUGAU | |
| | CCGGGCCGCCGAGAUUCGGGCCAGCGCCAACCUGGCCGCC | |
| | ACCAAGAUGAGCGAGUGCGUGCUGGGCCAGAGCAAGCGG | |
| | GUGGACUUCUGCGGCAAGGGCUACCACCUGAUGAGCUUU | |
| | CCCCAGAGCGCACCCCACGGAGUGGUGUUCCUGCACGUGA | |
| | CCUACGUGCCCGCCCAGGAGAAGAACUUCACCACCGCCCC | |
| | AGCCAUCUGCCACGACGGCAAGGCCCACUUUCCCCGGGAG | |
| | GGCGUGUUCGUGAGCAACGGCACCCACUGGUUCGUGACC | |
| | CAGCGGAACUUCUACGAGCCCCAGAUCAUCACCACCGACA | |
| | ACACCUUCGUGAGCGGCAACUGCGACGUGGUGAUCGGCA | |
| | UCGUGAACAACACCGUGUACGAUCCCCUGCAGCCCGAGCU | |
| | GGACAGCUUCAAGGAGGAGCUGGACAAGUACUUCAAGAA | |
| | UCACACCAGCCCCGACGUGGACCUGGGCGACAUCAGCGGC | |
| | AUCAACGCCAGCGUGGUGAACAUCCAGAAGGAGAUCGAU | |
| | CGGCUGAACGAGGUGGCCAAGAACCUGAACGAGAGCCUG | |
| | AUCGACCUGCAGGAGCUGGGCAAGUACGAGCAGUACAUC | |
| | AAGUGGCCCUGGUACAUCUGGCUGGGCUUCAUCGCCGGC | |
| | CUGAUCGCCAUCGUGAUGGUGACCAUCAUGCUGUGCUGC | |
| | AUGACCAGCUGCUGCAGCUGCCUGAAGGGCUGUUGCAGC | |
| | UGCGGCAGCUGCUGCAAGUUCGACGAGGACGACAGCGAG | |
| | CCCGUGCUGAAGGGCGUGAAGCUGCACUACACC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MFVFLVLLPLVSSQCVNLTTRTQLPPAYTNSFTRGVYYPDKVF | 5 |
| acid sequence | RSSVLHSTQDLFLPFFSNVTWFHAIHVSGTNGTKRFDNPVLPF | |
| | NDGVYFASTEKSNIIRGWIFGTTLDSKTQSLLIVNNATNVVIKV | |
| | CEFQFCNDPFLGVYYHKNNKSWMESEFRVYSSANNCTFEYVS | |
| | QPFLMDLEGKQGNFKNLREFVFKNIDGYFKIYSKHTPINLVRD | |
| | LPQGFSALEPLVDLPIGINITRFQTLLALHRSYLTPGDSSSGWT | |
| | AGAAAYYVGYLQPRTFLLKYNENGTITDAVDCALDPLSETKC | |
| | TLKSFTVEKGIYQTSNFRVQPTESIVRFPNITNLCPFGEVENATR | |
| | FASVYAWNRKRISNCVADYSVLYNSASFSTFKCYGVSPTKLN | |
| | DLCFTNVYADSFVIRGDEVRQIAPGQTGKIADYNYKLPDDFTG | |
| | CVIAWNSNNLDSKVGGNYNYLYRLFRKSNLKPFERDISTEIYQ | |
| | AGSTPCNGVKGFNCYFPLQSYGFQPTNGVGYQPYRVVVLSFE | |
| | LLHAPATVCGPKKSTNLVKNKCVNFNFNGLTGTGVLTESNKK | |
| | FLPFQQFGRDIADTTDAVRDPQTLEILDITPCSFGGVSVITPGTN | |
| | TSNQVAVLYQGVNCTEVPVAIHADQLTPTWRVYSTGSNVFQT | |
| | RAGCLIGAEHVNNSYECDIPIGAGICASYQTQTNSPRRARSVA | |
| | SQSIIAYTMSLGAENSVAYSNNSIAIPTNFTISVTTEILPVSMTK | |
| | TSVDCTMYICGDSTECSNLLLQYGSFCTQLNRALTGIAVEQDK | |
| | NTQEVFAQVKQIYKTPPIKDFGGFNFSQILPDPSKPSKRSFIEDL | |
| | LFNKVTLADAGFIKQYGDCLGDIAARDLICAQKFNGLTVLPPL | |
| | LTDEMIAQYTSALLAGTITSGWTFGAGAALQIPFAMQMAYRF | |
| | NGIGVTQNVLYENQKLIANQFNSAIGKIQDSLSSTASALGKLQ | |
| | DVVNQNAQALNTLVKQLSSNFGAISSVLNDILSRLDPPEAEVQ | |
| | IDRLITGRLQSLQTYVTQQLIRAAEIRASANLAATKMSECVLG | |
| | QSKRVDFCGKGYHLMSFPQSAPHGVVFLHVTYVPAQEKNFTT | |
| | APAICHDGKAHFPREGVFVSNGTHWFVTQRNFYEPQIITTDNT | |
| | FVSGNCDVVIGIVNNTVYDPLQPELDSFKEELDKYFKNHTSPD | |
| | VDLGDISGINASVVNIQKEIDRLNEVAKNLNESLIDLQELGKYE | |
| | QYIKWPWYIWLGFIAGLIAIVMVTIMLCCMTSCCSCLKGCCSC | |
| | GSCCKFDEDDSEPVLKGVKLHYT | |
| PolyA tail | 100 nt | |

WH2020_NatSP_2P_K417N_E484K_N501Y_D614G

| SEQ ID NO: 6 consists of from 5′ end to 3′ end: 5′ UTR SEQ ID NO: 2, | | 6 |
|---|---|---|
| mRNA ORF SEQ ID NO: 7, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGACCCCGGCGCCGCCACC | |
| ORF of mRNA | AUGUUCGUGUUCCUGGUGCUGCUGCCCCUGGUGAGCAGC | 7 |
| Construct | CAGUGCGUGAACCUGACCACCCGGACCCAGCUGCCACCAG | |
| (excluding the stop | CCUACACCAACAGCUUCACCCGGGGCGUCUACUACCCCGA | |
| codon) | CAAGGUGUUCCGGAGCAGCGUCCUGCACAGCACCCAGGA | |
| | CCUGUUCCUGCCCUUCUUCAGCAACGUGACCUGGUUCCAC | |
| | GCCAUCCACGUGAGCGGCACCAACGGCACCAAGCGGUUCG | |
| | ACAACCCCGUGCUGCCCUUCAACGACGGCGUGUACUUCGC | |
| | CAGCACCGAGAAGAGCAACAUCAUCCGGGGCUGGAUCUU | |
| | CGGCACCACCCUGGACAGCAAGACCCAGAGCCUGCUGAUC | |
| | GUGAAUAACGCCACCAACGUGGUGAUCAAGGUGUGCGAG | |
| | UUCCAGUUCUGCAACGACCCCUUCCUGGGCGUGUACUACC | |
| | ACAAGAACAACAAGAGCUGGAUGGAGAGCGAGUUCCGGG | |
| | UGUACAGCAGCGCCAACAACUGCACCUUCGAGUACGUGA | |
| | GCCAGCCCUUCCUGAUGGACCUGGAGGGCAAGCAGGGCA | |
| | ACUUCAAGAACCUGCGGGAGUUCGUGUUCAAGAACAUCG | |
| | ACGGCUACUUCAAGAUCUACAGCAAGCACACCCCAAUCA | |
| | ACCUGGUGCGGGAUCUGCCCCAGGGCUUCUCAGCCCUGG | |
| | AGCCCCUGGUGGACCUGCCCAUCGGCAUCAACAUCACCCG | |
| | GUUCCAGACCCUGCUGGCCCUGCACCGGAGCUACCUGACC | |
| | CCAGGCGACAGCAGCAGCGGGUGGACAGCAGGCGCGGCU | |
| | GCUUACUACGUGGGCUACCUGCAGCCCCGGACCUUCCUGC | |
| | UGAAGUACAACGAGAACGGCACCAUCACCGACGCCGUGG | |
| | ACUGCGCCCUGGACCCUCUGAGCGAGACCAAGUGCACCCU | |
| | GAAGAGCUUCACCGUGGAGAAGGGCAUCUACCAGACCAG | |
| | CAACUUCCGGGUGCAGCCCACCGAGAGCAUCGUGCGGUU | |
| | CCCCAACAUCACCAACCUGUGCCCCUUCGGCGAGGUGUUC | |
| | AACGCCACCCGGUUCGCCAGCGUGUACGCCUGGAACCGGA | |
| | AGCGGAUCAGCAACUGCGUGGCCGACUACAGCGUGCUGU | |
| | ACAACAGCGCCAGCUUCAGCACCUUCAAGUGCUACGGCG | |
| | UGAGCCCCACCAAGCUGAACGACCUGUGCUUCACCAACGU | |
| | GUACGCCGACAGCUUCGUGAUCCGUGGCGACGAGGUGCG | |
| | GCAGAUCGCACCCGGCCAGACAGGCAACAUCGCCGACUAC | |
| | AACUACAAGCUGCCCGACGACUUCACCGGCUGCGUGAUC | |
| | GCCUGGAACAGCAACAACCUCGACAGCAAGGUGGGCGGC | |
| | AACUACAACUACCUGUACCGGCUGUUCCGGAAGAGCAAC | |
| | CUGAAGCCCUUCGAGCGGGACAUCAGCACCGAGAUCUAC | |
| | CAAGCCGGCUCCACCCCUUGCAACGGCGUGAAGGGCUUCA | |
| | ACUGCUACUUCCCUCUGCAGAGCUACGGCUUCCAGCCCAC | |
| | CUACGGCGUGGGCUACCAGCCCUACCGGGUGGUGGUGCU | |
| | GAGCUUCGAGCUGCUGCACGCCCCAGCCACCGUGUGUGGC | |
| | CCCAAGAAGAGCACCAACCUGGUGAAGAACAAGUGCGUG | |
| | AACUUCAACUUCAACGGCCUUACCGGCACCGGCGUGCUG | |
| | ACCGAGAGCAACAAGAAAUUCCUGCCCUUUCAGCAGUUC | |
| | GGCCGGGACAUCGCCGACACCACCGACGCUGUGCGGGAUC | |
| | CCCAGACCCUGGAGAUCCUGGACAUCACCCCUUGCAGCUU | |
| | CGGCGGCGUGAGCGUGAUCACCCCAGGCACCAACACCAGC | |
| | AACCAGGUGGCCGUGCUGUACCAGGGCGUGAACUGCACC | |
| | GAGGUGCCCGUGGCCAUCCACGCCGACCAGCUGACACCCA | |
| | CCUGGCGGGUCUACAGCACCGGCAGCAACGUGUUCCAGA | |
| | CCCGGGCCGGUUGCCUGAUCGGCGCCGAGCACGUGAACA | |
| | ACAGCUACGAGUGCGACAUCCCCAUCGGCGCCGGCAUCUG | |
| | UGCCAGCUACCAGACCCAGACCAAUUCACCCCGGAGGGCA | |
| | AGGAGCGUGGCCAGCCAGAGCAUCAUCGCCUACACCAUG | |
| | AGCCUGGGCGCCGAGAACAGCGUGGCCUACAGCAACAAC | |
| | AGCAUCGCCAUCCCCACCAACUUCACCAUCAGCGUGACCA | |
| | CCGAGAUUCUGCCCGUGAGCAUGACCAAGACCAGCGUGG | |
| | ACUGCACCAUGUACAUCUGCGGCGACAGCACCGAGUGCA | |
| | GCAACCUGCUGCUGCAGUACGGCAGCUUCUGCACCCAGCU | |
| | GAACCGGGCCCUGACCGGCAUCGCCGUGGAGCAGGACAA | |
| | GAACACCCAGGAGGUGUUCGCCCAGGUGAAGCAGAUCUA | |
| | CAAGACCCCUCCCAUCAAGGACUUCGGCGGCUUCAACUUC | |
| | AGCCAGAUCCUGCCCGACCCCAGCAAGCCCAGCAAGCGGA | |
| | GCUUCAUCGAGGACCUGCUGUUCAACAAGGUGACCCUAG | |
| | CCGACGCCGGCUUCAUCAAGCAGUACGGCGACUGCCUCGG | |
| | CGACAUAGCCGCCCGGGACCUGAUCUGCGCCCAGAAGUUC | |
| | AACGGCCUGACCGUGCUGCCUCCCCUGCUGACCGACGAGA | |
| | UGAUCGCCCAGUACACCAGCGCCCUGUUAGCCGGAACCAU | |
| | CACCAGCGGCUGGACUUUCGGCGCUGGAGCCGCUCUGCA | |
| | GAUCCCCUUCGCCAUGCAGAUGGCCUACCGGUUCAACGGC | |
| | AUCGGCGUGACCCAGAACGUGCUGUACGAGAACCAGAAG | |
| | CUGAUCGCCAACCAGUUCAACAGCGCCAUCGGCAAGAUCC | |
| | AGGACAGCCUGAGCAGCACCGCUAGCGCCCUGGGCAAGC | |
| | UGCAGGACGUGGUGAACCAGAACGCCCAGGCCCUGAACA | |
| | CCCUGGUGAAGCAGCUGAGCAGCAACUUCGGCGCCAUCA | |
| | GCAGCGUGCUGAACGACAUCCUGAGCCGGCUGGACCCUCC | |
| | CGAGGCCGAGGUGCAGAUCGACCGGCUGAUCACUGGCCG | |
| | GCUGCAGAGCCUGCAGACCUACGUGACCCAGCAGCUGAU | |
| | CCGGGCCGCCGAGAUUCGGGCCAGCGCCAACCUGGCCGCC | |
| | ACCAAGAUGAGCGAGUGCGUGCUGGGCCAGAGCAAGCGG | |
| | GUGGACUUCUGCGGCAAGGGCUACCACCUGAUGAGCUUU | |
| | CCCCAGAGCGCACCCCACGGAGUGGUGUUCCUGCACGUGA | |
| | CCUACGUGCCCGCCCAGGAGAAGAACUUCACCACCGCCCC | |
| | AGCCAUCUGCCACGACGGCAAGGCCCACUUUCCCCGGGAG | |
| | GGCGUGUUCGUGAGCAACGGCACCCACUGGUUCGUGACC | |
| | CAGCGGAACUUCUACGAGCCCCAGAUCAUCACCACCGACA | |
| | ACACCUUCGUGAGCGGCAACUGCGACGUGGUGAUCGGCA | |
| | UCGUGAACAACACCGUGUACGAUCCCCUGCAGCCCGAGCU | |
| | GGACAGCUUCAAGGAGGAGCUGGACAAGUACUUCAAGAA | |
| | UCACACCAGCCCCGACGUGGACCUGGGCGACAUCAGCGGC | |
| | AUCAACGCCAGCGUGGUGAACAUCCAGAAGGAGAUCGAU | |
| | CGGCUGAACGAGGUGGCCAAGAACCUGAACGAGAGCCUG | |
| | AUCGACCUGCAGGAGCUGGGCAAGUACGAGCAGUACAUC | |
| | AAGUGGCCCUGGUACAUCUGGCUGGGCUUCAUCGCCGGC | |
| | CUGAUCGCCAUCGUGAUGGUGACCAUCAUGCUGUGCUGC | |
| | AUGACCAGCUGCUGCAGCUGCCUGAAGGGCUGUUGCAGC | |
| | UGCGGCAGCUGCUGCAAGUUCGACGAGGACGACAGCGAG | |
| | CCCGUGCUGAAGGGCGUGAAGCUGCACUACACC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MFVFLVLLPLVSSQCVNLTTRTQLPPAYTNSFTRGVYYPDKVF | 8 |
| acid sequence | RSSVLHSTQDLFLPFFSNVTWFHAIHVSGTNGTKRFDNPVLPF | |
| | NDGVYFASTEKSNIIRGWIFGTTLDSKTQSLLIVNNATNVVIKV | |
| | CEFQFCNDPFLGVYYHKNNKSWMESEFRVYSSANNCTFEYVS | |
| | QPFLMDLEGKQGNFKNLREFVFKNIDGYFKIYSKHTPINLVRD | |
| | LPQGFSALEPLVDLPIGINITRFQTLLALHRSYLTPGDSSSGWT | |
| | AGAAAYYVGYLQPRTFLLKYNENGTITDAVDCALDPLSETKC | |
| | TLKSFTVEKGIYQTSNFRVQPTESIVRFPNITNLCPFGEVENATR | |
| | FASVYAWNRKRISNCVADYSVLYNSASFSTFKCYGVSPTKLN | |
| | DLCFTNVYADSFVIRGDEVRQIAPGQTGNIADYNYKLPDDFTG | |
| | CVIAWNSNNLDSKVGGNYNYLYRLFRKSNLKPFERDISTEIYQ | |
| | AGSTPCNGVKGFNCYFPLQSYGFQPTYGVGYQPYRVVVLSFE | |
| | LLHAPATVCGPKKSTNLVKNKCVNFNFNGLTGTGVLTESNKK | |
| | FLPFQQFGRDIADTTDAVRDPQTLEILDITPCSFGGVSVITPGTN | |
| | TSNQVAVLYQGVNCTEVPVAIHADQLTPTWRVYSTGSNVFQT | |
| | RAGCLIGAEHVNNSYECDIPIGAGICASYQTQTNSPRRARSVA | |
| | SQSIIAYTMSLGAENSVAYSNNSIAIPTNFTISVTTEILPVSMTK | |
| | TSVDCTMYICGDSTECSNLLLQYGSFCTQLNRALTGIAVEQDK | |
| | NTQEVFAQVKQIYKTPPIKDFGGFNFSQILPDPSKPSKRSFIEDL | |
| | LFNKVTLADAGFIKQYGDCLGDIAARDLICAQKFNGLTVLPPL | |
| | LTDEMIAQYTSALLAGTITSGWTFGAGAALQIPFAMQMAYRF | |
| | NGIGVTQNVLYENQKLIANQFNSAIGKIQDSLSSTASALGKLQ | |
| | DVVNQNAQALNTLVKQLSSNFGAISSVLNDILSRLDPPEAEVQ | |
| | IDRLITGRLQSLQTYVTQQLIRAAEIRASANLAATKMSECVLG | |
| | QSKRVDFCGKGYHLMSFPQSAPHGVVFLHVTYVPAQEKNFTT | |
| | APAICHDGKAHFPREGVFVSNGTHWFVTQRNFYEPQIITTDNT | |
| | FVSGNCDVVIGIVNNTVYDPLQPELDSFKEELDKYFKNHTSPD | |
| | VDLGDISGINASVVNIQKEIDRLNEVAKNLNESLIDLQELGKYE | |
| | QYIKWPWYIWLGFIAGLIAIVMVTIMLCCMTSCCSCLKGCCSC | |
| | GSCCKFDEDDSEPVLKGVKLHYT | |
| PolyA tail | 100 nt | |

WH2020_NatSP_2P_L18F_D80A_D215G_L242_244del_R246I_K417N_E484K_N501Y_D614G_A701V

| SEQ ID NO: 9 consists of from 5′ end to 3′ end: 5′ UTR SEQ ID NO: 2, | | 9 |
|---|---|---|
| mRNA ORF SEQ ID NO: 10, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGACCCCGGCGCCGCCACC | |
| ORF of mRNA | AUGUUCGUGUUCCUGGUGCUGCUGCCCCUGGUGAGCAGC | 10 |
| Construct | CAGUGCGUGAACUUUACCACCCGGACCCAGCUGCCACCAG | |
| (excluding the stop | CCUACACCAACAGCUUCACCCGGGGCGUCUACUACCCCGA | |
| codon) | CAAGGUGUUCCGGAGCAGCGUCCUGCACAGCACCCAGGA | |
| | CCUGUUCCUGCCCUUCUUCAGCAACGUGACCUGGUUCCAC | |
| | GCCAUCCACGUGAGCGGCACCAACGGCACCAAGCGGUUCG | |
| | CCAACCCCGUGCUGCCCUUCAACGACGGCGUGUACUUCGC | |
| | CAGCACCGAGAAGAGCAACAUCAUCCGGGGCUGGAUCUU | |
| | CGGCACCACCCUGGACAGCAAGACCCAGAGCCUGCUGAUC | |
| | GUGAAUAACGCCACCAACGUGGUGAUCAAGGUGUGCGAG | |
| | UUCCAGUUCUGCAACGACCCCUUCCUGGGCGUGUACUACC | |
| | ACAAGAACAACAAGAGCUGGAUGGAGAGCGAGUUCCGGG | |
| | UGUACAGCAGCGCCAACAACUGCACCUUCGAGUACGUGA | |
| | GCCAGCCCUUCCUGAUGGACCUGGAGGGCAAGCAGGGCA | |
| | ACUUCAAGAACCUGCGGGAGUUCGUGUUCAAGAACAUCG | |
| | ACGGCUACUUCAAGAUCUACAGCAAGCACACCCCAAUCA | |
| | ACCUGGUGCGGGGCCUGCCCCAGGGCUUCUCAGCCCUGGA | |
| | GCCCCUGGUGGACCUGCCCAUCGGCAUCAACAUCACCCGG | |
| | UUCCAGACCCUGCACAUCAGCUACCUGACCCCAGGCGACA | |
| | GCAGCAGCGGGUGGACAGCAGGCGCGGCUGCUUACUACG | |
| | UGGGCUACCUGCAGCCCCGGACCUUCCUGCUGAAGUACA | |
| | ACGAGAACGGCACCAUCACCGACGCCGUGGACUGCGCCCU | |
| | GGACCCUCUGAGCGAGACCAAGUGCACCCUGAAGAGCUU | |
| | CACCGUGGAGAAGGGCAUCUACCAGACCAGCAACUUCCG | |
| | GGUGCAGCCCACCGAGAGCAUCGUGCGGUUCCCCAACAUC | |
| | ACCAACCUGUGCCCCUUCGGCGAGGUGUUCAACGCCACCC | |
| | GGUUCGCCAGCGUGUACGCCUGGAACCGGAAGCGGAUCA | |
| | GCAACUGCGUGGCCGACUACAGCGUGCUGUACAACAGCG | |
| | CCAGCUUCAGCACCUUCAAGUGCUACGGCGUGAGCCCCAC | |
| | CAAGCUGAACGACCUGUGCUUCACCAACGUGUACGCCGA | |
| | CAGCUUCGUGAUCCGUGGCGACGAGGUGCGGCAGAUCGC | |
| | ACCCGGCCAGACAGGCAACAUCGCCGACUACAACUACAAG | |
| | CUGCCCGACGACUUCACCGGCUGCGUGAUCGCCUGGAACA | |
| | GCAACAACCUCGACAGCAAGGUGGGCGGCAACUACAACU | |
| | ACCUGUACCGGCUGUUCCGGAAGAGCAACCUGAAGCCCU | |
| | UCGAGCGGGACAUCAGCACCGAGAUCUACCAAGCCGGCU | |
| | CCACCCCUUGCAACGGCGUGAAGGGCUUCAACUGCUACU | |
| | UCCCUCUGCAGAGCUACGGCUUCCAGCCCACCUACGGCGU | |
| | GGGCUACCAGCCCUACCGGGUGGUGGUGCUGAGCUUCGA | |
| | GCUGCUGCACGCCCCAGCCACCGUGUGUGGCCCCAAGAAG | |
| | AGCACCAACCUGGUGAAGAACAAGUGCGUGAACUUCAAC | |
| | UUCAACGGCCUUACCGGCACCGGCGUGCUGACCGAGAGC | |
| | AACAAGAAAUUCCUGCCCUUUCAGCAGUUCGGCCGGGAC | |
| | AUCGCCGACACCACCGACGCUGUGCGGGAUCCCCAGACCC | |
| | UGGAGAUCCUGGACAUCACCCCUUGCAGCUUCGGCGGCG | |
| | UGAGCGUGAUCACCCCAGGCACCAACACCAGCAACCAGGU | |
| | GGCCGUGCUGUACCAGGGCGUGAACUGCACCGAGGUGCC | |
| | CGUGGCCAUCCACGCCGACCAGCUGACACCCACCUGGCGG | |
| | GUCUACAGCACCGGCAGCAACGUGUUCCAGACCCGGGCCG | |
| | GUUGCCUGAUCGGCGCCGAGCACGUGAACAACAGCUACG | |
| | AGUGCGACAUCCCCAUCGGCGCCGGCAUCUGUGCCAGCUA | |
| | CCAGACCCAGACCAAUUCACCCCGGAGGGCAAGGAGCGU | |
| | GGCCAGCCAGAGCAUCAUCGCCUACACCAUGAGCCUGGGC | |
| | GUGGAGAACAGCGUGGCCUACAGCAACAACAGCAUCGCC | |
| | AUCCCCACCAACUUCACCAUCAGCGUGACCACCGAGAUUC | |
| | UGCCCGUGAGCAUGACCAAGACCAGCGUGGACUGCACCA | |
| | UGUACAUCUGCGGCGACAGCACCGAGUGCAGCAACCUGC | |
| | UGCUGCAGUACGGCAGCUUCUGCACCCAGCUGAACCGGG | |
| | CCCUGACCGGCAUCGCCGUGGAGCAGGACAAGAACACCCA | |
| | GGAGGUGUUCGCCCAGGUGAAGCAGAUCUACAAGACCCC | |
| | UCCCAUCAAGGACUUCGGCGGCUUCAACUUCAGCCAGAU | |
| | CCUGCCCGACCCCAGCAAGCCCAGCAAGCGGAGCUUCAUC | |
| | GAGGACCUGCUGUUCAACAAGGUGACCCUAGCCGACGCC | |
| | GGCUUCAUCAAGCAGUACGGCGACUGCCUCGGCGACAUA | |
| | GCCGCCCGGGACCUGAUCUGCGCCCAGAAGUUCAACGGCC | |
| | UGACCGUGCUGCCUCCCCUGCUGACCGACGAGAUGAUCGC | |
| | CCAGUACACCAGCGCCCUGUUAGCCGGAACCAUCACCAGC | |
| | GGCUGGACUUUCGGCGCUGGAGCCGCUCUGCAGAUCCCC | |
| | UUCGCCAUGCAGAUGGCCUACCGGUUCAACGGCAUCGGC | |
| | GUGACCCAGAACGUGCUGUACGAGAACCAGAAGCUGAUC | |
| | GCCAACCAGUUCAACAGCGCCAUCGGCAAGAUCCAGGAC | |
| | AGCCUGAGCAGCACCGCUAGCGCCCUGGGCAAGCUGCAG | |
| | GACGUGGUGAACCAGAACGCCCAGGCCCUGAACACCCUG | |
| | GUGAAGCAGCUGAGCAGCAACUUCGGCGCCAUCAGCAGC | |
| | GUGCUGAACGACAUCCUGAGCCGGCUGGACCCUCCCGAG | |
| | GCCGAGGUGCAGAUCGACCGGCUGAUCACUGGCCGGCUG | |
| | CAGAGCCUGCAGACCUACGUGACCCAGCAGCUGAUCCGG | |
| | GCCGCCGAGAUUCGGGCCAGCGCCAACCUGGCCGCCACCA | |
| | AGAUGAGCGAGUGCGUGCUGGGCCAGAGCAAGCGGGUGG | |
| | ACUUCUGCGGCAAGGGCUACCACCUGAUGAGCUUUCCCC | |
| | AGAGCGCACCCCACGGAGUGGUGUUCCUGCACGUGACCU | |
| | ACGUGCCCGCCCAGGAGAAGAACUUCACCACCGCCCCAGC | |
| | CAUCUGCCACGACGGCAAGGCCCACUUUCCCCGGGAGGGC | |
| | GUGUUCGUGAGCAACGGCACCCACUGGUUCGUGACCCAG | |
| | CGGAACUUCUACGAGCCCCAGAUCAUCACCACCGACAACA | |
| | CCUUCGUGAGCGGCAACUGCGACGUGGUGAUCGGCAUCG | |
| | UGAACAACACCGUGUACGAUCCCCUGCAGCCCGAGCUGG | |
| | ACAGCUUCAAGGAGGAGCUGGACAAGUACUUCAAGAAUC | |
| | ACACCAGCCCCGACGUGGACCUGGGCGACAUCAGCGGCAU | |
| | CAACGCCAGCGUGGUGAACAUCCAGAAGGAGAUCGAUCG | |
| | GCUGAACGAGGUGGCCAAGAACCUGAACGAGAGCCUGAU | |
| | CGACCUGCAGGAGCUGGGCAAGUACGAGCAGUACAUCAA | |
| | GUGGCCCUGGUACAUCUGGCUGGGCUUCAUCGCCGGCCU | |
| | GAUCGCCAUCGUGAUGGUGACCAUCAUGCUGUGCUGCAU | |
| | GACCAGCUGCUGCAGCUGCCUGAAGGGCUGUUGCAGCUG | |
| | CGGCAGCUGCUGCAAGUUCGACGAGGACGACAGCGAGCC | |
| | CGUGCUGAAGGGCGUGAAGCUGCACUACACC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MFVFLVLLPLVSSQCVNFTTRTQLPPAYTNSFTRGVYYPDKVF | 11 |
| acid sequence | RSSVLHSTQDLFLPFFSNVTWFHAIHVSGTNGTKRFANPVLPF | |
| | NDGVYFASTEKSNIIRGWIFGTTLDSKTQSLLIVNNATNVVIKV | |
| | CEFQFCNDPFLGVYYHKNNKSWMESEFRVYSSANNCTFEYVS | |
| | QPFLMDLEGKQGNFKNLREFVFKNIDGYFKIYSKHTPINLVRG | |
| | LPQGFSALEPLVDLPIGINITRFQTLHISYLTPGDSSSGWTAGAA | |
| | AYYVGYLQPRTFLLKYNENGTITDAVDCALDPLSETKCTLKSF | |
| | TVEKGIYQTSNFRVQPTESIVRFPNITNLCPFGEVENATRFASV | |
| | YAWNRKRISNCVADYSVLYNSASFSTFKCYGVSPTKLNDLCF | |
| | TNVYADSFVIRGDEVRQIAPGQTGNIADYNYKLPDDFTGCVIA | |
| | WNSNNLDSKVGGNYNYLYRLFRKSNLKPFERDISTEIYQAGS | |
| | TPCNGVKGFNCYFPLQSYGFQPTYGVGYQPYRVVVLSFELLH | |
| | APATVCGPKKSTNLVKNKCVNFNFNGLTGTGVLTESNKKFLP | |
| | FQQFGRDIADTTDAVRDPQTLEILDITPCSFGGVSVITPGTNTS | |
| | NQVAVLYQGVNCTEVPVAIHADQLTPTWRVYSTGSNVFQTR | |
| | AGCLIGAEHVNNSYECDIPIGAGICASYQTQTNSPRRARSVAS | |
| | QSIIAYTMSLGVENSVAYSNNSIAIPTNFTISVTTEILPVSMTKT | |
| | SVDCTMYICGDSTECSNLLLQYGSFCTQLNRALTGIAVEQDK | |
| | NTQEVFAQVKQIYKTPPIKDFGGFNFSQILPDPSKPSKRSFIEDL | |
| | LFNKVTLADAGFIKQYGDCLGDIAARDLICAQKFNGLTVLPPL | |
| | LTDEMIAQYTSALLAGTITSGWTFGAGAALQIPFAMQMAYRF | |
| | NGIGVTQNVLYENQKLIANQFNSAIGKIQDSLSSTASALGKLQ | |
| | DVVNQNAQALNTLVKQLSSNFGAISSVLNDILSRLDPPEAEVQ | |
| | IDRLITGRLQSLQTYVTQQLIRAAEIRASANLAATKMSECVLG | |
| | QSKRVDFCGKGYHLMSFPQSAPHGVVFLHVTYVPAQEKNFTT | |
| | APAICHDGKAHFPREGVFVSNGTHWFVTQRNFYEPQIITTDNT | |
| | FVSGNCDVVIGIVNNTVYDPLQPELDSFKEELDKYFKNHTSPD | |
| | VDLGDISGINASVVNIQKEIDRLNEVAKNLNESLIDLQELGKYE | |
| | QYIKWPWYIWLGFIAGLIAIVMVTIMLCCMTSCCSCLKGCCSC | |
| | GSCCKFDEDDSEPVLKGVKLHYT | |
| PolyA tail | 100 nt | |

WH2020_NatSP_2P_H69del_V70del_Y144del_N501Y_A570D_D614G_P681H_T716I_S982A_D1118H

| SEQ ID NO: 12 consists of from 5′ end to 3′ end: 5′ UTR SEQ ID NO: 2, | | 12 |
|---|---|---|
| mRNA ORF SEQ ID NO: 13, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGACCCCGGCGCCGCCACC | |
| ORF of mRNA | AUGUUCGUGUUCCUGGUGCUGCUGCCCCUGGUGAGCAGC | 13 |
| Construct | CAGUGCGUGAACCUGACCACCCGGACCCAGCUGCCACCAG | |
| (excluding the stop | CCUACACCAACAGCUUCACCCGGGGCGUCUACUACCCCGA | |
| codon) | CAAGGUGUUCCGGAGCAGCGUCCUGCACAGCACCCAGGA | |
| | CCUGUUCCUGCCCUUCUUCAGCAACGUGACCUGGUUCCAC | |
| | GCCAUCAGCGGCACCAACGGCACCAAGCGGUUCGACAACC | |
| | CCGUGCUGCCCUUCAACGACGGCGUGUACUUCGCCAGCAC | |
| | CGAGAAGAGCAACAUCAUCCGGGGCUGGAUCUUCGGCAC | |
| | CACCCUGGACAGCAAGACCCAGAGCCUGCUGAUCGUGAA | |
| | UAACGCCACCAACGUGGUGAUCAAGGUGUGCGAGUUCCA | |
| | GUUCUGCAACGACCCCUUCCUGGGCGUGUACCACAAGAA | |
| | CAACAAGAGCUGGAUGGAGAGCGAGUUCCGGGUGUACAG | |
| | CAGCGCCAACAACUGCACCUUCGAGUACGUGAGCCAGCCC | |
| | UUCCUGAUGGACCUGGAGGGCAAGCAGGGCAACUUCAAG | |
| | AACCUGCGGGAGUUCGUGUUCAAGAACAUCGACGGCUAC | |
| | UUCAAGAUCUACAGCAAGCACACCCCAAUCAACCUGGUG | |
| | CGGGAUCUGCCCCAGGGCUUCUCAGCCCUGGAGCCCCUGG | |
| | UGGACCUGCCCAUCGGCAUCAACAUCACCCGGUUCCAGAC | |
| | CCUGCUGGCCCUGCACCGGAGCUACCUGACCCCAGGCGAC | |
| | AGCAGCAGCGGGUGGACAGCAGGCGCGGCUGCUUACUAC | |
| | GUGGGCUACCUGCAGCCCCGGACCUUCCUGCUGAAGUAC | |
| | AACGAGAACGGCACCAUCACCGACGCCGUGGACUGCGCCC | |
| | UGGACCCUCUGAGCGAGACCAAGUGCACCCUGAAGAGCU | |
| | UCACCGUGGAGAAGGGCAUCUACCAGACCAGCAACUUCC | |
| | GGGUGCAGCCCACCGAGAGCAUCGUGCGGUUCCCCAACA | |
| | UCACCAACCUGUGCCCCUUCGGCGAGGUGUUCAACGCCAC | |
| | CCGGUUCGCCAGCGUGUACGCCUGGAACCGGAAGCGGAU | |
| | CAGCAACUGCGUGGCCGACUACAGCGUGCUGUACAACAG | |
| | CGCCAGCUUCAGCACCUUCAAGUGCUACGGCGUGAGCCCC | |
| | ACCAAGCUGAACGACCUGUGCUUCACCAACGUGUACGCC | |
| | GACAGCUUCGUGAUCCGUGGCGACGAGGUGCGGCAGAUC | |
| | GCACCCGGCCAGACAGGCAAGAUCGCCGACUACAACUACA | |
| | AGCUGCCCGACGACUUCACCGGCUGCGUGAUCGCCUGGA | |
| | ACAGCAACAACCUCGACAGCAAGGUGGGCGGCAACUACA | |
| | ACUACCUGUACCGGCUGUUCCGGAAGAGCAACCUGAAGC | |
| | CCUUCGAGCGGGACAUCAGCACCGAGAUCUACCAAGCCG | |
| | GCUCCACCCCUUGCAACGGCGUGGAGGGCUUCAACUGCU | |
| | ACUUCCCUCUGCAGAGCUACGGCUUCCAGCCCACCUACGG | |
| | CGUGGGCUACCAGCCCUACCGGGUGGUGGUGCUGAGCUU | |
| | CGAGCUGCUGCACGCCCCAGCCACCGUGUGUGGCCCCAAG | |
| | AAGAGCACCAACCUGGUGAAGAACAAGUGCGUGAACUUC | |
| | AACUUCAACGGCCUUACCGGCACCGGCGUGCUGACCGAG | |
| | AGCAACAAGAAAUUCCUGCCCUUUCAGCAGUUCGGCCGG | |
| | GACAUCGACGACACCACCGACGCUGUGCGGGAUCCCCAGA | |
| | CCCUGGAGAUCCUGGACAUCACCCCUUGCAGCUUCGGCGG | |
| | CGUGAGCGUGAUCACCCCAGGCACCAACACCAGCAACCAG | |
| | GUGGCCGUGCUGUACCAGGGCGUGAACUGCACCGAGGUG | |
| | CCCGUGGCCAUCCACGCCGACCAGCUGACACCCACCUGGC | |
| | GGGUCUACAGCACCGGCAGCAACGUGUUCCAGACCCGGG | |
| | CCGGUUGCCUGAUCGGCGCCGAGCACGUGAACAACAGCU | |
| | ACGAGUGCGACAUCCCCAUCGGCGCCGGCAUCUGUGCCAG | |
| | CUACCAGACCCAGACCAAUUCACACCGGAGGGCAAGGAG | |
| | CGUGGCCAGCCAGAGCAUCAUCGCCUACACCAUGAGCCUG | |
| | GGCGCCGAGAACAGCGUGGCCUACAGCAACAACAGCAUC | |
| | GCCAUCCCCAUCAACUUCACCAUCAGCGUGACCACCGAGA | |
| | UUCUGCCCGUGAGCAUGACCAAGACCAGCGUGGACUGCA | |
| | CCAUGUACAUCUGCGGCGACAGCACCGAGUGCAGCAACC | |
| | UGCUGCUGCAGUACGGCAGCUUCUGCACCCAGCUGAACC | |
| | GGGCCCUGACCGGCAUCGCCGUGGAGCAGGACAAGAACA | |
| | CCCAGGAGGUGUUCGCCCAGGUGAAGCAGAUCUACAAGA | |
| | CCCCUCCCAUCAAGGACUUCGGCGGCUUCAACUUCAGCCA | |
| | GAUCCUGCCCGACCCCAGCAAGCCCAGCAAGCGGAGCUUC | |
| | AUCGAGGACCUGCUGUUCAACAAGGUGACCCUAGCCGAC | |
| | GCCGGCUUCAUCAAGCAGUACGGCGACUGCCUCGGCGAC | |
| | AUAGCCGCCCGGGACCUGAUCUGCGCCCAGAAGUUCAAC | |
| | GGCCUGACCGUGCUGCCUCCCCUGCUGACCGACGAGAUGA | |
| | UCGCCCAGUACACCAGCGCCCUGUUAGCCGGAACCAUCAC | |
| | CAGCGGCUGGACUUUCGGCGCUGGAGCCGCUCUGCAGAU | |
| | CCCCUUCGCCAUGCAGAUGGCCUACCGGUUCAACGGCAUC | |
| | GGCGUGACCCAGAACGUGCUGUACGAGAACCAGAAGCUG | |
| | AUCGCCAACCAGUUCAACAGCGCCAUCGGCAAGAUCCAG | |
| | GACAGCCUGAGCAGCACCGCUAGCGCCCUGGGCAAGCUGC | |
| | AGGACGUGGUGAACCAGAACGCCCAGGCCCUGAACACCC | |
| | UGGUGAAGCAGCUGAGCAGCAACUUCGGCGCCAUCAGCA | |
| | GCGUGCUGAACGACAUCCUGGCCCGGCUGGACCCUCCCGA | |
| | GGCCGAGGUGCAGAUCGACCGGCUGAUCACUGGCCGGCU | |
| | GCAGAGCCUGCAGACCUACGUGACCCAGCAGCUGAUCCG | |
| | GGCCGCCGAGAUUCGGGCCAGCGCCAACCUGGCCGCCACC | |
| | AAGAUGAGCGAGUGCGUGCUGGGCCAGAGCAAGCGGGUG | |
| | GACUUCUGCGGCAAGGGCUACCACCUGAUGAGCUUUCCC | |
| | CAGAGCGCACCCCACGGAGUGGUGUUCCUGCACGUGACC | |
| | UACGUGCCCGCCCAGGAGAAGAACUUCACCACCGCCCCAG | |
| | CCAUCUGCCACGACGGCAAGGCCCACUUUCCCCGGGAGGG | |
| | CGUGUUCGUGAGCAACGGCACCCACUGGUUCGUGACCCA | |
| | GCGGAACUUCUACGAGCCCCAGAUCAUCACCACCCACAAC | |
| | ACCUUCGUGAGCGGCAACUGCGACGUGGUGAUCGGCAUC | |
| | GUGAACAACACCGUGUACGAUCCCCUGCAGCCCGAGCUG | |
| | GACAGCUUCAAGGAGGAGCUGGACAAGUACUUCAAGAAU | |
| | CACACCAGCCCCGACGUGGACCUGGGCGACAUCAGCGGCA | |
| | UCAACGCCAGCGUGGUGAACAUCCAGAAGGAGAUCGAUC | |
| | GGCUGAACGAGGUGGCCAAGAACCUGAACGAGAGCCUGA | |
| | UCGACCUGCAGGAGCUGGGCAAGUACGAGCAGUACAUCA | |
| | AGUGGCCCUGGUACAUCUGGCUGGGCUUCAUCGCCGGCC | |
| | UGAUCGCCAUCGUGAUGGUGACCAUCAUGCUGUGCUGCA | |
| | UGACCAGCUGCUGCAGCUGCCUGAAGGGCUGUUGCAGCU | |
| | GCGGCAGCUGCUGCAAGUUCGACGAGGACGACAGCGAGC | |
| | CCGUGCUGAAGGGCGUGAAGCUGCACUACACC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MFVFLVLLPLVSSQCVNLTTRTQLPPAYTNSFTRGVYYPDKVF | 14 |
| acid sequence | RSSVLHSTQDLFLPFFSNVTWFHAISGTNGTKRFDNPVLPEND | |
| | GVYFASTEKSNIIRGWIFGTTLDSKTQSLLIVNNATNVVIKVCE | |
| | FQFCNDPFLGVYHKNNKSWMESEFRVYSSANNCTFEYVSQPF | |
| | LMDLEGKQGNFKNLREFVFKNIDGYFKIYSKHTPINLVRDLPQ | |
| | GFSALEPLVDLPIGINITRFQTLLALHRSYLTPGDSSSGWTAGA | |
| | AAYYVGYLQPRTFLLKYNENGTITDAVDCALDPLSETKCTLK | |
| | SFTVEKGIYQTSNFRVQPTESIVRFPNITNLCPFGEVFNATRFAS | |
| | VYAWNRKRISNCVADYSVLYNSASFSTFKCYGVSPTKLNDLC | |
| | FTNVYADSFVIRGDEVRQIAPGQTGKIADYNYKLPDDFTGCVI | |
| | AWNSNNLDSKVGGNYNYLYRLFRKSNLKPFERDISTEIYQAG | |
| | STPCNGVEGFNCYFPLQSYGFQPTYGVGYQPYRVVVLSFELL | |
| | HAPATVCGPKKSTNLVKNKCVNFNFNGLTGTGVLTESNKKFL | |
| | PFQQFGRDIDDTTDAVRDPQTLEILDITPCSFGGVSVITPGTNTS | |
| | NQVAVLYQGVNCTEVPVAIHADQLTPTWRVYSTGSNVFQTR | |
| | AGCLIGAEHVNNSYECDIPIGAGICASYQTQTNSHRRARSVAS | |
| | QSIIAYTMSLGAENSVAYSNNSIAIPINFTISVTTEILPVSMTKTS | |
| | VDCTMYICGDSTECSNLLLQYGSFCTQLNRALTGIAVEQDKN | |
| | TQEVFAQVKQIYKTPPIKDFGGFNFSQILPDPSKPSKRSFIEDLL | |
| | FNKVTLADAGFIKQYGDCLGDIAARDLICAQKFNGLTVLPPLL | |
| | TDEMIAQYTSALLAGTITSGWTFGAGAALQIPFAMQMAYRFN | |
| | GIGVTQNVLYENQKLIANQFNSAIGKIQDSLSSTASALGKLQD | |
| | VVNQNAQALNTLVKQLSSNFGAISSVLNDILARLDPPEAEVQI | |
| | DRLITGRLQSLQTYVTQQLIRAAEIRASANLAATKMSECVLGQ | |
| | SKRVDFCGKGYHLMSFPQSAPHGVVFLHVTYVPAQEKNFTTA | |
| | PAICHDGKAHFPREGVFVSNGTHWFVTQRNFYEPQIITTHNTF | |
| | VSGNCDVVIGIVNNTVYDPLQPELDSFKEELDKYFKNHTSPDV | |
| | DLGDISGINASVVNIQKEIDRLNEVAKNLNESLIDLQELGKYEQ | |
| | YIKWPWYIWLGFIAGLIAIVMVTIMLCCMTSCCSCLKGCCSCG | |
| | SCCKFDEDDSEPVLKGVKLHYT | |
| PolyA tail | 100 nt | |

WH2020_NatSP_2P_L18F_H69del_V70del_D80A_Y144del_D215G_L242_244del_R246I_K417N_
E484K_N501Y_A570D_D614G_P681H_A701V_T716I_S982A_D1118H

| SEQ ID NO: 15 consists of from 5′ end to 3′ end: 5′ UTR SEQ ID NO: 2, | | 15 |
|---|---|---|
| mRNA ORF SEQ ID NO: 16, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGACCCCGGCGCCGCCACC | |
| ORF of mRNA | AUGUUCGUGUUCCUGGUGCUGCUGCCCCUGGUGAGCAGC | 16 |
| Construct | CAGUGCGUGAACUUUACCACCCGGACCCAGCUGCCACCAG | |
| (excluding the stop | CCUACACCAACAGCUUCACCCGGGGCGUCUACUACCCCGA | |
| codon) | CAAGGUGUUCCGGAGCAGCGUCCUGCACAGCACCCAGGA | |
| | CCUGUUCCUGCCCUUCUUCAGCAACGUGACCUGGUUCCAC | |
| | GCCAUCAGCGGCACCAACGGCACCAAGCGGUUCGCCAACC | |
| | CCGUGCUGCCCUUCAACGACGGCGUGUACUUCGCCAGCAC | |
| | CGAGAAGAGCAACAUCAUCCGGGGCUGGAUCUUCGGCAC | |
| | CACCCUGGACAGCAAGACCCAGAGCCUGCUGAUCGUGAA | |
| | UAACGCCACCAACGUGGUGAUCAAGGUGUGCGAGUUCCA | |
| | GUUCUGCAACGACCCCUUCCUGGGCGUGUACCACAAGAA | |
| | CAACAAGAGCUGGAUGGAGAGCGAGUUCCGGGUGUACAG | |
| | CAGCGCCAACAACUGCACCUUCGAGUACGUGAGCCAGCCC | |
| | UUCCUGAUGGACCUGGAGGGCAAGCAGGGCAACUUCAAG | |
| | AACCUGCGGGAGUUCGUGUUCAAGAACAUCGACGGCUAC | |
| | UUCAAGAUCUACAGCAAGCACACCCCAAUCAACCUGGUG | |
| | CGGGGCCUGCCCCAGGGCUUCUCAGCCCUGGAGCCCCUGG | |
| | UGGACCUGCCCAUCGGCAUCAACAUCACCCGGUUCCAGAC | |
| | CCUGCACAUCAGCUACCUGACCCCAGGCGACAGCAGCAGC | |
| | GGGUGGACAGCAGGCGCGGCUGCUUACUACGUGGGCUAC | |
| | CUGCAGCCCCGGACCUUCCUGCUGAAGUACAACGAGAAC | |
| | GGCACCAUCACCGACGCCGUGGACUGCGCCCUGGACCCUC | |
| | UGAGCGAGACCAAGUGCACCCUGAAGAGCUUCACCGUGG | |
| | AGAAGGGCAUCUACCAGACCAGCAACUUCCGGGUGCAGC | |
| | CCACCGAGAGCAUCGUGCGGUUCCCCAACAUCACCAACCU | |
| | GUGCCCCUUCGGCGAGGUGUUCAACGCCACCCGGUUCGCC | |
| | AGCGUGUACGCCUGGAACCGGAAGCGGAUCAGCAACUGC | |
| | GUGGCCGACUACAGCGUGCUGUACAACAGCGCCAGCUUC | |
| | AGCACCUUCAAGUGCUACGGCGUGAGCCCCACCAAGCUG | |
| | AACGACCUGUGCUUCACCAACGUGUACGCCGACAGCUUC | |
| | GUGAUCCGUGGCGACGAGGUGCGGCAGAUCGCACCCGGC | |
| | CAGACAGGCAACAUCGCCGACUACAACUACAAGCUGCCCG | |
| | ACGACUUCACCGGCUGCGUGAUCGCCUGGAACAGCAACA | |
| | ACCUCGACAGCAAGGUGGGCGGCAACUACAACUACCUGU | |
| | ACCGGCUGUUCCGGAAGAGCAACCUGAAGCCCUUCGAGC | |
| | GGGACAUCAGCACCGAGAUCUACCAAGCCGGCUCCACCCC | |
| | UUGCAACGGCGUGAAGGGCUUCAACUGCUACUUCCCUCU | |
| | GCAGAGCUACGGCUUCCAGCCCACCUACGGCGUGGGCUAC | |
| | CAGCCCUACCGGGUGGUGGUGCUGAGCUUCGAGCUGCUG | |
| | CACGCCCCAGCCACCGUGUGUGGCCCCAAGAAGAGCACCA | |
| | ACCUGGUGAAGAACAAGUGCGUGAACUUCAACUUCAACG | |
| | GCCUUACCGGCACCGGCGUGCUGACCGAGAGCAACAAGA | |
| | AAUUCCUGCCCUUUCAGCAGUUCGGCCGGGACAUCGACG | |
| | ACACCACCGACGCUGUGCGGGAUCCCCAGACCCUGGAGAU | |
| | CCUGGACAUCACCCCUUGCAGCUUCGGCGGCGUGAGCGU | |
| | GAUCACCCCAGGCACCAACACCAGCAACCAGGUGGCCGUG | |
| | CUGUACCAGGGCGUGAACUGCACCGAGGUGCCCGUGGCC | |
| | AUCCACGCCGACCAGCUGACACCCACCUGGCGGGUCUACA | |
| | GCACCGGCAGCAACGUGUUCCAGACCCGGGCCGGUUGCCU | |
| | GAUCGGCGCCGAGCACGUGAACAACAGCUACGAGUGCGA | |
| | CAUCCCCAUCGGCGCCGGCAUCUGUGCCAGCUACCAGACC | |
| | CAGACCAAUUCACACCGGAGGGCAAGGAGCGUGGCCAGC | |
| | CAGAGCAUCAUCGCCUACACCAUGAGCCUGGGCGUGGAG | |
| | AACAGCGUGGCCUACAGCAACAACAGCAUCGCCAUCCCCA | |
| | UCAACUUCACCAUCAGCGUGACCACCGAGAUUCUGCCCGU | |
| | GAGCAUGACCAAGACCAGCGUGGACUGCACCAUGUACAU | |
| | CUGCGGCGACAGCACCGAGUGCAGCAACCUGCUGCUGCA | |
| | GUACGGCAGCUUCUGCACCCAGCUGAACCGGGCCCUGACC | |
| | GGCAUCGCCGUGGAGCAGGACAAGAACACCCAGGAGGUG | |
| | UUCGCCCAGGUGAAGCAGAUCUACAAGACCCCUCCCAUCA | |
| | AGGACUUCGGCGGCUUCAACUUCAGCCAGAUCCUGCCCG | |
| | ACCCCAGCAAGCCCAGCAAGCGGAGCUUCAUCGAGGACCU | |
| | GCUGUUCAACAAGGUGACCCUAGCCGACGCCGGCUUCAU | |
| | CAAGCAGUACGGCGACUGCCUCGGCGACAUAGCCGCCCGG | |
| | GACCUGAUCUGCGCCCAGAAGUUCAACGGCCUGACCGUG | |
| | CUGCCUCCCCUGCUGACCGACGAGAUGAUCGCCCAGUACA | |
| | CCAGCGCCCUGUUAGCCGGAACCAUCACCAGCGGCUGGAC | |
| | UUUCGGCGCUGGAGCCGCUCUGCAGAUCCCCUUCGCCAUG | |
| | CAGAUGGCCUACCGGUUCAACGGCAUCGGCGUGACCCAG | |
| | AACGUGCUGUACGAGAACCAGAAGCUGAUCGCCAACCAG | |
| | UUCAACAGCGCCAUCGGCAAGAUCCAGGACAGCCUGAGC | |
| | AGCACCGCUAGCGCCCUGGGCAAGCUGCAGGACGUGGUG | |
| | AACCAGAACGCCCAGGCCCUGAACACCCUGGUGAAGCAGC | |
| | UGAGCAGCAACUUCGGCGCCAUCAGCAGCGUGCUGAACG | |
| | ACAUCCUGGCCCGGCUGGACCCUCCCGAGGCCGAGGUGCA | |
| | GAUCGACCGGCUGAUCACUGGCCGGCUGCAGAGCCUGCA | |
| | GACCUACGUGACCCAGCAGCUGAUCCGGGCCGCCGAGAU | |
| | UCGGGCCAGCGCCAACCUGGCCGCCACCAAGAUGAGCGAG | |
| | UGCGUGCUGGGCCAGAGCAAGCGGGUGGACUUCUGCGGC | |
| | AAGGGCUACCACCUGAUGAGCUUUCCCCAGAGCGCACCCC | |
| | ACGGAGUGGUGUUCCUGCACGUGACCUACGUGCCCGCCC | |
| | AGGAGAAGAACUUCACCACCGCCCCAGCCAUCUGCCACGA | |
| | CGGCAAGGCCCACUUUCCCCGGGAGGGCGUGUUCGUGAG | |
| | CAACGGCACCCACUGGUUCGUGACCCAGCGGAACUUCUAC | |
| | GAGCCCCAGAUCAUCACCACCCACAACACCUUCGUGAGCG | |
| | GCAACUGCGACGUGGUGAUCGGCAUCGUGAACAACACCG | |
| | UGUACGAUCCCCUGCAGCCCGAGCUGGACAGCUUCAAGG | |
| | AGGAGCUGGACAAGUACUUCAAGAAUCACACCAGCCCCG | |
| | ACGUGGACCUGGGCGACAUCAGCGGCAUCAACGCCAGCG | |
| | UGGUGAACAUCCAGAAGGAGAUCGAUCGGCUGAACGAGG | |
| | UGGCCAAGAACCUGAACGAGAGCCUGAUCGACCUGCAGG | |
| | AGCUGGGCAAGUACGAGCAGUACAUCAAGUGGCCCUGGU | |
| | ACAUCUGGCUGGGCUUCAUCGCCGGCCUGAUCGCCAUCG | |
| | UGAUGGUGACCAUCAUGCUGUGCUGCAUGACCAGCUGCU | |
| | GCAGCUGCCUGAAGGGCUGUUGCAGCUGCGGCAGCUGCU | |
| | GCAAGUUCGACGAGGACGACAGCGAGCCCGUGCUGAAGG | |
| | GCGUGAAGCUGCACUACACC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MFVFLVLLPLVSSQCVNFTTRTQLPPAYTNSFTRGVYYPDKVF | 17 |
| acid sequence | RSSVLHSTQDLFLPFFSNVTWFHAISGTNGTKRFANPVLPFND | |
| | GVYFASTEKSNIIRGWIFGTTLDSKTQSLLIVNNATNVVIKVCE | |
| | FQFCNDPFLGVYHKNNKSWMESEFRVYSSANNCTFEYVSQPF | |
| | LMDLEGKQGNFKNLREFVFKNIDGYFKIYSKHTPINLVRGLPQ | |
| | GFSALEPLVDLPIGINITRFQTLHISYLTPGDSSSGWTAGAAAY | |
| | YVGYLQPRTFLLKYNENGTITDAVDCALDPLSETKCTLKSFTV | |
| | EKGIYQTSNFRVQPTESIVRFPNITNLCPFGEVENATRFASVYA | |
| | WNRKRISNCVADYSVLYNSASFSTFKCYGVSPTKLNDLCFTN | |
| | VYADSFVIRGDEVRQIAPGQTGNIADYNYKLPDDFTGCVIAW | |
| | NSNNLDSKVGGNYNYLYRLFRKSNLKPFERDISTEIYQAGSTP | |
| | CNGVKGFNCYFPLQSYGFQPTYGVGYQPYRVVVLSFELLHAP | |
| | ATVCGPKKSTNLVKNKCVNFNFNGLTGTGVLTESNKKFLPFQ | |
| | QFGRDIDDTTDAVRDPQTLEILDITPCSFGGVSVITPGTNTSNQ | |
| | VAVLYQGVNCTEVPVAIHADQLTPTWRVYSTGSNVFQTRAG | |
| | CLIGAEHVNNSYECDIPIGAGICASYQTQTNSHRRARSVASQSI | |
| | IAYTMSLGVENSVAYSNNSIAIPINFTISVTTEILPVSMTKTSVD | |
| | CTMYICGDSTECSNLLLQYGSFCTQLNRALTGIAVEQDKNTQE | |
| | VFAQVKQIYKTPPIKDFGGFNFSQILPDPSKPSKRSFIEDLLENK | |
| | VTLADAGFIKQYGDCLGDIAARDLICAQKFNGLTVLPPLLTDE | |
| | MIAQYTSALLAGTITSGWTFGAGAALQIPFAMQMAYRENGIG | |
| | VTQNVLYENQKLIANQFNSAIGKIQDSLSSTASALGKLQDVVN | |
| | QNAQALNTLVKQLSSNFGAISSVLNDILARLDPPEAEVQIDRLI | |
| | TGRLQSLQTYVTQQLIRAAEIRASANLAATKMSECVLGQSKR | |
| | VDFCGKGYHLMSFPQSAPHGVVFLHVTYVPAQEKNFTTAPAI | |
| | CHDGKAHFPREGVFVSNGTHWFVTQRNFYEPQIITTHNTFVSG | |
| | NCDVVIGIVNNTVYDPLQPELDSFKEELDKYFKNHTSPDVDLG | |
| | DISGINASVVNIQKEIDRLNEVAKNLNESLIDLQELGKYEQYIK | |
| | WPWYIWLGFIAGLIAIVMVTIMLCCMTSCCSCLKGCCSCGSCC | |
| | KFDEDDSEPVLKGVKLHYT | |
| PolyA tail | 100 nt | |

WH2020_NatSP_2P

| SEQ ID NO: 18 consists of from 5′ end to 3′ end: 5′ UTR SEQ ID NO: 2, | | 18 |
|---|---|---|
| mRNA ORF SEQ ID NO: 19, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGACCCCGGCGCCGCCACC | |
| ORF of mRNA | AUGUUCGUGUUCCUGGUGCUGCUGCCCCUGGUGAGCAGC | 19 |
| Construct | CAGUGCGUGAACCUGACCACCCGGACCCAGCUGCCACCAG | |
| (excluding the stop | CCUACACCAACAGCUUCACCCGGGGCGUCUACUACCCCGA | |
| codon) | CAAGGUGUUCCGGAGCAGCGUCCUGCACAGCACCCAGGA | |
| | CCUGUUCCUGCCCUUCUUCAGCAACGUGACCUGGUUCCAC | |
| | GCCAUCCACGUGAGCGGCACCAACGGCACCAAGCGGUUCG | |
| | ACAACCCCGUGCUGCCCUUCAACGACGGCGUGUACUUCGC | |
| | CAGCACCGAGAAGAGCAACAUCAUCCGGGGCUGGAUCUU | |
| | CGGCACCACCCUGGACAGCAAGACCCAGAGCCUGCUGAUC | |
| | GUGAAUAACGCCACCAACGUGGUGAUCAAGGUGUGCGAG | |
| | UUCCAGUUCUGCAACGACCCCUUCCUGGGCGUGUACUACC | |
| | ACAAGAACAACAAGAGCUGGAUGGAGAGCGAGUUCCGGG | |
| | UGUACAGCAGCGCCAACAACUGCACCUUCGAGUACGUGA | |
| | GCCAGCCCUUCCUGAUGGACCUGGAGGGCAAGCAGGGCA | |
| | ACUUCAAGAACCUGCGGGAGUUCGUGUUCAAGAACAUCG | |
| | ACGGCUACUUCAAGAUCUACAGCAAGCACACCCCAAUCA | |
| | ACCUGGUGCGGGAUCUGCCCCAGGGCUUCUCAGCCCUGG | |
| | AGCCCCUGGUGGACCUGCCCAUCGGCAUCAACAUCACCCG | |
| | GUUCCAGACCCUGCUGGCCCUGCACCGGAGCUACCUGACC | |
| | CCAGGCGACAGCAGCAGCGGGUGGACAGCAGGCGCGGCU | |
| | GCUUACUACGUGGGCUACCUGCAGCCCCGGACCUUCCUGC | |
| | UGAAGUACAACGAGAACGGCACCAUCACCGACGCCGUGG | |
| | ACUGCGCCCUGGACCCUCUGAGCGAGACCAAGUGCACCCU | |
| | GAAGAGCUUCACCGUGGAGAAGGGCAUCUACCAGACCAG | |
| | CAACUUCCGGGUGCAGCCCACCGAGAGCAUCGUGCGGUU | |
| | CCCCAACAUCACCAACCUGUGCCCCUUCGGCGAGGUGUUC | |
| | AACGCCACCCGGUUCGCCAGCGUGUACGCCUGGAACCGGA | |
| | AGCGGAUCAGCAACUGCGUGGCCGACUACAGCGUGCUGU | |
| | ACAACAGCGCCAGCUUCAGCACCUUCAAGUGCUACGGCG | |
| | UGAGCCCCACCAAGCUGAACGACCUGUGCUUCACCAACGU | |
| | GUACGCCGACAGCUUCGUGAUCCGUGGCGACGAGGUGCG | |
| | GCAGAUCGCACCCGGCCAGACAGGCAAGAUCGCCGACUAC | |
| | AACUACAAGCUGCCCGACGACUUCACCGGCUGCGUGAUC | |
| | GCCUGGAACAGCAACAACCUCGACAGCAAGGUGGGCGGC | |
| | AACUACAACUACCUGUACCGGCUGUUCCGGAAGAGCAAC | |
| | CUGAAGCCCUUCGAGCGGGACAUCAGCACCGAGAUCUAC | |
| | CAAGCCGGCUCCACCCCUUGCAACGGCGUGGAGGGCUUCA | |
| | ACUGCUACUUCCCUCUGCAGAGCUACGGCUUCCAGCCCAC | |
| | CAACGGCGUGGGCUACCAGCCCUACCGGGUGGUGGUGCU | |
| | GAGCUUCGAGCUGCUGCACGCCCCAGCCACCGUGUGUGGC | |
| | CCCAAGAAGAGCACCAACCUGGUGAAGAACAAGUGCGUG | |
| | AACUUCAACUUCAACGGCCUUACCGGCACCGGCGUGCUG | |
| | ACCGAGAGCAACAAGAAAUUCCUGCCCUUUCAGCAGUUC | |
| | GGCCGGGACAUCGCCGACACCACCGACGCUGUGCGGGAUC | |
| | CCCAGACCCUGGAGAUCCUGGACAUCACCCCUUGCAGCUU | |
| | CGGCGGCGUGAGCGUGAUCACCCCAGGCACCAACACCAGC | |
| | AACCAGGUGGCCGUGCUGUACCAGGACGUGAACUGCACC | |
| | GAGGUGCCCGUGGCCAUCCACGCCGACCAGCUGACACCCA | |
| | CCUGGCGGGUCUACAGCACCGGCAGCAACGUGUUCCAGA | |
| | CCCGGGCCGGUUGCCUGAUCGGCGCCGAGCACGUGAACA | |
| | ACAGCUACGAGUGCGACAUCCCCAUCGGCGCCGGCAUCUG | |
| | UGCCAGCUACCAGACCCAGACCAAUUCACCCCGGAGGGCA | |
| | AGGAGCGUGGCCAGCCAGAGCAUCAUCGCCUACACCAUG | |
| | AGCCUGGGCGCCGAGAACAGCGUGGCCUACAGCAACAAC | |
| | AGCAUCGCCAUCCCCACCAACUUCACCAUCAGCGUGACCA | |
| | CCGAGAUUCUGCCCGUGAGCAUGACCAAGACCAGCGUGG | |
| | ACUGCACCAUGUACAUCUGCGGCGACAGCACCGAGUGCA | |
| | GCAACCUGCUGCUGCAGUACGGCAGCUUCUGCACCCAGCU | |
| | GAACCGGGCCCUGACCGGCAUCGCCGUGGAGCAGGACAA | |
| | GAACACCCAGGAGGUGUUCGCCCAGGUGAAGCAGAUCUA | |
| | CAAGACCCCUCCCAUCAAGGACUUCGGCGGCUUCAACUUC | |
| | AGCCAGAUCCUGCCCGACCCCAGCAAGCCCAGCAAGCGGA | |
| | GCUUCAUCGAGGACCUGCUGUUCAACAAGGUGACCCUAG | |
| | CCGACGCCGGCUUCAUCAAGCAGUACGGCGACUGCCUCGG | |
| | CGACAUAGCCGCCCGGGACCUGAUCUGCGCCCAGAAGUUC | |
| | AACGGCCUGACCGUGCUGCCUCCCCUGCUGACCGACGAGA | |
| | UGAUCGCCCAGUACACCAGCGCCCUGUUAGCCGGAACCAU | |
| | CACCAGCGGCUGGACUUUCGGCGCUGGAGCCGCUCUGCA | |
| | GAUCCCCUUCGCCAUGCAGAUGGCCUACCGGUUCAACGGC | |
| | AUCGGCGUGACCCAGAACGUGCUGUACGAGAACCAGAAG | |
| | CUGAUCGCCAACCAGUUCAACAGCGCCAUCGGCAAGAUCC | |
| | AGGACAGCCUGAGCAGCACCGCUAGCGCCCUGGGCAAGC | |
| | UGCAGGACGUGGUGAACCAGAACGCCCAGGCCCUGAACA | |
| | CCCUGGUGAAGCAGCUGAGCAGCAACUUCGGCGCCAUCA | |
| | GCAGCGUGCUGAACGACAUCCUGAGCCGGCUGGACCCUCC | |
| | CGAGGCCGAGGUGCAGAUCGACCGGCUGAUCACUGGCCG | |
| | GCUGCAGAGCCUGCAGACCUACGUGACCCAGCAGCUGAU | |
| | CCGGGCCGCCGAGAUUCGGGCCAGCGCCAACCUGGCCGCC | |
| | ACCAAGAUGAGCGAGUGCGUGCUGGGCCAGAGCAAGCGG | |
| | GUGGACUUCUGCGGCAAGGGCUACCACCUGAUGAGCUUU | |
| | CCCCAGAGCGCACCCCACGGAGUGGUGUUCCUGCACGUGA | |
| | CCUACGUGCCCGCCCAGGAGAAGAACUUCACCACCGCCCC | |
| | AGCCAUCUGCCACGACGGCAAGGCCCACUUUCCCCGGGAG | |
| | GGCGUGUUCGUGAGCAACGGCACCCACUGGUUCGUGACC | |
| | CAGCGGAACUUCUACGAGCCCCAGAUCAUCACCACCGACA | |
| | ACACCUUCGUGAGCGGCAACUGCGACGUGGUGAUCGGCA | |
| | UCGUGAACAACACCGUGUACGAUCCCCUGCAGCCCGAGCU | |
| | GGACAGCUUCAAGGAGGAGCUGGACAAGUACUUCAAGAA | |
| | UCACACCAGCCCCGACGUGGACCUGGGCGACAUCAGCGGC | |
| | AUCAACGCCAGCGUGGUGAACAUCCAGAAGGAGAUCGAU | |
| | CGGCUGAACGAGGUGGCCAAGAACCUGAACGAGAGCCUG | |
| | AUCGACCUGCAGGAGCUGGGCAAGUACGAGCAGUACAUC | |
| | AAGUGGCCCUGGUACAUCUGGCUGGGCUUCAUCGCCGGC | |
| | CUGAUCGCCAUCGUGAUGGUGACCAUCAUGCUGUGCUGC | |
| | AUGACCAGCUGCUGCAGCUGCCUGAAGGGCUGUUGCAGC | |
| | UGCGGCAGCUGCUGCAAGUUCGACGAGGACGACAGCGAG | |
| | CCCGUGCUGAAGGGCGUGAAGCUGCACUACACC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MFVFLVLLPLVSSQCVNLTTRTQLPPAYTNSFTRGVYYPDKVF | 20 |
| acid sequence | RSSVLHSTQDLFLPFFSNVTWFHAIHVSGTNGTKRFDNPVLPF | |
| | NDGVYFASTEKSNIIRGWIFGTTLDSKTQSLLIVNNATNVVIKV | |
| | CEFQFCNDPFLGVYYHKNNKSWMESEFRVYSSANNCTFEYVS | |
| | QPFLMDLEGKQGNFKNLREFVFKNIDGYFKIYSKHTPINLVRD | |
| | LPQGFSALEPLVDLPIGINITRFQTLLALHRSYLTPGDSSSGWT | |
| | AGAAAYYVGYLQPRTFLLKYNENGTITDAVDCALDPLSETKC | |
| | TLKSFTVEKGIYQTSNFRVQPTESIVRFPNITNLCPFGEVFNATR | |
| | FASVYAWNRKRISNCVADYSVLYNSASFSTFKCYGVSPTKLN | |
| | DLCFTNVYADSFVIRGDEVRQIAPGQTGKIADYNYKLPDDFTG | |
| | CVIAWNSNNLDSKVGGNYNYLYRLFRKSNLKPFERDISTEIYQ | |
| | AGSTPCNGVEGENCYFPLQSYGFQPTNGVGYQPYRVVVLSFE | |
| | LLHAPATVCGPKKSTNLVKNKCVNFNFNGLTGTGVLTESNKK | |
| | FLPFQQFGRDIADTTDAVRDPQTLEILDITPCSFGGVSVITPGTN | |
| | TSNQVAVLYQDVNCTEVPVAIHADQLTPTWRVYSTGSNVFQT | |
| | RAGCLIGAEHVNNSYECDIPIGAGICASYQTQTNSPRRARSVA | |
| | SQSIIAYTMSLGAENSVAYSNNSIAIPTNFTISVTTEILPVSMTK | |
| | TSVDCTMYICGDSTECSNLLLQYGSFCTQLNRALTGIAVEQDK | |
| | NTQEVFAQVKQIYKTPPIKDFGGFNFSQILPDPSKPSKRSFIEDL | |
| | LFNKVTLADAGFIKQYGDCLGDIAARDLICAQKFNGLTVLPPL | |
| | LTDEMIAQYTSALLAGTITSGWTFGAGAALQIPFAMQMAYRF | |
| | NGIGVTQNVLYENQKLIANQFNSAIGKIQDSLSSTASALGKLQ | |
| | DVVNQNAQALNTLVKQLSSNFGAISSVLNDILSRLDPPEAEVQ | |
| | IDRLITGRLQSLQTYVTQQLIRAAEIRASANLAATKMSECVLG | |
| | QSKRVDFCGKGYHLMSFPQSAPHGVVFLHVTYVPAQEKNFTT | |
| | APAICHDGKAHFPREGVFVSNGTHWFVTQRNFYEPQIITTDNT | |
| | FVSGNCDVVIGIVNNTVYDPLQPELDSFKEELDKYFKNHTSPD | |
| | VDLGDISGINASVVNIQKEIDRLNEVAKNLNESLIDLQELGKYE | |
| | QYIKWPWYIWLGFIAGLIAIVMVTIMLCCMTSCCSCLKGCCSC | |
| | GSCCKFDEDDSEPVLKGVKLHYT | |
| PolyA tail | 100 nt | |

S2P_IN_[T95I, G142D, E154K, L452R, E484Q, D614G, P681R, Q1071H]

| SEQ ID NO: 21 consists of from 5′ end to 3′ end: 5′ UTR SEQ ID NO: 2, | | 21 |
|---|---|---|
| mRNA ORF SEQ ID NO: 22, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGACCCCGGCGCCGCCACC | |
| ORF of mRNA | AUGUUCGUGUUCCUGGUGCUGCUGCCCCUGGUGAGCAGC | 22 |
| Construct | CAGUGCGUGAACCUGACCACCCGGACCCAGCUGCCACCAG | |
| (excluding the stop | CCUACACCAACAGCUUCACCCGGGGCGUCUACUACCCCGA | |
| codon) | CAAGGUGUUCCGGAGCAGCGUCCUGCACAGCACCCAGGA | |
| | CCUGUUCCUGCCCUUCUUCAGCAACGUGACCUGGUUCCAC | |
| | GCCAUCCACGUGAGCGGCACCAACGGCACCAAGCGGUUCG | |
| | ACAACCCCGUGCUGCCCUUCAACGACGGCGUGUACUUCGC | |
| | CAGCAUCGAGAAGAGCAACAUCAUCCGGGGCUGGAUCUU | |
| | CGGCACCACCCUGGACAGCAAGACCCAGAGCCUGCUGAUC | |
| | GUGAAUAACGCCACCAACGUGGUGAUCAAGGUGUGCGAG | |
| | UUCCAGUUCUGCAACGACCCCUUCCUGGACGUGUACUACC | |
| | ACAAGAACAACAAGAGCUGGAUGAAGAGCGAGUUCCGGG | |
| | UGUACAGCAGCGCCAACAACUGCACCUUCGAGUACGUGA | |
| | GCCAGCCCUUCCUGAUGGACCUGGAGGGCAAGCAGGGCA | |
| | ACUUCAAGAACCUGCGGGAGUUCGUGUUCAAGAACAUCG | |
| | ACGGCUACUUCAAGAUCUACAGCAAGCACACCCCAAUCA | |
| | ACCUGGUGCGGGAUCUGCCCCAGGGCUUCUCAGCCCUGG | |
| | AGCCCCUGGUGGACCUGCCCAUCGGCAUCAACAUCACCCG | |
| | GUUCCAGACCCUGCUGGCCCUGCACCGGAGCUACCUGACC | |
| | CCAGGCGACAGCAGCAGCGGGUGGACAGCAGGCGCGGCU | |
| | GCUUACUACGUGGGCUACCUGCAGCCCCGGACCUUCCUGC | |
| | UGAAGUACAACGAGAACGGCACCAUCACCGACGCCGUGG | |
| | ACUGCGCCCUGGACCCUCUGAGCGAGACCAAGUGCACCCU | |
| | GAAGAGCUUCACCGUGGAGAAGGGCAUCUACCAGACCAG | |
| | CAACUUCCGGGUGCAGCCCACCGAGAGCAUCGUGCGGUU | |
| | CCCCAACAUCACCAACCUGUGCCCCUUCGGCGAGGUGUUC | |
| | AACGCCACCCGGUUCGCCAGCGUGUACGCCUGGAACCGGA | |
| | AGCGGAUCAGCAACUGCGUGGCCGACUACAGCGUGCUGU | |
| | ACAACAGCGCCAGCUUCAGCACCUUCAAGUGCUACGGCG | |
| | UGAGCCCCACCAAGCUGAACGACCUGUGCUUCACCAACGU | |
| | GUACGCCGACAGCUUCGUGAUCCGUGGCGACGAGGUGCG | |
| | GCAGAUCGCACCCGGCCAGACAGGCAAGAUCGCCGACUAC | |
| | AACUACAAGCUGCCCGACGACUUCACCGGCUGCGUGAUC | |
| | GCCUGGAACAGCAACAACCUCGACAGCAAGGUGGGCGGC | |
| | AACUACAACUACAGAUACCGGCUGUUCCGGAAGAGCAAC | |
| | CUGAAGCCCUUCGAGCGGGACAUCAGCACCGAGAUCUAC | |
| | CAAGCCGGCUCCACCCCUUGCAACGGCGUGCAGGGCUUCA | |
| | ACUGCUACUUCCCUCUGCAGAGCUACGGCUUCCAGCCCAC | |
| | CAACGGCGUGGGCUACCAGCCCUACCGGGUGGUGGUGCU | |
| | GAGCUUCGAGCUGCUGCACGCCCCAGCCACCGUGUGUGGC | |
| | CCCAAGAAGAGCACCAACCUGGUGAAGAACAAGUGCGUG | |
| | AACUUCAACUUCAACGGCCUUACCGGCACCGGCGUGCUG | |
| | ACCGAGAGCAACAAGAAAUUCCUGCCCUUUCAGCAGUUC | |
| | GGCCGGGACAUCGCCGACACCACCGACGCUGUGCGGGAUC | |
| | CCCAGACCCUGGAGAUCCUGGACAUCACCCCUUGCAGCUU | |
| | CGGCGGCGUGAGCGUGAUCACCCCAGGCACCAACACCAGC | |
| | AACCAGGUGGCCGUGCUGUACCAGGGCGUGAACUGCACC | |
| | GAGGUGCCCGUGGCCAUCCACGCCGACCAGCUGACACCCA | |
| | CCUGGCGGGUCUACAGCACCGGCAGCAACGUGUUCCAGA | |
| | CCCGGGCCGGUUGCCUGAUCGGCGCCGAGCACGUGAACA | |
| | ACAGCUACGAGUGCGACAUCCCCAUCGGCGCCGGCAUCUG | |
| | UGCCAGCUACCAGACCCAGACCAAUUCAAGACGGAGGGC | |
| | AAGGAGCGUGGCCAGCCAGAGCAUCAUCGCCUACACCAU | |
| | GAGCCUGGGCGCCGAGAACAGCGUGGCCUACAGCAACAA | |
| | CAGCAUCGCCAUCCCCACCAACUUCACCAUCAGCGUGACC | |
| | ACCGAGAUUCUGCCCGUGAGCAUGACCAAGACCAGCGUG | |
| | GACUGCACCAUGUACAUCUGCGGCGACAGCACCGAGUGC | |
| | AGCAACCUGCUGCUGCAGUACGGCAGCUUCUGCACCCAGC | |
| | UGAACCGGGCCCUGACCGGCAUCGCCGUGGAGCAGGACA | |
| | AGAACACCCAGGAGGUGUUCGCCCAGGUGAAGCAGAUCU | |
| | ACAAGACCCCUCCCAUCAAGGACUUCGGCGGCUUCAACUU | |
| | CAGCCAGAUCCUGCCCGACCCCAGCAAGCCCAGCAAGCGG | |
| | AGCUUCAUCGAGGACCUGCUGUUCAACAAGGUGACCCUA | |
| | GCCGACGCCGGCUUCAUCAAGCAGUACGGCGACUGCCUCG | |
| | GCGACAUAGCCGCCCGGGACCUGAUCUGCGCCCAGAAGU | |
| | UCAACGGCCUGACCGUGCUGCCUCCCCUGCUGACCGACGA | |
| | GAUGAUCGCCCAGUACACCAGCGCCCUGUUAGCCGGAACC | |
| | AUCACCAGCGGCUGGACUUUCGGCGCUGGAGCCGCUCUG | |
| | CAGAUCCCCUUCGCCAUGCAGAUGGCCUACCGGUUCAACG | |
| | GCAUCGGCGUGACCCAGAACGUGCUGUACGAGAACCAGA | |
| | AGCUGAUCGCCAACCAGUUCAACAGCGCCAUCGGCAAGA | |
| | UCCAGGACAGCCUGAGCAGCACCGCUAGCGCCCUGGGCAA | |
| | GCUGCAGGACGUGGUGAACCAGAACGCCCAGGCCCUGAA | |
| | CACCCUGGUGAAGCAGCUGAGCAGCAACUUCGGCGCCAU | |
| | CAGCAGCGUGCUGAACGACAUCCUGAGCCGGCUGGACCC | |
| | UCCCGAGGCCGAGGUGCAGAUCGACCGGCUGAUCACUGG | |
| | CCGGCUGCAGAGCCUGCAGACCUACGUGACCCAGCAGCUG | |
| | AUCCGGGCCGCCGAGAUUCGGGCCAGCGCCAACCUGGCCG | |
| | CCACCAAGAUGAGCGAGUGCGUGCUGGGCCAGAGCAAGC | |
| | GGGUGGACUUCUGCGGCAAGGGCUACCACCUGAUGAGCU | |
| | UUCCCCAGAGCGCACCCCACGGAGUGGUGUUCCUGCACGU | |
| | GACCUACGUGCCCGCCCACGAGAAGAACUUCACCACCGCC | |
| | CCAGCCAUCUGCCACGACGGCAAGGCCCACUUUCCCCGGG | |
| | AGGGCGUGUUCGUGAGCAACGGCACCCACUGGUUCGUGA | |
| | CCCAGCGGAACUUCUACGAGCCCCAGAUCAUCACCACCGA | |
| | CAACACCUUCGUGAGCGGCAACUGCGACGUGGUGAUCGG | |
| | CAUCGUGAACAACACCGUGUACGAUCCCCUGCAGCCCGAG | |
| | CUGGACAGCUUCAAGGAGGAGCUGGACAAGUACUUCAAG | |
| | AAUCACACCAGCCCCGACGUGGACCUGGGCGACAUCAGCG | |
| | GCAUCAACGCCAGCGUGGUGAACAUCCAGAAGGAGAUCG | |
| | AUCGGCUGAACGAGGUGGCCAAGAACCUGAACGAGAGCC | |
| | UGAUCGACCUGCAGGAGCUGGGCAAGUACGAGCAGUACA | |
| | UCAAGUGGCCCUGGUACAUCUGGCUGGGCUUCAUCGCCG | |
| | GCCUGAUCGCCAUCGUGAUGGUGACCAUCAUGCUGUGCU | |
| | GCAUGACCAGCUGCUGCAGCUGCCUGAAGGGCUGUUGCA | |
| | GCUGCGGCAGCUGCUGCAAGUUCGACGAGGACGACAGCG | |
| | AGCCCGUGCUGAAGGGCGUGAAGCUGCACUACACC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MFVFLVLLPLVSSQCVNLTTRTQLPPAYTNSFTRGVYYPDKVF | 23 |
| acid sequence | RSSVLHSTQDLFLPFFSNVTWFHAIHVSGTNGTKRFDNPVLPF | |
| | NDGVYFASIEKSNIIRGWIFGTTLDSKTQSLLIVNNATNVVIKV | |
| | CEFQFCNDPFLDVYYHKNNKSWMKSEFRVYSSANNCTFEYV | |
| | SQPFLMDLEGKQGNFKNLREFVFKNIDGYFKIYSKHTPINLVR | |
| | DLPQGFSALEPLVDLPIGINITRFQTLLALHRSYLTPGDSSSGW | |
| | TAGAAAYYVGYLQPRTFLLKYNENGTITDAVDCALDPLSETK | |
| | CTLKSFTVEKGIYQTSNFRVQPTESIVRFPNITNLCPFGEVENAT | |
| | RFASVYAWNRKRISNCVADYSVLYNSASFSTFKCYGVSPTKL | |
| | NDLCFTNVYADSFVIRGDEVRQIAPGQTGKIADYNYKLPDDFT | |
| | GCVIAWNSNNLDSKVGGNYNYRYRLFRKSNLKPFERDISTEIY | |
| | QAGSTPCNGVQGFNCYFPLQSYGFQPTNGVGYQPYRVVVLSF | |
| | ELLHAPATVCGPKKSTNLVKNKCVNFNFNGLTGTGVLTESNK | |
| | KFLPFQQFGRDIADTTDAVRDPQTLEILDITPCSFGGVSVITPGT | |
| | NTSNQVAVLYQGVNCTEVPVAIHADQLTPTWRVYSTGSNVF | |
| | QTRAGCLIGAEHVNNSYECDIPIGAGICASYQTQTNSRRRARS | |
| | VASQSIIAYTMSLGAENSVAYSNNSIAIPTNFTISVTTEILPVSM | |
| | TKTSVDCTMYICGDSTECSNLLLQYGSFCTQLNRALTGIAVEQ | |
| | DKNTQEVFAQVKQIYKTPPIKDFGGFNFSQILPDPSKPSKRSFIE | |
| | DLLFNKVTLADAGFIKQYGDCLGDIAARDLICAQKFNGLTVLP | |
| | PLLTDEMIAQYTSALLAGTITSGWTFGAGAALQIPFAMQMAY | |
| | RFNGIGVTQNVLYENQKLIANQFNSAIGKIQDSLSSTASALGK | |
| | LQDVVNQNAQALNTLVKQLSSNFGAISSVLNDILSRLDPPEAE | |
| | VQIDRLITGRLQSLQTYVTQQLIRAAEIRASANLAATKMSECV | |
| | LGQSKRVDFCGKGYHLMSFPQSAPHGVVFLHVTYVPAHEKN | |
| | FTTAPAICHDGKAHFPREGVFVSNGTHWFVTQRNFYEPQIITT | |
| | DNTFVSGNCDVVIGIVNNTVYDPLQPELDSFKEELDKYFKNHT | |
| | SPDVDLGDISGINASVVNIQKEIDRLNEVAKNLNESLIDLQELG | |
| | KYEQYIKWPWYIWLGFIAGLIAIVMVTIMLCCMTSCCSCLKG | |
| | CCSCGSCCKFDEDDSEPVLKGVKLHYT | |
| PolyA tail | 100 nt | |

S2P_IN_B.1.617.2_[T19R, G142D, E156G, F157-, R158-, L452R,
T478K, D614G, P681R, D950N

| SEQ ID NO: 24 consists of from 5′ end to 3′ end: 5′ UTR SEQ ID NO: 2, | | 24 |
|---|---|---|
| mRNA ORF SEQ ID NO: 25, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGACCCCGGCGCCGCCACC | |
| ORF of mRNA | AUGUUCGUGUUCCUGGUGCUGCUGCCCCUGGUGAGCAGC | 25 |
| Construct | CAGUGCGUGAACCUGAGAACCCGGACCCAGCUGCCACCAG | |
| (excluding the stop | CCUACACCAACAGCUUCACCCGGGGCGUCUACUACCCCGA | |
| codon) | CAAGGUGUUCCGGAGCAGCGUCCUGCACAGCACCCAGGA | |
| | CCUGUUCCUGCCCUUCUUCAGCAACGUGACCUGGUUCCAC | |
| | GCCAUCCACGUGAGCGGCACCAACGGCACCAAGCGGUUCG | |
| | ACAACCCCGUGCUGCCCUUCAACGACGGCGUGUACUUCGC | |
| | CAGCACCGAGAAGAGCAACAUCAUCCGGGGCUGGAUCUU | |
| | CGGCACCACCCUGGACAGCAAGACCCAGAGCCUGCUGAUC | |
| | GUGAAUAACGCCACCAACGUGGUGAUCAAGGUGUGCGAG | |
| | UUCCAGUUCUGCAACGACCCCUUCCUGGACGUGUACUACC | |
| | ACAAGAACAACAAGAGCUGGAUGGAGAGCGGCGUGUACA | |
| | GCAGCGCCAACAACUGCACCUUCGAGUACGUGAGCCAGCC | |
| | CUUCCUGAUGGACCUGGAGGGCAAGCAGGGCAACUUCAA | |
| | GAACCUGCGGGAGUUCGUGUUCAAGAACAUCGACGGCUA | |
| | CUUCAAGAUCUACAGCAAGCACACCCCAAUCAACCUGGU | |
| | GCGGGAUCUGCCCCAGGGCUUCUCAGCCCUGGAGCCCCUG | |
| | GUGGACCUGCCCAUCGGCAUCAACAUCACCCGGUUCCAGA | |
| | CCCUGCUGGCCCUGCACCGGAGCUACCUGACCCCAGGCGA | |
| | CAGCAGCAGCGGGUGGACAGCAGGCGCGGCUGCUUACUA | |
| | CGUGGGCUACCUGCAGCCCCGGACCUUCCUGCUGAAGUAC | |
| | AACGAGAACGGCACCAUCACCGACGCCGUGGACUGCGCCC | |
| | UGGACCCUCUGAGCGAGACCAAGUGCACCCUGAAGAGCU | |
| | UCACCGUGGAGAAGGGCAUCUACCAGACCAGCAACUUCC | |
| | GGGUGCAGCCCACCGAGAGCAUCGUGCGGUUCCCCAACA | |
| | UCACCAACCUGUGCCCCUUCGGCGAGGUGUUCAACGCCAC | |
| | CCGGUUCGCCAGCGUGUACGCCUGGAACCGGAAGCGGAU | |
| | CAGCAACUGCGUGGCCGACUACAGCGUGCUGUACAACAG | |
| | CGCCAGCUUCAGCACCUUCAAGUGCUACGGCGUGAGCCCC | |
| | ACCAAGCUGAACGACCUGUGCUUCACCAACGUGUACGCC | |
| | GACAGCUUCGUGAUCCGUGGCGACGAGGUGCGGCAGAUC | |
| | GCACCCGGCCAGACAGGCAAGAUCGCCGACUACAACUACA | |
| | AGCUGCCCGACGACUUCACCGGCUGCGUGAUCGCCUGGA | |
| | ACAGCAACAACCUCGACAGCAAGGUGGGCGGCAACUACA | |
| | ACUACAGAUACCGGCUGUUCCGGAAGAGCAACCUGAAGC | |
| | CCUUCGAGCGGGACAUCAGCACCGAGAUCUACCAAGCCG | |
| | GCUCCAAGCCUUGCAACGGCGUGGAGGGCUUCAACUGCU | |
| | ACUUCCCUCUGCAGAGCUACGGCUUCCAGCCCACCAACGG | |
| | CGUGGGCUACCAGCCCUACCGGGUGGUGGUGCUGAGCUU | |
| | CGAGCUGCUGCACGCCCCAGCCACCGUGUGUGGCCCCAAG | |
| | AAGAGCACCAACCUGGUGAAGAACAAGUGCGUGAACUUC | |
| | AACUUCAACGGCCUUACCGGCACCGGCGUGCUGACCGAG | |
| | AGCAACAAGAAAUUCCUGCCCUUUCAGCAGUUCGGCCGG | |
| | GACAUCGCCGACACCACCGACGCUGUGCGGGAUCCCCAGA | |
| | CCCUGGAGAUCCUGGACAUCACCCCUUGCAGCUUCGGCGG | |
| | CGUGAGCGUGAUCACCCCAGGCACCAACACCAGCAACCAG | |
| | GUGGCCGUGCUGUACCAGGGCGUGAACUGCACCGAGGUG | |
| | CCCGUGGCCAUCCACGCCGACCAGCUGACACCCACCUGGC | |
| | GGGUCUACAGCACCGGCAGCAACGUGUUCCAGACCCGGG | |
| | CCGGUUGCCUGAUCGGCGCCGAGCACGUGAACAACAGCU | |
| | ACGAGUGCGACAUCCCCAUCGGCGCCGGCAUCUGUGCCAG | |
| | CUACCAGACCCAGACCAAUUCAAGACGGAGGGCAAGGAG | |
| | CGUGGCCAGCCAGAGCAUCAUCGCCUACACCAUGAGCCUG | |
| | GGCGCCGAGAACAGCGUGGCCUACAGCAACAACAGCAUC | |
| | GCCAUCCCCACCAACUUCACCAUCAGCGUGACCACCGAGA | |
| | UUCUGCCCGUGAGCAUGACCAAGACCAGCGUGGACUGCA | |
| | CCAUGUACAUCUGCGGCGACAGCACCGAGUGCAGCAACC | |
| | UGCUGCUGCAGUACGGCAGCUUCUGCACCCAGCUGAACC | |
| | GGGCCCUGACCGGCAUCGCCGUGGAGCAGGACAAGAACA | |
| | CCCAGGAGGUGUUCGCCCAGGUGAAGCAGAUCUACAAGA | |
| | CCCCUCCCAUCAAGGACUUCGGCGGCUUCAACUUCAGCCA | |
| | GAUCCUGCCCGACCCCAGCAAGCCCAGCAAGCGGAGCUUC | |
| | AUCGAGGACCUGCUGUUCAACAAGGUGACCCUAGCCGAC | |
| | GCCGGCUUCAUCAAGCAGUACGGCGACUGCCUCGGCGAC | |
| | AUAGCCGCCCGGGACCUGAUCUGCGCCCAGAAGUUCAAC | |
| | GGCCUGACCGUGCUGCCUCCCCUGCUGACCGACGAGAUGA | |
| | UCGCCCAGUACACCAGCGCCCUGUUAGCCGGAACCAUCAC | |
| | CAGCGGCUGGACUUUCGGCGCUGGAGCCGCUCUGCAGAU | |
| | CCCCUUCGCCAUGCAGAUGGCCUACCGGUUCAACGGCAUC | |
| | GGCGUGACCCAGAACGUGCUGUACGAGAACCAGAAGCUG | |
| | AUCGCCAACCAGUUCAACAGCGCCAUCGGCAAGAUCCAG | |
| | GACAGCCUGAGCAGCACCGCUAGCGCCCUGGGCAAGCUGC | |
| | AGAACGUGGUGAACCAGAACGCCCAGGCCCUGAACACCC | |
| | UGGUGAAGCAGCUGAGCAGCAACUUCGGCGCCAUCAGCA | |
| | GCGUGCUGAACGACAUCCUGAGCCGGCUGGACCCUCCCGA | |
| | GGCCGAGGUGCAGAUCGACCGGCUGAUCACUGGCCGGCU | |
| | GCAGAGCCUGCAGACCUACGUGACCCAGCAGCUGAUCCG | |
| | GGCCGCCGAGAUUCGGGCCAGCGCCAACCUGGCCGCCACC | |
| | AAGAUGAGCGAGUGCGUGCUGGGCCAGAGCAAGCGGGUG | |
| | GACUUCUGCGGCAAGGGCUACCACCUGAUGAGCUUUCCC | |
| | CAGAGCGCACCCCACGGAGUGGUGUUCCUGCACGUGACC | |
| | UACGUGCCCGCCCAGGAGAAGAACUUCACCACCGCCCCAG | |
| | CCAUCUGCCACGACGGCAAGGCCCACUUUCCCCGGGAGGG | |
| | CGUGUUCGUGAGCAACGGCACCCACUGGUUCGUGACCCA | |
| | GCGGAACUUCUACGAGCCCCAGAUCAUCACCACCGACAAC | |
| | ACCUUCGUGAGCGGCAACUGCGACGUGGUGAUCGGCAUC | |
| | GUGAACAACACCGUGUACGAUCCCCUGCAGCCCGAGCUG | |
| | GACAGCUUCAAGGAGGAGCUGGACAAGUACUUCAAGAAU | |
| | CACACCAGCCCCGACGUGGACCUGGGCGACAUCAGCGGCA | |
| | UCAACGCCAGCGUGGUGAACAUCCAGAAGGAGAUCGAUC | |
| | GGCUGAACGAGGUGGCCAAGAACCUGAACGAGAGCCUGA | |
| | UCGACCUGCAGGAGCUGGGCAAGUACGAGCAGUACAUCA | |
| | AGUGGCCCUGGUACAUCUGGCUGGGCUUCAUCGCCGGCC | |
| | UGAUCGCCAUCGUGAUGGUGACCAUCAUGCUGUGCUGCA | |
| | UGACCAGCUGCUGCAGCUGCCUGAAGGGCUGUUGCAGCU | |
| | GCGGCAGCUGCUGCAAGUUCGACGAGGACGACAGCGAGC | |
| | CCGUGCUGAAGGGCGUGAAGCUGCACUACACC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MFVFLVLLPLVSSQCVNLRTRTQLPPAYTNSFTRGVYYPDKVF | 26 |
| acid sequence | RSSVLHSTQDLFLPFFSNVTWFHAIHVSGTNGTKRFDNPVLPF | |
| | NDGVYFASTEKSNIIRGWIFGTTLDSKTQSLLIVNNATNVVIKV | |
| | CEFQFCNDPFLDVYYHKNNKSWMESGVYSSANNCTFEYVSQ | |
| | PFLMDLEGKQGNFKNLREFVFKNIDGYFKIYSKHTPINLVRDL | |
| | PQGFSALEPLVDLPIGINITRFQTLLALHRSYLTPGDSSSGWTA | |
| | GAAAYYVGYLQPRTFLLKYNENGTITDAVDCALDPLSETKCT | |
| | LKSFTVEKGIYQTSNFRVQPTESIVRFPNITNLCPFGEVFNATRF | |
| | ASVYAWNRKRISNCVADYSVLYNSASFSTFKCYGVSPTKLND | |
| | LCFTNVYADSFVIRGDEVRQIAPGQTGKIADYNYKLPDDFTGC | |
| | VIAWNSNNLDSKVGGNYNYRYRLFRKSNLKPFERDISTEIYQA | |
| | GSKPCNGVEGFNCYFPLQSYGFQPTNGVGYQPYRVVVLSFEL | |
| | LHAPATVCGPKKSTNLVKNKCVNFNFNGLTGTGVLTESNKKF | |
| | LPFQQFGRDIADTTDAVRDPQTLEILDITPCSFGGVSVITPGTNT | |
| | SNQVAVLYQGVNCTEVPVAIHADQLTPTWRVYSTGSNVFQT | |
| | RAGCLIGAEHVNNSYECDIPIGAGICASYQTQTNSRRRARSVA | |
| | SQSIIAYTMSLGAENSVAYSNNSIAIPTNFTISVTTEILPVSMTK | |
| | TSVDCTMYICGDSTECSNLLLQYGSFCTQLNRALTGIAVEQDK | |
| | NTQEVFAQVKQIYKTPPIKDFGGFNFSQILPDPSKPSKRSFIEDL | |
| | LFNKVTLADAGFIKQYGDCLGDIAARDLICAQKFNGLTVLPPL | |
| | LTDEMIAQYTSALLAGTITSGWTFGAGAALQIPFAMQMAYRF | |
| | NGIGVTQNVLYENQKLIANQFNSAIGKIQDSLSSTASALGKLQ | |
| | NVVNQNAQALNTLVKQLSSNFGAISSVLNDILSRLDPPEAEVQ | |
| | IDRLITGRLQSLQTYVTQQLIRAAEIRASANLAATKMSECVLG | |
| | QSKRVDFCGKGYHLMSFPQSAPHGVVFLHVTYVPAQEKNFTT | |
| | APAICHDGKAHFPREGVFVSNGTHWFVTQRNFYEPQIITTDNT | |
| | FVSGNCDVVIGIVNNTVYDPLQPELDSFKEELDKYFKNHTSPD | |
| | VDLGDISGINASVVNIQKEIDRLNEVAKNLNESLIDLQELGKYE | |
| | QYIKWPWYIWLGFIAGLIAIVMVTIMLCCMTSCCSCLKGCCSC | |
| | GSCCKFDEDDSEPVLKGVKLHYT | |
| PolyA tail | 100 nt | |
| WH2020_NatSP | MFVFLVLLPLVSSQCVNLTTRTQLPPAYTNSFTRGVYYPDKVF | 27 |
| | RSSVLHSTQDLFLPFFSNVTWFHAIHVSGTNGTKRFDNPVLPF | |
| | NDGVYFASTEKSNIIRGWIFGTTLDSKTQSLLIVNNATNVVIKV | |
| | CEFQFCNDPFLGVYYHKNNKSWMESEFRVYSSANNCTFEYVS | |
| | QPFLMDLEGKQGNFKNLREFVFKNIDGYFKIYSKHTPINLVRD | |
| | LPQGFSALEPLVDLPIGINITRFQTLLALHRSYLTPGDSSSGWT | |
| | AGAAAYYVGYLQPRTFLLKYNENGTITDAVDCALDPLSETKC | |
| | TLKSFTVEKGIYQTSNFRVQPTESIVRFPNITNLCPFGEVENATR | |
| | FASVYAWNRKRISNCVADYSVLYNSASFSTFKCYGVSPTKLN | |
| | DLCFTNVYADSFVIRGDEVRQIAPGQTGKIADYNYKLPDDFTG | |
| | CVIAWNSNNLDSKVGGNYNYLYRLFRKSNLKPFERDISTEIYQ | |
| | AGSTPCNGVEGENCYFPLQSYGFQPTNGVGYQPYRVVVLSFE | |
| | LLHAPATVCGPKKSTNLVKNKCVNFNFNGLTGTGVLTESNKK | |
| | FLPFQQFGRDIADTTDAVRDPQTLEILDITPCSFGGVSVITPGTN | |
| | TSNQVAVLYQDVNCTEVPVAIHADQLTPTWRVYSTGSNVFQT | |
| | RAGCLIGAEHVNNSYECDIPIGAGICASYQTQTNSPRRARSVA | |
| | SQSIIAYTMSLGAENSVAYSNNSIAIPTNFTISVTTEILPVSMTK | |
| | TSVDCTMYICGDSTECSNLLLQYGSFCTQLNRALTGIAVEQDK | |
| | NTQEVFAQVKQIYKTPPIKDFGGFNFSQILPDPSKPSKRSFIEDL | |
| | LFNKVTLADAGFIKQYGDCLGDIAARDLICAQKFNGLTVLPPL | |
| | LTDEMIAQYTSALLAGTITSGWTFGAGAALQIPFAMQMAYRF | |
| | NGIGVTQNVLYENQKLIANQFNSAIGKIQDSLSSTASALGKLQ | |
| | DVVNQNAQALNTLVKQLSSNFGAISSVLNDILSRLDKVEAEV | |
| | QIDRLITGRLQSLQTYVTQQLIRAAEIRASANLAATKMSECVL | |
| | GQSKRVDFCGKGYHLMSFPQSAPHGVVFLHVTYVPAQEKNF | |
| | TTAPAICHDGKAHFPREGVFVSNGTHWFVTQRNFYEPQIITTD | |
| | NTFVSGNCDVVIGIVNNTVYDPLQPELDSFKEELDKYFKNHTS | |
| | PDVDLGDISGINASVVNIQKEIDRLNEVAKNLNESLIDLQELGK | |
| | YEQYIKWPWYIWLGFIAGLIAIVMVTIMLCCMTSCCSCLKGCC | |
| | SCGSCCKFDEDDSEPVLKGVKLHYT | |

S2P_IN_B.1.617.2_[T19R, T95I, G142D, E156G, F157-, R158-, L452R, T478K,
D614G, P681R, D950N]

| SEQ ID NO: 28 consists of from 5′ end to 3′ end: 5′ UTR SEQ ID NO: 2, | | 28 |
|---|---|---|
| mRNA ORF SEQ ID NO: 29, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGUUCGUGUUCCUGGUGCUGCUGCCCCUGGUGAGCAGC | 29 |
| Construct | CAGUGCGUGAACCUGAGAACCCGGACCCAGCUGCCACCAG | |
| (excluding the stop | CCUACACCAACAGCUUCACCCGGGGCGUCUACUACCCCGA | |
| codon) | CAAGGUGUUCCGGAGCAGCGUCCUGCACAGCACCCAGGAC | |
| | CUGUUCCUGCCCUUCUUCAGCAACGUGACCUGGUUCCACG | |
| | CCAUCCACGUGAGCGGCACCAACGGCACCAAGCGGUUCGA | |
| | CAACCCCGUGCUGCCCUUCAACGACGGCGUGUACUUCGCC | |
| | AGCAUCGAGAAGAGCAACAUCAUCCGGGGCUGGAUCUUC | |
| | GGCACCACCCUGGACAGCAAGACCCAGAGCCUGCUGAUCG | |
| | UGAAUAACGCCACCAACGUGGUGAUCAAGGUGUGCGAGU | |
| | UCCAGUUCUGCAACGACCCCUUCCUGGACGUGUACUACCA | |
| | CAAGAACAACAAGAGCUGGAUGGAGAGCGGCGUGUACAG | |
| | CAGCGCCAACAACUGCACCUUCGAGUACGUGAGCCAGCCC | |
| | UUCCUGAUGGACCUGGAGGGCAAGCAGGGCAACUUCAAG | |
| | AACCUGCGGGAGUUCGUGUUCAAGAACAUCGACGGCUAC | |
| | UUCAAGAUCUACAGCAAGCACACCCCAAUCAACCUGGUGC | |
| | GGGAUCUGCCCCAGGGCUUCUCAGCCCUGGAGCCCCUGGU | |
| | GGACCUGCCCAUCGGCAUCAACAUCACCCGGUUCCAGACC | |
| | CUGCUGGCCCUGCACCGGAGCUACCUGACCCCAGGCGACA | |
| | GCAGCAGCGGGUGGACAGCAGGCGCGGCUGCUUACUACG | |
| | UGGGCUACCUGCAGCCCCGGACCUUCCUGCUGAAGUACAA | |
| | CGAGAACGGCACCAUCACCGACGCCGUGGACUGCGCCCUG | |
| | GACCCUCUGAGCGAGACCAAGUGCACCCUGAAGAGCUUCA | |
| | CCGUGGAGAAGGGCAUCUACCAGACCAGCAACUUCCGGG | |
| | UGCAGCCCACCGAGAGCAUCGUGCGGUUCCCCAACAUCAC | |
| | CAACCUGUGCCCCUUCGGCGAGGUGUUCAACGCCACCCGG | |
| | UUCGCCAGCGUGUACGCCUGGAACCGGAAGCGGAUCAGC | |
| | AACUGCGUGGCCGACUACAGCGUGCUGUACAACAGCGCCA | |
| | GCUUCAGCACCUUCAAGUGCUACGGCGUGAGCCCCACCAA | |
| | GCUGAACGACCUGUGCUUCACCAACGUGUACGCCGACAGC | |
| | UUCGUGAUCCGUGGCGACGAGGUGCGGCAGAUCGCACCC | |
| | GGCCAGACAGGCAAGAUCGCCGACUACAACUACAAGCUGC | |
| | CCGACGACUUCACCGGCUGCGUGAUCGCCUGGAACAGCAA | |
| | CAACCUCGACAGCAAGGUGGGGGCAACUACAACUACAG | |
| | AUACCGGCUGUUCCGGAAGAGCAACCUGAAGCCCUUCGA | |
| | GCGGGACAUCAGCACCGAGAUCUACCAAGCCGGCUCCAAG | |
| | CCUUGCAACGGCGUGGAGGGCUUCAACUGCUACUUCCCUC | |
| | UGCAGAGCUACGGCUUCCAGCCCACCAACGGCGUGGGCUA | |
| | CCAGCCCUACCGGGUGGUGGUGCUGAGCUUCGAGCUGCU | |
| | GCACGCCCCAGCCACCGUGUGUGGCCCCAAGAAGAGCACC | |
| | AACCUGGUGAAGAACAAGUGCGUGAACUUCAACUUCAAC | |
| | GGCCUUACCGGCACCGGCGUGCUGACCGAGAGCAACAAGA | |
| | AAUUCCUGCCCUUUCAGCAGUUCGGCCGGGACAUCGCCGA | |
| | CACCACCGACGCUGUGCGGGAUCCCCAGACCCUGGAGAUC | |
| | CUGGACAUCACCCCUUGCAGCUUCGGCGGCGUGAGCGUGA | |
| | UCACCCCAGGCACCAACACCAGCAACCAGGUGGCCGUGCU | |
| | GUACCAGGGCGUGAACUGCACCGAGGUGCCCGUGGCCAUC | |
| | CACGCCGACCAGCUGACACCCACCUGGCGGGUCUACAGCA | |
| | CCGGCAGCAACGUGUUCCAGACCCGGGCCGGUUGCCUGAU | |
| | CGGCGCCGAGCACGUGAACAACAGCUACGAGUGCGACAUC | |
| | CCCAUCGGCGCCGGCAUCUGUGCCAGCUACCAGACCCAGA | |
| | CCAAUUCAAGACGGAGGGCAAGGAGCGUGGCCAGCCAGA | |
| | GCAUCAUCGCCUACACCAUGAGCCUGGGCGCCGAGAACAG | |
| | CGUGGCCUACAGCAACAACAGCAUCGCCAUCCCCACCAAC | |
| | UUCACCAUCAGCGUGACCACCGAGAUUCUGCCCGUGAGCA | |
| | UGACCAAGACCAGCGUGGACUGCACCAUGUACAUCUGCG | |
| | GCGACAGCACCGAGUGCAGCAACCUGCUGCUGCAGUACGG | |
| | CAGCUUCUGCACCCAGCUGAACCGGGCCCUGACCGGCAUC | |
| | GCCGUGGAGCAGGACAAGAACACCCAGGAGGUGUUCGCC | |
| | CAGGUGAAGCAGAUCUACAAGACCCCUCCCAUCAAGGACU | |
| | UCGGCGGCUUCAACUUCAGCCAGAUCCUGCCCGACCCCAG | |
| | CAAGCCCAGCAAGCGGAGCUUCAUCGAGGACCUGCUGUUC | |
| | AACAAGGUGACCCUAGCCGACGCCGGCUUCAUCAAGCAGU | |
| | ACGGCGACUGCCUCGGCGACAUAGCCGCCCGGGACCUGAU | |
| | CUGCGCCCAGAAGUUCAACGGCCUGACCGUGCUGCCUCCC | |
| | CUGCUGACCGACGAGAUGAUCGCCCAGUACACCAGCGCCC | |
| | UGUUAGCCGGAACCAUCACCAGCGGCUGGACUUUCGGCGC | |
| | UGGAGCCGCUCUGCAGAUCCCCUUCGCCAUGCAGAUGGCC | |
| | UACCGGUUCAACGGCAUCGGCGUGACCCAGAACGUGCUG | |
| | UACGAGAACCAGAAGCUGAUCGCCAACCAGUUCAACAGC | |
| | GCCAUCGGCAAGAUCCAGGACAGCCUGAGCAGCACCGCUA | |
| | GCGCCCUGGGCAAGCUGCAGAACGUGGUGAACCAGAACG | |
| | CCCAGGCCCUGAACACCCUGGUGAAGCAGCUGAGCAGCAA | |
| | CUUCGGCGCCAUCAGCAGCGUGCUGAACGACAUCCUGAGC | |
| | CGGCUGGACCCUCCCGAGGCCGAGGUGCAGAUCGACCGGC | |
| | UGAUCACUGGCCGGCUGCAGAGCCUGCAGACCUACGUGAC | |
| | CCAGCAGCUGAUCCGGGCCGCCGAGAUUCGGGCCAGCGCC | |
| | AACCUGGCCGCCACCAAGAUGAGCGAGUGCGUGCUGGGCC | |
| | AGAGCAAGCGGGUGGACUUCUGCGGCAAGGGCUACCACC | |
| | UGAUGAGCUUUCCCCAGAGCGCACCCCACGGAGUGGUGU | |
| | UCCUGCACGUGACCUACGUGCCCGCCCAGGAGAAGAACUU | |
| | CACCACCGCCCCAGCCAUCUGCCACGACGGCAAGGCCCAC | |
| | UUUCCCCGGGAGGGCGUGUUCGUGAGCAACGGCACCCACU | |
| | GGUUCGUGACCCAGCGGAACUUCUACGAGCCCCAGAUCAU | |
| | CACCACCGACAACACCUUCGUGAGCGGCAACUGCGACGUG | |
| | GUGAUCGGCAUCGUGAACAACACCGUGUACGAUCCCCUGC | |
| | AGCCCGAGCUGGACAGCUUCAAGGAGGAGCUGGACAAGU | |
| | ACUUCAAGAAUCACACCAGCCCCGACGUGGACCUGGGCGA | |
| | CAUCAGCGGCAUCAACGCCAGCGUGGUGAACAUCCAGAA | |
| | GGAGAUCGAUCGGCUGAACGAGGUGGCCAAGAACCUGAA | |
| | CGAGAGCCUGAUCGACCUGCAGGAGCUGGGCAAGUACGA | |
| | GCAGUACAUCAAGUGGCCCUGGUACAUCUGGCUGGGCUU | |
| | CAUCGCCGGCCUGAUCGCCAUCGUGAUGGUGACCAUCAUG | |
| | CUGUGCUGCAUGACCAGCUGCUGCAGCUGCCUGAAGGGC | |
| | UGUUGCAGCUGCGGCAGCUGCUGCAAGUUCGACGAGGAC | |
| | GACAGCGAGCCCGUGCUGAAGGGCGUGAAGCUGCACUAC | |
| | ACC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MFVFLVLLPLVSSQCVNLRTRTQLPPAYTNSFTRGVYYPDKVF | 30 |
| acid sequence | RSSVLHSTQDLFLPFFSNVTWFHAIHVSGTNGTKRFDNPVLPF | |
| | NDGVYFASIEKSNIIRGWIFGTTLDSKTQSLLIVNNATNVVIKV | |
| | CEFQFCNDPFLDVYYHKNNKSWMESGVYSSANNCTFEYVSQ | |
| | PFLMDLEGKQGNFKNLREFVFKNIDGYFKIYSKHTPINLVRDL | |
| | PQGFSALEPLVDLPIGINITRFQTLLALHRSYLTPGDSSSGWTA | |
| | GAAAYYVGYLQPRTFLLKYNENGTITDAVDCALDPLSETKCT | |
| | LKSFTVEKGIYQTSNFRVQPTESIVRFPNITNLCPFGEVENATRF | |
| | ASVYAWNRKRISNCVADYSVLYNSASFSTFKCYGVSPTKLND | |
| | LCFTNVYADSFVIRGDEVRQIAPGQTGKIADYNYKLPDDFTGC | |
| | VIAWNSNNLDSKVGGNYNYRYRLFRKSNLKPFERDISTEIYQA | |
| | GSKPCNGVEGENCYFPLQSYGFQPTNGVGYQPYRVVVLSFEL | |
| | LHAPATVCGPKKSTNLVKNKCVNFNFNGLTGTGVLTESNKKF | |
| | LPFQQFGRDIADTTDAVRDPQTLEILDITPCSFGGVSVITPGTNT | |
| | SNQVAVLYQGVNCTEVPVAIHADQLTPTWRVYSTGSNVFQTR | |
| | AGCLIGAEHVNNSYECDIPIGAGICASYQTQTNSRRRARSVAS | |
| | QSIIAYTMSLGAENSVAYSNNSIAIPTNFTISVTTEILPVSMTKTS | |
| | VDCTMYICGDSTECSNLLLQYGSFCTQLNRALTGIAVEQDKNT | |
| | QEVFAQVKQIYKTPPIKDFGGFNFSQILPDPSKPSKRSFIEDLLF | |
| | NKVTLADAGFIKQYGDCLGDIAARDLICAQKFNGLTVLPPLLT | |
| | DEMIAQYTSALLAGTITSGWTFGAGAALQIPFAMQMAYRENG | |
| | IGVTQNVLYENQKLIANQFNSAIGKIQDSLSSTASALGKLQNV | |
| | VNQNAQALNTLVKQLSSNFGAISSVLNDILSRLDPPEAEVQIDR | |
| | LITGRLQSLQTYVTQQLIRAAEIRASANLAATKMSECVLGQSK | |
| | RVDFCGKGYHLMSFPQSAPHGVVFLHVTYVPAQEKNFTTAPA | |
| | ICHDGKAHFPREGVFVSNGTHWFVTQRNFYEPQIITTDNTFVS | |
| | GNCDVVIGIVNNTVYDPLQPELDSFKEELDKYFKNHTSPDVDL | |
| | GDISGINASVVNIQKEIDRLNEVAKNLNESLIDLQELGKYEQYI | |
| | KWPWYIWLGFIAGLIAIVMVTIMLCCMTSCCSCLKGCCSCGSC | |
| | CKFDEDDSEPVLKGVKLHYT | |
| PolyA tail | 100 nt | |

Botswana-variant-1273

| SEQ ID NO: 31 consists of from 5′ end to 3′ end: 5′ UTR SEQ ID NO: 2, | | 31 |
|---|---|---|
| mRNA ORF SEQ ID NO: 32, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGUUCGUGUUCCUGGUGCUGCUGCCCCUGGUGAGCAGC | 32 |
| Construct | CAGUGCGUGAACCUGACCACCCGGACCCAGCUGCCACCAG | |
| (excluding the stop | CCUACACCAACAGCUUCACCCGGGGCGUCUACUACCCCGA | |
| codon) | CAAGGUGUUCCGGAGCAGCGUCCUGCACAGCACCCAGGAC | |
| | CUGUUCCUGCCCUUCUUCAGCAACGUGACCUGGUUCCACG | |
| | UGAUCAGCGGCACCAACGGCACCAAGCGGUUCGACAACCC | |
| | CGUGCUGCCCUUCAACGACGGCGUGUACUUCGCCAGCAUC | |
| | GAGAAGAGCAACAUCAUCCGGGGCUGGAUCUUCGGCACC | |
| | ACCCUGGACAGCAAGACCCAGAGCCUGCUGAUCGUGAAU | |
| | AACGCCACCAACGUGGUGAUCAAGGUGUGCGAGUUCCAG | |
| | UUCUGCAACGACCCCUUCCUGGACCACAAGAACAACAAGA | |
| | GCUGGAUGGAGAGCGAGUUCCGGGUGUACAGCAGCGCCA | |
| | ACAACUGCACCUUCGAGUACGUGAGCCAGCCCUUCCUGAU | |
| | GGACCUGGAGGGCAAGCAGGGCAACUUCAAGAACCUGCG | |
| | GGAGUUCGUGUUCAAGAACAUCGACGGCUACUUCAAGAU | |
| | CUACAGCAAGCACACCCCAAUCAUCGUGCGGGAGCCCGAG | |
| | GAUCUGCCCCAGGGCUUCUCAGCCCUGGAGCCCCUGGUGG | |
| | ACCUGCCCAUCGGCAUCAACAUCACCCGGUUCCAGACCCU | |
| | GCUGGCCCUGCACCGGAGCUACCUGACCCCAGGCGACAGC | |
| | AGCAGCGGGUGGACAGCAGGCGCGGCUGCUUACUACGUG | |
| | GGCUACCUGCAGCCCCGGACCUUCCUGCUGAAGUACAACG | |
| | AGAACGGCACCAUCACCGACGCCGUGGACUGCGCCCUGGA | |
| | CCCUCUGAGCGAGACCAAGUGCACCCUGAAGAGCUUCACC | |
| | GUGGAGAAGGGCAUCUACCAGACCAGCAACUUCCGGGUG | |
| | CAGCCCACCGAGAGCAUCGUGCGGUUCCCCAACAUCACCA | |
| | ACCUGUGCCCCUUCGACGAGGUGUUCAACGCCACCCGGUU | |
| | CGCCAGCGUGUACGCCUGGAACCGGAAGCGGAUCAGCAAC | |
| | UGCGUGGCCGACUACAGCGUGCUGUACAACCUGGCCCCCU | |
| | UCUUCACCUUCAAGUGCUACGGCGUGAGCCCCACCAAGCU | |
| | GAACGACCUGUGCUUCACCAACGUGUACGCCGACAGCUUC | |
| | GUGAUCCGUGGCGACGAGGUGCGGCAGAUCGCACCCGGCC | |
| | AGACAGGCAACAUCGCCGACUACAACUACAAGCUGCCCGA | |
| | CGACUUCACCGGCUGCGUGAUCGCCUGGAACAGCAACAAG | |
| | CUCGACAGCAAGGUGAGCGGCAACUACAACUACCUGUACC | |
| | GGCUGUUCCGGAAGAGCAACCUGAAGCCCUUCGAGCGGG | |
| | ACAUCAGCACCGAGAUCUACCAAGCCGGCAACAAGCCUUG | |
| | CAACGGCGUGGCCGGCUUCAACUGCUACUUCCCUCUGCGG | |
| | AGCUACAGCUUCCGGCCCACCUACGGCGUGGGCCACCAGC | |
| | CCUACCGGGUGGUGGUGCUGAGCUUCGAGCUGCUGCACG | |
| | CCCCAGCCACCGUGUGUGGCCCCAAGAAGAGCACCAACCU | |
| | GGUGAAGAACAAGUGCGUGAACUUCAACUUCAACGGCCU | |
| | UAAGGGCACCGGCGUGCUGACCGAGAGCAACAAGAAAUU | |
| | CCUGCCCUUUCAGCAGUUCGGCCGGGACAUCGCCGACACC | |
| | ACCGACGCUGUGCGGGAUCCCCAGACCCUGGAGAUCCUGG | |
| | ACAUCACCCCUUGCAGCUUCGGCGGCGUGAGCGUGAUCAC | |
| | CCCAGGCACCAACACCAGCAACCAGGUGGCCGUGCUGUAC | |
| | CAGGGCGUGAACUGCACCGAGGUGCCCGUGGCCAUCCACG | |
| | CCGACCAGCUGACACCCACCUGGCGGGUCUACAGCACCGG | |
| | CAGCAACGUGUUCCAGACCCGGGCCGGUUGCCUGAUCGGC | |
| | GCCGAGUACGUGAACAACAGCUACGAGUGCGACAUCCCCA | |
| | UCGGCGCCGGCAUCUGUGCCAGCUACCAGACCCAGACCAA | |
| | GUCACACCGGAGGGCAAGGAGCGUGGCCAGCCAGAGCAU | |
| | CAUCGCCUACACCAUGAGCCUGGGCGCCGAGAACAGCGUG | |
| | GCCUACAGCAACAACAGCAUCGCCAUCCCCACCAACUUCA | |
| | CCAUCAGCGUGACCACCGAGAUUCUGCCCGUGAGCAUGAC | |
| | CAAGACCAGCGUGGACUGCACCAUGUACAUCUGCGGCGAC | |
| | AGCACCGAGUGCAGCAACCUGCUGCUGCAGUACGGCAGCU | |
| | UCUGCACCCAGCUGAAGCGGGCCCUGACCGGCAUCGCCGU | |
| | GGAGCAGGACAAGAACACCCAGGAGGUGUUCGCCCAGGU | |
| | GAAGCAGAUCUACAAGACCCCUCCCAUCAAGUACUUCGGC | |
| | GGCUUCAACUUCAGCCAGAUCCUGCCCGACCCCAGCAAGC | |
| | CCAGCAAGCGGAGCUUCAUCGAGGACCUGCUGUUCAACA | |
| | AGGUGACCCUAGCCGACGCCGGCUUCAUCAAGCAGUACGG | |
| | CGACUGCCUCGGCGACAUAGCCGCCCGGGACCUGAUCUGC | |
| | GCCCAGAAGUUCAAGGGCCUGACCGUGCUGCCUCCCCUGC | |
| | UGACCGACGAGAUGAUCGCCCAGUACACCAGCGCCCUGUU | |
| | AGCCGGAACCAUCACCAGCGGCUGGACUUUCGGCGCUGGA | |
| | GCCGCUCUGCAGAUCCCCUUCGCCAUGCAGAUGGCCUACC | |
| | GGUUCAACGGCAUCGGCGUGACCCAGAACGUGCUGUACG | |
| | AGAACCAGAAGCUGAUCGCCAACCAGUUCAACAGCGCCAU | |
| | CGGCAAGAUCCAGGACAGCCUGAGCAGCACCGCUAGCGCC | |
| | CUGGGCAAGCUGCAGGACGUGGUGAACCACAACGCCCAG | |
| | GCCCUGAACACCCUGGUGAAGCAGCUGAGCAGCAAGUUC | |
| | GGCGCCAUCAGCAGCGUGCUGAACGACAUCUUCAGCCGGC | |
| | UGGACCCUCCCGAGGCCGAGGUGCAGAUCGACCGGCUGAU | |
| | CACUGGCCGGCUGCAGAGCCUGCAGACCUACGUGACCCAG | |
| | CAGCUGAUCCGGGCCGCCGAGAUUCGGGCCAGCGCCAACC | |
| | UGGCCGCCACCAAGAUGAGCGAGUGCGUGCUGGGCCAGA | |
| | GCAAGCGGGUGGACUUCUGCGGCAAGGGCUACCACCUGA | |
| | UGAGCUUUCCCCAGAGCGCACCCCACGGAGUGGUGUUCCU | |
| | GCACGUGACCUACGUGCCCGCCCAGGAGAAGAACUUCACC | |
| | ACCGCCCCAGCCAUCUGCCACGACGGCAAGGCCCACUUUC | |
| | CCCGGGAGGGCGUGUUCGUGAGCAACGGCACCCACUGGU | |
| | UCGUGACCCAGCGGAACUUCUACGAGCCCCAGAUCAUCAC | |
| | CACCGACAACACCUUCGUGAGCGGCAACUGCGACGUGGUG | |
| | AUCGGCAUCGUGAACAACACCGUGUACGAUCCCCUGCAGC | |
| | CCGAGCUGGACAGCUUCAAGGAGGAGCUGGACAAGUACU | |
| | UCAAGAAUCACACCAGCCCCGACGUGGACCUGGGCGACAU | |
| | CAGCGGCAUCAACGCCAGCGUGGUGAACAUCCAGAAGGA | |
| | GAUCGAUCGGCUGAACGAGGUGGCCAAGAACCUGAACGA | |
| | GAGCCUGAUCGACCUGCAGGAGCUGGGCAAGUACGAGCA | |
| | GUACAUCAAGUGGCCCUGGUACAUCUGGCUGGGCUUCAU | |
| | CGCCGGCCUGAUCGCCAUCGUGAUGGUGACCAUCAUGCUG | |
| | UGCUGCAUGACCAGCUGCUGCAGCUGCCUGAAGGGCUGU | |
| | UGCAGCUGCGGCAGCUGCUGCAAGUUCGACGAGGACGAC | |
| | AGCGAGCCCGUGCUGAAGGGCGUGAAGCUGCACUACACC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MFVFLVLLPLVSSQCVNLTTRTQLPPAYTNSFTRGVYYPDKVF | 33 |
| acid sequence | RSSVLHSTQDLFLPFFSNVTWFHVISGTNGTKRFDNPVLPEND | |
| | GVYFASIEKSNIIRGWIFGTTLDSKTQSLLIVNNATNVVIKVCEF | |
| | QFCNDPFLDHKNNKSWMESEFRVYSSANNCTFEYVSQPFLMD | |
| | LEGKQGNFKNLREFVFKNIDGYFKIYSKHTPIIVREPEDLPQGF | |
| | SALEPLVDLPIGINITRFQTLLALHRSYLTPGDSSSGWTAGAAA | |
| | YYVGYLQPRTFLLKYNENGTITDAVDCALDPLSETKCTLKSFT | |
| | VEKGIYQTSNFRVQPTESIVRFPNITNLCPFDEVFNATRFASVY | |
| | AWNRKRISNCVADYSVLYNLAPFFTFKCYGVSPTKLNDLCFT | |
| | NVYADSFVIRGDEVRQIAPGQTGNIADYNYKLPDDFTGCVIA | |
| | WNSNKLDSKVSGNYNYLYRLFRKSNLKPFERDISTEIYQAGN | |
| | KPCNGVAGENCYFPLRSYSFRPTYGVGHQPYRVVVLSFELLH | |
| | APATVCGPKKSTNLVKNKCVNFNFNGLKGTGVLTESNKKFLP | |
| | FQQFGRDIADTTDAVRDPQTLEILDITPCSFGGVSVITPGTNTSN | |
| | QVAVLYQGVNCTEVPVAIHADQLTPTWRVYSTGSNVFQTRA | |
| | GCLIGAEYVNNSYECDIPIGAGICASYQTQTKSHRRARSVASQS | |
| | IIAYTMSLGAENSVAYSNNSIAIPTNFTISVTTEILPVSMTKTSV | |
| | DCTMYICGDSTECSNLLLQYGSFCTQLKRALTGIAVEQDKNTQ | |
| | EVFAQVKQIYKTPPIKYFGGFNFSQILPDPSKPSKRSFIEDLLEN | |
| | KVTLADAGFIKQYGDCLGDIAARDLICAQKFKGLTVLPPLLTD | |
| | EMIAQYTSALLAGTITSGWTFGAGAALQIPFAMQMAYRENGI | |
| | GVTQNVLYENQKLIANQFNSAIGKIQDSLSSTASALGKLQDVV | |
| | NHNAQALNTLVKQLSSKFGAISSVLNDIFSRLDPPEAEVQIDRL | |
| | ITGRLQSLQTYVTQQLIRAAEIRASANLAATKMSECVLGQSKR | |
| | VDFCGKGYHLMSFPQSAPHGVVFLHVTYVPAQEKNFTTAPAI | |
| | CHDGKAHFPREGVFVSNGTHWFVTQRNFYEPQIITTDNTFVSG | |
| | NCDVVIGIVNNTVYDPLQPELDSFKEELDKYFKNHTSPDVDLG | |
| | DISGINASVVNIQKEIDRLNEVAKNLNESLIDLQELGKYEQYIK | |
| | WPWYIWLGFIAGLIAIVMVTIMLCCMTSCCSCLKGCCSCGSCC | |
| | KFDEDDSEPVLKGVKLHYT | |
| PolyA tail | 100 nt | |

mRNA-1273.B.1.1.529_PBSko

| SEQ ID NO: 34 consists of from 5′ end to 3′ end: 5′ UTR SEQ ID NO: 2, | | 34 |
|---|---|---|
| mRNA ORF SEQ ID NO: 35, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGUUCGUGUUCCUGGUGCUGCUGCCCCUGGUGAGCAGC | 35 |
| Construct | CAGUGCGUGAACCUGACCACCCGGACCCAGCUGCCACCAG | |
| (excluding the stop | CCUACACCAACAGCUUCACCCGGGGCGUCUACUACCCCGA | |
| codon) | CAAGGUGUUCCGGAGCAGCGUCCUGCACAGCACCCAGGAC | |
| | CUGUUCCUGCCCUUCUUCAGCAACGUGACCUGGUUCCACG | |
| | UGAUCAGCGGCACCAACGGCACCAAGCGGUUCGACAACCC | |
| | CGUGCUGCCCUUCAACGACGGCGUGUACUUCGCCAGCAUC | |
| | GAGAAGAGCAACAUCAUCCGGGGCUGGAUCUUCGGCACC | |
| | ACCCUGGACAGCAAGACCCAGAGCCUGCUGAUCGUGAAU | |
| | AACGCCACCAACGUGGUGAUCAAGGUGUGCGAGUUCCAG | |
| | UUCUGCAACGACCCCUUCCUGGACCACAAGAACAACAAGA | |
| | GCUGGAUGGAGAGCGAGUUCCGGGUGUACAGCAGCGCCA | |
| | ACAACUGCACCUUCGAGUACGUGAGCCAGCCCUUCCUGAU | |
| | GGACCUGGAGGGCAAGCAGGGCAACUUCAAGAACCUGCG | |
| | GGAGUUCGUGUUCAAGAACAUCGACGGCUACUUCAAGAU | |
| | CUACAGCAAGCACACCCCAAUCAUCGUGCGGGAGCCCGAG | |
| | GAUCUGCCCCAGGGCUUCUCAGCCCUGGAGCCCCUGGUGG | |
| | ACCUGCCCAUCGGCAUCAACAUCACCCGGUUCCAGACCCU | |
| | GCUGGCCCUGCACCGGAGCUACCUGACCCCAGGCGACAGC | |
| | AGCAGCGGGUGGACAGCAGGCGCGGCUGCUUACUACGUG | |
| | GGCUACCUGCAGCCCCGGACCUUCCUGCUGAAGUACAACG | |
| | AGAACGGCACCAUCACCGACGCCGUGGACUGCGCCCUGGA | |
| | CCCUCUGAGCGAGACCAAGUGCACCCUGAAGAGCUUCACC | |
| | GUGGAGAAGGGCAUCUACCAGACCAGCAACUUCCGGGUG | |
| | CAGCCCACCGAGAGCAUCGUGCGGUUCCCCAACAUCACCA | |
| | ACCUGUGCCCCUUCGACGAGGUGUUCAACGCCACCCGGUU | |
| | CGCCAGCGUGUACGCCUGGAACCGGAAGCGGAUCAGCAAC | |
| | UGCGUGGCCGACUACAGCGUGCUGUACAACCUGGCCCCCU | |
| | UCUUCACCUUCAAGUGCUACGGCGUGAGCCCCACCAAGCU | |
| | GAACGACCUGUGCUUCACCAACGUGUACGCCGACAGCUUC | |
| | GUGAUCCGUGGCGACGAGGUGCGGCAGAUCGCACCCGGCC | |
| | AGACAGGCAACAUCGCCGACUACAACUACAAGCUGCCCGA | |
| | CGACUUCACCGGCUGCGUGAUCGCCUGGAACAGCAACAAG | |
| | CUCGACAGCAAGGUGAGCGGCAACUACAACUACCUGUACC | |
| | GGCUGUUCCGGAAGAGCAACCUGAAGCCCUUCGAGCGGG | |
| | ACAUCAGCACCGAGAUCUACCAAGCCGGCAACAAGCCUUG | |
| | CAACGGCGUGGCCGGCUUCAACUGCUACUUCCCUCUGCGG | |
| | AGCUACAGCUUCCGGCCCACCUACGGCGUGGGCCACCAGC | |
| | CCUACCGGGUGGUGGUGCUGAGCUUCGAGCUGCUGCACG | |
| | CCCCAGCCACCGUGUGUGGCCCCAAGAAGAGCACCAACCU | |
| | GGUGAAGAACAAGUGCGUGAACUUCAACUUCAACGGCCU | |
| | UAAGGGCACCGGCGUGCUGACCGAGAGCAACAAGAAAUU | |
| | CCUGCCCUUUCAGCAGUUCGGCCGGGACAUCGCCGACACC | |
| | ACCGACGCUGUGCGGGAUCCCCAGACCCUGGAGAUCCUGG | |
| | ACAUCACCCCUUGCAGCUUCGGCGGCGUGAGCGUGAUCAC | |
| | CCCAGGCACCAACACCAGCAACCAGGUGGCCGUGCUGUAC | |
| | CAGGGCGUGAACUGCACCGAGGUGCCCGUGGCCAUCCACG | |
| | CCGACCAGCUGACACCCACCUGGCGGGUCUACAGCACCGG | |
| | CAGCAACGUGUUCCAGACCCGGGCCGGUUGCCUGAUCGGC | |
| | GCCGAGUACGUGAACAACAGCUACGAGUGCGACAUCCCCA | |
| | UCGGCGCCGGCAUCUGUGCCAGCUACCAGACCCAGACCAA | |
| | UUCACCCGGCGGCGCAGGCAGCGUGGCCAGCCAGAGCAUC | |
| | AUCGCCUACACCAUGAGCCUGGGCGCCGAGAACAGCGUGG | |
| | CCUACAGCAACAACAGCAUCGCCAUCCCCACCAACUUCAC | |
| | CAUCAGCGUGACCACCGAGAUUCUGCCCGUGAGCAUGACC | |
| | AAGACCAGCGUGGACUGCACCAUGUACAUCUGCGGCGAC | |
| | AGCACCGAGUGCAGCAACCUGCUGCUGCAGUACGGCAGCU | |
| | UCUGCACCCAGCUGAAGCGGGCCCUGACCGGCAUCGCCGU | |
| | GGAGCAGGACAAGAACACCCAGGAGGUGUUCGCCCAGGU | |
| | GAAGCAGAUCUACAAGACCCCUCCCAUCAAGUACUUCGGC | |
| | GGCUUCAACUUCAGCCAGAUCCUGCCCGACCCCAGCAAGC | |
| | CCAGCAAGCGGAGCUUCAUCGAGGACCUGCUGUUCAACA | |
| | AGGUGACCCUAGCCGACGCCGGCUUCAUCAAGCAGUACGG | |
| | CGACUGCCUCGGCGACAUAGCCGCCCGGGACCUGAUCUGC | |
| | GCCCAGAAGUUCAAGGGCCUGACCGUGCUGCCUCCCCUGC | |
| | UGACCGACGAGAUGAUCGCCCAGUACACCAGCGCCCUGUU | |
| | AGCCGGAACCAUCACCAGCGGCUGGACUUUCGGCGCUGGA | |
| | GCCGCUCUGCAGAUCCCCUUCGCCAUGCAGAUGGCCUACC | |
| | GGUUCAACGGCAUCGGCGUGACCCAGAACGUGCUGUACG | |
| | AGAACCAGAAGCUGAUCGCCAACCAGUUCAACAGCGCCAU | |
| | CGGCAAGAUCCAGGACAGCCUGAGCAGCACCGCUAGCGCC | |
| | CUGGGCAAGCUGCAGGACGUGGUGAACCACAACGCCCAG | |
| | GCCCUGAACACCCUGGUGAAGCAGCUGAGCAGCAAGUUC | |
| | GGCGCCAUCAGCAGCGUGCUGAACGACAUCUUCAGCCGGC | |
| | UGGACCCUCCCGAGGCCGAGGUGCAGAUCGACCGGCUGAU | |
| | CACUGGCCGGCUGCAGAGCCUGCAGACCUACGUGACCCAG | |
| | CAGCUGAUCCGGGCCGCCGAGAUUCGGGCCAGCGCCAACC | |
| | UGGCCGCCACCAAGAUGAGCGAGUGCGUGCUGGGCCAGA | |
| | GCAAGCGGGUGGACUUCUGCGGCAAGGGCUACCACCUGA | |
| | UGAGCUUUCCCCAGAGCGCACCCCACGGAGUGGUGUUCCU | |
| | GCACGUGACCUACGUGCCCGCCCAGGAGAAGAACUUCACC | |
| | ACCGCCCCAGCCAUCUGCCACGACGGCAAGGCCCACUUUC | |
| | CCCGGGAGGGCGUGUUCGUGAGCAACGGCACCCACUGGU | |
| | UCGUGACCCAGCGGAACUUCUACGAGCCCCAGAUCAUCAC | |
| | CACCGACAACACCUUCGUGAGCGGCAACUGCGACGUGGUG | |
| | AUCGGCAUCGUGAACAACACCGUGUACGAUCCCCUGCAGC | |
| | CCGAGCUGGACAGCUUCAAGGAGGAGCUGGACAAGUACU | |
| | UCAAGAAUCACACCAGCCCCGACGUGGACCUGGGCGACAU | |
| | CAGCGGCAUCAACGCCAGCGUGGUGAACAUCCAGAAGGA | |
| | GAUCGAUCGGCUGAACGAGGUGGCCAAGAACCUGAACGA | |
| | GAGCCUGAUCGACCUGCAGGAGCUGGGCAAGUACGAGCA | |
| | GUACAUCAAGUGGCCCUGGUACAUCUGGCUGGGCUUCAU | |
| | CGCCGGCCUGAUCGCCAUCGUGAUGGUGACCAUCAUGCUG | |
| | UGCUGCAUGACCAGCUGCUGCAGCUGCCUGAAGGGCUGU | |
| | UGCAGCUGCGGCAGCUGCUGCAAGUUCGACGAGGACGAC | |
| | AGCGAGCCCGUGCUGAAGGGCGUGAAGCUGCACUACACC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MFVFLVLLPLVSSQCVNLTTRTQLPPAYTNSFTRGVYYPDKVF | 36 |
| acid sequence | RSSVLHSTQDLFLPFFSNVTWFHVISGTNGTKRFDNPVLPFND | |
| | GVYFASIEKSNIIRGWIFGTTLDSKTQSLLIVNNATNVVIKVCEF | |
| | QFCNDPFLDHKNNKSWMESEFRVYSSANNCTFEYVSQPFLMD | |
| | LEGKQGNFKNLREFVFKNIDGYFKIYSKHTPIIVREPEDLPQGF | |
| | SALEPLVDLPIGINITRFQTLLALHRSYLTPGDSSSGWTAGAAA | |
| | YYVGYLQPRTFLLKYNENGTITDAVDCALDPLSETKCTLKSFT | |
| | VEKGIYQTSNFRVQPTESIVRFPNITNLCPFDEVENATRFASVY | |
| | AWNRKRISNCVADYSVLYNLAPFFTFKCYGVSPTKLNDLCFT | |
| | NVYADSFVIRGDEVRQIAPGQTGNIADYNYKLPDDFTGCVIA | |
| | WNSNKLDSKVSGNYNYLYRLFRKSNLKPFERDISTEIYQAGN | |
| | KPCNGVAGFNCYFPLRSYSFRPTYGVGHQPYRVVVLSFELLH | |
| | APATVCGPKKSTNLVKNKCVNFNFNGLKGTGVLTESNKKFLP | |
| | FQQFGRDIADTTDAVRDPQTLEILDITPCSFGGVSVITPGTNTSN | |
| | QVAVLYQGVNCTEVPVAIHADQLTPTWRVYSTGSNVFQTRA | |
| | GCLIGAEYVNNSYECDIPIGAGICASYQTQTNSPGGAGSVASQS | |
| | IIAYTMSLGAENSVAYSNNSIAIPTNFTISVTTEILPVSMTKTSV | |
| | DCTMYICGDSTECSNLLLQYGSFCTQLKRALTGIAVEQDKNTQ | |
| | EVFAQVKQIYKTPPIKYFGGFNFSQILPDPSKPSKRSFIEDLLEN | |
| | KVTLADAGFIKQYGDCLGDIAARDLICAQKFKGLTVLPPLLTD | |
| | EMIAQYTSALLAGTITSGWTFGAGAALQIPFAMQMAYRENGI | |
| | GVTQNVLYENQKLIANQFNSAIGKIQDSLSSTASALGKLQDVV | |
| | NHNAQALNTLVKQLSSKFGAISSVLNDIFSRLDPPEAEVQIDRL | |
| | ITGRLQSLQTYVTQQLIRAAEIRASANLAATKMSECVLGQSKR | |
| | VDFCGKGYHLMSFPQSAPHGVVFLHVTYVPAQEKNFTTAPAI | |
| | CHDGKAHFPREGVFVSNGTHWFVTQRNFYEPQIITTDNTFVSG | |
| | NCDVVIGIVNNTVYDPLQPELDSFKEELDKYFKNHTSPDVDLG | |
| | DISGINASVVNIQKEIDRLNEVAKNLNESLIDLQELGKYEQYIK | |
| | WPWYIWLGFIAGLIAIVMVTIMLCCMTSCCSCLKGCCSCGSCC | |
| | KFDEDDSEPVLKGVKLHYT | |
| PolyA tail | 100 nt | |

mRNA-1273.B.1.1.529_PBSko_match

| SEQ ID NO: 37 consists of from 5′ end to 3′ end: 5′ UTR SEQ ID NO: 2, | | 37 |
|---|---|---|
| mRNA ORF SEQ ID NO: 38, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGUUCGUGUUCCUGGUGCUGCUGCCCCUGGUGAGCAGC | 38 |
| Construct | CAGUGCGUGAACCUGACCACCCGGACCCAGCUGCCACCAG | |
| (excluding the stop | CCUACACCAACAGCUUCACCCGGGGCGUGUACUACCCCGA | |
| codon) | CAAGGUGUUCCGGAGCAGCGUGCUGCACAGCACCCAGGAC | |
| | CUGUUCCUGCCCUUCUUCAGCAACGUGACCUGGUUCCACG | |
| | UGAUCAGCGGCACCAACGGCACCAAGCGGUUCGACAACCC | |
| | CGUGCUGCCCUUCAACGACGGCGUGUACUUCGCCAGCAUC | |
| | GAGAAGAGCAACAUCAUCCGGGGCUGGAUCUUCGGCACC | |
| | ACCCUGGACAGCAAGACCCAGAGCCUGCUGAUCGUGAACA | |
| | ACGCCACCAACGUGGUGAUCAAGGUGUGCGAGUUCCAGU | |
| | UCUGCAACGACCCCUUCCUGGACCACAAGAACAACAAGAG | |
| | CUGGAUGGAGAGCGAGUUCCGGGUGUACAGCAGCGCCAA | |
| | CAACUGCACCUUCGAGUACGUGAGCCAGCCCUUCCUGAUG | |
| | GACCUGGAGGGCAAGCAGGGCAACUUCAAGAACCUGCGG | |
| | GAGUUCGUGUUCAAGAACAUCGACGGCUACUUCAAGAUC | |
| | UACAGCAAGCACACCCCAAUCAUCGUGCGGGAGCCCGAGG | |
| | ACCUGCCCCAGGGCUUCAGCGCCCUGGAGCCCCUGGUGGA | |
| | CCUGCCCAUCGGCAUCAACAUCACCCGGUUCCAGACCCUG | |
| | CUGGCCCUGCACCGGAGCUACCUGACCCCAGGCGACAGCA | |
| | GCAGCGGCUGGACCGCCGGCGCCGCCGCCUACUACGUGGG | |
| | CUACCUGCAGCCCCGGACCUUCCUGCUGAAGUACAACGAG | |
| | AACGGCACCAUCACCGACGCCGUGGACUGCGCCCUGGACC | |
| | CUCUGAGCGAGACCAAGUGCACCCUGAAGAGCUUCACCGU | |
| | GGAGAAGGGCAUCUACCAGACCAGCAACUUCCGGGUGCA | |
| | GCCCACCGAGAGCAUCGUGCGGUUCCCCAACAUCACCAAC | |
| | CUGUGCCCCUUCGACGAGGUGUUCAACGCCACCCGGUUCG | |
| | CCAGCGUGUACGCCUGGAACCGGAAGCGGAUCAGCAACU | |
| | GCGUGGCCGACUACAGCGUGCUGUACAACCUGGCCCCUUU | |
| | CUUCACCUUCAAGUGCUACGGCGUGAGCCCCACCAAGCUG | |
| | AACGACCUGUGCUUCACCAACGUGUACGCCGACAGCUUCG | |
| | UGAUCCGGGGCGACGAGGUGCGGCAGAUCGCCCCAGGCCA | |
| | GACCGGCAACAUCGCCGACUACAACUACAAGCUGCCCGAC | |
| | GACUUCACCGGCUGCGUGAUCGCCUGGAACAGCAACAAGC | |
| | UGGACAGCAAGGUGAGCGGCAACUACAACUACCUGUACC | |
| | GGCUGUUCCGGAAGAGCAACCUGAAGCCCUUCGAGCGGG | |
| | ACAUCAGCACCGAGAUCUACCAGGCCGGCAACAAGCCCUG | |
| | CAACGGCGUGGCCGGCUUCAACUGCUACUUCCCUCUGCGG | |
| | AGCUACAGCUUCCGGCCCACCUACGGCGUGGGCCACCAGC | |
| | CCUACCGGGUGGUGGUGCUGAGCUUCGAGCUGCUGCACG | |
| | CCCCAGCCACCGUGUGCGGCCCCAAGAAGAGCACCAACCU | |
| | GGUGAAGAACAAGUGCGUGAACUUCAACUUCAACGGCCU | |
| | GAAGGGCACCGGCGUGCUGACCGAGAGCAACAAGAAGUU | |
| | CCUGCCCUUCCAGCAGUUCGGCCGGGACAUCGCCGACACC | |
| | ACCGACGCCGUGCGGGAUCCCCAGACCCUGGAGAUCCUGG | |
| | ACAUCACCCCUUGCAGCUUCGGCGGCGUGAGCGUGAUCAC | |
| | CCCAGGCACCAACACCAGCAACCAGGUGGCCGUGCUGUAC | |
| | CAGGGCGUGAACUGCACCGAGGUGCCCGUGGCCAUCCACG | |
| | CCGACCAGCUGACCCCAACCUGGCGGGUGUACAGCACCGG | |
| | CAGCAACGUGUUCCAGACCCGGGCCGGCUGCCUGAUCGGC | |
| | GCCGAGUACGUGAACAACAGCUACGAGUGCGACAUCCCCA | |
| | UCGGCGCCGGCAUCUGCGCCAGCUACCAGACCCAGACCAA | |
| | CAGCCCCGGCGGCGCCGGCAGCGUGGCCAGCCAGAGCAUC | |
| | AUCGCCUACACCAUGAGCCUGGGCGCCGAGAACAGCGUGG | |
| | CCUACAGCAACAACAGCAUCGCCAUCCCCACCAACUUCAC | |
| | CAUCAGCGUGACCACCGAGAUCCUGCCCGUGAGCAUGACC | |
| | AAGACCAGCGUGGACUGCACCAUGUACAUCUGCGGCGAC | |
| | AGCACCGAGUGCAGCAACCUGCUGCUGCAGUACGGCAGCU | |
| | UCUGCACCCAGCUGAAGCGGGCCCUGACCGGCAUCGCCGU | |
| | GGAGCAGGACAAGAACACCCAGGAGGUGUUCGCCCAGGU | |
| | GAAGCAGAUCUACAAGACCCCUCCCAUCAAGUACUUCGGC | |
| | GGCUUCAACUUCAGCCAGAUCCUGCCCGACCCCAGCAAGC | |
| | CCAGCAAGCGGAGCUUCAUCGAGGACCUGCUGUUCAACA | |
| | AGGUGACCCUGGCCGACGCCGGCUUCAUCAAGCAGUACGG | |
| | CGACUGCCUGGGCGACAUCGCCGCCCGGGACCUGAUCUGC | |
| | GCCCAGAAGUUCAAGGGCCUGACCGUGCUGCCUCCUCUGC | |
| | UGACCGACGAGAUGAUCGCCCAGUACACCAGCGCCCUGCU | |
| | GGCCGGCACCAUCACCAGCGGCUGGACCUUCGGCGCCGGC | |
| | GCCGCCCUGCAGAUCCCCUUCGCCAUGCAGAUGGCCUACC | |
| | GGUUCAACGGCAUCGGCGUGACCCAGAACGUGCUGUACG | |
| | AGAACCAGAAGCUGAUCGCCAACCAGUUCAACAGCGCCAU | |
| | CGGCAAGAUCCAGGACAGCCUGAGCAGCACCGCCAGCGCC | |
| | CUGGGCAAGCUGCAGGACGUGGUGAACCACAACGCCCAG | |
| | GCCCUGAACACCCUGGUGAAGCAGCUGAGCAGCAAGUUC | |
| | GGCGCCAUCAGCAGCGUGCUGAACGACAUCUUCAGCCGGC | |
| | UGGACCCUCCCGAGGCCGAGGUGCAGAUCGACCGGCUGAU | |
| | CACCGGCCGGCUGCAGAGCCUGCAGACCUACGUGACCCAG | |
| | CAGCUGAUCCGGGCCGCCGAGAUCCGGGCCAGCGCCAACC | |
| | UGGCCGCCACCAAGAUGAGCGAGUGCGUGCUGGGCCAGA | |
| | GCAAGCGGGUGGACUUCUGCGGCAAGGGCUACCACCUGA | |
| | UGAGCUUUCCCCAGAGCGCACCCCACGGCGUGGUGUUCCU | |
| | GCACGUGACCUACGUGCCCGCCCAGGAGAAGAACUUCACC | |
| | ACCGCCCCAGCCAUCUGCCACGACGGCAAGGCCCACUUUC | |
| | CCCGGGAGGGCGUGUUCGUGAGCAACGGCACCCACUGGU | |
| | UCGUGACCCAGCGGAACUUCUACGAGCCCCAGAUCAUCAC | |
| | CACCGACAACACCUUCGUGAGCGGCAACUGCGACGUGGUG | |
| | AUCGGCAUCGUGAACAACACCGUGUACGAUCCCCUGCAGC | |
| | CCGAGCUGGACAGCUUCAAGGAGGAGCUGGACAAGUACU | |
| | UCAAGAACCACACCAGCCCCGACGUGGACCUGGGCGACAU | |
| | CAGCGGCAUCAACGCCAGCGUGGUGAACAUCCAGAAGGA | |
| | GAUCGACAGACUGAACGAGGUGGCCAAGAACCUGAACGA | |
| | GAGCCUGAUCGACCUGCAGGAGCUGGGCAAGUACGAGCA | |
| | GUACAUCAAGUGGCCCUGGUACAUCUGGCUGGGCUUCAU | |
| | CGCCGGCCUGAUCGCCAUCGUGAUGGUGACCAUCAUGCUG | |
| | UGCUGCAUGACCAGCUGCUGCAGCUGCCUGAAGGGCUGC | |
| | UGCAGCUGCGGCAGCUGCUGCAAGUUCGACGAGGACGAC | |
| | AGCGAGCCCGUGCUGAAGGGCGUGAAGCUGCACUACACC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MFVFLVLLPLVSSQCVNLTTRTQLPPAYTNSFTRGVYYPDKVF | 39 |
| acid sequence | RSSVLHSTQDLFLPFFSNVTWFHVISGTNGTKRFDNPVLPEND | |
| | GVYFASIEKSNIIRGWIFGTTLDSKTQSLLIVNNATNVVIKVCEF | |
| | QFCNDPFLDHKNNKSWMESEFRVYSSANNCTFEYVSQPFLMD | |
| | LEGKQGNFKNLREFVFKNIDGYFKIYSKHTPIIVREPEDLPQGF | |
| | SALEPLVDLPIGINITRFQTLLALHRSYLTPGDSSSGWTAGAAA | |
| | YYVGYLQPRTFLLKYNENGTITDAVDCALDPLSETKCTLKSFT | |
| | VEKGIYQTSNFRVQPTESIVRFPNITNLCPFDEVFNATRFASVY | |
| | AWNRKRISNCVADYSVLYNLAPFFTFKCYGVSPTKLNDLCFT | |
| | NVYADSFVIRGDEVRQIAPGQTGNIADYNYKLPDDFTGCVIA | |
| | WNSNKLDSKVSGNYNYLYRLFRKSNLKPFERDISTEIYQAGN | |
| | KPCNGVAGENCYFPLRSYSFRPTYGVGHQPYRVVVLSFELLH | |
| | APATVCGPKKSTNLVKNKCVNFNFNGLKGTGVLTESNKKFLP | |
| | FQQFGRDIADTTDAVRDPQTLEILDITPCSFGGVSVITPGTNTSN | |
| | QVAVLYQGVNCTEVPVAIHADQLTPTWRVYSTGSNVFQTRA | |
| | GCLIGAEYVNNSYECDIPIGAGICASYQTQTNSPGGAGSVASQS | |
| | IIAYTMSLGAENSVAYSNNSIAIPTNFTISVTTEILPVSMTKTSV | |
| | DCTMYICGDSTECSNLLLQYGSFCTQLKRALTGIAVEQDKNTQ | |
| | EVFAQVKQIYKTPPIKYFGGFNFSQILPDPSKPSKRSFIEDLLEN | |
| | KVTLADAGFIKQYGDCLGDIAARDLICAQKFKGLTVLPPLLTD | |
| | EMIAQYTSALLAGTITSGWTFGAGAALQIPFAMQMAYRENGI | |
| | GVTQNVLYENQKLIANQFNSAIGKIQDSLSSTASALGKLQDVV | |
| | NHNAQALNTLVKQLSSKFGAISSVLNDIFSRLDPPEAEVQIDRL | |
| | ITGRLQSLQTYVTQQLIRAAEIRASANLAATKMSECVLGQSKR | |
| | VDFCGKGYHLMSFPQSAPHGVVFLHVTYVPAQEKNFTTAPAI | |
| | CHDGKAHFPREGVFVSNGTHWFVTQRNFYEPQIITTDNTFVSG | |
| | NCDVVIGIVNNTVYDPLQPELDSFKEELDKYFKNHTSPDVDLG | |
| | DISGINASVVNIQKEIDRLNEVAKNLNESLIDLQELGKYEQYIK | |
| | WPWYIWLGFIAGLIAIVMVTIMLCCMTSCCSCLKGCCSCGSCC | |
| | KFDEDDSEPVLKGVKLHYT | |
| PolyA tail | 100 nt | |

B.1.1.529-1273_m_mu

| SEQ ID NO: 40 consists of from 5′ end to 3′ end: 5′ UTR SEQ ID NO: 2, | | 40 |
|---|---|---|
| mRNA ORF SEQ ID NO: 41, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGUUCGUGUUCCUGGUGCUGCUGCCCCUGGUGAGCAGC | 41 |
| Construct | CAGUGCGUGAACCUGACCACCCGGACCCAGCUGCCACCAG | |
| (excluding the stop | CCUACACCAACAGCUUCACCCGGGGCGUGUACUACCCCGA | |
| codon) | CAAGGUGUUCCGGAGCAGCGUGCUGCACAGCACCCAGGAC | |
| | CUGUUCCUGCCCUUCUUCAGCAACGUGACCUGGUUCCACG | |
| | UGAUCAGCGGCACCAACGGCACCAAGCGGUUCGACAACCC | |
| | CGUGCUGCCCUUCAACGACGGCGUGUACUUCGCCAGCAUC | |
| | GAGAAGAGCAACAUCAUCCGGGGCUGGAUCUUCGGCACC | |
| | ACCCUGGACAGCAAGACCCAGAGCCUGCUGAUCGUGAACA | |
| | ACGCCACCAACGUGGUGAUCAAGGUGUGCGAGUUCCAGU | |
| | UCUGCAACGACCCCUUCCUGGACCACAAGAACAACAAGAG | |
| | CUGGAUGGAGAGCGAGUUCCGGGUGUACAGCAGCGCCAA | |
| | CAACUGCACCUUCGAGUACGUGAGCCAGCCCUUCCUGAUG | |
| | GACCUGGAGGGCAAGCAGGGCAACUUCAAGAACCUGCGG | |
| | GAGUUCGUGUUCAAGAACAUCGACGGCUACUUCAAGAUC | |
| | UACAGCAAGCACACCCCAAUCAUCGUGCGGGAGCCCGAGG | |
| | ACCUGCCCCAGGGCUUCAGCGCCCUGGAGCCCCUGGUGGA | |
| | CCUGCCCAUCGGCAUCAACAUCACCCGGUUCCAGACCCUG | |
| | CUGGCCCUGCACCGGAGCUACCUGACCCCAGGCGACAGCA | |
| | GCAGCGGCUGGACCGCCGGCGCCGCCGCCUACUACGUGGG | |
| | CUACCUGCAGCCCCGGACCUUCCUGCUGAAGUACAACGAG | |
| | AACGGCACCAUCACCGACGCCGUGGACUGCGCCCUGGACC | |
| | CUCUGAGCGAGACCAAGUGCACCCUGAAGAGCUUCACCGU | |
| | GGAGAAGGGCAUCUACCAGACCAGCAACUUCCGGGUGCA | |
| | GCCCACCGAGAGCAUCGUGCGGUUCCCCAACAUCACCAAC | |
| | CUGUGCCCCUUCGACGAGGUGUUCAACGCCACCCGGUUCG | |
| | CCAGCGUGUACGCCUGGAACCGGAAGCGGAUCAGCAACU | |
| | GCGUGGCCGACUACAGCGUGCUGUACAACCUGGCCCCUUU | |
| | CUUCACCUUCAAGUGCUACGGCGUGAGCCCCACCAAGCUG | |
| | AACGACCUGUGCUUCACCAACGUGUACGCCGACAGCUUCG | |
| | UGAUCCGGGGCGACGAGGUGCGGCAGAUCGCCCCAGGCCA | |
| | GACCGGCAACAUCGCCGACUACAACUACAAGCUGCCCGAC | |
| | GACUUCACCGGCUGCGUGAUCGCCUGGAACAGCAACAAGC | |
| | UGGACAGCAAGGUGAGCGGCAACUACAACUACCUGUACC | |
| | GGCUGUUCCGGAAGAGCAACCUGAAGCCCUUCGAGCGGG | |
| | ACAUCAGCACCGAGAUCUACCAGGCCGGCAACAAGCCCUG | |
| | CAACGGCGUGGCCGGCUUCAACUGCUACUUCCCUCUGCGG | |
| | AGCUACAGCUUCCGGCCCACCUACGGCGUGGGCCACCAGC | |
| | CCUACCGGGUGGUGGUGCUGAGCUUCGAGCUGCUGCACG | |
| | CCCCAGCCACCGUGUGCGGCCCCAAGAAGAGCACCAACCU | |
| | GGUGAAGAACAAGUGCGUGAACUUCAACUUCAACGGCCU | |
| | GAAGGGCACCGGCGUGCUGACCGAGAGCAACAAGAAGUU | |
| | CCUGCCCUUCCAGCAGUUCGGCCGGGACAUCGCCGACACC | |
| | ACCGACGCCGUGCGGGAUCCCCAGACCCUGGAGAUCCUGG | |
| | ACAUCACCCCUUGCAGCUUCGGCGGCGUGAGCGUGAUCAC | |
| | CCCAGGCACCAACACCAGCAACCAGGUGGCCGUGCUGUAC | |
| | CAGGGCGUGAACUGCACCGAGGUGCCCGUGGCCAUCCACG | |
| | CCGACCAGCUGACCCCAACCUGGCGGGUGUACAGCACCGG | |
| | CAGCAACGUGUUCCAGACCCGGGCCGGCUGCCUGAUCGGC | |
| | GCCGAGUACGUGAACAACAGCUACGAGUGCGACAUCCCCA | |
| | UCGGCGCCGGCAUCUGCGCCAGCUACCAGACCCAGACCAA | |
| | GAGCCACCGGCGGGCCCGGAGCGUGGCCAGCCAGAGCAUC | |
| | AUCGCCUACACCAUGAGCCUGGGCGCCGAGAACAGCGUGG | |
| | CCUACAGCAACAACAGCAUCGCCAUCCCCACCAACUUCAC | |
| | CAUCAGCGUGACCACCGAGAUCCUGCCCGUGAGCAUGACC | |
| | AAGACCAGCGUGGACUGCACCAUGUACAUCUGCGGCGAC | |
| | AGCACCGAGUGCAGCAACCUGCUGCUGCAGUACGGCAGCU | |
| | UCUGCACCCAGCUGAAGCGGGCCCUGACCGGCAUCGCCGU | |
| | GGAGCAGGACAAGAACACCCAGGAGGUGUUCGCCCAGGU | |
| | GAAGCAGAUCUACAAGACCCCUCCCAUCAAGUACUUCGGC | |
| | GGCUUCAACUUCAGCCAGAUCCUGCCCGACCCCAGCAAGC | |
| | CCAGCAAGCGGAGCUUCAUCGAGGACCUGCUGUUCAACA | |
| | AGGUGACCCUGGCCGACGCCGGCUUCAUCAAGCAGUACGG | |
| | CGACUGCCUGGGCGACAUCGCCGCCCGGGACCUGAUCUGC | |
| | GCCCAGAAGUUCAAGGGCCUGACCGUGCUGCCUCCUCUGC | |
| | UGACCGACGAGAUGAUCGCCCAGUACACCAGCGCCCUGCU | |
| | GGCCGGCACCAUCACCAGCGGCUGGACCUUCGGCGCCGGC | |
| | GCCGCCCUGCAGAUCCCCUUCGCCAUGCAGAUGGCCUACC | |
| | GGUUCAACGGCAUCGGCGUGACCCAGAACGUGCUGUACG | |
| | AGAACCAGAAGCUGAUCGCCAACCAGUUCAACAGCGCCAU | |
| | CGGCAAGAUCCAGGACAGCCUGAGCAGCACCGCCAGCGCC | |
| | CUGGGCAAGCUGCAGGACGUGGUGAACCACAACGCCCAG | |
| | GCCCUGAACACCCUGGUGAAGCAGCUGAGCAGCAAGUUC | |
| | GGCGCCAUCAGCAGCGUGCUGAACGACAUCUUCAGCCGGC | |
| | UGGACCCUCCCGAGGCCGAGGUGCAGAUCGACCGGCUGAU | |
| | CACCGGCCGGCUGCAGAGCCUGCAGACCUACGUGACCCAG | |
| | CAGCUGAUCCGGGCCGCCGAGAUCCGGGCCAGCGCCAACC | |
| | UGGCCGCCACCAAGAUGAGCGAGUGCGUGCUGGGCCAGA | |
| | GCAAGCGGGUGGACUUCUGCGGCAAGGGCUACCACCUGA | |
| | UGAGCUUUCCCCAGAGCGCACCCCACGGCGUGGUGUUCCU | |
| | GCACGUGACCUACGUGCCCGCCCAGGAGAAGAACUUCACC | |
| | ACCGCCCCAGCCAUCUGCCACGACGGCAAGGCCCACUUUC | |
| | CCCGGGAGGGCGUGUUCGUGAGCAACGGCACCCACUGGU | |
| | UCGUGACCCAGCGGAACUUCUACGAGCCCCAGAUCAUCAC | |
| | CACCGACAACACCUUCGUGAGCGGCAACUGCGACGUGGUG | |
| | AUCGGCAUCGUGAACAACACCGUGUACGAUCCCCUGCAGC | |
| | CCGAGCUGGACAGCUUCAAGGAGGAGCUGGACAAGUACU | |
| | UCAAGAACCACACCAGCCCCGACGUGGACCUGGGCGACAU | |
| | CAGCGGCAUCAACGCCAGCGUGGUGAACAUCCAGAAGGA | |
| | GAUCGACAGACUGAACGAGGUGGCCAAGAACCUGAACGA | |
| | GAGCCUGAUCGACCUGCAGGAGCUGGGCAAGUACGAGCA | |
| | GUACAUCAAGUGGCCCUGGUACAUCUGGCUGGGCUUCAU | |
| | CGCCGGCCUGAUCGCCAUCGUGAUGGUGACCAUCAUGCUG | |
| | UGCUGCAUGACCAGCUGCUGCAGCUGCCUGAAGGGCUGC | |
| | UGCAGCUGCGGCAGCUGCUGCAAGUUCGACGAGGACGAC | |
| | AGCGAGCCCGUGCUGAAGGGCGUGAAGCUGCACUACACC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MFVFLVLLPLVSSQCVNLTTRTQLPPAYTNSFTRGVYYPDKVF | 42 |
| acid sequence | RSSVLHSTQDLFLPFFSNVTWFHVISGTNGTKRFDNPVLPEND | |
| | GVYFASIEKSNIIRGWIFGTTLDSKTQSLLIVNNATNVVIKVCEF | |
| | QFCNDPFLDHKNNKSWMESEFRVYSSANNCTFEYVSQPFLMD | |
| | LEGKQGNFKNLREFVFKNIDGYFKIYSKHTPIIVREPEDLPQGF | |
| | SALEPLVDLPIGINITRFQTLLALHRSYLTPGDSSSGWTAGAAA | |
| | YYVGYLQPRTFLLKYNENGTITDAVDCALDPLSETKCTLKSFT | |
| | VEKGIYQTSNFRVQPTESIVRFPNITNLCPFDEVFNATRFASVY | |
| | AWNRKRISNCVADYSVLYNLAPFFTFKCYGVSPTKLNDLCFT | |
| | NVYADSFVIRGDEVRQIAPGQTGNIADYNYKLPDDFTGCVIA | |
| | WNSNKLDSKVSGNYNYLYRLFRKSNLKPFERDISTEIYQAGN | |
| | KPCNGVAGFNCYFPLRSYSFRPTYGVGHQPYRVVVLSFELLH | |
| | APATVCGPKKSTNLVKNKCVNFNFNGLKGTGVLTESNKKFLP | |
| | FQQFGRDIADTTDAVRDPQTLEILDITPCSFGGVSVITPGTNTSN | |
| | QVAVLYQGVNCTEVPVAIHADQLTPTWRVYSTGSNVFQTRA | |
| | GCLIGAEYVNNSYECDIPIGAGICASYQTQTKSHRRARSVASQS | |
| | IIAYTMSLGAENSVAYSNNSIAIPTNFTISVTTEILPVSMTKTSV | |
| | DCTMYICGDSTECSNLLLQYGSFCTQLKRALTGIAVEQDKNTQ | |
| | EVFAQVKQIYKTPPIKYFGGFNFSQILPDPSKPSKRSFIEDLLEN | |
| | KVTLADAGFIKQYGDCLGDIAARDLICAQKFKGLTVLPPLLTD | |
| | EMIAQYTSALLAGTITSGWTFGAGAALQIPFAMQMAYRENGI | |
| | GVTQNVLYENQKLIANQFNSAIGKIQDSLSSTASALGKLQDVV | |
| | NHNAQALNTLVKQLSSKFGAISSVLNDIFSRLDPPEAEVQIDRL | |
| | ITGRLQSLQTYVTQQLIRAAEIRASANLAATKMSECVLGQSKR | |
| | VDFCGKGYHLMSFPQSAPHGVVFLHVTYVPAQEKNFTTAPAI | |
| | CHDGKAHFPREGVFVSNGTHWFVTQRNFYEPQIITTDNTFVSG | |
| | NCDVVIGIVNNTVYDPLQPELDSFKEELDKYFKNHTSPDVDLG | |
| | DISGINASVVNIQKEIDRLNEVAKNLNESLIDLQELGKYEQYIK | |
| | WPWYIWLGFIAGLIAIVMVTIMLCCMTSCCSCLKGCCSCGSCC | |
| | KFDEDDSEPVLKGVKLHYT | |
| PolyA tail | 100 nt | |

1273.529.IDR14A

| SEQ ID NO: 43 consists of from 5′ end to 3′ end: 5′ UTR SEQ ID NO: 2, | | 43 |
|---|---|---|
| mRNA ORF SEQ ID NO: 41, and 3′ UTR SEQ ID NO: 44. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGUUCGUGUUCCUGGUGCUGCUGCCCCUGGUGAGCAGC | 41 |
| Construct | CAGUGCGUGAACCUGACCACCCGGACCCAGCUGCCACCAG | |
| (excluding the stop | CCUACACCAACAGCUUCACCCGGGGCGUGUACUACCCCGA | |
| codon) | CAAGGUGUUCCGGAGCAGCGUGCUGCACAGCACCCAGGAC | |
| | CUGUUCCUGCCCUUCUUCAGCAACGUGACCUGGUUCCACG | |
| | UGAUCAGCGGCACCAACGGCACCAAGCGGUUCGACAACCC | |
| | CGUGCUGCCCUUCAACGACGGCGUGUACUUCGCCAGCAUC | |
| | GAGAAGAGCAACAUCAUCCGGGGCUGGAUCUUCGGCACC | |
| | ACCCUGGACAGCAAGACCCAGAGCCUGCUGAUCGUGAACA | |
| | ACGCCACCAACGUGGUGAUCAAGGUGUGCGAGUUCCAGU | |
| | UCUGCAACGACCCCUUCCUGGACCACAAGAACAACAAGAG | |
| | CUGGAUGGAGAGCGAGUUCCGGGUGUACAGCAGCGCCAA | |
| | CAACUGCACCUUCGAGUACGUGAGCCAGCCCUUCCUGAUG | |
| | GACCUGGAGGGCAAGCAGGGCAACUUCAAGAACCUGCGG | |
| | GAGUUCGUGUUCAAGAACAUCGACGGCUACUUCAAGAUC | |
| | UACAGCAAGCACACCCCAAUCAUCGUGCGGGAGCCCGAGG | |
| | ACCUGCCCCAGGGCUUCAGCGCCCUGGAGCCCCUGGUGGA | |
| | CCUGCCCAUCGGCAUCAACAUCACCCGGUUCCAGACCCUG | |
| | CUGGCCCUGCACCGGAGCUACCUGACCCCAGGCGACAGCA | |
| | GCAGCGGCUGGACCGCCGGCGCCGCCGCCUACUACGUGGG | |
| | CUACCUGCAGCCCCGGACCUUCCUGCUGAAGUACAACGAG | |
| | AACGGCACCAUCACCGACGCCGUGGACUGCGCCCUGGACC | |
| | CUCUGAGCGAGACCAAGUGCACCCUGAAGAGCUUCACCGU | |
| | GGAGAAGGGCAUCUACCAGACCAGCAACUUCCGGGUGCA | |
| | GCCCACCGAGAGCAUCGUGCGGUUCCCCAACAUCACCAAC | |
| | CUGUGCCCCUUCGACGAGGUGUUCAACGCCACCCGGUUCG | |
| | CCAGCGUGUACGCCUGGAACCGGAAGCGGAUCAGCAACU | |
| | GCGUGGCCGACUACAGCGUGCUGUACAACCUGGCCCCUUU | |
| | CUUCACCUUCAAGUGCUACGGCGUGAGCCCCACCAAGCUG | |
| | AACGACCUGUGCUUCACCAACGUGUACGCCGACAGCUUCG | |
| | UGAUCCGGGGCGACGAGGUGCGGCAGAUCGCCCCAGGCCA | |
| | GACCGGCAACAUCGCCGACUACAACUACAAGCUGCCCGAC | |
| | GACUUCACCGGCUGCGUGAUCGCCUGGAACAGCAACAAGC | |
| | UGGACAGCAAGGUGAGCGGCAACUACAACUACCUGUACC | |
| | GGCUGUUCCGGAAGAGCAACCUGAAGCCCUUCGAGCGGG | |
| | ACAUCAGCACCGAGAUCUACCAGGCCGGCAACAAGCCCUG | |
| | CAACGGCGUGGCCGGCUUCAACUGCUACUUCCCUCUGCGG | |
| | AGCUACAGCUUCCGGCCCACCUACGGCGUGGGCCACCAGC | |
| | CCUACCGGGUGGUGGUGCUGAGCUUCGAGCUGCUGCACG | |
| | CCCCAGCCACCGUGUGCGGCCCCAAGAAGAGCACCAACCU | |
| | GGUGAAGAACAAGUGCGUGAACUUCAACUUCAACGGCCU | |
| | GAAGGGCACCGGCGUGCUGACCGAGAGCAACAAGAAGUU | |
| | CCUGCCCUUCCAGCAGUUCGGCCGGGACAUCGCCGACACC | |
| | ACCGACGCCGUGCGGGAUCCCCAGACCCUGGAGAUCCUGG | |
| | ACAUCACCCCUUGCAGCUUCGGCGGCGUGAGCGUGAUCAC | |
| | CCCAGGCACCAACACCAGCAACCAGGUGGCCGUGCUGUAC | |
| | CAGGGCGUGAACUGCACCGAGGUGCCCGUGGCCAUCCACG | |
| | CCGACCAGCUGACCCCAACCUGGCGGGUGUACAGCACCGG | |
| | CAGCAACGUGUUCCAGACCCGGGCCGGCUGCCUGAUCGGC | |
| | GCCGAGUACGUGAACAACAGCUACGAGUGCGACAUCCCCA | |
| | UCGGCGCCGGCAUCUGCGCCAGCUACCAGACCCAGACCAA | |
| | GAGCCACCGGCGGGCCCGGAGCGUGGCCAGCCAGAGCAUC | |
| | AUCGCCUACACCAUGAGCCUGGGCGCCGAGAACAGCGUGG | |
| | CCUACAGCAACAACAGCAUCGCCAUCCCCACCAACUUCAC | |
| | CAUCAGCGUGACCACCGAGAUCCUGCCCGUGAGCAUGACC | |
| | AAGACCAGCGUGGACUGCACCAUGUACAUCUGCGGCGAC | |
| | AGCACCGAGUGCAGCAACCUGCUGCUGCAGUACGGCAGCU | |
| | UCUGCACCCAGCUGAAGCGGGCCCUGACCGGCAUCGCCGU | |
| | GGAGCAGGACAAGAACACCCAGGAGGUGUUCGCCCAGGU | |
| | GAAGCAGAUCUACAAGACCCCUCCCAUCAAGUACUUCGGC | |
| | GGCUUCAACUUCAGCCAGAUCCUGCCCGACCCCAGCAAGC | |
| | CCAGCAAGCGGAGCUUCAUCGAGGACCUGCUGUUCAACA | |
| | AGGUGACCCUGGCCGACGCCGGCUUCAUCAAGCAGUACGG | |
| | CGACUGCCUGGGCGACAUCGCCGCCCGGGACCUGAUCUGC | |
| | GCCCAGAAGUUCAAGGGCCUGACCGUGCUGCCUCCUCUGC | |
| | UGACCGACGAGAUGAUCGCCCAGUACACCAGCGCCCUGCU | |
| | GGCCGGCACCAUCACCAGCGGCUGGACCUUCGGCGCCGGC | |
| | GCCGCCCUGCAGAUCCCCUUCGCCAUGCAGAUGGCCUACC | |
| | GGUUCAACGGCAUCGGCGUGACCCAGAACGUGCUGUACG | |
| | AGAACCAGAAGCUGAUCGCCAACCAGUUCAACAGCGCCAU | |
| | CGGCAAGAUCCAGGACAGCCUGAGCAGCACCGCCAGCGCC | |
| | CUGGGCAAGCUGCAGGACGUGGUGAACCACAACGCCCAG | |
| | GCCCUGAACACCCUGGUGAAGCAGCUGAGCAGCAAGUUC | |
| | GGCGCCAUCAGCAGCGUGCUGAACGACAUCUUCAGCCGGC | |
| | UGGACCCUCCCGAGGCCGAGGUGCAGAUCGACCGGCUGAU | |
| | CACCGGCCGGCUGCAGAGCCUGCAGACCUACGUGACCCAG | |
| | CAGCUGAUCCGGGCCGCCGAGAUCCGGGCCAGCGCCAACC | |
| | UGGCCGCCACCAAGAUGAGCGAGUGCGUGCUGGGCCAGA | |
| | GCAAGCGGGUGGACUUCUGCGGCAAGGGCUACCACCUGA | |
| | UGAGCUUUCCCCAGAGCGCACCCCACGGCGUGGUGUUCCU | |
| | GCACGUGACCUACGUGCCCGCCCAGGAGAAGAACUUCACC | |
| | ACCGCCCCAGCCAUCUGCCACGACGGCAAGGCCCACUUUC | |
| | CCCGGGAGGGCGUGUUCGUGAGCAACGGCACCCACUGGU | |
| | UCGUGACCCAGCGGAACUUCUACGAGCCCCAGAUCAUCAC | |
| | CACCGACAACACCUUCGUGAGCGGCAACUGCGACGUGGUG | |
| | AUCGGCAUCGUGAACAACACCGUGUACGAUCCCCUGCAGC | |
| | CCGAGCUGGACAGCUUCAAGGAGGAGCUGGACAAGUACU | |
| | UCAAGAACCACACCAGCCCCGACGUGGACCUGGGCGACAU | |
| | CAGCGGCAUCAACGCCAGCGUGGUGAACAUCCAGAAGGA | |
| | GAUCGACAGACUGAACGAGGUGGCCAAGAACCUGAACGA | |
| | GAGCCUGAUCGACCUGCAGGAGCUGGGCAAGUACGAGCA | |
| | GUACAUCAAGUGGCCCUGGUACAUCUGGCUGGGCUUCAU | |
| | CGCCGGCCUGAUCGCCAUCGUGAUGGUGACCAUCAUGCUG | |
| | UGCUGCAUGACCAGCUGCUGCAGCUGCCUGAAGGGCUGC | |
| | UGCAGCUGCGGCAGCUGCUGCAAGUUCGACGAGGACGAC | |
| | AGCGAGCCCGUGCUGAAGGGCGUGAAGCUGCACUACACC | |
| 3′ UTR | UGAUAAUAGGCUGGAGCCUCGGUGGCCUAGCUUCUUGCC | 44 |
| | CCUUGGGCCUCCCCCCAGCCCCUCCUCCCCUUCCUGCAGG | |
| | AUAGAUAGCGAAGUGGUCUUUGAAUAAAGUCUGAGUGGG | |
| | CGGC | |
| Corresponding amino | MFVFLVLLPLVSSQCVNLTTRTQLPPAYTNSFTRGVYYPDKVF | 42 |
| acid sequence | RSSVLHSTQDLFLPFFSNVTWFHVISGTNGTKRFDNPVLPEND | |
| | GVYFASIEKSNIIRGWIFGTTLDSKTQSLLIVNNATNVVIKVCEF | |
| | QFCNDPFLDHKNNKSWMESEFRVYSSANNCTFEYVSQPFLMD | |
| | LEGKQGNFKNLREFVFKNIDGYFKIYSKHTPIIVREPEDLPQGF | |
| | SALEPLVDLPIGINITRFQTLLALHRSYLTPGDSSSGWTAGAAA | |
| | YYVGYLQPRTFLLKYNENGTITDAVDCALDPLSETKCTLKSFT | |
| | VEKGIYQTSNFRVQPTESIVRFPNITNLCPFDEVFNATRFASVY | |
| | AWNRKRISNCVADYSVLYNLAPFFTFKCYGVSPTKLNDLCFT | |
| | NVYADSFVIRGDEVRQIAPGQTGNIADYNYKLPDDFTGCVIA | |
| | WNSNKLDSKVSGNYNYLYRLFRKSNLKPFERDISTEIYQAGN | |
| | KPCNGVAGENCYFPLRSYSFRPTYGVGHQPYRVVVLSFELLH | |
| | APATVCGPKKSTNLVKNKCVNFNFNGLKGTGVLTESNKKFLP | |
| | FQQFGRDIADTTDAVRDPQTLEILDITPCSFGGVSVITPGTNTSN | |
| | QVAVLYQGVNCTEVPVAIHADQLTPTWRVYSTGSNVFQTRA | |
| | GCLIGAEYVNNSYECDIPIGAGICASYQTQTKSHRRARSVASQS | |
| | IIAYTMSLGAENSVAYSNNSIAIPTNFTISVTTEILPVSMTKTSV | |
| | DCTMYICGDSTECSNLLLQYGSFCTQLKRALTGIAVEQDKNTQ | |
| | EVFAQVKQIYKTPPIKYFGGFNFSQILPDPSKPSKRSFIEDLLEN | |
| | KVTLADAGFIKQYGDCLGDIAARDLICAQKFKGLTVLPPLLTD | |
| | EMIAQYTSALLAGTITSGWTFGAGAALQIPFAMQMAYRENGI | |
| | GVTQNVLYENQKLIANQFNSAIGKIQDSLSSTASALGKLQDVV | |
| | NHNAQALNTLVKQLSSKFGAISSVLNDIFSRLDPPEAEVQIDRL | |
| | ITGRLQSLQTYVTQQLIRAAEIRASANLAATKMSECVLGQSKR | |
| | VDFCGKGYHLMSFPQSAPHGVVFLHVTYVPAQEKNFTTAPAI | |
| | CHDGKAHFPREGVFVSNGTHWFVTQRNFYEPQIITTDNTFVSG | |
| | NCDVVIGIVNNTVYDPLQPELDSFKEELDKYFKNHTSPDVDLG | |
| | DISGINASVVNIQKEIDRLNEVAKNLNESLIDLQELGKYEQYIK | |
| | WPWYIWLGFIAGLIAIVMVTIMLCCMTSCCSCLKGCCSCGSCC | |
| | KFDEDDSEPVLKGVKLHYT | |
| PolyA tail | 100 nt | |

1273.529.IDR14B

| SEQ ID NO: 45 consists of from 5′ end to 3′ end: 5′ UTR SEQ ID NO: 2, | | 45 |
|---|---|---|
| mRNA ORF SEQ ID NO: 41, and 3′ UTR SEQ ID NO: 46. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGUUCGUGUUCCUGGUGCUGCUGCCCCUGGUGAGCAGC | 41 |
| Construct | CAGUGCGUGAACCUGACCACCCGGACCCAGCUGCCACCAG | |
| (excluding the stop | CCUACACCAACAGCUUCACCCGGGGCGUGUACUACCCCGA | |
| codon) | CAAGGUGUUCCGGAGCAGCGUGCUGCACAGCACCCAGGAC | |
| | CUGUUCCUGCCCUUCUUCAGCAACGUGACCUGGUUCCACG | |
| | UGAUCAGCGGCACCAACGGCACCAAGCGGUUCGACAACCC | |
| | CGUGCUGCCCUUCAACGACGGCGUGUACUUCGCCAGCAUC | |
| | GAGAAGAGCAACAUCAUCCGGGGCUGGAUCUUCGGCACC | |
| | ACCCUGGACAGCAAGACCCAGAGCCUGCUGAUCGUGAACA | |
| | ACGCCACCAACGUGGUGAUCAAGGUGUGCGAGUUCCAGU | |
| | UCUGCAACGACCCCUUCCUGGACCACAAGAACAACAAGAG | |
| | CUGGAUGGAGAGCGAGUUCCGGGUGUACAGCAGCGCCAA | |
| | CAACUGCACCUUCGAGUACGUGAGCCAGCCCUUCCUGAUG | |
| | GACCUGGAGGGCAAGCAGGGCAACUUCAAGAACCUGCGG | |
| | GAGUUCGUGUUCAAGAACAUCGACGGCUACUUCAAGAUC | |
| | UACAGCAAGCACACCCCAAUCAUCGUGCGGGAGCCCGAGG | |
| | ACCUGCCCCAGGGCUUCAGCGCCCUGGAGCCCCUGGUGGA | |
| | CCUGCCCAUCGGCAUCAACAUCACCCGGUUCCAGACCCUG | |
| | CUGGCCCUGCACCGGAGCUACCUGACCCCAGGCGACAGCA | |
| | GCAGCGGCUGGACCGCCGGCGCCGCCGCCUACUACGUGGG | |
| | CUACCUGCAGCCCCGGACCUUCCUGCUGAAGUACAACGAG | |
| | AACGGCACCAUCACCGACGCCGUGGACUGCGCCCUGGACC | |
| | CUCUGAGCGAGACCAAGUGCACCCUGAAGAGCUUCACCGU | |
| | GGAGAAGGGCAUCUACCAGACCAGCAACUUCCGGGUGCA | |
| | GCCCACCGAGAGCAUCGUGCGGUUCCCCAACAUCACCAAC | |
| | CUGUGCCCCUUCGACGAGGUGUUCAACGCCACCCGGUUCG | |
| | CCAGCGUGUACGCCUGGAACCGGAAGCGGAUCAGCAACU | |
| | GCGUGGCCGACUACAGCGUGCUGUACAACCUGGCCCCUUU | |
| | CUUCACCUUCAAGUGCUACGGCGUGAGCCCCACCAAGCUG | |
| | AACGACCUGUGCUUCACCAACGUGUACGCCGACAGCUUCG | |
| | UGAUCCGGGGCGACGAGGUGCGGCAGAUCGCCCCAGGCCA | |
| | GACCGGCAACAUCGCCGACUACAACUACAAGCUGCCCGAC | |
| | GACUUCACCGGCUGCGUGAUCGCCUGGAACAGCAACAAGC | |
| | UGGACAGCAAGGUGAGCGGCAACUACAACUACCUGUACC | |
| | GGCUGUUCCGGAAGAGCAACCUGAAGCCCUUCGAGCGGG | |
| | ACAUCAGCACCGAGAUCUACCAGGCCGGCAACAAGCCCUG | |
| | CAACGGCGUGGCCGGCUUCAACUGCUACUUCCCUCUGCGG | |
| | AGCUACAGCUUCCGGCCCACCUACGGCGUGGGCCACCAGC | |
| | CCUACCGGGUGGUGGUGCUGAGCUUCGAGCUGCUGCACG | |
| | CCCCAGCCACCGUGUGCGGCCCCAAGAAGAGCACCAACCU | |
| | GGUGAAGAACAAGUGCGUGAACUUCAACUUCAACGGCCU | |
| | GAAGGGCACCGGCGUGCUGACCGAGAGCAACAAGAAGUU | |
| | CCUGCCCUUCCAGCAGUUCGGCCGGGACAUCGCCGACACC | |
| | ACCGACGCCGUGCGGGAUCCCCAGACCCUGGAGAUCCUGG | |
| | ACAUCACCCCUUGCAGCUUCGGCGGCGUGAGCGUGAUCAC | |
| | CCCAGGCACCAACACCAGCAACCAGGUGGCCGUGCUGUAC | |
| | CAGGGCGUGAACUGCACCGAGGUGCCCGUGGCCAUCCACG | |
| | CCGACCAGCUGACCCCAACCUGGCGGGUGUACAGCACCGG | |
| | CAGCAACGUGUUCCAGACCCGGGCCGGCUGCCUGAUCGGC | |
| | GCCGAGUACGUGAACAACAGCUACGAGUGCGACAUCCCCA | |
| | UCGGCGCCGGCAUCUGCGCCAGCUACCAGACCCAGACCAA | |
| | GAGCCACCGGCGGGCCCGGAGCGUGGCCAGCCAGAGCAUC | |
| | AUCGCCUACACCAUGAGCCUGGGCGCCGAGAACAGCGUGG | |
| | CCUACAGCAACAACAGCAUCGCCAUCCCCACCAACUUCAC | |
| | CAUCAGCGUGACCACCGAGAUCCUGCCCGUGAGCAUGACC | |
| | AAGACCAGCGUGGACUGCACCAUGUACAUCUGCGGCGAC | |
| | AGCACCGAGUGCAGCAACCUGCUGCUGCAGUACGGCAGCU | |
| | UCUGCACCCAGCUGAAGCGGGCCCUGACCGGCAUCGCCGU | |
| | GGAGCAGGACAAGAACACCCAGGAGGUGUUCGCCCAGGU | |
| | GAAGCAGAUCUACAAGACCCCUCCCAUCAAGUACUUCGGC | |
| | GGCUUCAACUUCAGCCAGAUCCUGCCCGACCCCAGCAAGC | |
| | CCAGCAAGCGGAGCUUCAUCGAGGACCUGCUGUUCAACA | |
| | AGGUGACCCUGGCCGACGCCGGCUUCAUCAAGCAGUACGG | |
| | CGACUGCCUGGGCGACAUCGCCGCCCGGGACCUGAUCUGC | |
| | GCCCAGAAGUUCAAGGGCCUGACCGUGCUGCCUCCUCUGC | |
| | UGACCGACGAGAUGAUCGCCCAGUACACCAGCGCCCUGCU | |
| | GGCCGGCACCAUCACCAGCGGCUGGACCUUCGGCGCCGGC | |
| | GCCGCCCUGCAGAUCCCCUUCGCCAUGCAGAUGGCCUACC | |
| | GGUUCAACGGCAUCGGCGUGACCCAGAACGUGCUGUACG | |
| | AGAACCAGAAGCUGAUCGCCAACCAGUUCAACAGCGCCAU | |
| | CGGCAAGAUCCAGGACAGCCUGAGCAGCACCGCCAGCGCC | |
| | CUGGGCAAGCUGCAGGACGUGGUGAACCACAACGCCCAG | |
| | GCCCUGAACACCCUGGUGAAGCAGCUGAGCAGCAAGUUC | |
| | GGCGCCAUCAGCAGCGUGCUGAACGACAUCUUCAGCCGGC | |
| | UGGACCCUCCCGAGGCCGAGGUGCAGAUCGACCGGCUGAU | |
| | CACCGGCCGGCUGCAGAGCCUGCAGACCUACGUGACCCAG | |
| | CAGCUGAUCCGGGCCGCCGAGAUCCGGGCCAGCGCCAACC | |
| | UGGCCGCCACCAAGAUGAGCGAGUGCGUGCUGGGCCAGA | |
| | GCAAGCGGGUGGACUUCUGCGGCAAGGGCUACCACCUGA | |
| | UGAGCUUUCCCCAGAGCGCACCCCACGGCGUGGUGUUCCU | |
| | GCACGUGACCUACGUGCCCGCCCAGGAGAAGAACUUCACC | |
| | ACCGCCCCAGCCAUCUGCCACGACGGCAAGGCCCACUUUC | |
| | CCCGGGAGGGCGUGUUCGUGAGCAACGGCACCCACUGGU | |
| | UCGUGACCCAGCGGAACUUCUACGAGCCCCAGAUCAUCAC | |
| | CACCGACAACACCUUCGUGAGCGGCAACUGCGACGUGGUG | |
| | AUCGGCAUCGUGAACAACACCGUGUACGAUCCCCUGCAGC | |
| | CCGAGCUGGACAGCUUCAAGGAGGAGCUGGACAAGUACU | |
| | UCAAGAACCACACCAGCCCCGACGUGGACCUGGGCGACAU | |
| | CAGCGGCAUCAACGCCAGCGUGGUGAACAUCCAGAAGGA | |
| | GAUCGACAGACUGAACGAGGUGGCCAAGAACCUGAACGA | |
| | GAGCCUGAUCGACCUGCAGGAGCUGGGCAAGUACGAGCA | |
| | GUACAUCAAGUGGCCCUGGUACAUCUGGCUGGGCUUCAU | |
| | CGCCGGCCUGAUCGCCAUCGUGAUGGUGACCAUCAUGCUG | |
| | UGCUGCAUGACCAGCUGCUGCAGCUGCCUGAAGGGCUGC | |
| | UGCAGCUGCGGCAGCUGCUGCAAGUUCGACGAGGACGAC | |
| | AGCGAGCCCGUGCUGAAGGGCGUGAAGCUGCACUACACC | |
| 3′ UTR | UGAUAAUAGGCUGGAGCCUCGGUGGCCUAGCUUCUUGCC | 46 |
| | CCUUGGGCCUCCCCCCAGCCCCUCCUCCCCUUCCUGCAGG | |
| | CCACAUAGCGAAGUGGUCUUUGAAUAAAGUCUGAGUGGG | |
| | CGGC | |
| Corresponding amino | MFVFLVLLPLVSSQCVNLTTRTQLPPAYTNSFTRGVYYPDKVF | 42 |
| acid sequence | RSSVLHSTQDLFLPFFSNVTWFHVISGTNGTKRFDNPVLPEND | |
| | GVYFASIEKSNIIRGWIFGTTLDSKTQSLLIVNNATNVVIKVCEF | |
| | QFCNDPFLDHKNNKSWMESEFRVYSSANNCTFEYVSQPFLMD | |
| | LEGKQGNFKNLREFVFKNIDGYFKIYSKHTPIIVREPEDLPQGF | |
| | SALEPLVDLPIGINITRFQTLLALHRSYLTPGDSSSGWTAGAAA | |
| | YYVGYLQPRTFLLKYNENGTITDAVDCALDPLSETKCTLKSFT | |
| | VEKGIYQTSNFRVQPTESIVRFPNITNLCPFDEVFNATRFASVY | |
| | AWNRKRISNCVADYSVLYNLAPFFTFKCYGVSPTKLNDLCFT | |
| | NVYADSFVIRGDEVRQIAPGQTGNIADYNYKLPDDFTGCVIA | |
| | WNSNKLDSKVSGNYNYLYRLFRKSNLKPFERDISTEIYQAGN | |
| | KPCNGVAGENCYFPLRSYSFRPTYGVGHQPYRVVVLSFELLH | |
| | APATVCGPKKSTNLVKNKCVNFNFNGLKGTGVLTESNKKFLP | |
| | FQQFGRDIADTTDAVRDPQTLEILDITPCSFGGVSVITPGTNTSN | |
| | QVAVLYQGVNCTEVPVAIHADQLTPTWRVYSTGSNVFQTRA | |
| | GCLIGAEYVNNSYECDIPIGAGICASYQTQTKSHRRARSVASQS | |
| | IIAYTMSLGAENSVAYSNNSIAIPTNFTISVTTEILPVSMTKTSV | |
| | DCTMYICGDSTECSNLLLQYGSFCTQLKRALTGIAVEQDKNTQ | |
| | EVFAQVKQIYKTPPIKYFGGFNFSQILPDPSKPSKRSFIEDLLEN | |
| | KVTLADAGFIKQYGDCLGDIAARDLICAQKFKGLTVLPPLLTD | |
| | EMIAQYTSALLAGTITSGWTFGAGAALQIPFAMQMAYRENGI | |
| | GVTQNVLYENQKLIANQFNSAIGKIQDSLSSTASALGKLQDVV | |
| | NHNAQALNTLVKQLSSKFGAISSVLNDIFSRLDPPEAEVQIDRL | |
| | ITGRLQSLQTYVTQQLIRAAEIRASANLAATKMSECVLGQSKR | |
| | VDFCGKGYHLMSFPQSAPHGVVFLHVTYVPAQEKNFTTAPAI | |
| | CHDGKAHFPREGVFVSNGTHWFVTQRNFYEPQIITTDNTFVSG | |
| | NCDVVIGIVNNTVYDPLQPELDSFKEELDKYFKNHTSPDVDLG | |
| | DISGINASVVNIQKEIDRLNEVAKNLNESLIDLQELGKYEQYIK | |
| | WPWYIWLGFIAGLIAIVMVTIMLCCMTSCCSCLKGCCSCGSCC | |
| | KFDEDDSEPVLKGVKLHYT | |
| PolyA tail | 100 nt | |

### EQUIVALENTS

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between and including the upper and lower ends of the range are specifically contemplated and described herein.

1.-54. (canceled)

55. A pharmaceutical composition comprising a messenger RNA (mRNA) comprising a nucleotide sequence encoding a SARS-CoV-2 Spike(S) protein, wherein the SARS-CoV-2 S protein comprises: (a) at least 10 of the following mutations as compared to SEQ ID NO:18: A67V, Δ69-70, T95I, G142D/Δ143-145, Δ211/L212I, ins214EPE, G339D, S371L, S373P, S375F, K417N, N440K, G446S, S477N, T478K, E484A, Q493K, G496S, Q498R, N501Y, Y505H, T547K, D614G, H655Y, N679K, P681H, N764K, D796Y, N856K, Q954H, N969K, and L981F; and (b) an amino acid sequence that is at least 97% identical to SEQ ID NO:33, or to an immunogenic fragment thereof; wherein the mRNA comprises 1-methyl-pseudouridine substitutions at one or more uridine positions of the mRNA.

56. The pharmaceutical composition of claim 55, wherein the nucleotide sequence is at least 90% identical to SEQ ID NO:32.

57. The pharmaceutical composition of claim 55, wherein the nucleotide sequence is at least about 90% identical to SEQ ID NO:32.

58. The pharmaceutical composition of claim 55, wherein the mRNA comprises 1-methyl-pseudouridine substitutions at all uridine positions of the mRNA.

59. The pharmaceutical composition of claim 55, wherein the mRNA comprises one or more of the following: (a) a 5′-cap that comprises 7 mG(5′)ppp(5′) N1mpNp; (b) a poly(A) sequence comprising 50 to 250 A nucleotides; (c) a 5′-UTR that comprises a modified human alpha-globin 5′-UTR; and (d) a 3′-UTR.

60. The pharmaceutical composition of claim 55, wherein the mRNA is formulated in lipid nanoparticles.

61. The pharmaceutical composition of claim 60, wherein the mRNA is present in an amount within a range of about 25 μg to about 100 μg.

62. The pharmaceutical composition of claim 60, wherein the mRNA is present in an amount of about 10 μg.

63. The pharmaceutical composition of claim 60, wherein the mRNA is present in an amount of about 30 μg.

64. A pharmaceutical composition comprising a messenger RNA (mRNA) comprising a nucleotide sequence encoding a SARS-CoV-2 Spike(S) protein, wherein the SARS-CoV-2 S protein comprises: (a) at least 10 of the following mutations as compared to SEQ ID NO:18: Δ69-70, T95I, G142D, ΔY144, R190S, K417N, N439K, E484K, S477N, T478K, N501Y, D614G, H655Y, and P681H; and (b) an amino acid sequence that is at least 97% identical to SEQ ID NO:14, or to an immunogenic fragment thereof; wherein the mRNA comprises 1-methyl-pseudouridine substitutions at one or more uridine positions of the mRNA.

65. The pharmaceutical composition of claim 64, wherein the nucleotide sequence is at least 90% identical to SEQ ID NO:13.

66. The pharmaceutical composition of claim 64, wherein the nucleotide sequence is at least about 90% identical to SEQ ID NO:13.

67. The pharmaceutical composition of claim 64, wherein the mRNA comprises 1-methyl-pseudouridine substitutions at all uridine positions of the nucleotide sequence.

68. The pharmaceutical composition of claim 64, wherein the mRNA is formulated in lipid nanoparticles.

69. The pharmaceutical composition of claim 68, wherein the mRNA is present in an amount within a range of about 25 μg to about 100 μg.

70. The pharmaceutical composition of claim 68, wherein the mRNA is present in an amount of about 10 μg.

71. The pharmaceutical composition of claim 68, wherein the mRNA is present in an amount of about 30 μg.

72. A method of eliciting an immune response against SARS-CoV-2 in a human subject, comprising administering to the human subject the pharmaceutical composition of claim 55 in an effective amount to induce the immune response.

73. A method of eliciting an immune response against SARS-CoV-2 in a human subject, comprising administering to the human subject the pharmaceutical composition of claim 64 in an effective amount to induce the immune response.
